SEQUENCE LISTING (82 sequences)

SEQ ID NO: 1 - RNA, 19 nt, Artificial Sequence
  Description of Artificial Sequence Synthetic oligonucleotide
cgcaucugacugaccaaaa

SEQ ID NO: 2 - RNA, 20 nt, Artificial Sequence
  Description of Artificial Sequence Synthetic oligonucleotide
auagcccagucagucuacgc

SEQ ID NO: 3 - RNA, 20 nt, Artificial Sequence
  Description of Artificial Sequence Synthetic oligonucleotide
aaaccguuaccauuacugag

SEQ ID NO: 4 - RNA, 20 nt, Artificial Sequence
  Description of Artificial Sequence Synthetic oligonucleotide
gcguacaucuuccccuuuau

SEQ ID NO: 5 - RNA, 21 nt, Artificial Sequence
  Description of Artificial Sequence Synthetic oligonucleotide
gcggauuuagcucaguuggga

SEQ ID NO: 6 - RNA, 20 nt, Artificial Sequence
  Description of Artificial Sequence Synthetic oligonucleotide
  modified_base (8)..(8): pseudouridine
  modified_base (16)..(16): 5-methylcytidine
aaaccguuaccauuacugag

SEQ ID NO: 7 - DNA, 18 bp, Artificial Sequence
  Description of Artificial Sequence Synthetic primer
tggtgcgaattctgtgga

SEQ ID NO: 8 - RNA, 13 nt, Artificial Sequence
  Description of Artificial Sequence Synthetic oligonucleotide
uccacagaguucg

SEQ ID NO: 9 - RNA, 29 nt, Artificial Sequence
  Description of Artificial Sequence Synthetic oligonucleotide
  modified_base (10)..(10): N2-methylguanosine
  modified_base (13)..(13): pseudouridine
  modified_base (19)..(19): Dihydrouridine
  modified_base (20)..(20): pseudouridine
ucccugguggucuagugguuaggauucgg

SEQ ID NO: 10 - RNA, 19 nt, Artificial Sequence
  Description of Artificial Sequence Synthetic oligonucleotide
cgcaucugacugaccaaaa

SEQ ID NO: 11 - RNA, 20 nt, Artificial Sequence
  Description of Artificial Sequence Synthetic oligonucleotide
auagcccagucagucuacgc

SEQ ID NO: 12 - RNA, 20 nt, Artificial Sequence
  Description of Artificial Sequence Synthetic oligonucleotide
  modified_base (8)..(8): converted or unconverted pseudouridine
  modified_base (16)..(16): 5-methylcytidine
aaaccguuaccauuacugag

SEQ ID NO: 13 - RNA, 20 nt, Artificial Sequence
  Description of Artificial Sequence Synthetic oligonucleotide
aaaccguuaccauuacugag

SEQ ID NO: 14 - RNA, 20 nt, Artificial Sequence
  Description of Artificial Sequence Synthetic oligonucleotide
gcguacaucuuccccuuuau

SEQ ID NO: 15 - RNA, 21 nt, Artificial Sequence
  Description of Artificial Sequence Synthetic oligonucleotide
gcggauuuagcucaguuggga

SEQ ID NO: 16 - RNA, 19 nt, Artificial Sequence
  Description of Artificial Sequence Synthetic oligonucleotide
  modified_base (1)..(1): 1-methyladenosine
auccacagaauucgcacca

SEQ ID NO: 17 - RNA, 18 nt, Artificial Sequence
  Description of Artificial Sequence Synthetic oligonucleotide
  modified_base (1)..(1): 1-methyladenosine
auccacagaauucgcacc

SEQ ID NO: 18 - RNA, 15 nt, Artificial Sequence
  Description of Artificial Sequence Synthetic oligonucleotide
  modified_base (1)..(1): 1-methyladenosine
auccacagaauucgc

SEQ ID NO: 19 - RNA, 37 nt, Artificial Sequence
  Description of Artificial Sequence Synthetic oligonucleotide
  modified_base (6)..(6): N2, N2 dimethylguanosine
  modified_base (12)..(12): 2′-O-methylcytidine
  modified_base (14)..(14): 2′-O-methylguanosine
  modified_base (17)..(17): ribose
  modified_base (20)..(20): 5-methylcytidine
  modified_base (26)..(26): 2′-O-methylguanosine
  modified_base (29)..(29): 5-methylcytidine
  modified_base (34)..(34): 5-methyluridine
agagcgccagacugaanaucuggagguccuguguucg

SEQ ID NO: 20 - RNA, 37 nt, Artificial Sequence
  Description of Artificial Sequence Synthetic oligonucleotide
  modified_base (6)..(6): N2, N2 dimethylguanosine
  modified_base (12)..(12): 2′-O-methylcytidine
  modified_base (14)..(14): 2′-O-methylguanosine
  modified_base (17)..(17): ribose
  modified_base (20)..(20): 5-methylcytidine
  modified_base (26)..(26): 2′-O-methylguanosine
  modified_base (29)..(29): 5-methylcytidine
  modified_base (34)..(34): 5-methyluridine
agagcgccagacugaanaucugggaguccuguguucg

SEQ ID NO: 21 - RNA, 20 nt, Artificial Sequence
  Description of Artificial Sequence Synthetic oligonucleotide
  modified_base (10)..(10): 2′-O-methylguanosine
  modified_base (16)..(17): Dihydrouridine
gcggauuuagcucaguuggg

SEQ ID NO: 22 - RNA, 18 nt, Artificial Sequence
  Description of Artificial Sequence Synthetic oligonucleotide
  modified_base (10)..(10): 2′-O-methylguanosine
  modified_base (16)..(17): Dihydrouridine
gcggauuuagcucaguug

SEQ ID NO: 23 - RNA, 37 nt, Artificial Sequence; identical to SEQ ID NO: 19
  Description of Artificial Sequence Synthetic oligonucleotide
  modified_base (6)..(6): N2, N2 dimethylguanosine
  modified_base (12)..(12): 2′-O-methylcytidine
  modified_base (14)..(14): 2′-O-methylguanosine
  modified_base (17)..(17): ribose
  modified_base (20)..(20): 5-methylcytidine
  modified_base (26)..(26): 2′-O-methylguanosine
  modified_base (29)..(29): 5-methylcytidine
  modified_base (34)..(34): 5-methyluridine

SEQ ID NO: 24 - RNA, 37 nt, Artificial Sequence; identical to SEQ ID NO: 20
  Description of Artificial Sequence Synthetic oligonucleotide
  modified_base (6)..(6): N2, N2 dimethylguanosine
  modified_base (12)..(12): 2′-O-methylcytidine
  modified_base (14)..(14): 2′-O-methylguanosine
  modified_base (17)..(17): ribose
  modified_base (20)..(20): 5-methylcytidine
  modified_base (26)..(26): 2′-O-methylguanosine
  modified_base (29)..(29): 5-methylcytidine
  modified_base (34)..(34): 5-methyluridine

SEQ ID NO: 25 - RNA, 32 nt, Artificial Sequence
  Description of Artificial Sequence Synthetic oligonucleotide
  modified_base (2)..(2): 2′-O-methylguanosine
  modified_base (5)..(5): 5-methylcytidine
  modified_base (10)..(10): 5-methyluridine
  modified_base (14)..(14): 1-methyladenosine
gguccuguguucgauccacagaauucgcacca

SEQ ID NO: 26 - RNA, 31 nt, Artificial Sequence
  Description of Artificial Sequence Synthetic oligonucleotide
  modified_base (10)..(10): 2′-O-methylguanosine
  modified_base (16)..(17): Dihydrouridine
  modified_base (26)..(26): N2, N2 dimethylguanosine
gcggauuuagcucaguugggagagcgccaga

SEQ ID NO: 27 - RNA, 25 nt, Artificial Sequence
  Description of Artificial Sequence Synthetic oligonucleotide
  modified_base (6)..(6): N2, N2 dimethylguanosine
  modified_base (12)..(12): 2′-O-methylcytidine
  modified_base (14)..(14): 2′-O-methylguanosine
  modified_base (17)..(17): ribose
  modified_base (20)..(20): 5-methylcytidine
agagcgccagacugaanaucuggag

SEQ ID NO: 28 - RNA, 25 nt, Artificial Sequence
  Description of Artificial Sequence Synthetic oligonucleotide
  modified_base (6)..(6): N2, N2 dimethylguanosine
  modified_base (12)..(12): 2′-O-methylcytidine
  modified_base (14)..(14): 2′-O-methylguanosine
  modified_base (17)..(17): ribose
  modified_base (20)..(20): 5-methylcytidine
agagcgccagacugaanaucuggga

SEQ ID NO: 29 - RNA, 11 nt, Artificial Sequence
  Description of Artificial Sequence Synthetic oligonucleotide
  modified_base (3)..(3): 5-methylcytidine
  modified_base (8)..(8): 5-methyluridine
uccuguguucg

SEQ ID NO: 30 - RNA, 22 nt, Artificial Sequence
  Description of Artificial Sequence Synthetic oligonucleotide
  modified_base (1)..(1): 2′-O-methylguanosine
  modified_base (4)..(4): ribose
  modified_base (6)..(6): pseudouridine
  modified_base (7)..(7): 5-methylcytidine
  modified_base (13)..(13): 7-methylguanosine
  modified_base (16)..(16): 5-methylcytidine
  modified_base (21)..(21): 5-methyluridine
  modified_base (22)..(22): pseudouridine
gaanaucuggagguccuguguu

SEQ ID NO: 31 - RNA, 22 nt, Artificial Sequence
  Description of Artificial Sequence Synthetic oligonucleotide
  modified_base (1)..(1): 2′-O-methylguanosine
  modified_base (4)..(4): ribose
  modified_base (6)..(6): pseudouridine
  modified_base (7)..(7): 5-methylcytidine
  modified_base (13)..(13): 7-methylguanosine
  modified_base (16)..(16): 5-methylcytidine
  modified_base (21)..(21): 5-methyluridine
  modified_base (22)..(22): pseudouridine
gaanaucugggaguccuguguu

SEQ ID NO: 32 - RNA, 26 nt, Artificial Sequence
  Description of Artificial Sequence Synthetic oligonucleotide
  modified_base (3)..(3): 2′-O-methylcytidine
  modified_base (5)..(5): 2′-O-methylguanosine
  modified_base (8)..(8): ribose
  modified_base (10)..(10): pseudouridine
  modified_base (11)..(11): 5-methylcytidine
  modified_base (17)..(17): 7-methylguanosine
  modified_base (20)..(20): 5-methylcytidine
  modified_base (25)..(25): 5-methyluridine
  modified_base (26)..(26): pseudouridine
gacugaanaucuggagguccuguguu

SEQ ID NO: 33 - RNA, 26 nt, Artificial Sequence
  Description of Artificial Sequence Synthetic oligonucleotide
  modified_base (3)..(3): 2′-O-methylcytidine
  modified_base (5)..(5): 2′-O-methylguanosine
  modified_base (8)..(8): ribose
  modified_base (10)..(10): pseudouridine
  modified_base (11)..(11): 5-methylcytidine
  modified_base (17)..(17): 7-methylguanosine
  modified_base (20)..(20): 5-methylcytidine
  modified_base (25)..(25): 5-methyluridine
  modified_base (26)..(26): pseudouridine
gacugaanaucugggaguccuguguu

SEQ ID NO: 34 - RNA, 17 nt, Artificial Sequence
  Description of Artificial Sequence Synthetic oligonucleotide
caucugacugaccaaaa

SEQ ID NO: 35 - RNA, 20 nt, Artificial Sequence
  Description of Artificial Sequence Synthetic oligonucleotide
cgcaucugacugacccgaua

SEQ ID NO: 36 - RNA, 19 nt, Artificial Sequence
  Description of Artificial Sequence Synthetic oligonucleotide
  modified_base (7)..(7): converted or unconverted pseudouridine
  modified_base (15)..(15): 5-methylcytidine
aaccguuaccauuacugag

SEQ ID NO: 37 - RNA, 19 nt, Artificial Sequence
  Description of Artificial Sequence Synthetic oligonucleotide
cgcaucugacugaccaaaa

SEQ ID NO: 38 - RNA, 18 nt, Artificial Sequence
  Description of Artificial Sequence Synthetic oligonucleotide
gcaucugacugaccaaaa

SEQ ID NO: 39 - RNA, 21 nt, Artificial Sequence
  Description of Artificial Sequence Synthetic oligonucleotide
gcggauuuagcucaguuggga

SEQ ID NO: 40 - RNA, 20 nt, Artificial Sequence
  Description of Artificial Sequence Synthetic oligonucleotide
gcguacaucuuccccuuuau

SEQ ID NO: 41 - RNA, 20 nt, Artificial Sequence
  Description of Artificial Sequence Synthetic oligonucleotide
aaaccguuaccauuacugag

SEQ ID NO: 42 - RNA, 20 nt, Artificial Sequence
  Description of Artificial Sequence Synthetic oligonucleotide
auagcccagucagucuacgc

SEQ ID NO: 43 - RNA, 19 nt, Artificial Sequence
  Description of Artificial Sequence Synthetic oligonucleotide
  modified_base (1)..(1): 1-methyladenosine
auccacagaauucgcacca

SEQ ID NO: 44 - RNA, 37 nt, Artificial Sequence; identical to SEQ ID NO: 19
  Description of Artificial Sequence Synthetic oligonucleotide
  modified_base (6)..(6): N2, N2 dimethylguanosine
  modified_base (12)..(12): 2′-O-methylcytidine
  modified_base (14)..(14): 2′-O-methylguanosine
  modified_base (17)..(17): ribose
  modified_base (20)..(20): 5-methylcytidine
  modified_base (26)..(26): 2′-O-methylguanosine
  modified_base (29)..(29): 5-methylcytidine
  modified_base (34)..(34): 5-methyluridine

SEQ ID NO: 45 - RNA, 20 nt, Artificial Sequence
  Description of Artificial Sequence Synthetic oligonucleotide
  modified_base (10)..(10): 2′-O-methylguanosine
  modified_base (16)..(17): Dihydrouridine
gcggauuuagcucaguuggg

SEQ ID NO: 46 - RNA, 76 nt, Artificial Sequence
  Description of Artificial Sequence Synthetic oligonucleotide
  modified_base (10)..(10): N2-methylguanosine
  modified_base (16)..(17): Dihydrouridine
  modified_base (26)..(26): N2, N2 dimethylguanosine
  modified_base (32)..(32): 2′-O-methylcytidine
  modified_base (34)..(34): 2′-O-methylguanosine
  modified_base (37)..(37): wubutosine
  modified_base (39)..(39): pseudouridine
  modified_base (40)..(40): 5-methylcytidine
  modified_base (46)..(46): 7-methylguanosine
  modified_base (49)..(49): 5-methylcytidine
  modified_base (54)..(54): 5-methyluridine
  modified_base (55)..(55): pseudouridine
  modified_base (58)..(58): 1-methyladenosine
gcggauuuagcucaguugggagagcgccagacugaanaucuggagguccuguguucgauccacagaauucgcacca

SEQ ID NO: 47 - RNA, 76 nt, Artificial Sequence; identical to SEQ ID NO: 46
  Description of Artificial Sequence Synthetic oligonucleotide
  modified_base (10)..(10): methylguanosine
  modified_base (16)..(17): Dihydrouridine
  modified_base (26)..(26): N2, N2 dimethylguanosine
  modified_base (32)..(32): 2′-O-methylcytidine
  modified_base (34)..(34): 2′-O-methylguanosine
  modified_base (37)..(37): ribose
  modified_base (40)..(40): 5-methylcytidine
  modified_base (46)..(46): 2′-O-methylguanosine
  modified_base (49)..(49): 5-methylcytidine
  modified_base (54)..(54): 5-methyluridine
  modified_base (58)..(58): 1-methyladenosine

SEQ ID NO: 48 - RNA, 17 nt, Artificial Sequence
  Description of Artificial Sequence Synthetic oligonucleotide
  modified_base (1)..(1): 1-methyladenosine
auccacagaauucgcac

SEQ ID NO: 49 - RNA, 18 nt, Artificial Sequence
  Description of Artificial Sequence Synthetic oligonucleotide
  modified_base (1)..(1): 1-methyladenosine
auccacagaauucgcacc

SEQ ID NO: 50 - RNA, 17 nt, Artificial Sequence
  Description of Artificial Sequence Synthetic oligonucleotide
  modified_base (1)..(1): 1-methyladenosine
auccacagaauucgcac

SEQ ID NO: 51 - RNA, 19 nt, Artificial Sequence
  Description of Artificial Sequence Synthetic oligonucleotide
  modified_base (1)..(1): 1-methyladenosine
auccacagaauucgcacca

SEQ ID NO: 52 - RNA, 18 nt, Artificial Sequence
  Description of Artificial Sequence Synthetic oligonucleotide
  modified_base (1)..(1): 1-methyladenosine
auccacagaauucgcacc

SEQ ID NO: 53 - RNA, 17 nt, Artificial Sequence
  Description of Artificial Sequence Synthetic oligonucleotide
  modified_base (1)..(1): 1-methyladenosine
auccacagaauucgcac

SEQ ID NO: 54 - RNA, 76 nt, Artificial Sequence
  Description of Artificial Sequence Synthetic oligonucleotide
  modified_base (10)..(10): N2-methylguanosine
  modified_base (16)..(17): Dihydrouridine
  modified_base (26)..(26): N2, N2 dimethylguanosine
  modified_base (32)..(32): 2′-O-methylcytidine
  modified_base (34)..(34): 2′-O-methylguanosine
  modified_base (37)..(37): wubutosine
  modified_base (39)..(39): pseudouridine
  modified_base (40)..(40): 5-methylcytidine
  modified_base (46)..(46): 7-methylguanosine
  modified_base (49)..(49): 5-methylcytidine
  modified_base (54)..(54): 5-methyluridine
  modified_base (55)..(55): pseudouridine
  modified_base (58)..(58): 1-methyladenosine
gcggauuuagcucaguugggagagcgccagacugaanaucuggrrguccuguguucgauccacagaauucgcacca

SEQ ID NO: 55 - RNA, 37 nt, Artificial Sequence; identical to SEQ ID NO: 19
  Description of Artificial Sequence Synthetic oligonucleotide
  modified_base (6)..(6): N2, N2 dimethylguanosine
  modified_base (12)..(12): 2′-O-methylcytidine
  modified_base (14)..(14): 2′-O-methylguanosine
  modified_base (17)..(17): ribose
  modified_base (19)..(19): pseudouridine
  modified_base (20)..(20): 5-methylcytidine
  modified_base (26)..(26): 2′-O-methylguanosine
  modified_base (29)..(29): 5-methylcytidine
  modified_base (34)..(34): 5-methyluridine
  modified_base (35)..(35): pseudouridine

SEQ ID NO: 56 - RNA, 37 nt, Artificial Sequence; identical to SEQ ID NO: 19
  Description of Artificial Sequence Synthetic oligonucleotide
  modified_base (6)..(6): 2′-O-methylguanosine
  modified_base (12)..(12): 2′-O-methylcytidine
  modified_base (14)..(14): 2′-O-methylguanosine
  modified_base (17)..(17): ribose
  modified_base (20)..(20): 5-methylcytidine
  modified_base (26)..(26): 2′-O-methylguanosine
  modified_base (29)..(29): 5-methylcytidine
  modified_base (34)..(34): 5-methyluridine

SEQ ID NO: 57 - RNA, 18 nt, Artificial Sequence
  Description of Artificial Sequence Synthetic oligonucleotide
  modified_base (10)..(10): 2′-O-methylguanosine
  modified_base (16)..(17): Dihydrouridine
gcggauuuagcucaguug

SEQ ID NO: 58 - RNA, 20 nt, Artificial Sequence
  Description of Artificial Sequence Synthetic oligonucleotide
  modified_base (10)..(10): 2′-O-methylguanosine
  modified_base (16)..(17): Dihydrouridine
gcggauuuagcucaguuggg

SEQ ID NO: 59 - RNA, 19 nt, Artificial Sequence
  Description of Artificial Sequence Synthetic oligonucleotide
  modified_base (1)..(1): 1-methyladenosine
auccacagaauucgcacca

SEQ ID NO: 60 - RNA, 19 nt, Artificial Sequence
  Description of Artificial Sequence Synthetic oligonucleotide
  modified_base (1)..(1): 6-methyladenosine
auccacagaauucgcacca

SEQ ID NO: 61 - RNA, 19 nt, Artificial Sequence
  Description of Artificial Sequence Synthetic oligonucleotide
auccacagaauucgcacca

SEQ ID NO: 62 - DNA, 19 bp, Artificial Sequence
  Description of Artificial Sequence Synthetic oligonucleotide
  modified_base (19)..(19): Dideoxythymidine
tggtgcgaattctgtggat

SEQ ID NO: 63 - RNA, 25 nt, Artificial Sequence
  Description of Artificial Sequence Synthetic oligonucleotide
  modified_base (6)..(6): N2, N2 dimethylguanosine
  modified_base (12)..(12): 2′-O-methylcytidine
  modified_base (14)..(14): 2′-O-methylguanosine
  modified_base (17)..(17): ribose
  modified_base (19)..(19): converted or unconverted pseudouridine
  modified_base (20)..(20): 5-methylcytidine
  misc_feature (25)..(25): May or may not be present
agagcgccagacugaanaucuggag

SEQ ID NO: 64 - RNA, 11 nt, Artificial Sequence
  Description of Artificial Sequence Synthetic oligonucleotide
  modified_base (3)..(3): converted or unconverted pseudouridine
  modified_base (4)..(4): 5-methyluridine
  modified_base (9)..(9): 5-methylcytidine
gcuuguguccu

SEQ ID NO: 65 - RNA, 33 nt, Artificial Sequence
  Description of Artificial Sequence Synthetic oligonucleotide
  modified_base (4)..(4): wubutosine
  modified_base (6)..(6): pseudouridine
  modified_base (21)..(21): 5-methyluridine
  modified_base (22)..(22): pseudouridine
gaanaucuggagguccuguguucgauccacaga

SEQ ID NO: 66 - DNA, 23 bp, Artificial Sequence
  Description of Artificial Sequence Synthetic oligonucleotide
  modified_base (23)..(23): Dideoxythymidine
tctgtggatcgaacacaggacct

SEQ ID NO: 67 - DNA, 22 bp, Artificial Sequence
  Description of Artificial Sequence Synthetic oligonucleotide
  modified_base (22)..(22): Dideoxycytosine
tctgtggatcgaacacaggacc

SEQ ID NO: 68 - RNA, 10 nt, Artificial Sequence
  Description of Artificial Sequence Synthetic oligonucleotide
  modified_base (2)..(2): 2′-O-methylcytidine
  modified_base (4)..(4): 2′-O-methylguanosine
  modified_base (7)..(7): ribose
  modified_base (10)..(10): 5-methylcytidine
acugaanauc

SEQ ID NO: 69 - RNA, 37 nt, Artificial Sequence; identical to SEQ ID NO: 20
  Description of Artificial Sequence Synthetic oligonucleotide
  modified_base (6)..(6): N2, N2 dimethylguanosine
  modified_base (12)..(12): 2′-O-methylcytidine
  modified_base (14)..(14): 2′-O-methylguanosine
  modified_base (17)..(17): ribose
  modified_base (20)..(20): 5-methylcytidine
  modified_base (26)..(26): 2′-O-methylguanosine
  modified_base (29)..(29): 5-methylcytidine
  modified_base (34)..(34): 5-methyluridine

SEQ ID NO: 70 - RNA, 76 nt, Artificial Sequence
  Description of Artificial Sequence Synthetic oligonucleotide
  modified_base (10)..(10): N2-methylguanosine
  modified_base (16)..(17): Dihydrouridine
  modified_base (32)..(32): 2′-O-methylcytidine
  modified_base (34)..(34): 2′-O-methylguanosine
  modified_base (37)..(37): wubutosine
  modified_base (39)..(39): pseudouridine
  modified_base (40)..(40): 5-methylcytidine
  modified_base (46)..(46): 7-methylguanosine
  modified_base (49)..(49): 5-methylcytidine
  modified_base (54)..(54): 5-methyluridine
  modified_base (55)..(55): pseudouridine
  modified_base (58)..(58): 1-methyladenosine
gcggauuuagcucaguugggagagcrccagacugaanaucuggagguccuguguucgauccacagaauucgcacca

SEQ ID NO: 71 - RNA, 75 nt, Artificial Sequence
  Description of Artificial Sequence Synthetic oligonucleotide
  modified_base (10)..(10): N2-methylguanosine
  modified_base (16)..(17): Dihydrouridine
  modified_base (32)..(32): 2′-O-methylcytidine
  modified_base (34)..(34): 2′-O-methylguanosine
  modified_base (37)..(37): wubutosine
  modified_base (39)..(39): pseudouridine
  modified_base (40)..(40): 5-methylcytidine
  modified_base (46)..(46): 7-methylguanosine
  modified_base (49)..(49): 5-methylcytidine
  modified_base (54)..(54): 5-methyluridine
  modified_base (55)..(55): pseudouridine
  modified_base (58)..(58): 1-methyladenosine
gcggauuuagcucaguugggagagcrccagacugaanaucuggagguccuguguucgauccacagaauucgcacc

SEQ ID NO: 72 - RNA, 75 nt, Artificial Sequence
  Description of Artificial Sequence Synthetic oligonucleotide
  modified_base (10)..(10): N2-methylguanosine
  modified_base (16)..(17): Dihydrouridine
  modified_base (32)..(32): 2′-O-methylcytidine
  modified_base (34)..(34): 2′-O-methylguanosine
  modified_base (37)..(37): wubutosine
  modified_base (39)..(39): pseudouridine
  modified_base (40)..(40): 5-methylcytidine
  modified_base (46)..(46): 7-methylguanosine
  modified_base (49)..(49): 5-methylcytidine
  modified_base (54)..(54): 5-methyluridine
  modified_base (55)..(55): pseudouridine
  modified_base (58)..(58): 1-methyladenosine
gcggacuuagcucaguugggagagcrccagacugaanaucuggagguccuguguucgauccacagaguucgcacc

SEQ ID NO: 73 - RNA, 76 nt, Artificial Sequence
  Description of Artificial Sequence Synthetic oligonucleotide
  modified_base (10)..(10): 2′-O-methylguanosine
  modified_base (16)..(17): Dihydrouridine
  modified_base (26)..(26): N2, N2 dimethylguanosine
  misc_feature (32)..(33): May or may not be methylated
  misc_feature (34)..(35): May or may not be methylated
  Variation (37)..(38): a or ribose
  modified_base (40)..(40): 5-methylcytidine
  modified_base (46)..(46): 2′-O-methylguanosine
  modified_base (49)..(49): 5-methylcytidine
  modified_base (54)..(54): 5-methyluridine
  modified_base (58)..(58): 1-methyladenosine
  See specification as filed for detailed description of substitutions and preferred embodiments
ssggauuuagcucaguugggagrrcgccagayyrrannucuggagguccuguguucgauccacagawwyyvvvcca

SEQ ID NO: 74 - RNA, 69 nt, Artificial Sequence
  Description of Artificial Sequence Synthetic oligonucleotide
  misc_feature (6)..(8): May or may not be methylated
  modified_base (12)..(12): Dihydrouridine
  Variation (13)..(15): g or dihydrouridine
  misc_feature (24)..(26): May or may not be methylated
  Variation (24)..(26): c or s
  misc_feature (30)..(31): May or may not be methylated
  modified_base (32)..(32): 2′-O-methylguanosine
  modified_base (35)..(35): ribose
  modified_base (38)..(38): 5-methylcytidine
  modified_base (44)..(44): 2′-O-methylguanosine
  modified_base (47)..(47): 5-methylcytidine
  modified_base (52)..(52): methyluridine
  modified_base (56)..(56): 1-methyladenosine
  See specification as filed for detailed description of substitutions and preferred embodiments
wwuuabbbcagunnnggagasscnnnagayygaanaucuggagguccuguguucgauccacagaauucg

SEQ ID NO: 75 - RNA, 76 nt, Artificial Sequence
  Description of Artificial Sequence Synthetic oligonucleotide
  modified_base (10)..(10): 2′-O-methylguanosine
  modified_base (16)..(17): Dihydrouridine
  modified_base (26)..(26): N2, N2 dimethylguanosine
  misc_feature (32)..(33): May or may not be methylated
  modified_base (35)..(35): 2′-O-methylguanosine
  Variation (37)..(38): a or ribose
  modified_base (40)..(40): 5-methylcytidine
  modified_base (46)..(46): 2′-O-methylguanosine
  modified_base (49)..(49): 5-methylcytidine
  modified_base (54)..(54): 5-methyluridine
  modified_base (58)..(58): 1-methyladenosine
  See specification as filed for detailed description of substitutions and preferred embodiments
ssggauuuagcucaguugggagagcgccagayyagannucuggagguccuguguucgauccacagaauucgaccca

SEQ ID NO: 76 - RNA, 17 nt, Artificial Sequence
  Description of Artificial Sequence Synthetic oligonucleotide
  modified_base (12)..(13): Dihydrouridine
uggucuagugguuagga

SEQ ID NO: 77 - RNA, 75 nt, Artificial Sequence
  Description of Artificial Sequence Synthetic oligonucleotide
ucccugguggucuagugguuaggauucggcgcucucaccgccgcggcccggguucgauucccggucagggaacca

SEQ ID NO: 78 - RNA, 75 nt, Artificial Sequence; identical to SEQ ID NO: 77
  Description of Artificial Sequence Synthetic oligonucleotide
  modified_base (19)..(20): Dihydrouridine
  modified_base (48)..(49): 5-methylcytidine

SEQ ID NO: 79 - RNA, 76 nt, Artificial Sequence
  Description of Artificial Sequence Synthetic oligonucleotide
  modified_base (16)..(17): Dihydrouridine
  modified_base (37)..(37): wubutosine
  modified_base (39)..(39): pseudouridine
  modified_base (54)..(54): 5-methyluridine
  modified_base (55)..(55): pseudouridine
gcggauuuagcucaguugggagagcgccagacugaanaucuggrgguccuguguucgauccacagaauucgcacca

SEQ ID NO: 80 - DNA, 22 bp, Artificial Sequence
  Description of Artificial Sequence Synthetic oligonucleotide
  modified_base (22)..(22): Dideoxycytosine
tctgtggatcgaacacaggacc

SEQ ID NO: 81 - DNA, 24 bp, Artificial Sequence
  Description of Artificial Sequence Synthetic oligonucleotide
  modified_base (24)..(24): Dideoxycytosine
tctgtggatcgaacacaggacctc

SEQ ID NO: 82 - RNA, 11 nt, Artificial Sequence
  Description of Artificial Sequence Synthetic oligonucleotide
  modified_base (10)..(10): N2-methylguanosine
gcggauuuagc

(19) United States
(12) Patent Application Publication
(10) Pub. No.: US 2022/0220552 A1
(43) Pub. Date: Jul. 14, 2022

(54) METHODS FOR DIRECT SEQUENCING OF RNA

(71) Applicant: New York Institute of Technology, Old Westbury, NY (US)

(72) Inventors: Shenglong ZHANG, Fort Lee, NJ (US); Xiaohong YUAN, Flushing, NY (US)

(21) Appl. No.: 17/235,621

(22) Filed: Apr. 20, 2021

Related U.S. Application Data
(60) Provisional application No. 63/012,521, filed on Apr. 20, 2020.
(60) Provisional application No. 63/012,539, filed on Apr. 20, 2020.

(51) Int. Cl. C12Q 1/6869; C12Q 1/6806; G16B 30/10

(52) U.S. Cl. CPC C12Q 1/6869; G16B 30/10; C12Q 1/6806

(57) ABSTRACT

The present disclosure provides methods for direct sequencing of RNA, including but not limited to any coding RNA and non-coding RNA such as tRNA, rRNA, mRNA, short or long non-coding RNA as well as any of their modified forms/versions, without the need for generation of a cDNA intermediate and/or intensive sample preparation.

### CROSS REFERENCE TO RELATED APPLICATIONS

This application claims the benefit of and priority to U.S. Provisional Application No. 63/012,521, filed on Apr. 20, 2020 and U.S. Provisional Application No. 63/012,539, filed Apr. 20, 2020, the entire contents of which being incorporated by reference herein in their entireties.

### SEQUENCE LISTING

The instant application contains a Sequence Listing which has been submitted electronically in ASCII format and is hereby incorporated by reference in its entirety. Said ASCII copy, created on Nov. 29, 2021, is named 2637-5_SL.txt and is 41,991 bytes in size.

### TECHNICAL FIELD

The present disclosure provides methods for direct sequencing of RNA, including but not limited to any coding RNA and non-coding RNA such as tRNA, rRNA, mRNA, short or long non-coding RNA as well as any of their modified forms/versions, without the need for generation of a cDNA intermediate and/or intensive sample preparation.

### BACKGROUND

Post-transcriptional modifications are intrinsic to RNA structure and function. However, methods to sequence RNA typically require a cDNA intermediate and are either not able to sequence these modifications or are tailored to sequence one specific nucleotide modification only. Typically, methods used to sequence RNAs are indirect and require prior complementary DNA (cDNA). However, cDNA synthesis results in a loss of endogenous base modification information originally carried by RNAs and significant errors, resulting in the inability to accurately sequence base modifications, for example, to sequence the rich and dynamic base modifications in RNAs which are an inseparable part of the RNAs structure and function. Other methods that do not involve cDNA can detect base modifications, but these techniques usually require harsh treatments to the RNA sample such as intensive enzymatic or chemical hydrolysis, resulting in spatial modification information loss. Thus, methods to date do not efficiently permit the efficient sequencing of modification-containing RNA, including mixtures of RNA molecules such as those derived from a biological sample.

Mass spectrometry (MS) has been reviewed as one of the most promising tools for studying RNA modifications in the field of epitranscriptomics. MS-based methods can complement the current high-throughput NGS-based methods to provide additional information for identification and quantification of not only one single RNA modification type, but also different/combinatorial types of RNA modifications.

Unlike RNA mapping methods, MS-based de novo sequencing methods are typically based on mass laddering, which relies on a complete set of MS ladders, and each ladder is required to be perfect without missing any fragments in order to read all nucleotides from the first to the last in an RNA strand. As such, MS laddering methods can provide de novo sequence information themselves, and do not need prior sequence information and thus are independent from any other method, like NGS.

MS-based sequencing has limited applications for de novo sequencing of biological RNA, mainly due to its limitations in read length, throughput, and rigor requirements on sample preparation/quality. Compounding these difficulties, MS-based sequencing is based on a complete set of MS ladders, and each ladder requires to be perfect without missing any fragments in order to read all nucleotides from the first to the last in an RNA strand. As such, MS ladder sequencing is mainly limited to short synthetic RNA and/or dominating RNA species in a mixed sample and cannot be used to sequencing RNA samples in large scale.

As an essential component of protein synthesis machinery, RNA is present in all living cells. Despite the significance of RNAs, including tRNAs, to the regular function of all cells, structural and functional studies to understand the underlying biochemistry of RNA itself have been hindered due to the lack of efficient RNA sequencing methods. tRNA has different iso-acceptors (tRNAs with different anticodons but incorporating the same amino acid in protein synthesis) and tRNA can exist as different isoforms as a result of different chemical modifications. Some of these modifications occur with <100% frequency at their particular sites, and site-specific quantification of their stoichiometries is another challenge. For some modifications, every tRNA transcript copy will be modified at a certain position (i.e. 100% stoichiometry). In other cases, the nucleotide modification stoichiometries may be variable, and may therefore confer different properties onto the tRNA depending on the modification status. Thus, tRNAs can exist as distinct isoforms as a result of different chemical modifications. As such, it is not possible to separate any tRNA isoform with current available separate techniques.

With regard specifically to tRNA, although the first transfer RNA (tRNA) was sequenced in 1965, tRNAs are currently the only class of small cellular RNAs that cannot be efficiently sequenced with current sequencing techniques, despite more than 600 different tRNA sequences and a large breadth of different post-transcriptional base modifications that have been reported and sequenced.

Aberrant nucleic acid modifications, especially methylations and pseudouridylations in RNA, have been correlated to the development of major diseases like breast cancer, type-2 diabetes, and obesity each of which affects millions of people around of the world. Despite their significance, the available tools to reliably identify, locate, and quantify modifications in RNA are very limited. As a result, the function of most of such modifications remains largely unknown.

Accordingly, methods are needed to facilitate the efficient sequencing of various RNA molecules, including, for example, tRNAs, siRNAs, therapeutic synthetic oligoribonucleotides having pharmacokinetic properties, mixtures of RNA molecules, as well as identification, location, and quantification of nucleotide modifications of such RNA molecules.

MS-based sequencing is based on a complete set of MS ladders, and each ladder requires to be perfect without missing any fragments in order to read all nucleotides from the first to the last in an RNA strand. As such, the rigor sample requirement limits MS ladder sequencing's applications mainly to high-quality and highly abundant RNA samples such as short synthetic RNA and dominating RNA species in a mixed sample.

Accordingly, methods are needed to allow imperfect/faulted MS ladders for sequencing, which will be a paradigm shift for de novo MS sequencing of RNA. Methods are also needed to sequence not only predominant RNA species but also minor species simultaneously in an RNA mixture.

### SUMMARY

The current disclosure is related to direct, liquid-chromatography-mass spectrometry (herein referred to as LC-MS) based RNA sequencing methods which can be used to directly sequence RNA, without the need for prior cDNA synthesis, to simultaneously determine the nucleotide sequence of an RNA molecule with single nucleotide resolution, as well as, reveal the presence, type, location and quantity of different nucleotide modifications that the RNA molecule carries. The disclosed methods can be used to determine the type, location and quantity of each modification within the RNA sample. Such techniques can be used advantageously to correlate the biological functions of any given RNA molecule with its associated modifications and for quality control of RNA-based therapeutics.

The LC-MS-based RNA sequencing methods disclosed herein, advantageously provide methods that enable sequencing of purified RNA samples, as well as samples containing multiple RNA species, including mixtures of RNA derived from a biological sample. This strategy can be applied to the de novo sequencing of RNA sequences carrying both canonical and structurally atypical nucleosides. The methods provide a simplified means for sequencing of nucleotide modifications together with RNA sequences through, in some instances, efficient labeling of RNA at its 3′ and/or 5′ ends, thus enabling separation of 3′ ladder and 5′ ladder RNA pools for MS-based sequencing and analysis.

The current disclosure provides direct, liquid-chromatography-mass spectrometry (herein referred to as LC-MS) based RNA sequencing methods which can be used to simultaneously determine the nucleotide sequence of an RNA molecule with single nucleotide resolution, as well as, reveal the presence, type, location and quantity of different RNA modifications (alone or in combinations). The disclosed methods can be used to determine the type, location and quantity of each modification within the RNA sample while simultaneously sequencing the RNA molecules that carry these modification. Such techniques can be used advantageously to correlate the biological functions of any given RNA molecule with its associated modifications and for quality control of RNA-based therapeutics.

The present disclosure provides a method for generating the sequence of one or more RNA molecules and detecting the presence, identity, location, and quantity of RNA nucleotide modifications on said one or more RNA molecules, said method RNA comprising the steps of (i) controlled fragmentation of the RNA to form sequencable ladder fragments such as 5′ and 3′ MS ladder fragments; (ii) mass measurement of resultant degraded RNA samples containing RNAs and their fragmented fragments; and (iii) data processing, including identification and separation of 3′ and/or 5′ MS ladder fragments thereby generating the sequence of one or more RNA molecules and detecting the presence, identity, location, and quantity of RNA nucleotide modifications. In an embodiment, the controlled fragmentation of the RNA is achieved by chemical degradation, enzymatic degradation, or physical degradation. In another embodiment, the mass measurement is achieved by LC-MS, gas chromatography, capillary electrophoresis, ion mobility spectrometry, or other methods coupled with mass spectrometry. In an embodiment, the data processing may include a homology searching before, or after, fragmentation of RNA for identification of related RNA isoforms. In another embodiment, a MassSum data processing step may be performed which identifies and isolates the 3′, 5′ ladder fragments as well as other related fragments into subsets for each RNA in a mixed sample. Said method may further comprise the step of Gap Filling data processing to rescue 3′ and 5′ ladder fragments missed by Mass/Sum separation. Said method may further comprise data processing which includes the step of ladder complementation where the ladder fragments from one or more related RNA isoforms are used to perfect an imperfect ladder. In another embodiment, the data processing includes the step of identifying acid labile nucleotide modifications by comparing the mass change of intact RNA before and after acid degradation.

In another embodiment, a method is provided for generating the sequence of one or more RNA molecules and detecting the presence, identity, location, and quantity of RNA nucleotide modifications on said one or more RNA molecules, said method RNA comprising the steps of (i) identifying a specific chemical moiety associated with the RNA or labeling the RNA with a tag thereby imparting an identifiable property on the RNA (ii) controlled fragmentation of the RNA to form 5′ and 3′ MS ladder fragments; (iii) mass measurement of resultant degraded RNA samples containing RNAs and their degraded fragments; and (iv) data processing, including identification of 3′ and/or 5′ MS ladder fragments thereby generating the sequence of one or more RNA molecules and detecting the presence, identity, location, and quantity of RNA nucleotide modifications. In such a method the specific chemical moiety or the labeling tag has a known mass. In a specific embodiment, the chemical moiety is a 5′ phosphate and 3′ CCA of tRNA. Still further, the chemical moiety results in a change in retention time and/or mass/MS. In another embodiment the identifiable property results in an alteration in mass measurement. In an embodiment, the label may be selected from the group consisting of a hydrophobic tag, biotin, a Cy3 tag, a Cy5 tag and a cholesterol. In an embodiment, the controlled fragmentation of the RNA is achieved by chemical degradation, enzymatic degradation, or physical degradation. In an embodiment, the mass measurement is achieved by LC-MS, gas chromatography, capillary electrophoresis, ion mobility spectrometry or others coupled with mass spectrometry. In one aspect, the data processing step identifies the RNA fragments based on the specific chemical moiety associated with the RNA or the labeled tag thereby imparting an identifiable property on the RNA and/or fragments. In another aspect, the data processing step includes implementation of the anchoring-based algorithm to identify the labeled RNA and/or fragments.

The present disclosure further provides methods for generating the sequence of one or more RNA molecules and detecting the presence, identity, location, and quantity of RNA nucleotide modifications on said one or more RNA molecules said methods further comprising the implementation of non-MS-based sequencing methods such as next generation sequencing (NGS) methods.

The present provides a computer-implemented method for determining an order of nucleotides and/or nucleotide modifications of an RNA molecule, wherein the method includes: receiving/exporting liquid chromatography-mass-spectrometry (LC-MS) data of an RNA sample, the LC-MS data including but not limited to a mass (e.g., m/z, monoisotopic mass, average mass), charge states, retention time (RT), Height, width, volume, relative abundance, and quality score (QS); filtering/selecting the LC-MS data based on mass and/or other parameters, the filtering/selecting including removing masses smaller than a predetermined size; analyzing the filtered LC-MS data, to determine a plurality of RNA sequences, analyzing the filtered/chosen LC-MS data including: determining a mass difference between at least two RNA and/or adjacent ladder fragments; and determining whether the mass difference is equal to at least one of a canonical nucleotide, or a modified nucleotide (known or unknown); and reading-out an RNA sequence as a sequence read after determining no remaining valid nucleotides in the remaining LC-MS data, the RNA sequence including a sequence order of each identified canonical nucleotide and any identified modified nucleotides

In an embodiment, a computer-implemented sequencing method is provided for determining the Mass Sum of any of two fragments including but not limited to 3′/5′ ladder fragments; and if the mass sum is equal to the mass of the intact RNA (detected in homology search) and/or RNA segments/fragments plus the mass of a water, isolating these two fragments into a pair based on the determined MassSum for sequencing of the RNA molecule and/or segment/fragment. In an embodiment, MassSum may not be related to any two adjacent ladder fragments. Further, MassSum may not be limited to computational separate ladder fragments generated by one cleave per RNA molecule but may also be used to separate other fragments of RNA that gets cleaved more than once.

In another embodiment, a computer-implemented method is provided comprising the step of determining if any of the two ladder fragments cannot pair based on the mass sum value for a given RNA, and if so finding one of them by use of a GapFill algorithm, configured to search for ladder fragments missed by MassSum determination.

In yet another embodiment, the computer-implemented method comprises a step for identifying RNA isoforms based on a homology search function configured to divide the intact RNA molecules into two or more groups with each group representing one specific RNA species and its related isoforms. In such an embodiment, the homology search can be performed before or after degradation of the RNA. In another embodiment, the computer-implemented method comprises the step of determining presence, type, location, or quantity of the modified nucleotides within the RNA molecule. In an embodiment, a computer-implemented method is provided comprising the step of separating the 5′- and 3′end fragments of each identified tRNA isoform based on breaking two adjacent sigmoidal curves into two isolated curves. In an embodiment of the invention, a computer-implemented method is provided comprising the step of perfecting a faulted mass ladder by complementing the missing ladder fragments from related RNA isoforms identified in a homology search.

The present disclosure provides a kit for use in generating the sequence of one or more RNA molecules and detecting the presence, identity, location, and quantity of RNA nucleotide modifications on said one or more RNA molecules, said kit comprising one or more components for performance of a method comprising one or more of the steps of (i) controlled fragmentation of the RNA to form sequencable ladder fragments such as 5′ and 3′ MS ladder fragments; (ii) mass measurement of resultant degraded RNA samples containing RNAs and their fragmented fragments; and (iii) data processing, including identification and separation of 3′ and/or 5′ MS ladder fragments thereby generating the sequence of one or more RNA molecules and detecting the presence, identity, location, and quantity of RNA nucleotide modifications.

The present disclosure provides a kit for use in generating the sequence of one or more RNA molecules and detecting the presence, identity, location, and quantity of RNA nucleotide modifications on said one or more RNA molecules, said kit comprising one or more components for performance of a method comprising one or more of the steps of (i) identifying a specific chemical moiety associated with the RNA or labeling the RNA with a tag thereby imparting an identifiable property on the RNA (ii) controlled fragmentation of the RNA to form 5′ and 3′ MS ladder fragments; (iii) mass measurement of resultant degraded RNA samples containing RNAs and their degraded fragments; and (iv) data processing, including identification of 3′ and/or 5′ MS ladder fragments thereby generating the sequence of one or more RNA molecules and detecting the presence, identity, location, and quantity of RNA nucleotide modifications.

In another embodiment an MS based sequencing instrument is provided for use in generating the sequence of one or more RNA molecules and detecting the presence, identity, location, and quantity of RNA nucleotide modifications on said one or more RNA molecules, said instrument comprising one or more components for performance of the method comprising the steps of (i) controlled fragmentation of the RNA to form sequencable ladder fragments such as 5′ and 3′ MS ladder fragments; (ii) mass measurement of resultant degraded RNA samples containing RNAs and their fragmented fragments; and (iii) data processing, including identification and separation of 3′ and/or 5′ MS ladder fragments thereby generating the sequence of one or more RNA molecules and detecting the presence, identity, location, and quantity of RNA nucleotide modifications.

In another aspect, an MS based sequencing instrument for use in generating the sequence of one or more RNA molecules and detecting the presence, identity, location, and quantity of RNA nucleotide modifications on said one or more RNA molecules, said instrument comprising one or more components for performance of the method comprising the steps of (i) identifying a specific chemical moiety associated with the RNA or labeling the RNA with a tag thereby imparting an identifiable property on the RNA (ii) controlled fragmentation of the RNA to form 5′ and 3′ MS ladder fragments; (iii) mass measurement of resultant degraded RNA samples containing RNAs and their degraded fragments; and (iv) data processing, including identification of 3′ and/or 5′ MS ladder fragments thereby generating the sequence of one or more RNA molecules and detecting the presence, identity, location, and quantity of RNA nucleotide modifications.

Provided herein is a non-transitory computer-readable medium storing instructions that, when executed by a processor, cause the processor to perform method for generating the sequence of one or more RNA molecules and detecting the presence, identity, location, and quantity of RNA nucleotide modifications on said one or more RNA molecules, said method RNA comprising the steps of (i) controlled fragmentation of the RNA to form 5′ and 3′ MS ladder fragments; (ii) mass measurement of resultant degraded RNA samples containing RNAs and their fragmented fragments; and (iii) data processing, including identification and separation of 3′ and/or 5′ MS ladder fragments thereby generating the sequence of one or more RNA molecules and detecting the presence, identity, location, and quantity of RNA nucleotide modifications.

Also provided is a non-transitory computer-readable medium storing instructions that, when executed by a processor, cause the processor to perform a method for generating the sequence of one or more RNA molecules and detecting the presence, identity, location, and quantity of RNA nucleotide modifications on said one or more RNA molecules, the method comprising the steps of (i) identifying a specific chemical moiety associated with the RNA or labeling the RNA with a tag thereby imparting an identifiable property on the RNA (ii) controlled fragmentation of the RNA to form 5′ and 3′ MS ladder fragments; (iii) mass measurement of resultant degraded RNA samples containing RNAs and their degraded fragments; and (iv) data processing, including identification of 3′ and/or 5′ MS ladder fragments thereby generating the sequence of one or more RNA molecules and detecting the presence, identity, location, and quantity of RNA nucleotide modifications.

In one non-limiting embodiment an RNA sequencing method, referred to herein as the 2D-HELS MS Seq method, is provided for determining the primary RNA sequence, including the presence, identification, location, and quantification of RNA modifications of both single and mixed RNA sequences. Said method is based on the use of a two-dimensional hydrophobic end labeling strategy coupled with acid hydrolysis and MS-based measurement of RNA fragments. In an embodiment, an RNA sequencing method, for determining the primary RNA sequence and/or detecting the presence/identification of RNA modifications, is provided comprising the steps of: (i) labeling the 5′ and/or 3′ end of the RNA to be sequenced with a hydrophobic tag; (ii) conducting well-controlled acid hydrolysis of the RNA; (iii) LC-MS measurement of the resultant RNA fragment properties; and (iv) data analysis of resulting LC-MS data for sequence determination and modification analysis.

In a further embodiment, an RNA sequencing method, for determining the primary RNA sequence and the presence/identification/location/quantification of RNA modifications, is provided comprising the steps of: (i) treatment of RNA to be sequenced with N-cyclohexyl-N′-(2-morpholinoethyl)-carbodiimide metho-p-toluenesulfonate (CMC); (ii) labeling the 5′ and/or 3′ end of the RNA to be sequenced with a hydrophobic tag; (iii) acid hydrolysis of the RNA; (iv) LC-MS measurement of the resultant RNA fragment properties; and (v) data analysis resulting in sequence determination and modification identification/analysis.

In specific aspects, the 5′ and/or 3′ end of the RNA are labeled with affinity-based moieties and/or size shifting moieties. In an aspect, the fragment properties are detected through the use of one or more separation methods including, for example, high performance liquid chromatography, gas chromatography, capillary electrophoresis, and ion mobility spectrometry coupled with mass spectrometry.

The disclosed hydrophobic end-labelling sequencing method is based on the introduction of 2-D mass-retention time (t_{R}) shifts for ladder identification. Specifically, mass-t_{R} labels, or tags, are added to the 5′ and/or 3′ end of the RNA to be sequenced, and said moieties result in a retention time shift to longer times, causing all of the ladder fragments (5′ and/or 3′) to have a markedly delayed t_{R} compared to non-labelled RNA fragments. Hydrophobic label tags not only result in mass-t_{R} shifts of labelled ladders, making it much easier to identify each of the 2-D mass ladders needed for MS sequencing of RNA and thus simplifying base-calling procedures, but labelled tags also inherently increase the masses of the RNA ladder fragments so that the terminal bases can even be identified, thus allowing the complete reading of a sequence from one single ladder, rather than requiring paired-end reads as an additional step.

Although not a required step, in certain aspects of the present disclosure, the 3′ end labeled RNA may be physical separated from the 5′ unlabeled fragments prior to degradation of the RNA which are then subjected to LC/MS for HPLC and MS determination of the RNA and RNA modifications. The physical separation of the 5′ and 3′ ladder pools can be accomplished through the use of a variety of different molecular affinity interactions, such as for example, the affinity of biotin for streptavidin.

In one aspect, the RNA sequencing method disclosed herein comprises the steps of: (i) labeling of the 5′ and/or 3′ end of the RNA molecules with a hydrophobic tag; (ii) random acid mediated hydrolysis degradation of the labeled RNA; (iii) LC-MS measurement of the resultant RNA fragment properties to produce data for sequence/modification determination/identification. In a further embodiment, the additional step of data analysis based on one or more computer-implemented methods that extract, align and process relevant mass peaks or MS data from the LC-MS data may be conducted.

In another specific example, the method consists of (i) 5′ end chemical labeling of RNA with a bulky hydrophobic tag, like Cy3, which is designed to increase the size of the RNA fragment to increase retention time, (ii) formic acid-mediated RNA degradation, (iii) LC-MS measurement of the resultant RNA fragment properties, and (iv) data analysis based on one or more computer-implemented methods that extracts, aligns and processes relevant mass peaks from the mass spectrum.

In another embodiment, an RNA sequencing technique is provided that allows direct and simultaneous sequencing of each RNA in complex mixed RNA sample, including predominantly major RNA as well as even low stoichiometric RNA, such as for example tRNA, tRNA-derived small RNA (tsRNA), tRNA isoforms/species directly form complex samples without intensive sample preparation/separation and in the presence of imperfect/faulted mass ladder. The provided method comprises the steps of (i) controlled acid hydrolysis of the RNA to form mass/MS ladders; (ii) LC-MS measurement of resultant acid degraded RNA samples, containing RNAs (intact, degraded) and all their acid degraded fragments; and (iii) data processing and generation of RNA sequences and analysis of modified nucleotides, including their identification, location, and quantification. In an embodiment, the data processing and generation of sequences and identification of modified nucleotides employs one or more of different computational methods and tools including for example, algorithms for conducting homology searches, identification of acid-labile nucleotide, mass-sum-based data separation, gap-filling, ladder separation, ladder complementing, and RNA sequence (canonical and modified) generation.

In another embodiment, an RNA sequencing technique is provided that enhances the read length and throughput, allowing direct and simultaneous sequencing of tRNA isoform mixtures (˜80 nt long each) with T1 or any enzymatic digestion and physical sample separation in a single LC-MS run, such as tRNA, tRNA-derived small RNA (tsRNA), tRNA isoforms/species directly form complex samples without intensive sample preparation. The provided method comprises the steps of (i) controlled acid hydrolysis of the RNA to form MS ladders; (ii) LC-MS detection of resultant acid degraded RNA samples, containing RNAs (intact, degraded) and all their acid degraded fragments; and (iii) data processing and generation of sequences and identification of modified nucleotides. In an embodiment, the data processing and generation of sequences and identification of modified nucleotides employs one or more of different computational methods and tools including for example, algorithms for conducting homology searches, identification of acid-labile nucleotide, mass-sum-based data separation, gap-filling, ladder separation, ladder complementing, and sequence generation.

In another embodiment, an RNA sequencing technique is provided that allows direct and simultaneous sequencing of each tRNA isoform in a complex mixed RNA sample even in the absence a perfect mass ladder corresponding from the first to the last nucleotide in an RNA sequence. The RNA samples include any RNA nucleotide-modified, edited, or terminal truncated RNA, such as for example tRNA, tRNA-derived small RNA (tsRNA), tRNA isoforms/species directly form complex samples without intensive sample preparation/separation and in the presence of imperfect/faulted mass ladder. Taking tRNA samples as an example, the provided method comprises the steps of i) well-control acid hydrolysis to generate MS ladders, ii) homology search of intact tRNAs to first identify the related tRNA isoforms caused by partial RNA modifications and/or 3′ end truncations, iii) implementation of a mass-sum-based strategy to computationally isolate MS ladders for each tRNA isoform/species from the RNA mixture, and iv) implement ladder complementary sequencing in which broken/imperfect ladders of different isoforms are complementary and contribute to the completion of a perfect MS ladder for sequencing of the tRNA and related isoforms.

Further details and aspects of exemplary embodiments of the disclosure are described in more detail below with reference to the appended figures. Any of the above aspects and embodiments of the disclosure may be combined without departing from the scope of the disclosure.

### BRIEF DESCRIPTION OF THE DRAWINGS

Various embodiment of methods are described herein with reference to the drawings wherein:

FIG. 1A-D. 2D-HELS MS Seq of representative RNA samples. (FIG. 1A) Workflow for 2D-HELS MS Seq. The major steps include 1) hydrophobic tag-labeling of RNA to be sequenced, 2) acid hydrolysis, 3) LC-MS measurement, 4) extraction and analysis of MFE data, and 5) sequence generation via algorithms or manual calculation. (FIG. 1B) Sample preparation protocol including introducing a biotin tag to the 3′-end of RNA for 2D-HELS MS Seq. (FIG. 1C) Separation of the 3′-ladder from the 5′-ladder and other undesired fragments in a 2D mass-retention time (t_{R}) plot based on systematic changes in t_{R}s of 3′-biotin-labeled mass-t_{R} ladder fragments of RNA #1 (19 nt). The sequences are de novo and automatically read out directly by a base-calling algorithm⁹. FIG. 1C discloses SEQ ID NOS 10 and 34, respectively, in order of appearance. (FIG. 1D) Simultaneous sequencing of 5′-biotin labeled RNA #1 and RNA #2, 19 nt and 20 nt, respectively. FIG. 1D discloses SEQ ID NOS 35 and 10, respectively, in order of appearance.

FIG. 2A-B. Converting pseudouridine (ψ) to its CMC-ψ adduct for 2D-HELS MS Seq. (FIG. 2A) HPLC profile of the crude product of the reaction converting ψ to its CMC adduct in a 20 nt RNA (RNA #6) that contains one ψ. (FIG. 2B) Sequencing of a ψ-containing RNA #6. The conversion of the ψ to the CMC-ψ adducts (ψ*) results in a 252.2076 Dalton increase in mass and a significant increase in t_{R} because of its mass and hydrophobicity of the CMC. Thus, a dramatic shift starting at the position of 8 can be observed in the mass-t_{R} plot, indicating that this is a ψ at the position of 8 in the RNA sequence. The sequences are manually acquired based on the computational algorithm-processed data. This figure has been modified from Zhang et al. FIG. 2B discloses SEQ ID NO: 36.

FIG. 3. Sequencing RNA mixtures containing five distinct RNAs. A biotin is used to label each RNA at their 3′-end before 2D-HELS MS Seq. For each sequence, the starting t_{R} values are normalized systematically to start at 7 min intervals for ease of visualization. The absolute differences between the starting t_{R} value and subsequent t_{R}s remain unchanged for each of the five RNAs, and thus it is easier to visualize each of them in the same plot. All bases are identified by manually calculating the mass differences of two adjacent ladder components and matching them with the theoretical mass differences in the RNA nucleotide and modification database; plots for FIG. 3 are re-constructed using OriginLab based on manual base-calling and sequencing data. FIG. 3 discloses SEQ ID NOS 15, 14, 13, 11 and 10, respectively, in order of appearance.

FIG. 4. 2D-HELS MS sequencing of 5 mixed RNA strands simultaneously using a biotin tag to label the 3′-ends. Original t_{R} was displayed without any normalization.

FIG. 5A-B. FIG. 5A Each cleavage of an RNA phosphodiester bond by acid-mediated hydrolysis generates two fragments, one containing the original 5′ hydroxyl (OH) and a newly-formed phosphate at the 3′ end, and the other containing the original 3′OH and a newly-formed OH at the 5′ end. FIG. 5B A schematic picture using a short oligonucleotide 5′HO-ACGUAC-OH 3′ as an example to illustrate the potential overlap of mass peaks of ladder fragments that contribute to formation of 5′ ladder and 3′ ladders in traditional 1D MS sequencing.

FIG. 6A-B. (FIG. 6A) Workflow for 2D-HELS MS Seq (Introduction of a two-dimensional hydrophobic end-labeling strategy to MS-based sequencing). The major steps include hydrophobic tag labeling of RNA to be sequenced, acid hydrolysis, LC-MS measurement and sequence generation by a computer-implemented method. (FIG. 6B) The chemical structure of a hydrophobic tag, AppCp-biotin.

FIG. 7A-C. 2D mass-t_{R} plot of sequencing of representative RNA samples. (FIG. 7A) Sequencing of RNA #1 (19 nt). The 3′ end is biotin-labeled during sample preparation before acid degradation. All the 3′-ladder fragments are welled separated from the unlabeled 5′-ladder fragments and other undesired fragments in the 2D plot due to a systematic increase in their t_{R}s. The sequences are automatically generated by an anchor-based computer-implemented method. FIG. 7A discloses SEQ ID NOS 37-38, respectively, in order of appearance. (FIG. 7B) Sequencing of a mixture of RNA containing five different RNA sequences (RNAs #1-#5). A biotin tag is used to label each RNA at the 3′-end, and t_{R}s of each RNA ladder are normalized to begin at 7 min intervals for ease of visualization. All base-calls are performed manually by calculating the mass differences of two adjacent ladder components and matching them with the theoretical mass differences in the RNA nucleotide and modification database. With base-by-base base-calling, all sequences of the five RNA are correctly read out. FIG. 7B discloses SEQ ID NOS 39-42 and 37, respectively in order of appearance. (FIG. 7C) Sequencing of RNA #6, which contains one ψ. The increase in mass and hydrophobicity caused by conversion of the ψ to the CMC-ψ adduct (ψ*) results in a systematic mass-t_{R} shift on all CMC-ψ-containing ladder fragments beginning at the w position. This site-specific shift indicates that a ψ is at position 8 in the RNA sequence. The other modification, m⁵C, can be simultaneously identified and located at position 16 based on its unique mass. The sequences are acquired by an anchor-based computer-implemented method. All three 2D plots are re-constructed by OriginLab based on sequences read out by the anchor-based algorithm or manual calculation. FIG. 7C discloses SEQ ID NO: 12.

FIG. 8A-C 2D-HELS-AA MS Seq of Yeast tRNA^{Phe}. FIG. 8A. 1)-6): Sequencing workflow. FIG. 8B. A 2D plot of the entire tRNA sequenced from a single LC-MS run, showing the identity and location of all modifications. FIG. 8B discloses SEQ ID NOS 43-44, 69 and 45, respectively, in order of appearance. FIG. 8C. Assembly of the full-length tRNA^{Phe} sequence based on overlapping sequence reads from different LC-MS runs, showing 100% coverage and accuracy as compared to the reported tRNA^{Phe} reference sequence. All output sequence reads are converted to FASTA format in the 5′ to 3′ order (44 and 45 AG conversion output reads not included). *Ts: the Table S where the sequencing data of that particular strand can be found. FIG. 8C discloses SEQ ID NOS 46-47, 19, 19, 26, 25, 21, 48, 22, 17 and 16 respectively, in order of appearance.

FIG. 9A-C. Sequencing of all 11 RNA modifications. FIG. 9A. A proposed mechanism for the conversion of wybutosine (Y) to its depurinated form (Y′) in acidic conditions. FIG. 9B. The mass of Y was found in the crude products after acid degradation. The relative percentages of Y and Y′ were quantified and can be found in Table S3-18. FIG. 9C. Summary of all 11 RNA modifications sequenced by 2D-HELS-AA MS Seq. The relative percentages of modifications at each position were quantified by integrating the EIC peaks of their corresponding ladder fragments (Table S3-19). The percentages of partially modified nucleotides are highlighted in pink.

FIG. 10A-B. Identification of 3′ truncation isoforms. FIG. 10A. 2D-HELS-AA MS sequencing of segment III, showing two other truncated isoforms of tRNA^{Phe} at the 3′ end (74 nt and 75 nt). t_{R} was normalized for ease of visualization of the 74 nt and 75 nt isoforms. FIG. 10A discloses SEQ ID NOS 49, 43 and 50, respectively, in order of appearance. FIG. 10B. The terminal base of 76 nt tRNA^{Phe} and its two tail-truncated isoforms; all three isoforms contain a free OH at the 3′ end, which is required for introducing the biotin tag, suggesting that the isoforms were not generated during acid degradation but came together with the full-length 76 nt tRNA original. FIG. 10B discloses SEQ ID NOS 51-53, respectively, in order of appearance.

FIG. 11A-D Discovering a new 44g45a isoform in the tRNA variable loop. FIG. 11A. A schematic of sequence ladder fragments shows a transition/editing g (sharing an identical mass as G) co-exists with A at position 44 when reading from the 5′ direction (Table S3-4 through Table S3-5 and Table S3-8 through Table S3-9). FIG. 11B. Least squares fitted mass spectrum to the calibrated mass spectrum (t_{R}=31.9-32.9 min) when reading from the 5′ direction. The full-spectral analysis confirms that the ions of the 44g and 44A fragments (with 10 charges) co-exist and that their relative abundances are 57% and 43%, respectively. The theoretical trace of the two combined ion profiles fits well with the calibrated mass spectrum as observed, resulting in a good spectral accuracy of 87%. FIG. 11C. A single transition/editing a (one oxygen less than G) co-exists with G at position 45 when reading from the 3′ direction (Table S3-19 through Table S3-22). FIG. 11D. Similar to B, full-spectral analysis confirms that the ions of the 45a and 45G fragments (both with four charges; spectral accuracy: 71%) also co-exist and their relative abundances are 47% and 53%, respectively, when reading from the 3′ direction (t_{R}=16.5-18.6 min).

FIG. 12. Summary of different RNA isoforms, base modifications, and base editing as well as their stoichiometries in the tRNA^{phe}. FIG. 12 discloses SEQ ID NO: 54.

FIG. 13A-C. 2D-HELS-AA MS Seq (2-dimensional hydrophobic RNA end-labeling strategy with an anchor-based algorithm in mass spectrometry-based sequencing) of three segments digested by RNase T1. As part of HELS, based on the unique chemical moieties on the termini of the three segments, a single biotin label was selectively introduced to each of the three segments on either their 5′- or their 3′-end followed by streptavidin bead-based isolation and release of each segment for acid degradation by formic acid. After liquid chromatography (LC)-MS and data collection, data were subsequently exported by a molecular feature extraction (MFE, Agilent, USA) algorithm for sequence generation using an anchor-based algorithm. A sequence of 19 bases (58m¹A to 76A) corresponding to segment III (FIG. 13A), a sequence of 37 bases (21A to 57G) corresponding to segment II (FIG. 13B), and a sequence of 18 bases corresponding to segment I (1G to 18G) (FIG. 13C) were determined, respectively. The location of all 11 mass-altering tRNA modifications in the three segments were also successfully detected. FIG. 13A discloses SEQ ID NOS 51 and 51, respectively, in order of appearance. FIG. 13B discloses SEQ ID NOS 55-56, respectively, in order of appearance. FIG. 13C discloses SEQ ID NOS 57-58, respectively, in order of appearance.

FIG. 14A-B. MS analysis of methylated nucleotide dimers by collision induced dissociation (CID) MS/MS. Samples were prepared by intensive acid hydrolysis (80° C., 75% (v/v) formic acid, 2 hrs) to generate the dimers. MS/MS data were collected for the modified dimer and fragment ions were used to confirm that the methylation is on the ribose 2′ position of cytidine. The sequences are (FIG. 14A) CmU and (FIG. 14B) GmA, respectively. Assignable fragment labels are indicated on the dimer structures, and the peaks representing the fragments match by color.

FIG. 15. Reverse transcription single base extension (rtSBE) experiment to differentiate m¹A and m⁶A (N⁶-methyladenosine). A pause was observed in the rtSBE experiment, indicating that m¹A, rather than m⁶A, exists at position 58, because m¹A is not able to form base-pairing interactions, thus causing a pause during reverse transcription. FIG. 15 discloses SEQ ID NOS 59, 7, 60, 7 and 61-62, respectively, in order of columns.

FIG. 16A-B. The conversion of pseudouridine (ψ) to CMC-labeled pseudouridine (ψ*) results in a shift in both t_{R} and mass of relevant data points, allowing facile identification and location of ψ at this position due to a single drastic jump in the mass-t_{R} ladder. For ease of visualization, only the sequences of the (A) 5′-mass-t_{R} ladder (22G to 44A) and (B) 3′-mass-t_{R} ladder (57G to 47U) are presented. The sequences presented were manually acquired based on the mass-t_{R} ladders identified from the algorithm-processed data. The structures in (A) show the chemical conversion of ψ by reaction with CMC to form the CMC-ψ adduct, shifting CMC-ψ-containing mass-t_{R} ladders in both mass and t_{R} compared to mass-t_{R} ladders containing unconverted ψ. FIG. 16A discloses SEQ ID NO: 63. FIG. 16B discloses SEQ ID NO: 64.

FIG. 17A-C. (FIG. 17A) Chemistry for distinguishing m⁷G from other isomeric base modifications, such as m²G (N²-methylguanosine), that share an identical mass. (FIG. 17B) The plot of Intensity vs. Mass after chemical cleavage of the RNA at m⁷G site-specifically. The mass of the three major fragments observed were 9587.3076 Da, 9258.2538 Da, and 8953.2171 Da, corresponding to their 76 nt, 75 nt, and 74 nt isoforms, respectively, indicating that there is a m⁷G at the 46 position. (FIG. 17C) Specific fragments cleaved at m⁷G were analyzed by LC-MS and quantified by integrating EIC peaks of their corresponding fragments.

FIG. 18A-B. MALDI-TOF results of rtSBE experiments. (FIG. 18A) For cDNA primer 1, only ddT (position 44) was incorporated. (FIG. 18B) For cDNA primer 2, only ddC (position 45) was incorporated. The results suggest that the tRNA template in the rtSBE experiment was the 44A and 45G wild-type isoform. FIG. 18A discloses SEQ ID NOS 65-67, respectively, in order of appearance.

FIG. 19A-B. (FIG. 19A) Chemical structure of isoG (2-oxoadenine) and 8-oxo-A (8-oxoadenine). (FIG. 19B) The EIC profile confirms the existence of both G monophosphate and g monophosphate (lower case g is used to differentiate it from the canonical G in position 44) at different t_{R}.

FIG. 20A-H. Workflow of de novo sequencing of tRNA isoform mixtures, including The steps of: 1) acid hydrolysis of tRNA samples (single-stranded or mixed) in well-controlled conditions to general ladder fragments, 2) LC-MS detection of the resultant acid-degraded tRNA samples, containing tRNAs (intact or degraded) and all their acid-hydrolyzed fragments, and 3) data processing and generation of sequences made of both canonic and modified nucleotides (if they exist). The last step requires a complete set of step-wise innovative computational methods/tools, including algorithms mainly for homology search, identifying acid-labile nucleotide, mass-sum-based data separation, gap-filling, ladder separation, ladder complementing, and sequence generation. FIG. 20G discloses SEQ ID NO: 68. FIG. 20H discloses SEQ ID NO: 70.

FIG. 21 A-C. FIG. 21A. Homology search before acid degradation for identifying the related tRNA isoforms. FIG. 21B. Identify each tRNA containing acid-labile nucleotide modifications by comparing the mass changes of the intact tRNA before and after acid degradation. FIG. 21C A mechanism illustrating a 358. 14 Dalton mass decrease due to the conversion of acid-labile wybutosine (Y) to its depurinated form (Y′) in acidic conditions.

FIG. 22A-F. MassSum strategy and MassSum-based computational data separation. FIG. 22A-F. An isolated/mixed RNA starting material is partially digested in a manner that predominantly generates single-cut fragments. Taking a 9 nt RNA strand as an example to illustrate the idea, two ladder fragments are generated as a result of an acid-mediated cleavage of the phosphodiester bond between 1st nucleotide and 2nd nucleotide of the 9 nt RNA strand. One of them carries the original 5′-end of the RNA strand and has a newly-formed ribonucleotide 3′(2)-monophosphate at its 3′-end (denoting as F1). The other one carries the original 3′-end of the RNA strand and has a newly-formed hydroxyl at its 5′-end (denoting as T8). FIG. 22B. The mass sum of any one-cut fragment pair, e.g., mass sum of F2 and T7 equal to the mass sum of F1 and T8, is constant and equals to the mass of 9 nt RNA plus the mass of a water molecule. Since the mass sum is unique to each RNA sequence/strand, and it can be used to computationally separate all paired fragments of the RNA sequence/strand out of complex MS datasets. FIG. 22C. computationally isolate MS data of all ladder fragments derived/degraded from the same tRNA isoform sequence in both the 5′- and 3′-ladders out of the complex MS data of mixed samples with multiple distinct RNA strands using a 75 nt tRNA-Phe (monoisotopic mass: 24252; Relative abundance: 100% compared to the 75 nt tRNA-Phe (2^{nd} isoform)). Separated data of 5′- and 3′-ladder fragments for 75 nt tRNA-Phe (major in the sample mixture) (FIG. 22C) and 76 nt tRNA-Phe (2^{nd} isoform with 6C and 67G) (1% abundance; minor in the sample mixture) (FIG. 22E), respectively. FIGS. 22 D and F. de novo MS sequencing and generating sequence of tRNA-Phe completely (FIG. 22D) and tRNA-Phe (2^{nd} isoform) in part (FIG. 22F), respectively. FIG. 22D discloses SEQ ID NO: 71. FIG. 22F discloses SEQ ID NO: 72.

FIG. 23A-C. Completion/fixing of a faulted mass ladder by complementing the missing ladders from other isoforms identified in homology search for 5′-ladder (FIG. 23A), 3′-ladders (FIG. 23B), and complementing original 5′-ladders and converted 5′-ladders (FIG. 23C) of the tRNA-Phe. FIG. 23A discloses SEQ ID NO: 73. FIG. 23B discloses SEQ ID NO: 74. FIG. 23C discloses SEQ ID NO: 75.

FIG. 24 A-F. Sequencing of minor tRNA-Glu isoforms/species (<1% relative abundance) in complex RNA mixture samples prepared from A549 cells (with or without RSV infection). FIG. 24A Homology search to find different methylated tRNA-Glu isoforms in the mass range of >24K Dalton in the 2D mass-t_{R} plot for RNA samples with Mock (in blue) or RSV infection (in green). FIG. 24B MassSum data separation of one of the most abundant tRNA-Glu isoforms out of the complex MS mixture, and find ladders missed during MassSum data separation via a GapFill algorithm. FIG. 24C de novo MS sequencing and generating sequence of tRNA-Glu in part. FIG. 24C discloses SEQ ID NO: 76. FIG. 24D blasted out one tRNA with a complete 75 nt sequence form massive NGS sequencing results (>10 million reads) performed in parallel. FIG. 24D discloses SEQ ID NO: 77. FIG. 24E Sequencing of RNA modifications by mass shift between observed monoisotopic masses and in silico calculated theoretical exact mass for each ladder fragment. FIG. 24F tRNA-Glu sequence containing RNA modifications. FIG. 24F discloses SEQ ID NO: 78.

FIG. 25A-B. Possible fragmentation sites in oligonucleotides and nomenclature proposed by Mcluckey et al. (FIG. 25A) Of five possible cleavage sites, a-B cleavage can remove the nucleobase of RNA. Four other possible MS cleavage sites, denoted a, b, c, and d, when fragmented ion contains 5′ terminus, or ψ, x, y, and z when fragmented ion contains the 3′ terminus. The numerical subscript gives the number of bases from the respective termini. The letter B represents the position of the bases and the numerical subscript indicates their position relative to the 5′ terminus. (FIG. 25B) After acid treatment in 2D-HELS MS sequencing, possible fragmentation sites of oligonucleotides occur at one specific position of phosphodiester backbone.

FIG. 26A-B. (FIG. 26A) A full-range Monoisotopic Mass-Abundance chart for LC-MS data of yeast tRNA-Phe sample. (FIG. 26B) A Monoisotopic Mass-Retention Time (min) chart at around 25 kDa before acid degradation for homology search. The most abundant masses became the initial sequencing targets.

FIG. 27 A complete 2D mass-t_{R} plot of LC-MS data for yeast tRNA-Phe after acid hydrolysis. Circled area was analyzed during the homology search.

FIG. 28A-C. A general categorization for the data points from the complete 2D mass-t_{R} plot of LC-MS data of acid-degraded yeast tRNA-Phe. (FIG. 28A) Data points representing 5′ fragments for ladder separation are highlighted. (FIG. 28B) Data points representing 5′ fragments for ladder separation are highlighted. (FIG. 28C) Inevitable overlapped data points are highlighted. Mass pair searches (MassSum) were then applied based on this general categorization of data points.

FIGS. 29A-1-29A-4, 29B-1-29B-2 and 29C. Data processing using 24581.381 Da (76 nt) and 24252.311 Da (75nt), 23947.31 Da (74t), 24597.36 Da (76nt+O) and 24268.31 Da (75nt+O) as sequencing targets. (FIG. 29A-1-FIG. 29A-4) MassSum was applied to extract fragmental mass pairs out of complex MS data of mixed RNA sample, upon which GapFill was applied to search for more ladders missed by MassSum data separation. (29B-1-29B-2) 3′-end complementary laddering. After converting the 3′ ladders to 5′ using the MassSum equation, the fragments were complemented to become more continuous. (FIG. 29C) Final sequence generated from complementary laddering. 5′-end complementary laddering. 5′-end ladders were complemented without further adjustments. FIG. 29C discloses SEQ ID NO: 74.

FIG. 30. Pseudocode for MassSum algorithm.

FIG. 31. Pseudocode for GapFill algorithm.

FIG. 32. Possible cleavage sites observed in tRNA-Glu RSV infected samples. (A) All the data points existed only in RSV infected samples (not mock samples). The strongest masses were highlighted with red color. (B) 3 cleavage sites were marked with red line on the tRNA Glu structure. FIG. 32 discloses SEQ ID NO: 78.

FIG. 33A-B. (FIG. 33A) Workflow of the 2D-HELS-AA MS Seq for direct sequencing of RNAs, and a modified RNA was chosen as one example to illustrate the method's concept. A hydrophobic tag such as biotin was introduced to the RNA's 3′ end. After controlled acid degradation to generate ladder fragments and the subsequent LC-MS measurement, the 3′ biotinylated ladder with a biotin on the termini of all its ladder fragments was shifted to the top and to the right in the 2D mass-retention time (t_{R}) plot because the biotin tag helped to increase the t_{R} values and masses of the ladder fragments comparting to their unlabeled counterparts. The trend of biotin-induced shift is known and was used to identify the 3′ ladder for sequencing of the RNA as well as its base modifications. The hydrophobic tag can be a different moiety such as Cy3, and can be introduced to at least one end of the RNA (3′ and/or 5′) to generate the mass-t_{R} shift. (FIG. 33B) Workflow of data analysis using an anchor-based sequencing algorithm with the global hierarchical ranking strategy. The MS data shown in the work flow is simulated with a purified sample, and the intensity of the color indicates the associated volume of each data point with darker blue points indicating higher volume and vice versa. Na+, 2Na+, Na++K+ and other mass adducts were hierarchically clustered to augment compound intensity and to reduce data complexity in step 2. The processed data were subsetted by filtering t_{R} and mass value, so that only data points in the zone of labeled fragments were passed on in the algorithm in step 1. An anchor-based algorithm was applied for de novo sequence generation automatically. All draft reads were ranked by read length, average volume, average QS and average PPM in this order, and the top-ranking draft read for each fragment was output and chosen as the final output read.

FIG. 34. Design of reverse transcription single base extension experiments for confirming 45G position. FIG. 34 discloses SEQ ID NOS 79-80, respectively, in order of appearance.

FIG. 35. Design of reverse transcription single base extension experiments for confirming 44A position. FIG. 35 discloses SEQ ID NOS 79 and 66, respectively, in order of appearance.

FIG. 36. Design of reverse transcription single base extension experiments for confirming 43G position. FIG. 36 discloses SEQ ID NOS 79 and 81, respectively, in order of appearance.

FIG. 37. The pseudocode for base-calling step of the global hierarchical ranking algorithm. In this step the algorithm stores all possible tuples of (Mᵢ BASE, Mⱼ) recoding the mass from MS data as Mᵢ and Mⱼ and the base identity matching with the mass difference of Mᵢ and Mⱼ as BASE.

FIG. 38. The pseudocode for sequence generation step of the global hierarchical ranking algorithm. In this step the algorithm takes the tuples stored in base-calling as nodes and connects the nodes to build paths corresponding to draft reads.

FIG. 39. The pseudocode of the draft read selection step of the global hierarchical ranking algorithm. The draft reads are evaluated by four parameters in order: read length, average volume, average QS and average PPM, which each parameter the algorithm performs a round of ranking of the draft reads. The draft read at the top ranking becomes the final output.

FIG. 40. The pseudocode of the local best score algorithm. Instead of generating all possible tuples during base calling, the local best score algorithm only stores the base identity and corresponding mass with the highest volume. Thus, the local best score algorithm generates only one draft read.

FIGS. 41-1-41-6. The algorithm implementing the local best score strategy, performed by a Python coding system. FIGS. 41-1-41-6 discloses SEQ ID NO: 82.

FIG. 42. The pseudocode of a revised Smith-Waterman alignment similarity algorithm for assembling overlapping tRNA sequences into a complete tRNA sequence.

FIG. 43. The pseudocode of a computer-implemented method for identifying acid-labile nucleotides.

FIG. 44. The pseudocode of a computer-implemented method for homology search of related tRNA isoforms.

FIG. 45. The pseudocode of a computer-implemented method for ladder complementing.

FIGS. 46-1-46-2. The tool for computational ladder separation.

FIG. 47 is a block diagram of a controller configured for use with the disclosed methods.

### DETAILED DESCRIPTION

Although the present disclosure will be described in terms of specific embodiments, it will be readily apparent to those skilled in this art that various modifications, rearrangements, and substitutions may be made without departing from the spirit of the present disclosure. The scope of the present disclosure is defined by the claims appended hereto.

For purposes of promoting an understanding of the principles of the present disclosure, reference will now be made to exemplary embodiments illustrated in the drawings, and specific language will be used to describe the same. It will nevertheless be understood that no limitation of the scope of the present disclosure is thereby intended. Any alterations and further modifications of the inventive features illustrated herein, and any additional applications of the principles of the present disclosure as illustrated herein, which would occur to one skilled in the relevant art and having possession of this disclosure, are to be considered within the scope of the present disclosure.

The current disclosure is related to direct, liquid-chromatography-mass spectrometry (herein referred to as LC-MS) based RNA sequencing methods which can be used to directly sequence RNA without cDNA synthesis, simultaneously determine the nucleotide sequence of RNA molecules with single nucleotide resolution as well as detection of the presence of any nucleotide modifications that an RNA molecule carries. The disclosed methods can be used to determine the type, location and quantity of nucleotide modifications within the RNA sample. The RNA to be sequenced may be a purified RNA sample of limited diversity, as well as samples of RNA containing complex mixtures of RNA, such as RNA derived from a biological sample. Such techniques can be used to determine the nucleotide (modified or canonical) sequence of an RNA molecule and to advantageously correlate the biological functions of any given RNA molecule with its associated modifications.

As used herein, ribonucleic acid (RNA) refers to oligoribonucleotides or polyribonucleotides as well as any analogs of RNA, for example, made from nucleotide analogs. The RNA will typically have a base moiety of adenine (A), guanine (G), cytosine (C) and uracil (U), a sugar moiety of a ribose and a phosphate moiety of phosphate bonds. RNA molecules include both natural RNA and artificial RNA analogs. The RNA can be synthetic or can be isolated from a particular biological sample using any number of procedures which are well known in the art, wherein the particular chosen procedure is appropriate for the particular biological sample. RNA samples include for example, coding RNA and non-coding RNA such as mRNA, rRNA, tRNA, antisense-RNA, and siRNA, to name a few. No limitations are imposed on the base length of RNA. The LC-MS-based sequencing methods disclosed herein enable the sequencing of not only purified RNA samples, but also more complicated RNA samples containing mixtures of different RNAs.

In a specific embodiment, the structure of synthetic oligoribonucleotides of therapeutic value can be determined using the sequencing methods disclosed herein. Such methods will be of special valuable to those engaged in research, manufacture, and quality control of RNA-based therapeutics, as well as the regulatory entities. Incorporation of structural modifications into synthetic oligoribonucleotides has been a proven strategy for improving the polymer's physical properties and pharmacokinetic parameters. However, the characterization and the structure elucidation of synthetic and highly-modified oligonucleotides remains a significant hurdle.

In one aspect, the sequencing method of the present disclosure comprises the steps of: (i) partial degradation of the RNA (ii) affinity labeling of the 5′ and 3′ end of the RNA sample to facilitate subsequent separation of the 5′ and 3′ end labeled RNA pools; (ii) random non-specific cleavage of the RNA; (iii) physical separation of resultant target RNA fragments using affinity based interactions before LC-MS or separation during LC section of LC-MS; (iv) LC-MS measurement, and (v) sequence generation and modification analysis. Such affinity interactions are well known to those skilled in the art and included, for example, those interactions based on affinities such as those between antigen and antibody, enzyme and substrate, receptor and ligand, or protein and nucleic acid, to name a few. Labeling of the 5′ and 3′ ends of the fragmented RNA for use in affinity separation may be achieved using a variety of different methods well known to those skilled in the art. Such labeling is designed to achieve separation of fragmented RNA for subsequent MS analysis. RNA end-labeling may be performed before or after the chemical cleavage of the RNA.

In one embodiment, the biotin/streptavidin interaction may be utilized to enrich for the ladder RNA fragments. As one example, the 3′ and 5′ RNA ends may be labeled with biotin for subsequent separation of RNA fragments based on the biotin/streptavidin interaction through use of streptavidin beads. In yet another aspect, short DNA adapters may be ligated to each end of the RNA sample. In a specific embodiment, a biotin tag is added via a two-step reaction, at each end of the RNA sample. As a first step, a thiol-containing phosphate is introduced at the 5′-end by reacting T4 polynucleotide kinase with adenosine 5′-[γ-thio]triphosphate (ATP-γ-S) to add a thiophosphate to the 5′ hydroxyl group of the to-be-sequenced RNA and then a conjugation addition is made between the resultant thiolphosphorylated RNA and the biotin (Long Arm) Maleimide (Vector Laboratories, USA), which is designed for biotinylating proteins, nucleic acids, or other molecules containing one or more thiol groups. The resulting 5′-biotinylated-RNA is then treated with formic acid, similar to the previous procedure (13). After acid degradation, streptavidin-coupled beads (Thermo Fisher Scientific, USA) are used to single out the 5′ ladder pool, which will be released for subsequent LC-MS analysis after breaking the biotin-streptavidin interaction.

In yet another embodiment, the poly (A) oligonucleotide/dT interaction may be used to separate fragmented RNA. In instances where the end of the RNA is labeled with a biotin moiety, streptavidin beads may be used to purify the desired RNA ladder fragments. Alternatively, where the RNA has been labeled with a poly (A) DNA oligonucleotide, oligopoly (dT) immobilized beads such as (dT) 25-cellulose beads (New England Biolabs) may be used to enrich for the RNA fragments. The choice of chromatography material will be dependent on the 5′ and 3′ RNA labeling used and selection of such chromatography/separation material is well known to those skilled in the art.

The 3′ end of the RNA may be ligated to a 5′ phosphate-terminated, pentamer-capped photocleavable poly(A) DNA oligonucleotide with T4 RNA ligase to form a phosphodiester-linked RNA-DNA hybrid. The 5′ end of the RNA-DNA hybrid may then be ligated to 5′ biotinylated DNA after phosphorylation via T4 polynucleotide kinase using T4 RNA ligase.

In a specific embodiment, two short DNA adapters may be ligated to each end of the RNA sample, to physically select the desired fragment into either the 5′ or 3′ ladder pool from the undesired fragments with more than one phosphodiester bond cleavage in the crude degraded product mixture, followed by a well-controlled formic acid degradation time resulting in most of the RNA sample being degraded, most of which turn into the desired fragments needed to obtain a complete sequence ladder. The 3′ end of the RNA sample is ligated to a 5′-phosphate-terminated, pentamer-capped photocleavable poly (A) DNA oligonucleotide with T4 RNA ligase 1 (New England Biolabs) to form a phosphodiester-linked RNA-DNA hybrid. Likewise, the 5′ end of the RNA-DNA hybrid is ligated to 5′-biotinylated DNA after phosphorylation via T4 polynucleotide kinase with the same ligase. The resulting 5′ DNA-RNA-DNA-3′ hybrid is treated with formic acid for approximately 5-15 min. Following formic acid treatment, streptavidin-coupled beads (ThermoFisher Scientific) can be used to isolate the 5′ ladder fragment pool followed by oligomer-release for subsequent LC/MS analysis. Similarly, oligopoly (dT) immobilized beads such as (dT) 25-Cellulose beads (New England Biolabs) can be used to enrich the 5′ ladder, which can then be eluted for LC/MS analysis after photocleavage by UV light (300-350 nm). Only the RNA section of the hybrid will be hydrolyzed, while the DNA section will remain intact as DNA lacks the 2′-OH group.

In a specific embodiment, to increase the retention time shift, the RNA may be labeled with bulky moieties such as, for example, a hydrophobic Cy3 or Cy5 tag or other fluorescent tag at the 5′- or 3′-end. Such a tag is added via a two-step reaction, at the 5′-end of the RNA sample. As a first step, a thiol-containing phosphate is introduced at the 5′-end by reacting T4 polynucleotide kinase with adenosine 5′-[γ-thio]triphosphate (ATP-γ-S) to add a thiophosphate to the 5′ hydroxyl group of the to-be-sequenced RNA and then a conjugation addition is made between the resultant thiolphosphorylated RNA and the Cy3 or Cy5 Maleimide (Tenova Pharmaceuticals, USA), which is designed for biotinylating proteins, nucleic acids, or other molecules containing one or more thiol groups. After 3′ end biotin labeling and acid degradation, the resultant two-end-labeled RNA maybe directly subjected for LC/MS without any affinity-based physical separation. For a two-step labeling RNAs at their 3′-ends, biotinylated cytidine bisphosphate (pCp-biotin) is activated by adenylation using ATP and Mth RNA ligase to produce AppCp-biotin. Then, the RNAs with a free 3′-terminal hydroxyl (OH) were ligated to the activated AppCp-biotin via T4 RNA ligase. Streptavidin-coupled beads were used to isolate the 3′-biotin-labeled RNAs, which were released for acid degradation and subsequent LC-MS analysis after breaking the biotin-streptavidin interaction. For one step labeling RNAs at their 3′ end, pCp-biotin was replaced with AppCp-biotin by performing a one-step ligation reaction. The 3′-end labeling efficiency increased from 60%, using a two-step protocol, to 95% using a one-step protocol, when activated AppCp-biotin was used to avoid the additional adenylation step. A higher labeling efficiency/yield also helps to reduce data complexity.

For 3′ end labeling, biotinylated cytidine bisphosphate (pCp-biotin) may be utilized. For this purpose, biotinylated cytidine bisphosphate (pCp-biotin) is activated by adenylation using ATP and Mth RNA ligase to produce AppCp-biotin. Then the members of the 3′ ladder pool with a free 3′ terminal hydroxyl are then ligated to the activated 5′-biotinylated AppCp via T4 RNA ligase, thus resulting in the 3′ end of each sequence in the 3′ ladder pool becoming biotin-labeled. Similarly, streptavidin-coupled beads may be used to isolate the 3′ ladder pool, which will be released for subsequent LC/MS analysis (separate from the 5′ ladder pool) after breaking the biotin-streptavidin interaction.

Although, the sequencing methods disclosed herein are generally based on the formation and sequential physical separation of 5′ and 3′ ladder pools of degraded target RNA fragments for MS analysis, the physical separation of ladder pools is not a required step. The biotin/Cy3/5 labeled RNA degraded fragments are, in some instances, more hydrophobic as compared to unlabeled RNA degraded fragments with the same length which can be differentiated by their retention time shift via the LC/MS step.

As one step in the sequence methods disclosed herein, the RNA to be sequenced is subjected to well-controlled acid hydrolysis degradation. As used herein, the terms degradation and cleavage may be used interchangeably. It is understood that the degradation, or cleavage, of RNA refers to breaks in the RNA strand resulting in fragmentation of the RNA into two or more fragments. In general, such fragmentation for purposes of the present disclosure are random along any of RNA phosphodiester bonds. However, cleavage site of any of the RNA phosphodiester bonds are specific between one nucleotide's 3′ phosphate and the adjacent nucleotide's 5′-O. Each phosphodiester hydrolysis event produces a 5′ fragment with terminal 3′(2′)-monophosphate isomers and a 3′ fragment with a 5′-hydroxyl. The reaction proceeds by nucleophilic attack of the ribose 2′-hydroxyl on the vicinal 3′-phosphodiester, resulting in a pentacoordinate transition state that can, in part, resolve by cleavage of the 5′-ester of the subsequent nucleotide, releasing a newly generated 5′-hydroxyl and yielding a cyclic 2′,3′-phosphate intermediate. Water addition to this cyclic species then gives a fragment terminating in a ribonucleotide 3′(2′)-monophosphate with a forward rate that is substantially faster than the equivalent hydroxide mediated reaction. RNA's natural tendency to be degraded can be advantageously used to generate a sequence ladder, i.e., a mass latter, for subsequent sequence determination via liquid chromatography-mass spectrometry (LC-MS). By controlling the timing of exposure to a degradation reagent, single but randomized cleavage along the target RNA molecule backbone may be achieved, thus simplifying downstream MS data analysis.

In an embodiment, chemical cleavage is accomplished through use of formic acid. Formic acid degradation is preferred because its boiling point is approximately 100° C. like water and the formic acid can be easily remove it e.g., by lyophilizer or speedvac. Such cleavage is designed to cleave the RNA molecule at its 5′-ribose positions throughout the molecule. In addition to formic acid degradation, alkaline degradation may also be used. For example, the following alkaline buffers may be used to degrade the RNA sample: 1× Alkaline Hydrolysis Buffer (e.g., 50 mM Sodium Carbonate [NaHCO₃/Na₂CO₃] pH 9.2, 1 mM EDTA; or the Alkaline Hydrolysis Buffer supplied with Ambion's RNA Grade Ribonucleases). In addition to chemical cleavage, RNAs may be subjected to enzymatic degradation. Enzymes that may be used to degrade the RNA include for example, Crotalus phosphodiesterase I, bovine spleen phosphodiesterase II and XRN-1 exoribonuclease. Such RNA degradation treatment is carried out under conditions where a desired single cleavage event occurs on the RNA molecule resulting in a pool of differently sized RNA fragments resulting in a complete ladder. Similarly, DNA can also be enzymatically degraded into ladder fragments, which can be sequenced using the MS-based sequencing.

The current disclosure provides a specific LC-MS based RNA sequencing method which can be used to simultaneously sequence different RNA nucleotide modifications together with RNA molecules with single nucleotide resolution, and to provide the information of the presence, identity, location, and quantity of each RNA modifications. The disclosed sequencing method enables complete reading of an RNA sequence from a single ladder of an RNA strand, without the need for paired-end reading from the other ladder of the RNA, and additionally allows MS sequencing of RNA mixtures with multiple different strands that contain combinatorial nucleotide modifications. By adding a hydrophobic tag at the end of the RNA, such as the 3′ end of the RNA, the labeled ladder fragments display a significant delay of t_{R}, which can help to distinguish the two mass ladders from each other and also from the noisy low-mass region. The mass-t_{R} shift caused by adding the hydrophobic tag facilitates mass ladder identification and simplifies data analysis and quantity of modifications within the RNA sample.

Together with well-controlled acid degradation, the RNA sequencing method relies on introduction of a hydrophobic end labeling strategy (HELS) into the MS-based sequencing technique. The method creates an “ideal” sequence ladder from RNA wherein each ladder fragment derives from site-specific RNA cleavage exclusively at each phosphodiester bond, and the mass difference between two adjacent ladder fragments is the exact mass of either the nucleotide or nucleotide modification at that position⁸⁻¹⁰. MS ladder derivation of the RNA sequence is facilitated because a controlled acidic hydrolysis step is included which fragments the RNA, on average, once per molecule, before it is injected into the LC-MS instrument. As a result, each degradation fragment product is detected on the mass spectrometer and all fragments together form a sequencing ladder.

Accordingly, in one aspect, a sequencing method is provided that comprises the steps of: (i) labeling of the 3′- or 5′-end of the RNA with a hydrophobic tag; (ii) well-controlled cleavage of the RNA; (iii) LC/MS measurement of resultant mass ladders with liquid chromatography (LC) and high-resolution mass spectrometry (MS); and (iv) sequence generation and modification analysis. In a specific embodiment, the 3′ end of the RNA is labeled with a hydrophobic tag.

In an embodiment, for determining presence/identification of RNA modifications an additional step may be employed that is directed to treatment of RNA with CMC. Such a method comprises the steps of: (i) treatment of RNA to be sequenced with N-cyclohexyl-N′-(2-morpholinoethyl)-carbodiimide metho-p-toluenesulfonate (CMC); (ii) labeling of the 3′ or 5′ end of the RNA with a hydrophobic tag; (iii) random non-specific cleavage of the RNA; (iv) LC-MS measurement of resultant mass ladders with liquid chromatography (LC) and high resolution mass spectrometry (MS); and (v) sequence generation and modification analysis.

To be paired with the chemical 2-D HELS method, two computational anchor algorithms are used to accomplish automated sequencing of RNAs. The signature t_{R}-mass value of the hydrophobic tag specifies the exact starting data point, the anchor, for the algorithm to accurately determine data points corresponding to the desired ladder fragments, significantly simplifying data reduction and enhancing the accuracy of sequence generation. The use of such an anchor to identify sequence ladder start-points can be generalized and extended to any known chemical moiety beyond hydrophobic tags, e.g., PO₄⁻ at the beginning of the RNA or any nucleotide with a known mass, and one can program its mass as a tag mass and use anchor algorithms for sequencing, addressing the issue of complicated MS data analysis and making 2-D HELS MS Seq more robust and accurate.

Such, non-limiting computer-implemented methods that may be used in the practice of the invention include, Anchor-based algorithm: global hierarchical ranking and local best score strategy. Because the outputs from LC-MS contain a large number of data points (>500), graph G contains the same number of vertices but a large number of edges, resulting in a large number of total paths, each representing a draft read. To effectively filter out undesired draft reads and select the desired ones, two read selection strategies were developed, global hierarchical ranking and the local best score. With either strategy, the same parameters acquired from the LC-MS dataset, e.g., volume and quality score (QS), are used to score the draft reads. With the global hierarchical ranking strategy, the draft reads are ranked after the sequence generation step with the following criteria: read length (the number of nucleobases in a draft read), average volume, average QS, and average PPM. Average volume is calculated by summing the volume associated with each data point in a draft read and dividing the sum by read length. Average QS is calculated by dividing the sum of QS by read length for each draft read. Average PPM is the sum of all PPM values associated with data points contained in a draft read divided by read length. The first step of the global hierarchical ranking strategy groups all draft reads into clusters based on their read length, and each cluster is assigned a ranking score for read length. The cluster receiving the highest ranking contains draft reads of the top read length, and the algorithm focuses on this cluster in the following steps. Within this cluster, the draft reads are assigned secondary ranking scores based on average volume values, with drafts reads of higher average volumes receiving higher rankings. In the case where more than one draft read has the same read length and average volume value, thus receiving an identical ranking, the algorithm uses the average QS value to re-rank these draft reads, with higher average QS values resulting in higher ranks. If there are still multiple draft reads receiving the same rank, the algorithm uses average PPM value to re-rank these draft reads again, but higher ranks are assigned to draft reads with lower average PPM values since PPM reflects the difference between experimental mass and theoretical mass for each data point from LC-MS. In the end, the draft read with longest read length, highest average volume, highest average QS, and lowest average PPM wins over all other draft reads in the global hierarchical ranking procedure and will be outputted as the final read for the targeted RNA fragment. Subsetting of the dataset was implemented by refining the t_{R} and mass value of the input dataset in selected windows, and specifying the starting data point of each fragment. After subsetting the dataset, the algorithm performs base-calling. The theoretical mass, calculated from the chemical formula, of all known ribonucleotides, including those with modifications to the base, is stored as a list of M_{BASE}. In the first iteration, the algorithm finds the mass corresponding to the molecular tag (anchor) and sets M_{experimental_i} equal to this mass. The algorithm tests each M_{BASE} from the list by adding it to M_{experimental_i} and generating a theoretical sum mass M_{theoretical_j}. The algorithm searches through the dataset for a mass value that matches with M_{theoretical_j}. If there exists a matching mass value M_{experimental_j}, a tuple (M_{experimental_i}, BASE, M_{experimental_j}) is stored in the result set V. Since the algorithm tests all M_{BASE} in the list and looks for all possible matches, multiple tuples with same M_{experimental_i} but a different BASE identity and M_{experimental_j} are stored in set V. When the algorithm decides if there is a match, it takes into consideration that the experimental/observed mass may slightly deviate from the theoretical mass for an identical ribonucleotide unit. A calculated parameter PPM (parts per million) was implemented that allows M_{experimental_j} be matched with M_{theoretical_j} within a customizable to range (typically <10 PPM). The algorithm performs base calling for all data points in the dataset until all possible tuples are found and stored in set V. Note that each tuple in set V represents an individual base-calling possibility. After base calling, the algorithm builds trajectories linking tuples in set V to generate draft sequence reads of the RNA. Taking tuples from set V as vertices, the algorithm finds and stores all edges by examining pairs of tuples such that for a given pair of tuples (Mᵢ, BASE, Mⱼ) and (Mₖ, BASE, Mₗ), Mₖ=Mⱼ. The algorithm generates a graph G=(V, E) after finding the edges. When graph G is completed, the algorithm finds all paths in graph G by a depth first search (DFS)^{[6]}. Since the vertices contained in the path are tuples (M_{experimental_i}, BASE, M_{experimental_j}), BASE can be outputted as a ribonucleotide unit in the RNA. All paths are stored as sets of vertices and output as a draft RNA sequence read.

Alternatively, the local best score strategy algorithm applies the anchor-based method to a specific subset of the LC-MS dataset presorted by ascending mass order. The local best score strategy differs from the previous strategy from the step of base calling. It pins down the starting ribonucleotide by a user defined anchor mass and locates data points from the entire fragment by the anchor. Focusing on these data points, the algorithm then performs base calling and simultaneously evaluates each data point. All data points in the desired zone are now considered as nodes, and the algorithm completes a single path as the final read based on the evaluation of each node. For a current node, its mass difference from the previous node (initialized as the anchor) is compared to the list of all known ribonucleotide masses for a match of identity. The match is only accepted if the PPM value of this node is below a certain threshold. In the test data with tRNA samples, a threshold was specified as 10 PPM, but it may be varied slightly to better fit the actual LC-MS dataset. After accepting or rejecting the match (or mismatch otherwise), the algorithm stores the identity of the matched ribonucleotide, and moves on to the next node. In case there are several possible proceeding nodes based on their t_{R}, the node with the highest volume will be chosen, with the exception that if a node has a significantly small PPM value (close to 0, as defined by the user) then this node will be chosen over other nodes with higher volumes. The algorithm then searches for a match of identity of the chosen node, evaluates the match, and stores the ribonucleotide identity. This process is repeated until the full sequence in the desired data zone is read out.

The presently disclosed sequencing method, where the end of the RNA is tagged with hydrophobic molecule, has the advantage that the physical separation of ladder pools is not a required step as the labeled RNA degraded fragments, i.e., a 3′ end labeled RNA, will have a retention time shift as compared to unlabeled RNA degraded fragments which can be differentiated in 2-dimensional mass-retention time plot after the LC-MS step.

Once RNA fragment pools are formed, the RNA fragments can be analyzed by any of a variety of means including liquid chromatography coupled with mass spectrometry, or gas chromatography coupled with mass spectrometry, or ion-mobility spectrometry coupled with mass spectrometry, or capillary electrophoresis coupled with mass spectrometry, or other methods known in the art. Preferred mass spectrometer formats include continuous or pulsed electrospray (ESI) and related methods or other mass spectrometer that can detect RNA fragments like MALDI-MS. HPLC-MS measurements can be performed using high resolution time-of-flight or Orbitrap mass spectrometers that have a mass accuracy of less than 5 ppm. The use of such mass spectrometers facilitates accurate discernment between cytosine and uridine bases in the RNA sequence. In one aspect of the present disclosure, the mass spectrometer is an Agilent 6550 and 1200 series HPLC with a Waters)(Bridge C18 column (3.5 μm, 1×100 mm). Mobile phase A may be aqueous 200 mM HFIP (1,1,1,3,3,3-Hexafluoro-2-propanol) and 1-3 mM TEA (Triethylamine) at pH 7.0 and mobile phase B methanol. In a specific non-limiting embodiment, the HPLC method for a 20 μL of a 10 μM sample solution was a linear increase of 2%-5% to 20%-40% B over 20-40 min at 0.1 mL/min, with the column heated to 50 or 60° C. Sample elution was monitored by absorbance at 260 nm and the eluate was passed directly to an ESI source with 325° C. drying with nitrogen gas flowing at 8.0 L/min, a nebulizer pressure of 35 psig and a capillary voltage of 3500 V in negative mode.

LC-MS data is converted into RNA ladder sequence information. The unique mass tag of each canonical ribonucleotide and its associated modifications on the RNA molecule, allows one to not only determine the primary nucleotide sequence of the RNA but also to determine the presence, type and location of RNA modifications. When an RNA is not 100%, each of the RNA ladder fragments carries stoichiometry information, which allows stoichiometric quantification of each nucleotide modification site-specifically.

Mass adducts can be removed from the deconvoluted data and the sequences will be predicted/generated using both mass and retention time data. The retention time-coupled mass data for the fragments is analyzed to determine which data points are “valid” and to be used for subsequent sequence determination and which data points are to be filtered out. After data reduction step, the mass difference (m) between two adjacent RNA fragments [m=m (i)−m(i−l), l<i<n, n=RNA length], where m(i) is the mass of any ladder fragment and m(i−l) is the preceding lower mass ladder fragment, and match such mass differences with the exact masses of known nucleotide fragments to correlate the derived RNA sequencing information based on mass differences to determine the RNA sequence and its modification. As long as the structural modification on an RNA nucleoside is mass-altering, the disclosed sequencing method will permit identification of the RNA sequence and its modification to be identified. The mass of all the known modified ribonucleosides can be conveniently retrieved from known RNA modification databases (12).

In another embodiment, an RNA sequencing technique is provided that enhances the read length and throughput, allowing direct and simultaneous sequencing of not only predominantly major RNA but also at the same time even low stoichiometric RNA, such as tRNA, tsRNA, tRNA isoforms/species directly from a complex sample without intensive sample preparation and in the presence of imperfect ladder formation. The method is based on the use of novel computational methods and tools for determining the sequence and presence of modified bases in mixtures of RNA, including those of tRNA samples.

The provided method comprises the steps of (i) controlled acid hydrolysis of the RNA to form MS ladders; and (ii) LC-MS detection of resultant acid degraded RNA samples. Additional steps are added to the method for data processing and generation of sequences and identification of modified nucleotides. Such steps include the use of one or more of different computational methods and tools including for example, conducting homology searches, identification of acid-labile nucleotide, mass-sum-based data separation, gap-filling, ladder separation, ladder complementing, and sequence generation. Details of the sequencing method are described below for tRNA molecules but it is to be understood that said method can be applied equally as well to any RNA.

The method provided herein includes as a first step, controlled RNA degradation by exposure to acid hydrolysis. In a specific embodiment of the present disclosure, formic acid, may be applied to degrade tRNA samples for producing mass ladders, according to reported experimental protocols. In a non-limiting embodiment, the tRNA sample solution may be divided into three equal aliquots for formic acid degradation using 50% (v/v) formic acid at 40° C., with one reaction running for 2 min, one for 5 min and one for 15 min. for controlled exposure of the RNA to different levels of acid hydrolysis. Ideally, the goal of the degradation step is a single cleavage of each RNA molecule resulting in a ladder of 5′- and 3-ladders that are subsequently measured thorough an LC-MS step.

In another step, the acid-hydrolyzed tRNA samples are separated and analyzed through LC-MS measurements well known to those of skill in the art. In an embodiment, on a Orbitrap Exploris 240 mass spectrometer coupled to a reversed-phase ion-pair liquid chromatography (ThermoFisher Scientific, USA) can be used using 200 mM HFIP and 10 mM DIPEA as eluent A, and methanol, 7.5 mM HFIP, and 3.75 mM DIPEA as eluent B. A gradient of 2% to 38% B in 15 minutes was used to elute RNA samples across a 2.1×50 mm DNAPac reversed-phase column. The flow rate was 0.4 mL/min, and all separates were performed with the column temperature maintained at 40° C. Injection volumes were 5-25 μL, and sample amounts were 20-200 pmol of tRNA. tRNAs were analyzed in a negative ion full MS mode from 410 m/z to 3200 m/z with a scan rate of 2 spectrum/s at 120 k resolution. The sample data is processed using the Thermo BioPharma Finder 4.0 (ThermoFisher Scientific, USA), and a workflow of compound detection with deconvolution algorithm is used to extract relevant spectral and chromatographic information from the LC-MS experiments as described previously.

One or more additional steps may be used in data processing after outputting/exporting LC-MS data of acid hydrolyzed RNA samples. One such method includes the performance of a homology search for identification of closely related tRNA isoforms that may share the same/identical precursor tRNA before post-transcriptional modifications/editing/extension/truncations, but co-exist in the RNA mixture of which are exposed to the general sequencing method. Candidate compounds are chosen based on their monoisotopic masses around the ˜24 k Da area from both before and after an acid degradation dataset (described below), and are then analyzed using a computational tool implemented in Python that divides those compounds into various groups with each group representing one specific RNA species and its related isoforms. The tool iterates over each compound in the datasets output from each LC-MS run and exams it's correlation with neighbor compounds. Compound pairs with mass differences match to specific nucleotides or modifications, such as A(329.0525 Da), C(305.0413 Da) and Methylation (14.0157 Da) get filtered out as a match, if the monoisotopic mass difference between observed value and theoretical value is within 10 ppm of for the specific known nucleotide or modification in the RNA modification database¹. Because very often, tRNAs are end with CCA at 3′ end, compounds with monoisotopic mass differences match/fit with intact mass difference 329.0525 Da would be considered as related isoforms, corresponding like to one a CCA-tailed and another CC-tailed and thus be placed into the same specific tRNA group. Similarly, compounds with monoisotopic mass differences match/fit intact mass difference 305.0413 Da would be treated as related isoforms, corresponding to CC-tailed tRNA and C-tailed tRNA and thus also be placed into the same specific tRNA group. Partial methylated/modified intact tRNA species with monoisotopic mass differences of 14.0157 Da (corresponding to a methyl) (or other specific mass value corresponding to a nucleotide modification) would be treated as related isoforms and placed into a group for sequencing.

In another embodiment, the presence of acid-labile nucleotides is identified using another computational tool implemented in Python. The tool analyzes the connections between the compounds before acid degradation and the ones after acid degradation. For each compound pair, one is before acid degradation and the other is after acid degradation, if the monoisotopic mass difference can match a mass difference calculated from the possible structural change to a specific nucleotide modification during acid hydrolysis or match the mass difference sum of a subset of different acid-labile nucleotide modifications' structural changes, the compound pair would be selected and further considered that they may contain acid-labile nucleotide modifications.

In yet another embodiment of the present disclosure, 5′- and 3′-Ladder separation of tRNAs and their acid-hydrolyzed ladder fragments in datasets output from each LC-MS run are divided into two portions, one with all 5′-ladder fragments and the other with all 3′-ladder fragments. Because every tRNA 5′ ladder fragments carry with a PO₄H₂ both at the end (5′ and 3′ end), they have relative bigger t_{R} than their counterparts 3′ fragments with the same lengths after LC separation, having an up-shift in the 2D mass-t_{R} plot. As such, most 5′ ladder fragments are located above their 3′ counterparts that have the same length in the 2D mass-t_{R} graph, forming a collective curve toward the upper right corner. Due to large amount of RNA/fragment compounds, the dividing line between two subsets of 5′- and 3′-ladder fragments is not visionally decisive in the 2D plot. Thus, a computational tool was developed to separate the 5′ and 3′ fragments. All the compounds in each LC-MS data pool are divided into two subgroup areas by circling compounds in the top collective curve of the 2D mass-t_{R} plot and marking the compounds as 5′-ladder fragment compounds, while the compounds in the bottom one as 3′-ladder fragment compounds. The purpose of selecting the top area is to include as many 5′ fragment compounds as possible while as few 3′ fragments as possible. Accordingly, the purpose of the second one is to include as many 3′ fragment compounds as possible while as few 5′ fragments as possible. Overlap between two selected ladder subgroups is inevitable, due to limited t_{R} differences between these two subgroups. The aim in the manual selection step is not to separate the 5′ and 3′ fragments with a high precision but served as two input ladder fragments for another algorithm to output 5′ and 3′ ladder fragments separately for each tRNA isoform/species. Specific ladder separation examples are described in detail below.

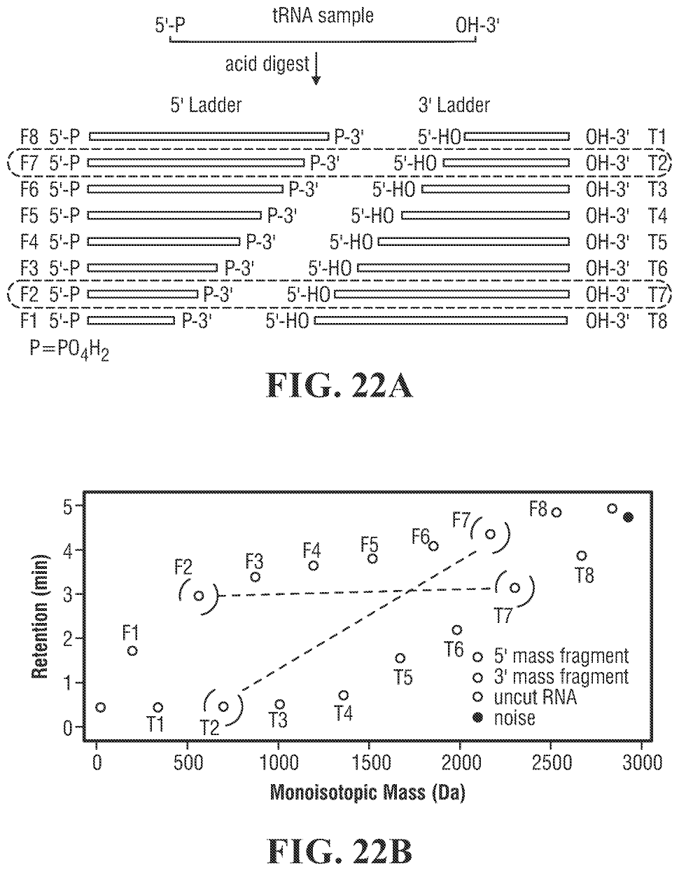

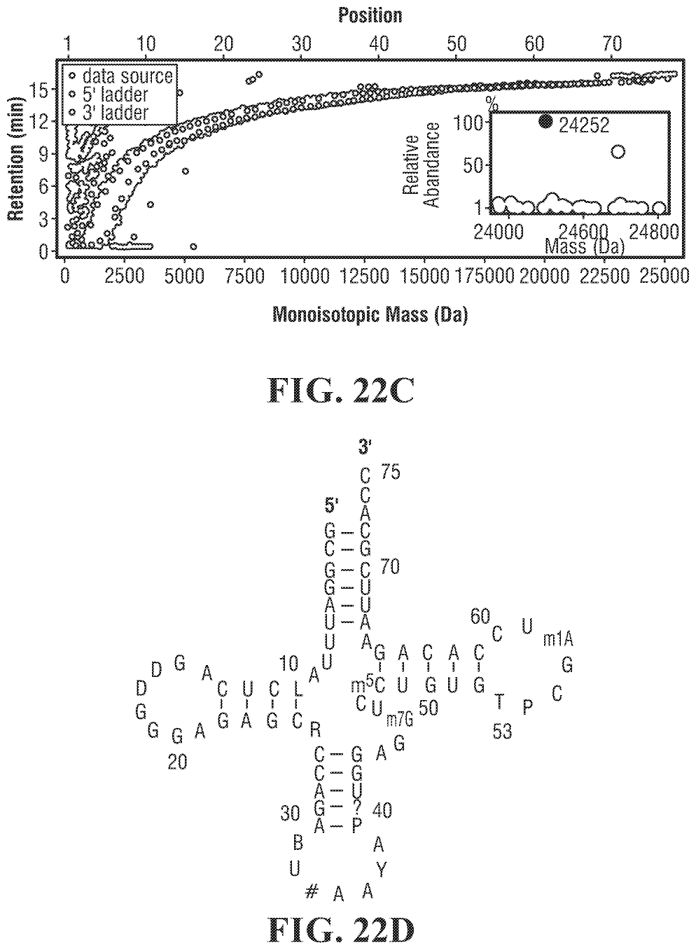

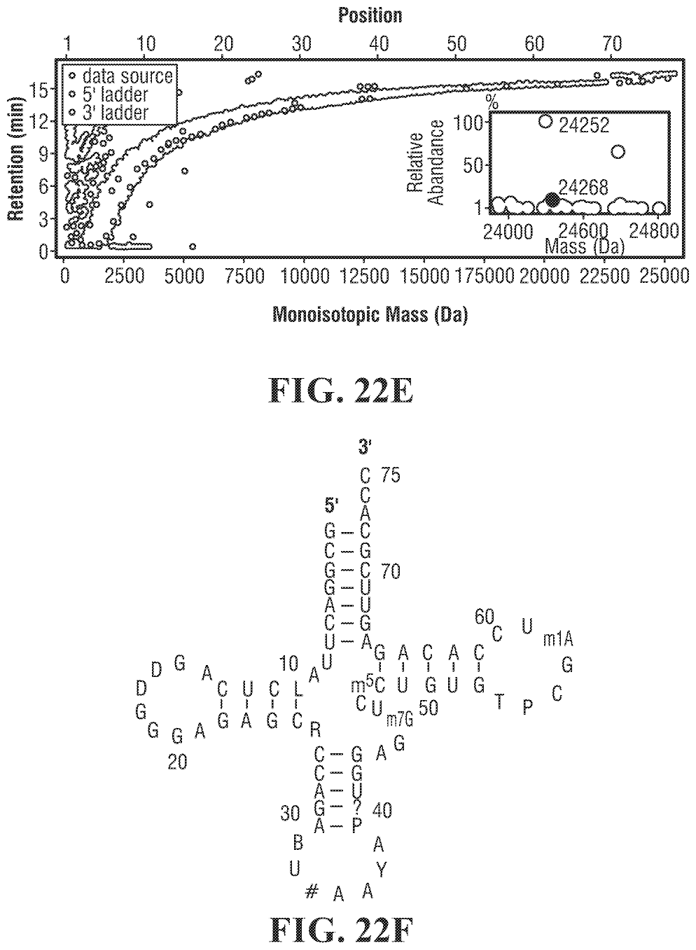

In another aspect of the present disclosure, a MassSum data separation step may be employed. MassSum is an algorithm developed based upon the acid degradation principle presented in FIG. 22. Taking advantage of the fact that each fragmented pair from two ladder groups (5′ and 3′ groups) sums up to a constant mass value that is unique to each specific tRNA isoform/specifies, the algorithm can isolate ladder compounds corresponding to a specific tRNA isoform. MassSum simplifies the dataset by grouping mass ladder components into subsets for each tRNA form/species based on its unique intact mass. Since the well-controlled acid degradation reaction cleaves RNA oligonucleotides at one specific site of the phosphodiester bond, on average, one cut per RNA, the masses of two RNA fragments (Mass_{3′ portion} and Mass_{5′ portion}) from the same strand add up to a constant value (Massₛᵤₘ).

Mass_{3′portion}+Mass_{5′portion}=Mass_{intact}+Mass_{H}_{₂}_{O}=Massₛᵤₘ (1)

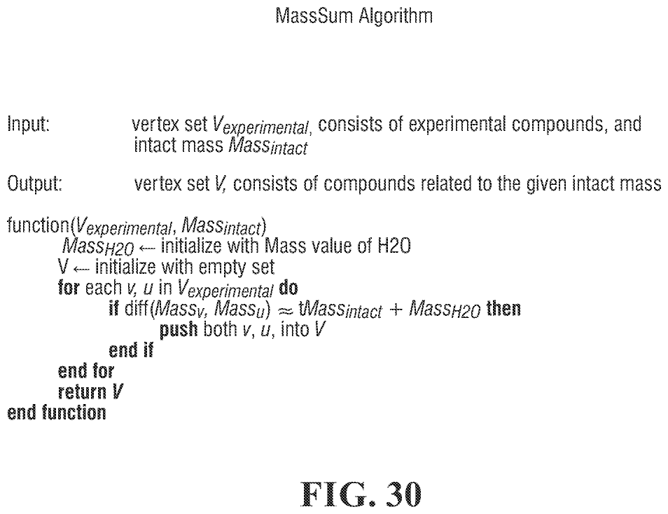

Taking the advantage of this relation between the 3′ portion and 5′ portion (Equation 1), the algorithm chooses two random compounds from the acid-degraded LC-MS dataset and adds their mass values together, one pair at a time. If the sum of the selected two compounds equals a specific Massₛᵤₘ, these two compounds will be set into the pools accordingly. The process repeats until all compound pairs have been inspected. In the end, MassSum will cluster the dataset into several groups with Mass., each group is a subset that contains 3′ and 5′ ladders of one RNA sequence. MassSum pseudocode can be found in FIG. 30.

In another embodiment of the present disclosure, a GapFill algorithm developed as a complementary of MassSum may be utilized. From the above section, it is known that MassSum handles compounds in pair, if one compound was missing from the pair, MassSum will ignore this compound as well. GapFill is designed to address this issue and can save those compounds that have counterparts missing in either 3′- or 5′-ladder (but not both). Suppose Mass_{5′i} and Mass_{5′j} are two non-adjacent compounds from the 5′ ladder, the area between these two ending compounds is defined as a gap. Among the gap there exists many compounds in degraded LC-MS dataset but not one got selected out after MassSum data separation. GapFill iterates over each potential compound in the gap in the original LC-MS dataset before MassSum, exams the mass differences of this compound and the two ending compounds with Mass_{5′i} and Mass_{5′j}. If the mass difference equal to the sum of one or more nucleobase/modifications in the RNA modification database¹, it is defined as a connection. If the compound in the gap has connections with both ending ones, this compound is kept in a candidate pool in the process later for sequencing. After iteration, GapFill calculates connections of the compounds pairwise in the candidate pool and assigns weights to them based on the frequency of each connection. The compounds that contain the highest weights would be the ones chosen to fill in the gap (See, Table S4-1).

In yet another embodiment, RNA ladders from different but related isoforms containing canonical and modified nucleotides can be used for ladder complementing in pairs or different combinations so as to obtain a complete/perfect (or close to complete) ladder that consisting of all the ladder fragments corresponding to from the 1^{st} to the last nucleotide in the RNA. After MassSum and GapFilling, each tRNA isoform has its own 5′- and 3′-ladders separately (not combined). Each ladder (5′- or 3′-) consists of a ladder sequence, and it can be read out if these ladders are perfect without missing any ladder fragment corresponding to the first to the last nucleotide in the RNA. Otherwise, if not, the ladders can be complemented from other related isoforms in order to get a more complete ladder needed for sequencing. For this step, a computational tool is used to align these ladders based on the position from the 5′→3′ direction, as long as the position has a mass/base from any ladder, this base will be called and put into the result for reporting the RNA sequence. Initially, a ladder is done complementarity separately on 5′ and 3′ ladders, resulting in one final 5′ ladder and one final 3′ ladder separately.

Dependent on the sample quality and quantity, there are cases where ladder fragments are still missing in the 5′-ladder even if ladder complementing from all other isoforms. In such cases, the 3′-ladder can also be used to fix the missing fragments site-specifically for sequence completion of the tRNA, or fix the missing piece of sequence after reading out sequences from both ladders (5′- and 3′-).

Besides 5′ and 3′ isoform ladders ladder complementing inside the 5′ or 3′ ladders (without crossing between 5′ and 3′ ladders), one may also computationally convert the 3′ ladder into its 5′ ladder based on the MassSum of each RNA isoform, and complementing converted 5′ ladder with original 5′ ladder of each RNA isoform for a perfect or better ladder needed for MS-based sequencing of RNA. Alternatively, the two 5′ and 3′ ladders can be read out separately and their overlapping sequence can be used to re-affirm each other, producing the final sequence ladder.

In some cases, it is observed that more than one ladder fragments can fit into one position when complementing ladders from different isoforms. Then one may look into the same position in the other tRNA isoform ladders (either 5′- or 3′-ladder) to ensure the one with higher confidence (the one supported more by other isoform' ladders) to get selected. This ambiguity can also be addressed later when using anchor-based sequencing algorithm to read out the final sequence based on a global hierarchical ranking strategy which is tailored to report only top-ranked sequences.

Once data separation is accomplished, an RNA sequence can be generated by manually calculating the mass differences between the two adjacent ladder components for base-calling to confirm the order of each nucleotide in the RNA sequence. The structures of RNA modifications can be found in RNA modification databases (Bjorkbom A, et al., (2015) J Am Chem Soc 137:14430-14438), and their corresponding theoretical masses are obtained by ChemDraw. PPM (parts per million) mass difference to compare the observed mass to the theoretical mass for a specific ladder component, and a value less than 10 PPM is considered a good match for base-calling.

Alternatively, an anchor based algorithm, e.g. using a phosphate as the 5′anchor, can be used to automate sequence generation separately for each tRNA isoform in mixture. The following algorithms to be used to performed the disclosed methods are described in further detail below.

Homology search algorithm. Candidate compounds were chosen based on their monoisotopic masses around the ˜24 k Da area from both before and after acid degradation dataset, and then are analyzed using a computational tool implemented in Python that divides those compounds into various groups with each group representing one specific RNA species and its related isoforms. The tool iterates over each compound in the datasets output from each LC-MS run and exams it's correlation with neighbor compounds. Compound pairs with mass differences match to specific nucleotides or modifications, such as A(329.0525 Da), C(305.0413 Da) and Methylation(14.0157 Da) get filtered out as a match, if the monoisotopic mass difference between observed value and theoretical value is within 10 ppm of for the specific known nucleotide or modification in the RNA modification database¹. Because very often, tRNAs are end with CCA at 3′ end, compounds with monoisotopic mass differences match/fit with intact mass difference 329.0525 Da would be considered as related isoforms, corresponding like to one a CCA-tailed and another CC-tailed and thus be placed into the same specific tRNA group. Similarly, compounds with monoisotopic mass differences match/fit intact mass difference 305.0413 Da would be treated as related isoforms, corresponding to CC-tailed tRNA and C-tailed tRNA and thus also be placed into the same specific tRNA group. Partial methylated/modified intact tRNA species with monoisotopic mass differences of 14.0157 Da (or other specific mass value corresponding to a nucleotide modification) would be treated as related isoforms and placed into a group for sequencing.

Algorithm for identify acid-labile nucleotides. Acid-labile nucleotides are identified using another computational tool implemented in Python. The tool analyzes the connections between the compounds before acid degradation and the ones after acid degradation. For each compound pair, one is before acid degradation and the other is after acid degradation, if the monoisotopic mass difference can match a mass difference calculated from the possible structural change to a specific nucleotide modification during acid hydrolysis or match the mass difference sum of a subset of different acid-labile nucleotide modifications, the compound pair would be selected and further considered that they may contain acid-labile nucleotide modifications.

Algorithm for 5′- and 3′-Ladder separation. A computational tool was developed to separate the 5′ and 3′ fragments. tRNAs and their acid-hydrolyzed ladder fragments in datasets output from each LC-MS run are divided into two portions, one with all 5′-ladder fragments and the other with all 3′-ladder fragments. Because every tRNA 5′ ladder fragment carries with a PO₄H₂ both at the end (5′ and 3′ end), they have relative bigger t_{R} than their counterparts 3′ fragments with the same lengths after LC separation, having an up-shift in the 2D mass-t_{R} plot. As such, most 5′ ladder fragments are located above their 3′ counterparts that have the same length in the 2D mass-t_{R} graph, forming a collective curve toward the upper right corner. Due to large amount of RNA/fragment compounds, the dividing line between two subsets of 5′- and 3′-ladder fragments is not visionally decisive in the 2D plot. Thus, a computational tool was developed to separate the 5′ and 3′ fragments. All the compounds in each LC-MS data pool were divided into two subgroup areas by circling compounds in the top collective curve of the 2D mass-t_{R} plot and marking the compounds as 5′-ladder fragment compounds, while the compounds in the bottom one as 3′-ladder fragment compounds. The purpose of selecting the top area is to include as many 5′ fragment compounds as possible while as few 3′ fragments as possible. Accordingly, the purpose of the second one is to include as many 3′ fragment compounds as possible while as few 5′ fragments as possible. Overlap between two selected ladder subgroups is inevitable, due to limited t_{R} differences between these two subgroups. The aim in the manual selection step is not to separate the 5′ and 3′ fragments with a high precision, but served as two input ladder fragments for another algorithm to output 5′ and 3′ ladder fragments separately for each tRNA isoform/species. More specific ladder separation example can be found in the Examples presented below.

Algorithm for MassSum data separation. MassSum is an algorithm developed based upon the acid degradation principle presented in FIG. 22. Taking advantage of the fact that each fragmented pair from two ladder groups (5′ and 3′ groups) sums up to a constant mass value that is unique to each specific tRNA isoform/specifies, the algorithm can isolate ladder compounds corresponding to a specific tRNA isoform. MassSum simplifies the dataset by grouping mass ladder components into subsets for each tRNA form/species based on its unique intact mass. Since the well-controlled acid degradation reaction cleaves RNA oligonucleotides at one specific site of the phosphodiester bond, on average, one cut per RNA, the masses of two RNA fragments (Mass_{3′ portion} and Mass_{5′ portion}) from the same strand add up to a constant value (Massₛᵤₘ).

Mass_{3′portion}+Mass_{5′portion}=Mass_{intact}+Mass_{H}_{₂}_{O}=Massₛᵤₘ (1)

Taking the advantage of this relation between the 3′ portion and 5′ portion (Equation 1), the algorithm chooses two random compounds from the acid-degraded LC-MS dataset and adds their mass values together, one pair at a time. If the sum of the selected two compounds equals a specific Massₛᵤₘ, these two compounds will be set into the pools accordingly. The process repeats until all compound pairs have been inspected. In the end, MassSum will cluster the dataset into several groups with Mass., each group is a subset that contains 3′ and 5′ ladders of one RNA sequence.

Algorithm for Gap Filling. GapFill is another algorithm developed as a complementary of MassSum. From the previous section it is known that MassSum handles compounds in pair, if one compound was missing from the pair, MassSum will ignore this compound as well. GapFill was designed for this case and can save those compounds have counterparts missing in either 3′- or 5′-ladder (but not both). Suppose Mass_{5′i} and Mass_{5′j} are two non-adjacent compounds from the 5′ ladder, the area between these two ending compounds is defined as a gap. Among the gap there exists many compounds in degraded LC-MS dataset but not one got selected out after MassSum data separation. GapFill iterates over each potential compound in the gap in the original LC-MS dataset before MassSum, exams the mass differences of this compound and the two ending compounds with Mass_{5′i} and Mass_{5′j}. If the mass difference equal to the sum of one or more nucleobase/modifications in the RNA modification database¹, one defines it as a connection. If the compound in the gap has connections with both ending ones, this compound would be kept into a candidate pool in the process later for sequencing. After iteration, GapFill calculates connections of the compounds pairwise in the candidate pool and assigns weights to them based on the frequency of each connection. The compounds that contain the highest weights would be the ones chosen to fill in the gap.

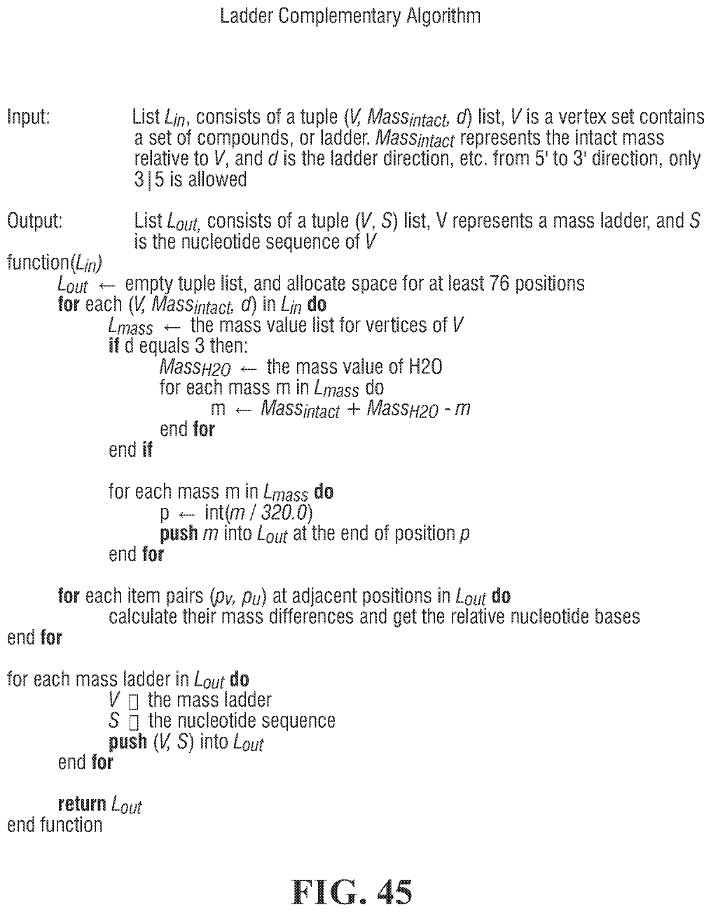

Algorithm for Ladder complementing. After MassSum and GapFilling, each tRNA isoform has its own 5′- and 3′-ladders separately (not combined). Each ladder (5′- or 3′-) consists of a ladder sequence, and one can read out if these ladders are perfect without missing any ladder fragment corresponding to the first to the last nucleotide in the RNA. Otherwise, if not, one can complement ladders from other related isoforms in order to get a more complete ladder needed for sequencing. An algorithm for ladder complementing, (FIG. 45) is used to align these ladders based on the position from the 5′ 43′ direction, as long as the position has a mass/base from any ladder, this base will be called and put into the complementary result. First, ladder complementarity is done separately on 5′ and 3′ ladders, resulting in one final 5′ ladder and one final 3′ ladder separately. If needed, the two ladders are made as a complementary to each other, producing the final sequence ladder.

Anchor-based sequencing Algorithm for RNA sequence generation. To validate and confirm the RNA sequence reads that are obtained from the previous step, the Anchor-based Sequencing Algorithm is used to read out the RNA sequence from the above-ladder complemented data. There are three main steps in the Anchor-based Sequencing Algorithm: (1) Anchor-based base calling, which detects and outputs all the canonical and modified nucleotides starting from the anchor node; (2) Depth-First Search (DFS)-based draft sequence reads generation, which connects the adjacent canonical and modified nucleotides together and outputs them as draft sequence reads; and (3) final sequence identification based on the Global Hierarchical Ranking Strategy (GHRS), in which the draft sequence reads will be ranked according to a set of ordered criteria, such as the number of canonical and modified nucleotides (a.k.a, read length), average volume, and average PPM.

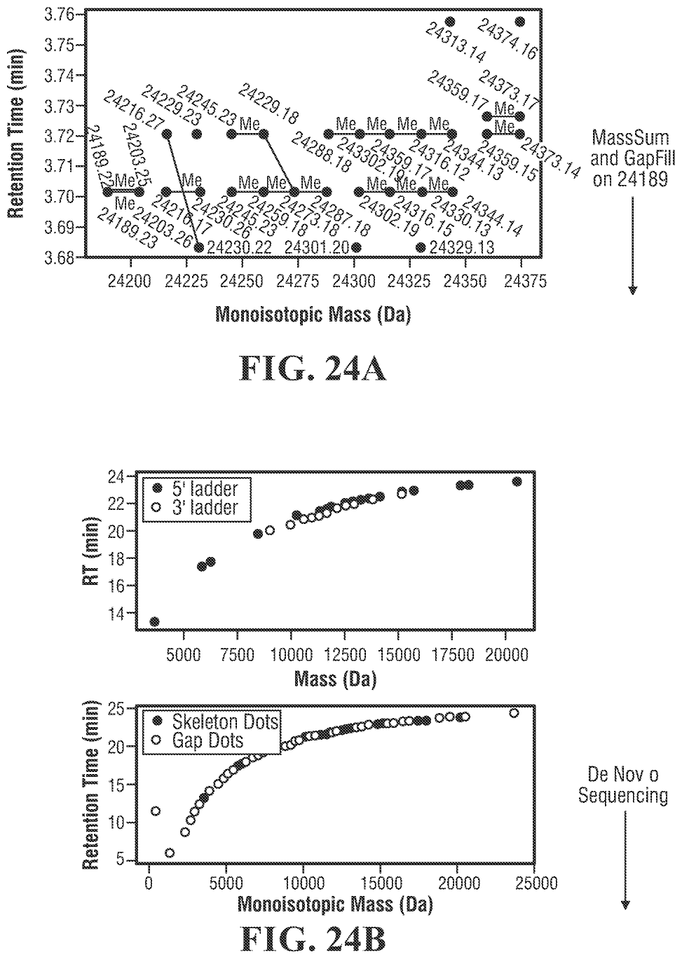

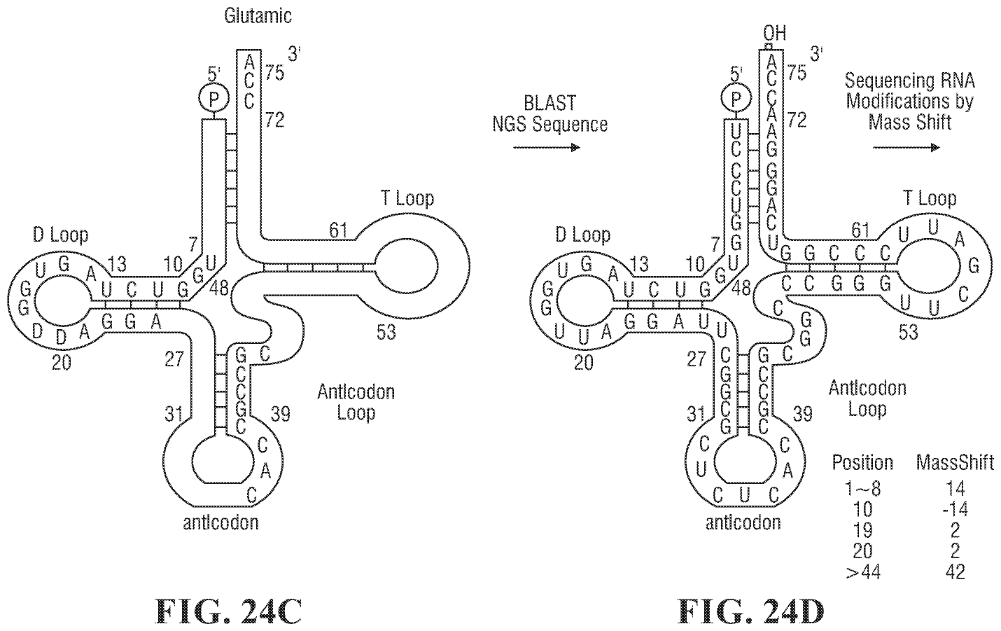

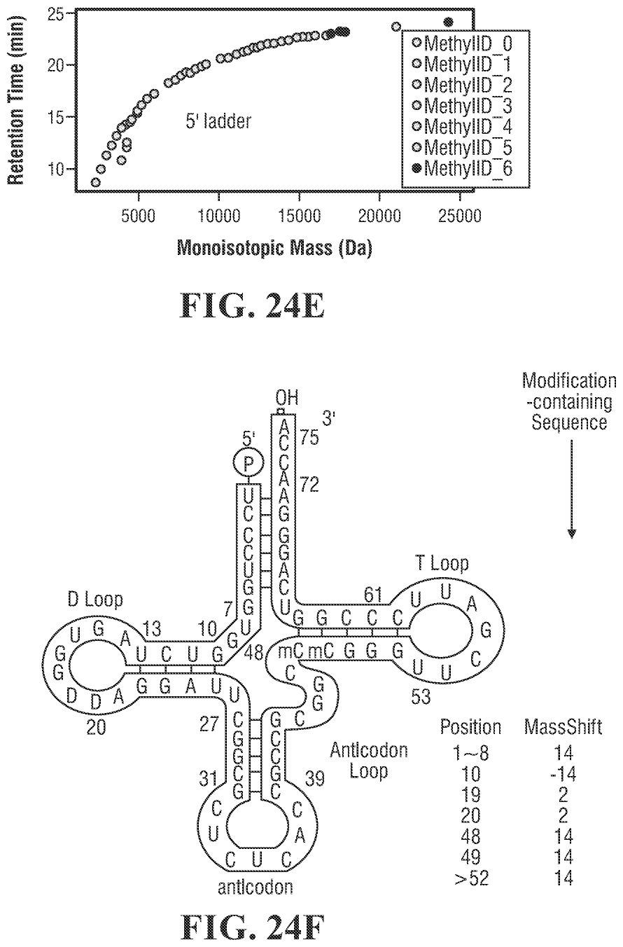

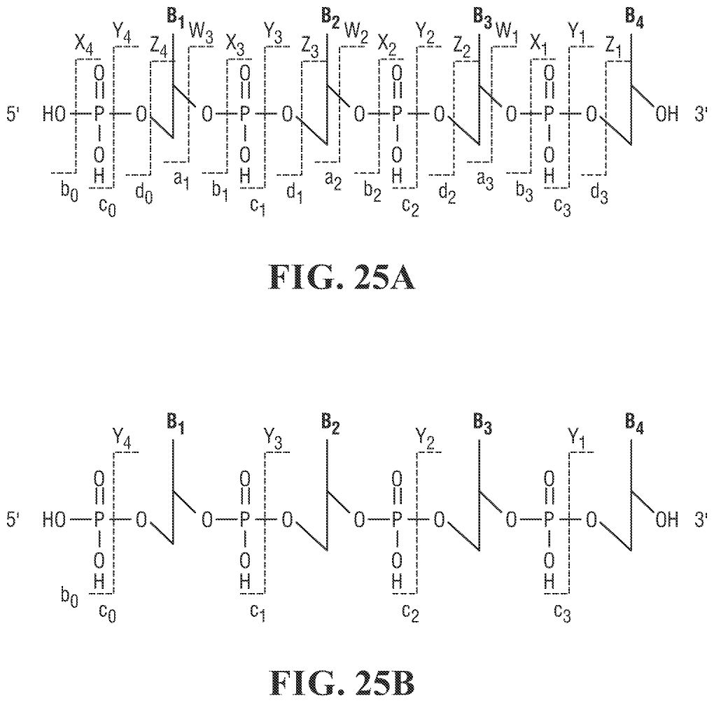

In an embodiment of the invention, Next Generation Sequencing (NGS) techniques may be combined with MS for sequencing of RNA samples such as, for example, low-abundant tRNA-Glu sample. For example, as described in detail below, after a homology search was conducted on tRNA-Glu dataset, it was noticed that most of the tRNA-Glu isoforms are related to each other, and they have either a methylation difference or a 1 Dalton mass shift. After MassSum and GapFill on the degraded dataset, one can de novo read out a couple of sequence segments (see FIG. 24A-F), e.g., 8U to 24A, and 36C to 44C. With the de novo sequencing information, one can BLAST NGS sequences dataset. Matched NGS sequences were found and the one with highest intensity was first used. One can apply different mass shifts, based on the patterns of mass differences observed, directly onto the NGS sequence and filter out the observed compounds from degraded dataset. As a result, one can sequence the entire tRNA-Glu with the different modifications from those observed compounds, which contains novel information that was not previously reported for the tRNA-Glu (see FIG. 24F).

In an embodiment, 2D-HELS MS Seq can be used reveals stoichiometry of modifications site-specifically in tRNA^{Phe}. 2D-HELS MS Seq was used to sequence commercially available yeast tRNA^{Phe} with 100% accuracy (26). tRNA^{Phe} was digested into 3 fragments with RNase T1, and each fragment was sequenced separately. The results reveal identity, position, and stoichiometry of nucleotides at the 11 known modification sites in tRNA^{Phe}. Of these 11 RNA modification sites, five positions that were not 100% modified. For example, the wobble Gm at position 34 (60% modified), has regulatory implications since the lack of Gm could affect codon recognition and thus stalling of the ribosome. Other partially modified nucleotides include m⁷G at position 46, m¹A at position 58, and wybutosine (Y-base) at position 37. An a basic form called Y′ was found, in which the wybutosine base is replaced with a OH. The method discovered unexpected nucleotides in this tRNA. Position 26 in tRNA^{Phe} is thought to be m²₂G; however, clear evidence shows G co-exists at this position, but no evidence was found for any monomethyled G (mG) co-existing at this position. The stoichiometries were quantified by integrating extracted-ion current (EIC) peaks of their corresponding ladder fragments (24, 45), which revealed that m²₂G and G were present at 58% and 42%, respectively. Furthermore, both m⁷G at position 46 (46% m⁷G vs. 54% G) in the variable loop and m¹A at position 58 (94% m¹A vs. 6% A) in the TψC loop were partially modified, suggesting that the methylation process is highly regulated. Several tRNA^{Phe} isoforms were discovered that were missing one 3′ residue, and some missing two 3′ residues.

The present disclosure provides a computer-implemented method for determining an order of nucleotides and/or modifications of an RNA molecule, wherein the method includes: receiving/exporting liquid chromatography-mass-spectrometry (LC-MS) data of an RNA sample, the LC-MS data including but not limited to a mass (e.g., m/z, monoisotopic mass, average mass), charge states, retention time (RT), Height, width, volume, relative abundance, and quality score (QS); filtering the LC-MS data based on mass, the filtering including removing masses smaller than a predetermined size; analyzing the filtered LC-MS data, to determine a plurality of RNA sequences, analyzing the filtered LC-MS data including: determining a mass difference between at least two adjacent ladder fragments; and determining whether the mass difference is equal to at least one of a canonical nucleotide, or a modified nucleotide (known or unknown); and reading-out an RNA sequence as a sequence read after determining no remaining valid nucleotides in the remaining LC-MS data, the RNA sequence including a sequence order of each identified canonical nucleotide and any identified modified nucleotides.

In an embodiment of the invention, a computer-implemented sequencing method is provided for determining the Mass Sum of any of two ladder fragments; and if the mass sum is equal to the mass of the intact RNA (detected in homology search) plus the mass of a water, isolating these two fragments into a pair based on the determined MassSum for sequencing of the RNA molecule. In an embodiment, MassSum may not be related to any two adjacent ladder fragments. Further, MassSum may not be limited to computational separate ladder fragments generated by one cleave per RNA molecule but may also be used to separate other fragments of RNA that gets cleaved more than once.

In another embodiment, a computer-implemented method is provided comprising the step of determining if any of the two ladder fragments cannot pair based on the mass sum value for a given RNA, and if so finding one of them by use of a GapFill algorithm, configured to search for ladder fragments missed by MassSum determination.

In yet another embodiment, the computer-implemented method comprises a step for identifying tRNA isoforms based on a homology search function configured to divide the intact RNA molecules into two or more groups with each group representing one specific RNA species and its related isoforms. In such an embodiment, the homology search can be performed before or after degradation of the RNA.

In another embodiment, the computer-implemented method comprises the step of determining presence, type, location, or quantity of the modified nucleotides within the RNA molecule.

In an embodiment, a computer-implemented method is provided comprising the step of separating the 5′- and 3′ end fragments of each identified tRNA isoform based on breaking two adjacent sigmoidal curves into two isolated curves.

In an embodiment of the invention, a computer-implemented method is provided comprising the step of completing a faulted mass ladder by complementing the missing ladder fragments from related tRNA isoforms identified in a homology search.

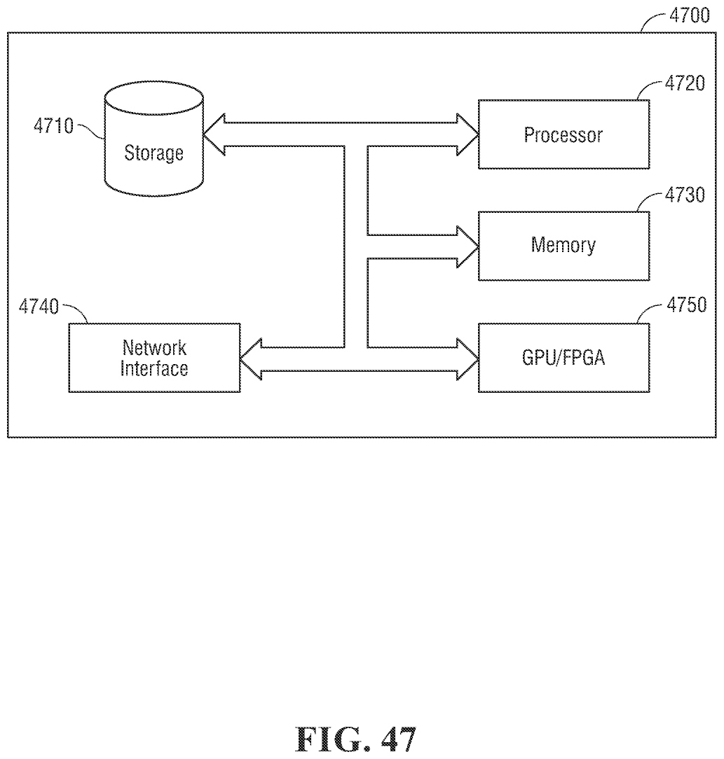

FIG. 47 illustrates that controller 4700 includes a processor 4720 connected to a computer-readable storage medium or a memory 4730 configured for performing various functions of the present disclosure. The computer-readable storage medium or memory 4730 may be a volatile type of memory, e.g., RAM, or a non-volatile type memory, e.g., flash media, disk media, etc. In various aspects of the disclosure, the processor 4720 may be another type of processor such as a digital signal processor, a microprocessor, an ASIC, a graphics processing unit (GPU), a field-programmable gate array (FPGA), or a central processing unit (CPU). In certain aspects of the disclosure, network inference may also be accomplished in systems that have weights implemented as memristors, chemically, or other inference calculations, as opposed to processors.

In aspects of the disclosure, the memory 4730 can be random access memory, read-only memory, magnetic disk memory, solid-state memory, optical disc memory, and/or another type of memory. In some aspects of the disclosure, the memory 4730 can be separate from the controller 4700 and can communicate with the processor 4720 through communication buses of a circuit board and/or through communication cables such as serial ATA cables or other types of cables. The memory 4730 includes computer-readable instructions that are executable by the processor 4720 to operate the controller 4700. In other aspects of the disclosure, the controller 4700 may include a network interface 4740 to communicate with other computers or to a server. A storage device 4710 may be used for storing data.

The disclosed method may run on the controller 4700 or on a user device, including, for example, on a mobile device, an IoT device, an embedded processor, and/or a server system.

In various aspects, the controller can be coupled to a mesh network. As used herein, a “mesh network” is a network topology in which each node relays data for the network. All mesh nodes cooperate in the distribution of data in the network. It can be applied to both wired and wireless networks. Wireless mesh networks can be considered a type of “Wireless ad hoc” network. Thus, wireless mesh networks are closely related to Mobile ad hoc networks (MANETs). Although MANETs are not restricted to a specific mesh network topology, Wireless ad hoc networks or MANETs can take any form of network topology. Mesh networks can relay messages using either a flooding technique or a routing technique. With routing, the message is propagated along a path by hopping from node to node until it reaches its destination. To ensure that all its paths are available, the network must allow for continuous connections and must reconfigure itself around broken paths, using self-healing algorithms such as Shortest Path Bridging. Self-healing allows a routing-based network to operate when a node breaks down or when a connection becomes unreliable. As a result, the network is typically quite reliable, as there is often more than one path between a source and a destination in the network. This concept can also apply to wired networks and to software interaction. A mesh network whose nodes are all connected to each other is a fully connected network.

In some aspects, the controller may include one or more modules. As used herein, the term “module” and like terms are used to indicate a self-contained hardware component of the central server, which in turn includes software modules. In software, a module is a part of a program. Programs are composed of one or more independently developed modules that are not combined until the program is linked. A single module can contain one or several routines, or sections of programs that perform a particular task.

Any of the herein described methods, programs, algorithms or codes may be converted to, or expressed in, a programming language or computer program. The terms “programming language” and “computer program,” as used herein, each include any language used to specify instructions to a computer, and include (but is not limited to) the following languages and their derivatives: Python, Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions

Each of the reference cited within the specification are hereby incorporated by reference in their entirety. Incorporated by reference herein in their entirety are WO2019/226990 and WO2019/226976.

### Example 1

Mass spectrometry (MS)-based sequencing approaches have been shown to be useful in direct sequencing of RNA without the need for a complementary DNA (cDNA) intermediate. However, such approaches are rarely applied as a de novo RNA sequencing method but used mainly as a tool that can assist in quality assurance for confirming known sequences of purified single-stranded RNA samples. A direct RNA sequencing method has been developed by integrating a 2-dimensional mass-retention time hydrophobic end-labeling strategy into MS-based sequencing (2D-HELS MS Seq). This method is capable of accurately sequencing single RNA sequences as well as mixtures containing up to 12 distinct RNA sequences. In addition to the four canonical ribonucleotides (A, C, G, and U), the method has the capacity to sequence RNA oligonucleotides containing modified nucleotides. This is possible because the modified nucleobase either has an intrinsically unique mass that can help in its identification and its location in the RNA sequence, or it can be converted into a product with a unique mass. As described in this example, RNA has been used, incorporating two representative modified nucleotides (pseudouridine (T) and 5-methylcytosine (m⁵C)), to illustrate the application of the method for the de novo sequencing of a single RNA oligonucleotide as well as a mixture of RNA oligonucleotides, each with a different sequence and/or modified nucleotides. The procedures and protocols described herein for sequencing these RNAs is applicable to other short RNA samples (<35 nt) when using a standard high-resolution LC-MS system, and can also be used for sequence verification of modified therapeutic RNA oligonucleotides.

### Materials and Methods

Design RNA oligonucleotides. Synthetic RNA oligonucleotides were designed with different lengths (19 nt, 20 nt and 21 nt), including one (RNA #6) with both canonical and modified nucleotides. ψ is employed as a model for non-mass-altering modifications, which is challenging for MS sequencing because it has an identical mass to U. m⁵C is chosen as a model for mass-altering modifications to demonstrate the robustness of the approach.

RNA #1:
(SEQ ID NO: 1)
5′-HO-CGCAUCUGACUGACCAAAA-OH-3′
RNA #2:
(SEQ ID NO: 2)
5′-HO-AUAGCCCAGUCAGUCUACGC-OH-3′
RNA #3:
(SEQ ID NO: 3
5′-HO-AAACCGUUACCAUUACUGAG-OH-3′
RNA #4:
(SEQ ID NO: 4)
5′-HO-GCGUACAUCUUCCCCUUUAU-OH-3′
RNA #5:
(SEQ ID NO: 5)
5′-HO-GCGGAUUUAGCUCAGUUGGGA-OH-3′
RNA #6:
(SEQ ID NO: 6)
5′-HO-AAACCGUψACCAUUAm⁵CUGAG-OH-3′

Each synthetic RNA was dissolved in nuclease-free diethyl pyrocarbonate (DEPC)-treated water (expressed as DEPC-treated H₂O unless otherwise indicated) to obtain a 100 μM RNA stock solution. Stock solutions are stored long-term at −20° C. To avoid possible RNA sample degradation, RNase-free experimental supplies are used including DEPC-treated water, microcentrifuge tubes, and pipette tips. Frequently wipe down OF surfaces of lab supplies using RNase elimination wipes.

Label the 3′-end of RNAs with biotin. A two-step reaction protocol (adenylation and ligation) was used as follows. Add 1 μL of 10× adenylation reaction buffer containing 50 mM sodium acetate, pH 6.0, 10 mM MgCl2, 5 mM dichlorodiphenyltrichloroethane (DTT), 0.1 mM ethylenediaminetetraacetic acid (EDTA), 1 μL of 1 mM ATP, 1 μL of 100 μM biotinylated cytidine bisphosphate (pCp-biotin), 1 μL of 50 μM Mth RNA ligase, and 6 μL of DEPC-treated H₂O (a total volume of 10 μL) into an RNase-free thin-walled 0.2 mL PCR tube. Reagents were stored at −20° C. before the two-step reaction. Thaw the reagents at room temperature and mix well by vortexing and centrifuging before adding to the reaction. Incubate the reaction in a PCR machine at 65° C. for 1 h and inactivate the reaction at 85° C. for 5 min. Conduct the ligation step in an RNase-free, thin walled 0.2 mL PCR tube containing 10 μL of reaction solution from the previous step by adding 3 μL of 10× T4 RNA ligase reaction buffer containing 50 mM tris(hydroxymethyl)aminomethane (Tris)-HCl, pH 7.8, 10 mM MgCl₂, 1 mM DTT, 1.5 μL of the 100 μM sample stock of the RNA to be sequenced, 3 μL of anhydrous dimethyl sulfoxide (DMSO) to reach 10% (v/v), 1 μL of T4 RNA ligase (10 units/μL), and 11.5 μL of DEPC-treated H₂O (for a total volume of 30 μL). Incubate the reaction overnight at 16° C. in a PCR machine. Combine reaction components at room temperature due to the high freezing point of DMSO (18.45° C.). Incubate the reaction overnight at 16° C. Quench and purify the reaction by column purification to remove enzymes and free pCp-biotin using Oligo Clean & Concentrator (Zymo Research, Irvine, Calif., USA). Oligo Binding Buffer, DNA Wash Buffer, spin columns and collection tubes are provided in the kit. Add 20 μl, of DEPC-treated H₂O to the reaction solution to reach a 50 μl, sample volume prior to adding the Binding Buffer. Add 100 μl, of binding buffer to each reaction solution. Add 400 μL of ethanol, mix by pipetting, and transfer the mixture to the column. Centrifuge at 10,000×g for 30 s. Discard the flow-through. Add 750 μL of DNA Wash Buffer to the column. Centrifuge at 10,000×g and maximum speed for 30 s and 1 minute, respectively. Transfer the column to a 1.5 mL microcentrifuge tube. Add 15 μL of DEPC-treated H₂O to the column and centrifuge at 10,000×g for 30 s to elute the RNA product.

Samples can be stored at −20° C. at this stage until the next step is performed.

A one-step reaction protocol may be used as follows. Performance of a one-step labeling reaction was conducted by combining 2 μL of 150 μM adenosine-5′-5′-diphosphate-{5′-(cytidine-2′-O-methyl-3′-phosphate-TEG}C-biotin (AppCp-biotin), 3 μL of 10× ligase reaction buffer, 1.5 μL of the 100 μM sample stock of the RNA to be sequenced, 3 μL of anhydrous DMSO to reach 10% (v/v), 1 μL of T4 RNA ligase (10 units/μL), and 19.5 μL of DEPC-treated H₂O (for a total volume of 30 μL) in a 1.5 mL RNase-free microcentrifuge tube. The reaction was incubated overnight at 16° C. in a PCR machine. Column purification was performed as described above. A separate/exclusive reaction tube was prepared for each RNA sample (150 pmol scale of RNA). Labeling of the 5′-end of the RNA(s) with sulfo-Cyanine3 (Cy3) or Cy3 may be needed (e.g., for bidirectional sequencing verification). The method is different than that of 3′-biotinylation and is described in a previous publication⁹.

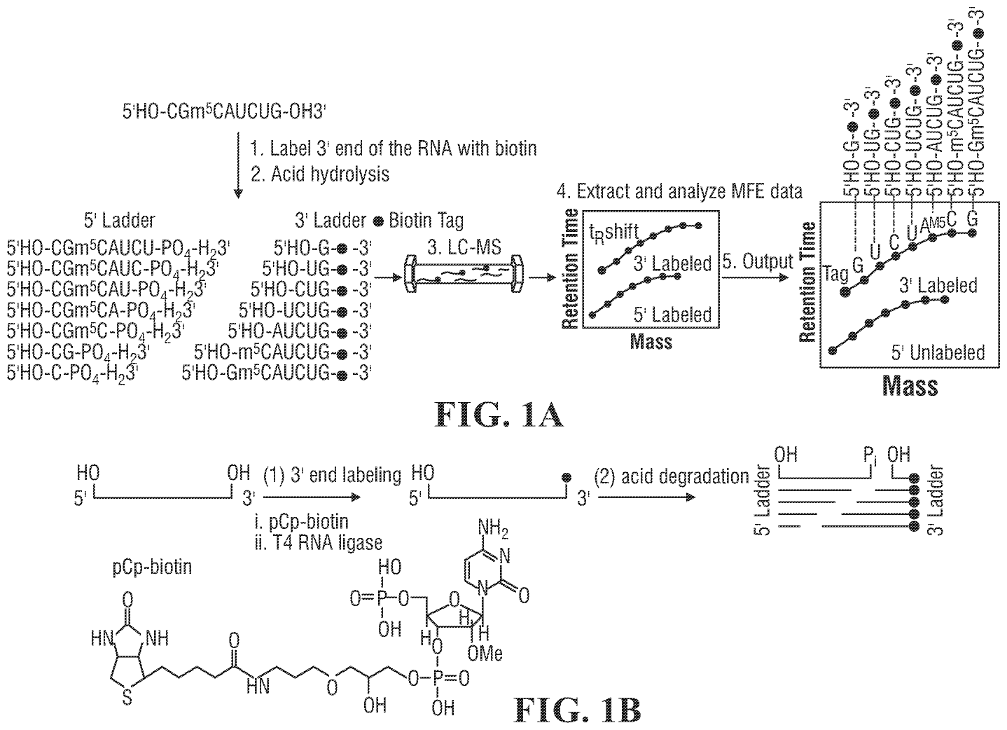

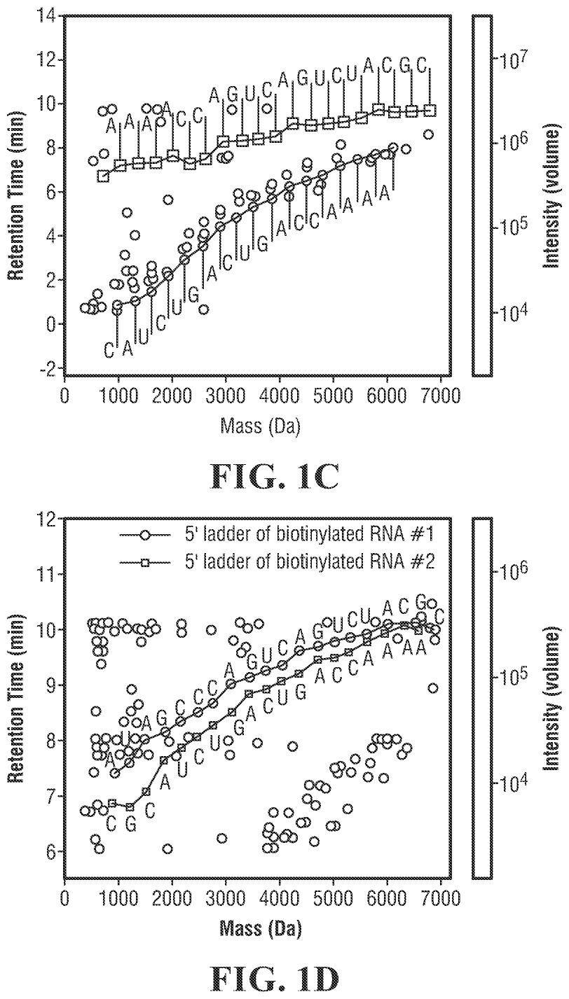

Capture of biotinylated RNA sample on streptavidin beads. Capture was achieved as follows. Activate 200 μL of streptavidin Cl magnet beads by adding 200 μL of 1× B&W buffer (5 mM Tris-HCl, pH 7.5, 0.5 mM EDTA, 1 M NaCl) in a 1.5 mL RNase-free microcentrifuge tube. Vortex this solution and place it on a magnet stand for 2 min. Then discard the supernatant by carefully pipetting out the solution. Wash the beads twice with 200 μL of Solution A (DEPC-treated 0.1 M NaOH and DEPC-treated 0.05 M NaCl) and once in 200 μL of Solution B (DEPC-treated 0.1 M NaCl). For each wash step, vortex the solution and place it on a magnet stand for 2 min, followed by discarding of the supernatant. Then add 100 μL of 2× B&W buffer (10 mM Tris-HCl, pH 7.5, 1 mM EDTA, 2 M NaCl). Add 1× B&W buffer to the biotinylated RNA sample until the volume is 100 μL. Then add this solution to the washed beads stored in 100 μL of 2× B&W buffer. Incubate for 30 min at room temperature on a rocking platform shaker at 100 rpm. Place the tube on a magnet stand for 2 min and discard the supernatant. Wash the coated beads 3 times in 1× B&W buffer and measure the final concentration of supernatant in each wash step by Nanodrop for recovery analysis, to confirm that the target RNA molecules remain on the beads. Incubate the beads in 10 mM EDTA, pH 8.2 with 95% formamide at 65° C. for 5 min in a PCR machine. Keep the tube on the magnet stand for 2 min and collect the supernatant (containing the biotinylated RNAs released from the streptavidin beads) by pipet. This physical separation step prior to acid degradation is only used for sequencing of RNA #1 in FIG. 1C and is not mandatory for the 2D-HELS MS Seq since the hydrophobic biotin label can cause the 3′-labeled ladder fragments to have a significantly delayed t_{R} during LC-MS measurement, which can clearly distinguish the labeled 3′-ladder fragments from the unlabeled 5′-ladder fragments in the 2D mass-t_{R} plot.

Acid hydrolysis of RNA to generate MS ladders for sequencing. Hydrolysis of RNA was done as follows. Divide each RNA sample into three equal aliquots. For instance, divide an RNA sample with a volume of 15 μL RNA sample into three aliquots of 5 μL. Add an equal volume of formic acid to achieve 50% (v/v) formic acid in the reaction mixture (Bjorkbom, A. et al, 2015 Journal of the American Chemical Society 137 (45) 1443014438) Incubate the reaction at 40° C. in a PCR machine, with one reaction running for 2 min, one for 5 min, and one for 15 min, respectively. Quench the acid degradation by immediately freezing the sample on dry ice after each reaction finishes. Use a centrifugal vacuum concentrator to dry the sample. The sample is typically completely dried within 30 min, and formic acid is removed together with H₂O during the drying process because formic acid has a boiling point (100.8° C.) similar to that of H₂O (100° C.). Suspend and combine a total of three dried samples in 20 μL of DEPC-treated H₂O for LC-MS measurement. Samples can be stored at −20° C. at this stage while waiting for LC-MS measurement.

Conversion of ψ to CMC-ψ adduct. Conversion was achieved as follows. Add 80 of DEPC-treated H₂O into a 1.5 mL RNase-free microcentrifuge tube containing 0.0141 g of N-cyclohexyl-N′-(2-morpholinoethyl)-carbodiimide metho-p-toluenesulfonate (CMC) and 0.07 g of urea. Add 10 μL of the 100 μM sample stock of the RNA to be sequenced, 8 μL of 1 M bicine buffer (pH 8.3), and 1.28 μL of 0.5 M EDTA. Add DEPC-treated H₂O to reach a total volume of 160 μL. Final concentrations are 0.17 M CMC, 7 M urea, and 4 mM EDTA in 50 mM bicine (pH 8.3)¹¹. This protocol is applicable to either a single synthetic RNA sequence or RNA mixtures. Divide the 160 μL reaction solution into four equal aliquots in RNase-free, thin walled 0.2 mL PCR tubes and incubate at 37° C. for 20 min in a PCR machine. 50 μL per tube is the maximum reaction volume that can be used in a PCR machine. Quench each reaction with 10 μL of 1.5 M sodium acetate and 0.5 mM EDTA (pH 5.6). Perform column purification with four parallel spin columns to remove excessive reactants according to the procedure as described in steps 2.1.5-2.1.8. Dissolve the purified product in 15 μL of DEPC-treated H₂O in each 1.5 mL RNase-free microcentrifuge tube. Transfer the purified product to four RNase-free, thin walled 0.2 mL PCR tubes. Add 20 of 0.1 M Na₂CO₃ buffer (pH 10.4) into each 15 μL of purified product and add DEPC-treated H₂O to make a final volume of 40 μL for each reaction tube (in total four tubes). Incubate the reaction at 37° C. for 2 h in a PCR machine. Quench and purify the reaction by column purification with four parallel spin columns as described above. Elute the CMC-ψ converted product to a 1.5 mL RNase-free microcentrifuge tube each with 15 μL of DEPC-treated H₂O. Combine the purified CMC-ψ converted sample from four collection tubes into one tube. Perform formic acid degradation 50% (v/v) according to the procedures as described above to generate MS ladders for sequencing.

LC-MS measurement. LC-MS measurement was done as follows. Prepare mobile phases for LC-MS measurement. Mobile phase A is 25 mM hexafluoro-2-propanol with 10 mM diisopropylamine in LC-MS grade water; mobile phase B is methanol. Transfer the sample to LC-MS sample vial for analysis. Each sample injection volume is 20 μL containing 100-400 pmol of RNA. Use the following LC conditions: column temperature of 35° C., flow rate of 0.3 mL/min; a linear gradient from 2-20% mobile phase B over 15 min followed by a 2 min wash step with 90% mobile phase B. For more hydrophobic end-labels such as Cy3 and sulfo-Cy3 as mentioned in Section 2, a higher percentage of organic solvent may be necessary for sample elution (i.e., a similar gradient can be used but with an increased percentage range of mobile phase B). For instance, 2-38% mobile phase B over 30 min with a 2 min wash step with 90% mobile phase B. Separate and analyze samples on an Agilent Q-TOF (Quadrupole Time-of-Flight) mass spectrometer coupled to an LC system equipped with an autosampler and an MS HPLC (High Performance Liquid Chromatography) system. The LC column is a 50 mm×2.1 mm C18 column with a particle size of 1.7 μm. Use the following MS settings: negative ion mode; range, 350 m/z to 3200 m/z; scan rate, 2 spectra/s; drying gas flow, 17 L/min; drying gas temperature, 250° C.; nebulizer pressure, 30 psig; capillary voltage, 3500 V; and fragmentor voltage, 365 V. Please note that these parameters are specific to the type or model of mass spectrometer being used. Acquire data with Agilent MassHunter acquisition software. Use Agilent molecular feature extraction (MFE) workflow to extract compound information including mass, retention time, volume (the MFE abundance for the respective ion species), and quality score, etc. Use the following MFE settings: “centroid data format, small molecules (chromatographic), peak with height ≥100, up to a maximum of 1000, quality score ≥50”. Optimize MFE settings to extract as many potential compounds as possible, up to a maximum of 1000, with quality scores of ≥50.

Automate RNA sequence generation by a computer-implemented method. This procedure is shown for sequencing of RNA #1 in FIG. 1C. Sort MFE extracted compounds in order of decreasing volume (peak intensity) and t_{R}. Perform data pre-selection via 1) setting t_{R} from 4 to 10 min to select the RNA fragments labeled by the biotin, since the t_{R}s of the biotin-labeled mass ladder components are shifted to this t_{R} window (4 min to 10 min), and 2) using an order-of-magnitude higher of input compounds than the number of ladder fragments for algorithm computation to reduce data amount based on volume. For instance, for a 20 nt RNA, 20 labeled mass-t_{R} ladder components will be required for sequencing of the 20 nt RNA, and thus, 200 compounds from MFE data file will be selected based on volume. Please note that the t_{R} window may be different when a different type or model of mass spectrometer is used. Perform data processing and sequence generation of RNA #1 using a revised version of a published algorithm (Bjorkbom, A. et al, 2015 Journal of the American Chemical Society 137 (45) 1443014438). The source codes of the revised algorithm are described previously by Zhang, N. et al. Nucleic Acids Research. 47 (20), e125 (2019).

In addition to automating sequence generation using the algorithm, manually calculate the mass differences between two adjacent ladder components for base calling. All bases in the RNA can be called manually and matched with the theoretical ones in the RNA nucleotide and modification database (Bjorkbom, A. et al, 2015 Journal of the American Chemical Society 137 (45) 1443014438); thus, the complete sequence of the RNA strand can be accurately read out manually, which is used to confirm the accuracy of the algorithm-reported sequence read. More structures of RNA modifications can be found in RNA modification databases¹², and their corresponding theoretical masses are obtained by ChemBioDraw. In Table S1-1 through S1-6, the ppm (parts-per-million) mass difference is shown when comparing the observed mass to its theoretical mass for a specific ladder component, and a value less than 10 ppm is considered a good match for each base calling. See, Table S1-1 and Table S2-2

Sequencing RNA mixtures. Label a mixture of five RNA strands (RNA #1 to #5) at their 3′-ends with A(5)pp(5′)Cp-TEG-biotin using a one-step protocol described in step 2.2. In a total volume of 150 μL reaction solution, add 15 μL of 10× T4 RNA ligase reaction buffer, 1.5 μL of each RNA strand (100 μM stock of RNA #1 to #5, respectively, for a total volume of 7.5 μL), 10 μL of 150 μM A(5′)pp(5′)Cp-TEG-biotin, 15 μL of anhydrous DMSO, 5 μL of T4 RNA ligase (10 units/μL), and 97.5 μL of DEPC-treated H₂O. Equally distribute the reaction solution into five aliquots. Each RNase-free microcentrifuge tube contains 30 μL of reaction solution. Incubate the reaction overnight at 16° C. in a PCR machine. Perform column purification according to the procedure as described above with five parallel spin columns. Elute a mixture sample of 3′-biotinylated 5 RNA strands (mixture of RNA #1 to #5) to a 1.5 mL RNase-free microcentrifuge tube each with 15 μL of DEPC-treated H₂O. Combine the purified mixture samples from the five collection tubes into one tube. Perform formic acid degradation according to the procedure described above. Measure samples by LC-MS as described above, and analyze the data using the data analysis software with optimized MFE settings to extract data containing mass, t_{R}, and volume as described above. The typical processing and base-calling algorithm is not applied due to the significantly increased data complexity resulting from the mixture. All bases in the RNA of the mixed sample are called manually in a method similar to above and match well with the theoretical ones in the RNA nucleotide and modification database (Bjorkbom, A. et al, 2015 Journal of the American Chemical Society 137 (45) 1443014438), thus the complete sequences of all five RNA strands in the mixed sample are accurately read out. In Table S1-7 through S1-11, all information is listed including observed mass, t_{R}, volume, quality score and ppm mass difference.

### Results

Introducing a biotin tag to the 3′-end of RNA to produce easily-identifiable mass-t_{R} ladders. The workflow of the 2D-HELS MS Seq approach is demonstrated in FIG. 1A. The hydrophobic biotin label introduced to the 3′-end of the RNA increases the masses and t_{R}s of the 3′-labeled ladder components when compared to those of their unlabeled counterparts. Thus, the 3′-ladder curve is shifted to greater y-axis values (due to the increase in the t_{R}s) and shifted to greater x-axis values (due to the increase in masses) in the 2D mass-t_{R} plot. FIG. 1B shows the sample preparation protocol including introducing a biotin tag to the 3′-end of RNA for 2D-HELS MS Seq. FIG. 1C demonstrates separation of the 3′-ladder from the 5′-ladder and other undesired fragments on a 2D mass-t_{R} plot based on systematic changes in t_{R}s of the 3′-biotin-labeled mass-t_{R} ladder fragments of RNA #1. The 3′-ladder curve alone gives a complete sequence of RNA #1, and the 5′-ladder curve that does not show a t_{R} shift provides the reverse sequence, but it requires end-pairing for reading the terminal base (Bjorkbom, A. et al, 2015 Journal of the American Chemical Society 137 (45) 1443014438). With this strategy of 2D-HELS, end-pairing is not required as reported before and the entire RNA sequence can be read out completely from only one labeled ladder curve (Bjorkbom, A. et al, 2015 Journal of the American Chemical Society 137 (45) 1443014438). As such, it is possible to sequence mixed samples containing multiple RNAs, e.g., two RNA strands of different lengths (RNA #1 and RNA #2, 19 nt and 20 nt, respectively) with a 5′-biotin label at each RNA (FIG. 1D).

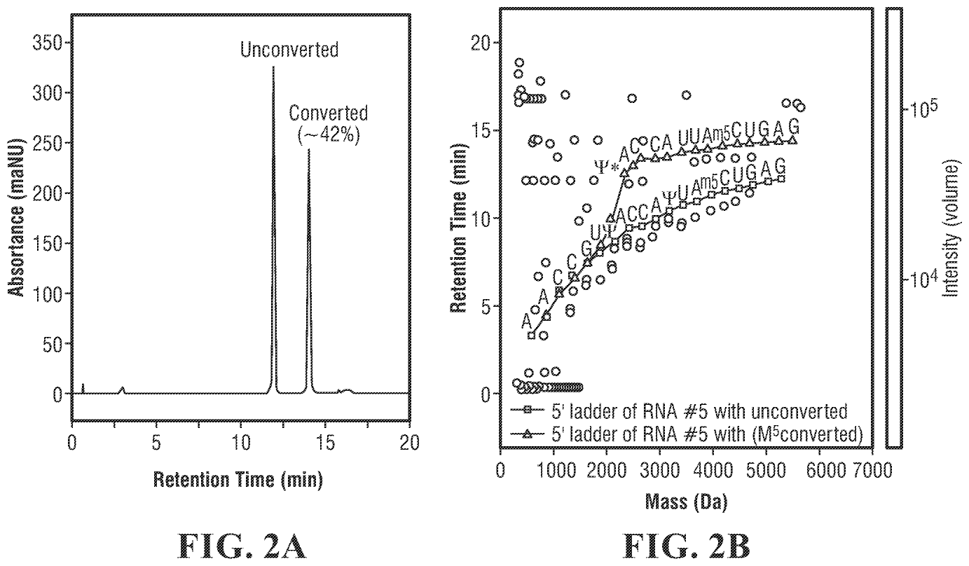

Converting ψ to its CMC-ψ adduct for 2D-HELS MS Seq. ψ is a difficult nucleotide modification for MS-based sequencing because it has the same mass as uridine (U). To differentiate these two bases from each other, the RNA was treated with CMC, which converts a ψ to a CMC-ψ adduct. The adduct has a different mass than U and can be differentiated in the 2D-HELS MS Seq. FIG. 2A shows the HPLC profile of the crude product of the reaction converting ψ to its CMC-adduct in RNA #6. By integrating their UV peaks, the percent conversion was calculated and 42% ψ is converted to its CMC-ψ adduct after the process illustrated in Section 5. After acid degradation and LC-MS measurement, the sequence was manually acquired based on both non-CMC-converted ladders and CMC-converted ladders identified from the algorithm-processed data (Bjorkbom, A. et al, 2015 Journal of the American Chemical Society 137 (45) 1443014438); Zhang, N. et al. Nucleic Acids Research. 47 (20), e125 (2019). A red curve branches up off of the grey curve starting from ψ at position 8 in RNA #6 (FIG. 2B) due to partial conversion of ψ to the CMC-ψ adduct. Because of the mass and hydrophobicity of the CMC, this conversion results in a 252.2076 Dalton increase in mass and a significant increase in t_{R} for each CMC-ψ adduct-containing ladder component when compared to its unconverted counterpart. Thus, a dramatic shift starting at position 8 in RNA #6 can be observed in the 2D mass-t_{R} plot, indicating that position 8 is indeed a ψ in RNA #6.

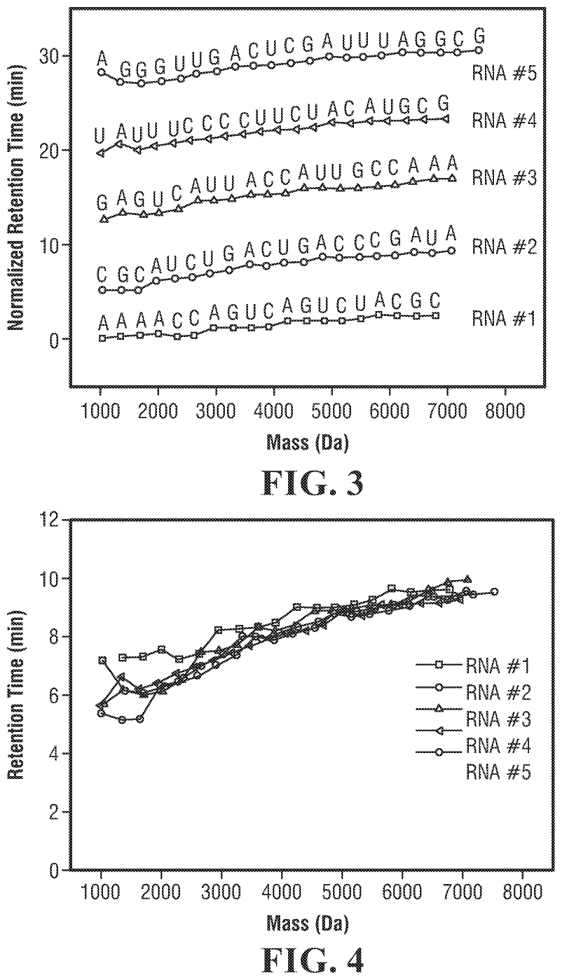

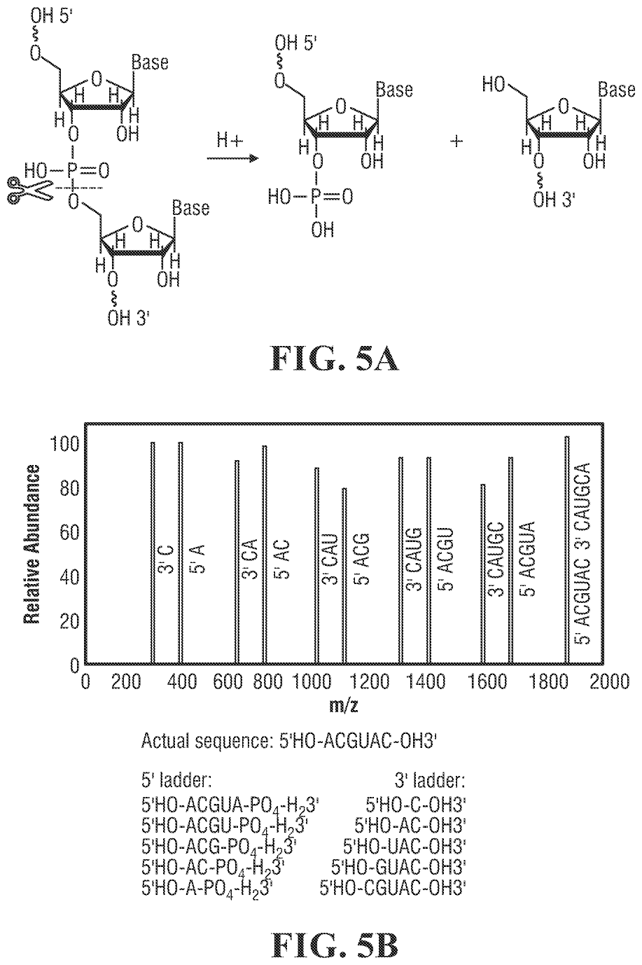

Sequencing RNA mixtures. A mixture of five different RNA strands is sequenced by the 2D-HELS MS Seq approach with 3′-end labeling. The concern for sequencing mixed RNAs is that multiple ladder curves in the 2D mass-t_{R} plot may overlap with each other when they all share the same starting points (the hydrophobic tag in the 2D mass-t_{R} plot). However, base calling is made one by one, each based on a mass difference between two adjacent ladder fragments in the MFE data. The correct base call can be made as long as each mass difference matches well (a PPM MS difference <10) with one of the theoretical masses of canonical or modified nucleotides in the data pool (Bjorkbom, A. et al, 2015 Journal of the American Chemical Society 137 (45) 1443014438); Zhang, N. et al. Nucleic Acids Research. 47 (20), e125 (2019)). In the analysis of the multiplexed RNA samples, the typical processing and base-calling algorithm used in FIG. 1 and FIG. 2 is not used mainly due to the significantly increased data complexity resulting from the mixture. These sequences are base-called manually via calculating the mass difference between two adjacent mass ladder fragments, and comparing it to the theoretical mass of the nucleotide in the data pool⁹. Any matched base with a mass PPM <10 is chosen as the base identity at this position. With this base-by-base manual calculation for base-calling, all sequences in the mixture are accurately sequenced. OriginLab software is used to re-construct a 2D mass-t_{R} plot, in which the starting t_{R} for each sequence is normalized systematically for better visualizing five different RNA sequences (FIG. 3). Without such normalization, the letter codes (i.e., A, C, G, and U) for the sequences of all five RNA would be crowded together on the plot (FIG. 4), resulting in less ease of visualization compared to that reported in FIG. 3. The sequencing results demonstrate that 2D-HELS MS Seq approach is not just limited to sequencing of purified single-stranded RNAs, but also, more importantly, RNA mixtures with multiple RNA strands.

### Example 2

### Materials and Methods

Prepare all solutions using nuclease-free, diethyl pyrocarbonate (DEPC)-treated water (Thermo Fisher Scientific, Waltham, Mass., USA) (expressed as DEPC-treated H₂O unless otherwise indicated). All reagents are of analytical grade and are used as received without further purification. Use RNase-free microcentrifuge tubes and pipette tips and use RNaseZap™ to wipe RNases off surfaces of lab equipment or apparatuses to avoid possible RNA sample degradation. Stock solutions are stored long-term at −20° C. unless otherwise indicated, and are allowed to equilibrate to the appropriate temperatures, as indicated, immediately prior to the relevant procedure.

Synthetic RNA oligonucleotides. Design six short synthetic RNA oligonucleotides with different lengths (19 nt, 20 nt and 21 nt). These RNA oligonucleotides are randomly selected as representative sequences to demonstrate how to use the sequencing method. RNA #6 contains both canonical and modified nucleotides. Similarly, pseudouridine (ψ) is employed as a representative non-mass-altering modification having an identical mass to U; m⁵C is selected as a representative mass-altering modification to demonstrate the robustness of the approach. The following RNA oligonucleotides are obtained from IDT (Integrated DNA Technologies, Coralville, Iowa, USA) and used without further purification.

RNA #1:
(SEQ ID NO: 1)
5′-HO-CGCAUCUGACUGACCAAAA-OH-3′
RNA #2:
(SEQ ID NO: 2)
5′-HO-AUAGCCCAGUCAGUCUACGC-OH-3′
RNA #3:
(SEQ ID NO: 3)
5′-HO-AAACCGUUACCAUUACUGAG-OH-3′
RNA #4:
(SEQ ID NO: 4)
5′-HO-GCGUACAUCUUCCCCUUUAU-OH-3′
RNA #5:
(SEQ ID NO: 5)
5′-HO-GCGGAUUUAGCUCAGUUGGGA-OH-3′
RNA #6:
(SEQ ID NO: 6)
5′-HO-AAACCGUψACCAUUAm⁵CUGAG-OH-3′

Dissolve each synthetic RNA in nuclease-free, DEPC-treated water to obtain respective RNA stock solutions with a concentration of 100 μM (based on the amount provided by IDT). Store at −20° C. Thaw the reagents in water bath at room temperature and mix well by vortexing and centrifuging before adding to the reaction.

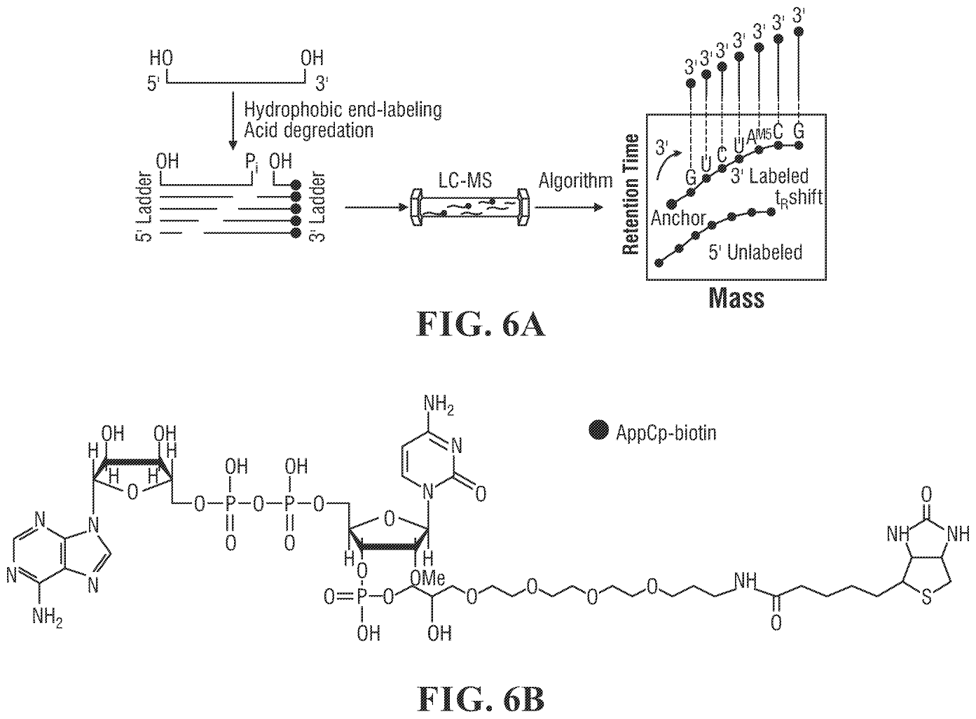

Reagents for labeling the 3′-end of RNA. Biotinylated cytidine bisphosphate (pCp-biotin, TriLink Bio Technologies, San Diego, Calif., USA) (used for the two-step 3′-end labeling protocol): 100 μM stock solution. Add 1.3 mL of DEPC-treated H₂O to 0.1 mg pCp-biotin and mix it well by vortexing and centrifuging. Store at −20° C. Adenosine-5′-5′-diphosphate-{5-(cytidine-2′-O-methyl-3-phosphate-TEG}-biotin (A(5′)pp(5′)Cp-TEG-biotin-3′, ChemGenes, Wilmington, Mass., USA) (used for the one-step 3′-end labeling protocol) (FIG. 6B): 150 μM stock solution. Add 2.7 mL of DEPC-treated H₂O to 0.5 mg A(5)pp(5′)Cp-TEG-biotin-3′ and mix it well by vortexing and centrifuging. Store at −20° C. Other reagents needed for the labeling reaction at the 3′-end: 1 mM ATP, 50 μM Mth RNA ligase, 10× adenylation buffer (New England Biolabs, Ipswich, Mass., USA), DMSO (anhydrous dimethyl sulfoxide, 99.9%), T4 RNA ligase 1 (10 units/μL), 10× ligation buffer (New England Biolabs, Ipswich, Mass., USA). Store at −20° C. until use.

Materials for biotin/streptavidin capture/release. Streptavidin beads (10 mg/mL, 7-10×10⁹ beads/mL) in PBS buffer, pH 7.4, 0.01% Tween™ 20, and 0.09% sodium azide (Thermo Fisher Scientific (Waltham, Mass., USA). Store at 4° C. Binding and Washing (B&W) buffer (2×): 10 mM Tris-HCl, pH 7.5, 1 mM EDTA, 2 M NaCl. Add 0.5 mL of 1 M Tris-HCl buffer to 49.4 mL DEPC-treated H₂O. Add 0.1 ml of 0.5 M EDTA. Add 5.844 g NaCl and mix well by vortexing Dilute 2× B&W buffer to 1× B&W buffer by adding 25 mL of 2× B&W buffer into 25 mL of DEPC-treated H₂O. Store at 4° C. Solution A: DEPC-treated 0.1 M NaOH and DEPC-treated 0.05 M NaCl. Weigh 0.2 g NaOH and 0.15 g NaCl and add to 50 mL DEPC-treated H₂O and mix well by vortexing. Store at 4° C. Solution B: DEPC-treated 0.1 M NaCl. Weigh 0.3 g NaCl and add to 50 mL DEPC-treated H₂O and mix well by vortexing. Store at 4° C.

Chemicals for CMC conversion. CMC (N-cyclohexyl-N′-(2-morpholinoethyl)-carbodiimide metho-p-toluenesulfonate, Sigma-Aldrich, St. Louis, Mo., USA): Weigh 0.0141 g in a 1.5 mL RNase-free microcentrifuge tube. Store at −20° C. Urea (Sigma-Aldrich, St. Louis, Mo., USA): Weigh 0.07 g in a 1.5 mL RNase-free microcentrifuge tube. Store at 4°. Bicine buffer (1 M, pH 8.3): Weigh 1.6317 g bicine in a 15 mL RNase-free microcentrifuge tube and add 8 mL DEPC-treated H₂O. Adjust solution to pH 8.3 with 10 N NaOH. Make up to 10 mL with DEPC-treated H₂O. Store at 4° C. Sodium acetate (NaOAc) solution: 1.5 M, pH 5.6. Add 500 μL of 3 M NaOAc to 499 μL DEPC-treated H₂O. Then add 1 μL of 0.5 M EDTA and mix well by vortexing. Store at 4° C. Sodium bicarbonate (Na₂CO₃) buffer (0.1 M, pH 10.4): Weigh 1.992 g Na₂CO₃ and 8.086 g sodium carbonate (anhydrous) in a 15 mL RNase-free falcon centrifuge tube and add 8 mL of DEPC-treated H₂O. Make up to 10 mL with DEPC-treated H₂O. Store at 4° C.

LC-MS elution buffers. Mobile phase A: 25 mM hexafluoro-2-propanol (HFIP) with 10 mM diisopropylamine (DIPA) in LC-MS grade water. Add 2.6 mL HFIP into 996 mL of LC-MS grade water and mix well by hand shaking. Add 1.4 mL DIPA (1.0 g) and mix well. Store at room temperature. Mobile phase B: LC-MS grade methanol.

Perform all experimental procedures at room temperature unless otherwise specified.

Labeling 3′-end of RNA with biotin (see Note 1 below). Add 1 μL of 10× adenylation reaction buffer, 1 μL of 1 mM ATP, 1 μL of 100 μM pCp-biotin, 1 μL of 50 μM Mth RNA ligase and 6 μL DEPC-treated H₂O (total volume of 10 μL) in an RNase-free, thin walled 0.2 mL PCR tube. Incubate the reaction in a GeneAmp™ PCR System 9700 (Thermo Fisher Scientific, USA) (express as a PCR machine unless otherwise indicated) at 65° C. for 1 hour and inactivate the enzyme by incubation at 85° C. for 5 min (see Note 2 below).

Conduct the ligation step containing by adding the 10 μL reaction solution from the previous step to 3 μL of 10× ligation buffer, 1.5 μL of a 100 μM stock of the RNA sample to be sequenced (for example, RNA #1), 3 μL anhydrous DMSO to reach 10% (v/v), 1 μL T4 RNA ligase (10 units) and 11.5 μL DEPC-treated H₂O (total volume of 30 μL). Add reaction components at room temperature due to the high freezing point of DMSO (18.45° C.). Incubate the reaction in a PCR machine overnight (˜16 hrs) at 16° C.

Quench and purify the reaction by column purification to remove enzymes and free pCp-biotin using Oligo Clean & Concentrator (Zymo Research, Irvine, Calif., USA). Oligo Binding Buffer, DNA Wash Buffer, spin columns and collection tubes are provided in the kit. Add 20 μL DEPC-treated H₂O to the reaction solution to reach a 50 μL sample volume prior to adding Oligo Binding Buffer. Add 100 μL Oligo Binding Buffer to each reaction solution. Add 400 μL ethanol, mix by pipetting at least three times, and transfer the mixture to the provided column. Centrifuge at 10,000 g for 30 seconds. Discard the flow-through. Add 750 μL DNA Wash Buffer to the column. Centrifuge at 10,000 g and maximum speed for 30 seconds and 1 minute, respectively. Lastly, transfer the column to a 1.5 mL RNase-free microcentrifuge tube. Add 15 μL DEPC-treated H₂O to the column and centrifuge at 10,000 g for 30 seconds to elute the RNA product. Store at −20° C. prior to usage.

Replace pCp-biotin with AppCp-biotin (see Note 3). Perform a one-step ligation reaction containing 2 μL of 150 μM AppCp-biotin, 3 μL of 10× ligase reaction buffer, 1.5 μL of a 100 μM stock of the RNA sample to be sequenced, 3 μL anhydrous DMSO (to reach 10% (v/v)), 1 μL T4 RNA ligase (10 units) and 19.5 μL DEPC-treated H₂O with (total volume of 30 μL). Incubate the reaction overnight (˜16 hrs) at 16° C. Perform column purification as described above to elute the 3′-biotinylated RNA sample with 15 μL DEPC-treated H₂O in a 1.5 mL RNase-free microcentrifuge tube.

Streptavidin beads for physical separation of biotinylated RNA (see Note 4). Activate streptavidin beads by adding 200 μL of 1× B&W buffer to 200 μL streptavidin beads. Vortex this solution for 30 s and place it on a magnet stand for 2 min, then discard the supernatant. Wash the beads twice with 200 μL Solution A and once in 200 μL Solution B. For each wash step, vortex the solution for 30 s and place it on a magnet stand for 2 min, then discard the supernatant. Finally, after all wash steps, add 100 μL of 2× B&W buffer to the washed beads.

Add 1× B&W buffer to the biotinylated RNA sample until the volume is 100 μL. Then add this solution to the washed beads stored in 100 μL of 2× B&W buffer. Incubate for 30 min at room temperature on a rocking platform shaker at 300 rpm (VWR, Radnor, Pa., USA). Place the tube in on a magnet stand for 2-3 min and discard the supernatant. Wash the biotin-coated beads 3 times in 1× B&W buffer (same wash procedure as before) and measure the final concentration of the supernatant during each wash step by Nanodrop for recovery analysis to confirm that the biotinylated RNAs remain on the beads (see Note 5). Incubate the beads in 10 mM EDTA, pH 8.2 with 95% formamide in a PCR machine 9700 at 65° C. for 5 min. Put the tube on the magnet stand for 2 min and collect the supernatant by pipet, carefully avoiding the beads. The supernatant contains the biotinylated RNAs released from the streptavidin beads. Measure the final concentration of the supernatant by Nanodrop ((ND-1000 UV-Vis spectrophotometer, Thermo Fisher Scientific, Waltham, Mass., USA).

Generation of MS sequence ladders by controlled acid degradation of RNA. Divide the collected biotinylated RNA sample into three equal aliquots in RNase-free, thin walled 0.2 mL PCR tubes. For instance, divide an RNA sample with a volume of 15 μL into 5 μL×3 aliquots. Add an equal volume of formic acid (98-100%) to achieve 50% (v/v) formic acid in each reaction tube (see Note 6). Incubate the reaction at 40° C. in a PCR machine, with one reaction for 2 min, one for 5 min, and one for 15 min. Immediately freeze the sample on dry ice after each specified time interval to quench the acid degradation reaction. Use Centrifugal Vacuum Concentrator (Labconco, Kansas City, Mo.) to dry the sample. The sample is typically completely dried within 30 min. Resuspend each dried sample in 20 μL DEPC-treated H₂O and combine them in a LC-MS sample vial for LC-MS measurement.

Sequencing a mixed RNA sample (see Note 7). A mixture of five different RNA sequences (RNA #1 to #5) are used here as an example to demonstrate the experimental procedures. Mix 15 μL of 10× ligase reaction buffer, 1.5 μL of each RNA strand (100 μM stock of RNA #1 to #5, respectively, for a total volume of 7.5 μL), 10 μL of 150 μM A(5′)pp(5′)Cp-TEG-biotin-3′ (one-step protocol), 15 μL anhydrous DMSO, 5 μL T4 RNA ligase (10 units/μL) and 97.5 μL DEPC-treated H₂O to produce a reaction solution with a total volume of 150 μL in a 1.5 mL RNase-free microcentrifuge tube. Distribute the reaction solution into five equal-volume aliquots; each microcentrifuge tube now contains 30 reaction solution.

Incubate the reaction overnight (˜16 hrs) at 16° C. as described above. Conduct column purification according to the procedure as described above with five parallel spin columns provided by Oligo Clean & Concentrator. A mixed sample of 3′-biotinylated 5 RNA strands (RNA #1 to #5) should be eluted with 15 μL DEPC-treated H₂O in each 1.5 mL RNase-free microcentrifuge tube.

Combine the purified mixture samples from each of the five tubes into one 1.5 mL RNase-free microcentrifuge tube. Perform formic acid degradation (50% (v/v)) according to the procedures as described above to generate MS ladders for sequencing.

CMC conversion for identifying and locating pseudouridine (see Note 8 and Note 9). Add 80 μL DEPC-treated H₂O to a 1.5 mL RNase-free microcentrifuge tube containing 0.0141 g CMC and 0.07 g urea. Then add 10 μL RNA (100 μM) to be sequenced, 8 μL bicine buffer (1 M, pH 8.3) and 1.28 μL EDTA (0.5 M). Bring a total reaction volume of 160 μL by adding 60.72 μL DEPC-treated H₂O. The final concentrations of CMC, urea, EDTA and bicine are 0.17 M, 7 M, 4 mM and 50 mM bicine (pH 8.3), respectively (15). Divide the 160 reaction solution into four equal aliquots of 40 μL each and incubate in a PCR machine at 37° C. for 20 min. The maximum reaction volume is 50 μL per tube based on the PCR machine used in this procedure. Add 10 μL of 1.5 M sodium acetate and 0.5 mM EDTA (pH 5.6) to quench each reaction. Perform column purification with four parallel spin columns provided by Oligo Clean & Concentrator to remove excessive reactants according to the procedure as described above in Section 3.1.3. Transfer the purified product to four RNase-free, thin walled 0.2 mL PCR tubes. In each 15 μL purified product add 20 μL of 0.1 M Na₂CO₃ buffer (pH 10.4) and make up the volume to 40 μL with 5 μL DEPC-treated H₂O. Incubate these four reaction tubes in a PCR machine at 37° C. for 2 h. Use four parallel spin columns provided by Oligo Clean & Concentrator to purify the reaction products. The CMC-w converted product should be eluted with 15 μL DEPC-treated H₂O in each 1.5 mL RNase-free microcentrifuge tube. Transfer the purified CMC-Φ-converted sample to four RNase-free, thin walled 0.2 mL PCR tubes. Add an equal volume of formic acid to achieve 50% (v/v) formic acid in each reaction tube. Perform acid degradation according to the procedures as described above in Section 3.3 to generate MS ladders for sequencing.

LC-MS measurement and analysis of RNA samples. Transfer the RNA samples, stored in DEPC-treated H₂O prior to LC-MS analysis, to a conical bottomed micro-insert (250 μL) in a 2 mL glass HPLC sample vial for analysis (Agilent, Santa Clara, USA). The maximum injection volume for each sample is 20 μL containing 100-400 pmol of RNA. Use LC conditions as follows: a column temperature of 35° C. and flow rate of 0.3 mL/min as well as a linear gradient from 2-20% mobile phase B over 15 min followed by a 2 min wash step with 90% mobile phase B (see Note 10). Set MS analysis for data recording with following settings: negative ion mode; range, 350 m/z to 3200 m/z; scan rate, 2 spectra/s; drying gas flow, 17 L/min; drying gas temperature, 250° C.; nebulizer pressure, 30 psig; capillary voltage, 3500 V; and fragmentor voltage, 365 V (see Note 11). Extract data files with MassHunter acquisition software provided by Agilent Technologies (Santa Clara, Calif., USA). Use the molecular feature extraction (MFE) algorithm (Agilent Technologies, USA)”) to export compound information to an Excel spreadsheet file, which includes mass, retention time, volume (the MFE abundance for the respective ion species) and quality score, etc. The MFE settings are as follows: “centroid data format, small molecules (chromatographic), peak with height ≥100, up to a maximum of 1000, quality score ≥50” (see Note 12).

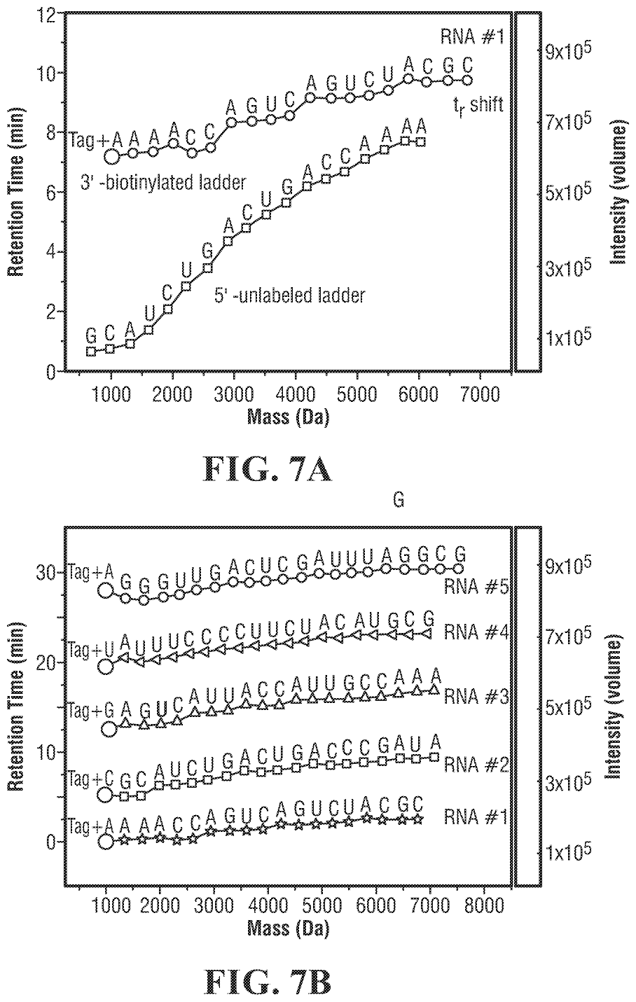

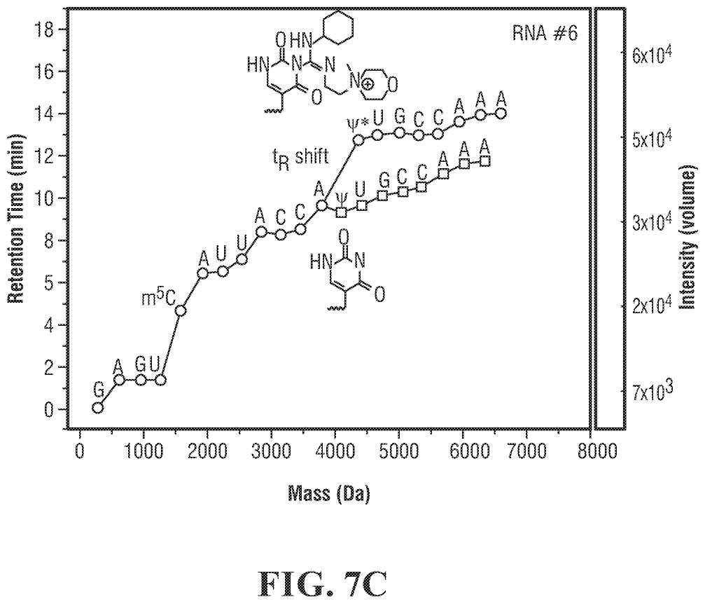

Generate RNA sequence by an anchor-based computer-implemented method (see Note 13). Use a minorly revised version of a previously published anchor-based algorithm (Zhang et al., 2019 BioRxiv:1-10) to process the MFE files of RNA #1 and CMC-converted RNA #6, respectively. Re-construct 2D mass-t_{R} plots for better visualization for each sequence in FIG. 7A and FIG. 7C using OriginLab, based on the sequence read out by the algorithm (See, Table S2-1 through Table S2-4). The observed masses, t_{R}, volume and quality score are reported in the MFE file obtained in as set forth above). Related MFE data and a revised version of anchor-based algorithm (including both the web-based sequencing application and the source code). Manually calculate the mass differences between two adjacent ladder components for base calling to confirm the order of each nucleotide in each algorithm-reported sequence. The structures of RNA modifications can be found in RNA modification databases (Drury D J, 2000, Formic Acid. Kirk-Othmer encyclopedia of chemical technology), and their corresponding theoretical masses are obtained by ChemDraw. Calculate the PPM (parts per million) mass difference to compare the observed mass to the theoretical mass for a specific ladder component, and a value less than 10 PPM is considered a good match for base calling (Bjorkbom et al., 2015, J Am Chem Soc 137:14430-14438; Zhang et al., 2019, Nucleic Acids Res. 47; c125) (see Note 14). Manually verify each nucleotide in each RNA sequence using base-by-base manual calculation.

Manually reading sequences in an RNA sample mixture (FIG. 7B) (see Note 15). Perform all base-calling procedures manually as described above and match well with the theoretical bases in the RNA nucleotide and modification database (Drury D J, 2000, Formic Acid. Kirk-Othmer encyclopedia of chemical technology). The matched bases with a mass PPM <10 are reported as the base identity at each position. With the base-by-base manual calculation for base-calling, the complete sequences of all five RNA strands in the mixed sample can be accurately read out (FIG. 7B) based on the MFE file obtained as set forth above. In Table S2-5 through S2-9, all manual read information is listed, including observed mass, t_{R}, volume, quality score and PPM mass difference.

The following notes are referred to above. Note 1. Label the 5′-end of RNA with biotin or sulfonated Cyanine3 maleimide (sulfo-Cy3) if needed. The method is different than that of 3′-biotinylation and is described in the previous publication (Zhang et al., 2019 Nucleic Acids Research 47:c125)). Note 2. This is the adenylation step through use of pCp-biotin, ATP and Mth RNA ligase to form the activated 5′-adenylated product (5′-AppCp-biotin) (see structure in FIG. 6B). Note 3. It is crucial to improve the labeling efficiency because a high labeling efficiency can increase sample loading efficiency and lower the minimum required sample loading amount. The 3′-end labeling efficiency increased from 60%, using a two-step protocol, to 95%, using a one-step protocol, when activated AppCp-biotin was applied to avoid the additional adenylation step. A higher labeling efficiency/yield can also help to reduce the data complexity (Zhang et al., 2019 Nucleic Acids Research 47:c125). Note 4. This physical separation step for obtaining biotinylated RNAs using streptavidin beads is not mandatory. In order to describe the protocols used in the physical separation, the step is included when sequencing of RNA #1 (FIG. 7A). The hydrophobicity from the biotin tag causes each 3′-labeled sequence ladder fragment to be significant delayed in t_{R} (i.e., a larger t_{R}) during LC-MS measurement, which can help to clearly separate the labeled 3′-ladder fragments from the unlabeled 5′-ladder fragments in the 2-D mass-t_{R} plot. Note 5. The concentration of RNAs was measured at each wash step until there is no RNAs containing in the discarded supernatant, indicating that all (or most) biotinylated RNAs are captured by streptavidin beads. Note 6. Formic acid, and its associated vapor, is strongly corrosive and an irritant to skin, eyes and mucous membranes (Drury D J, 2000, Formic Acid. Kirk-Othmer encyclopedia of chemical technology). Use a fume hood to minimize exposure to this substance. Note 7. To enable sequencing of RNA mixtures, the 3′-end of the RNA was selectively label with a hydrophobic tag such as biotin before LC-MS. All fragments with biotin at the 3′-end are markedly delayed when eluting out of the LC column, each with a larger t_{R} than its unlabeled counterpart in a 2D mass-t_{R} plot (FIG. 6 and FIG. 7A). As such, each labeled fragment in the sequence ladder systematically shifts to larger mass values on the mass axis (due to a mass increase caused by the biotin tag) and to the higher values on the t_{R} axis (due to the t_{R} delay caused by biotin's hydrophobicity) in the 2D plot. This mass-t_{R} ladder makes it possible to read a complete RNA sequence using one labeled 3′-ladder alone without the need to combine two ladders (3′- and 5′-ladders) together through end pairing (Zhang et al., 2019 Nucleic Acids Research 47:c125). This advance also makes it possible to de novo sequence not only a single RNA sequence, but also mixed RNA each with a distinct sequence, because each RNA now has its own unique mass-t_{R} ladder, allowing each RNA in the mixture to be sequenced independently. Even if there are overlaps in terms of mass and t_{R} among labeled ladder fragments that share an identical hydrophobic tag at the 3′ end, the correct base call, and subsequently correct sequence, can be obtained as long as a given mass difference matches well with a theoretical mass difference in the data pool (Bjorkbom et al., 2015, J Am Chem Soc 137:14430-14438). Different tags with different hydrophobicity (e.g., Cyanine3, and biotin) can be employed to label both the 3′- and/or the 5′-end using different chemistries as a mechanism to magnify the t_{R} differences. Note 8. To address the challenge in sequencing of ψ, advantage was taken of established chemistry where CMC can selectively react with ψ, to form a CMC-ψ adduct (ψ*), but not with U. Similar to the biotin tag used in 2D-HELS, this CMC-ψ adduct has a unique mass 252.2076 Daltons larger than U, and the hydrophobicity of each CMC-ψ-containing ladder fragment increases systematically (Bjorkbom et al., 2015, J Am Chem Soc 137:14430-14438) when compared to its non-CMC-converted counterpart. As such, a new mass-t_{R} ladder curve branches off of the original curve that consists of non-CMC-converted-ψ ladder fragments at the ψ position, assisting in site-specifically identifying and locating ψ in the ψ-containing RNAs (FIG. 7C). Note 9. This reaction protocol applies to either a single RNA sequence or RNA mixtures containing one or multiple pseudouridine bases as described previously (Zhang et al., 2019 Nucleic Acids Research 47:c125). Note 10. For more hydrophobic end-labels such as Cyanine3, an increased percentage range of organic solvent mobile phase B can be applied. For instance, a 2-38% mobile phase B over 30 min with a 2 min wash step with 90% mobile phase B is used for an RNA sample containing a Cyanine3 end-label. Note 11. In the study, a 6550 Q-TOF mass spectrometer was used coupled to a 1290 Infinity LC system equipped with a MicroAS autosampler and Surveyor MS Pump Plus HPLC system (Agilent Technologies, Santa Clara, Calif., USA). Please note that these specifications will change depending on each mass spectrometer. Note 12. MFE settings were optimized to extract all potential compounds, up to a maximum, with the settings “peak with height of 1000, and with quality scores of ≥50”. Note 13. For sequencing 3′-biotinylated RNAs only, pre-processing was performed based on a retention time range from 4 to 10 min, which contains only 3′-labeled RNA mass ladder compounds for algorithmic processing. Values of the retention times for 3′-biotinylated RNAs and their ladder fragments may be different when a different type or model of mass spectrometer is used. Note 14. The manually identified sequences used to compare the observed mass to theoretical mass for mass ladder components are provided in Table S2-10 through Table S2-13). Note 15. To read sequences in a mixture of five RNAs, the anchor-based algorithm does not apply due to increased data complexity.

### Example 3

### Materials and Methods

All chemicals were purchased from commercial sources and used without further purification. tRNA (phenylalanine specific from brewer's yeast), ATPγS (adenosine-5′-(γ-thio)-triphosphate), and T4 polynucleotide kinase (3′-phosphatase free) were obtained from Sigma-Aldrich (St. Louis, Mo., USA). RNase T1, 10×RNA structure buffer, polynucleotide kinase (3′-phosphatase free) and SuperScript IV reverse transcriptase were obtained from Thermo Fisher Scientific (Waltham, Mass., USA). Formic acid (98-100%) was purchased from Merck KGaA (Darmstadt, Germany). Adenosine-5′-5′-diphosphate-{5′-(cytidine-2′-O-methyl-3′-phosphate-TEG}-biotin (AppCpB) was synthesized by ChemGenes (Wilmington, Mass., USA). T4 DNA ligase (400 units/μL) and T4 DNA ligase buffer (10×) were purchased from New England Biolabs (Ipswich, Mass., USA). Biotin (long arm) maleimide was purchased from Vector Laboratories (Burlingame, Calif., USA). AlkB homolog 3, alpha-ketoglutaratedependent dioxygenase (ALKBH3, 2 μg/μL) was purchased from Active Motif (Carlsbad, Calif., USA). All other chemicals, including N-cyclohexyl-N′-(2-morpholinoethyl)-carbodiimide metho-p-toluenesulfonate (CMC), bicine, urea, ethylenediaminetetraacetic acid (EDTA), sodium carbonate (Na₂CO₃), sodium acetate (NaOAc), borohydride (NaBH₄), aniline, Tris (2-amino-2-(hydroxymethyl)propane-1,3-diol)-HCl buffer (1 M, pH 7.5), magnesium chloride (MgCl₂), and potassium chloride (KCl), were obtained from Sigma-Aldrich unless indicated otherwise.

tRNA sample preparation for LC-MS. To ensure that each degraded fragment in the tRNA can be detected on a standard high-resolution liquid chromatography quadrupole time-of-flight mass spectrometry (LC-Q-TOF-MS), an amount of approximately 350 pmol tRNA sample is required for each liquid chromatography-mass spectrometry (LC-MS) run. For preparation of this amount of tRNA sample for the LC-MS analysis, the following experiments were performed.

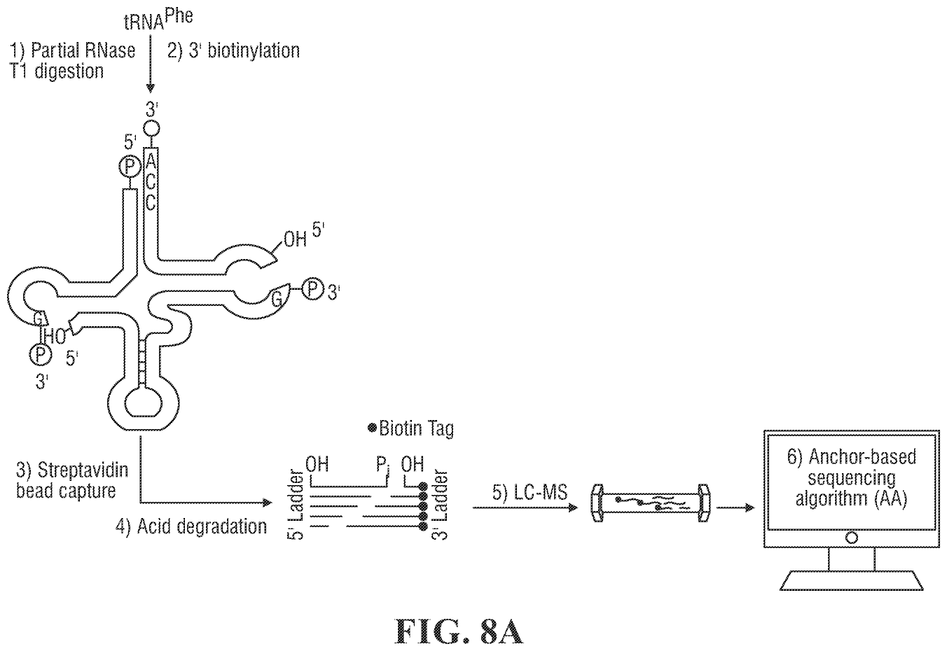

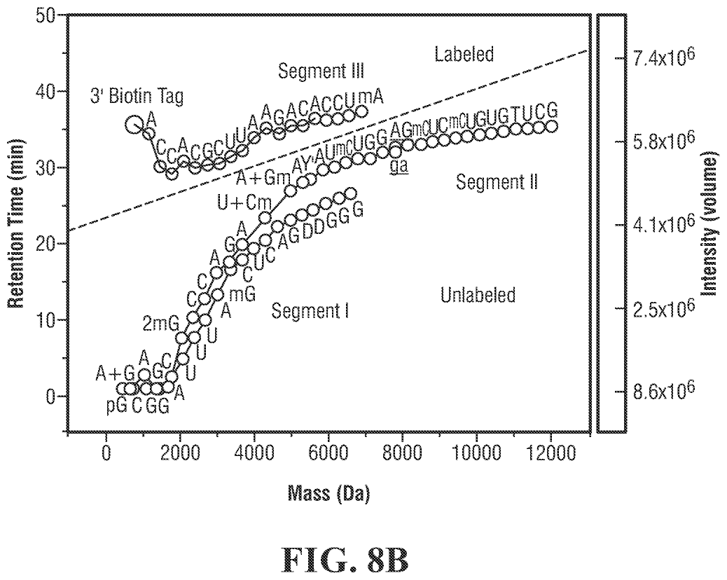

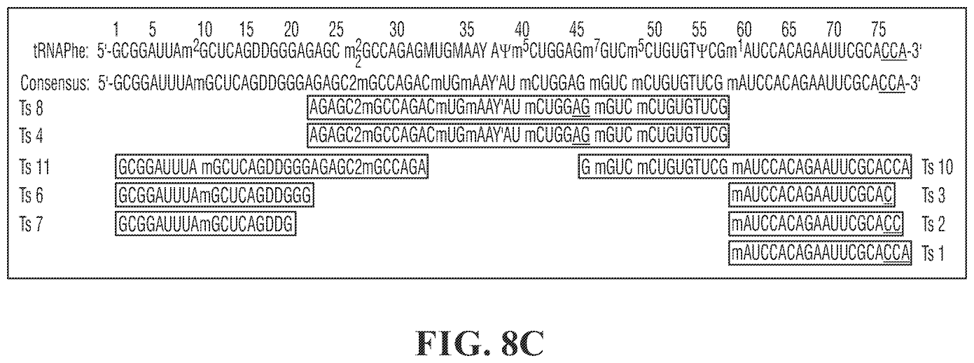

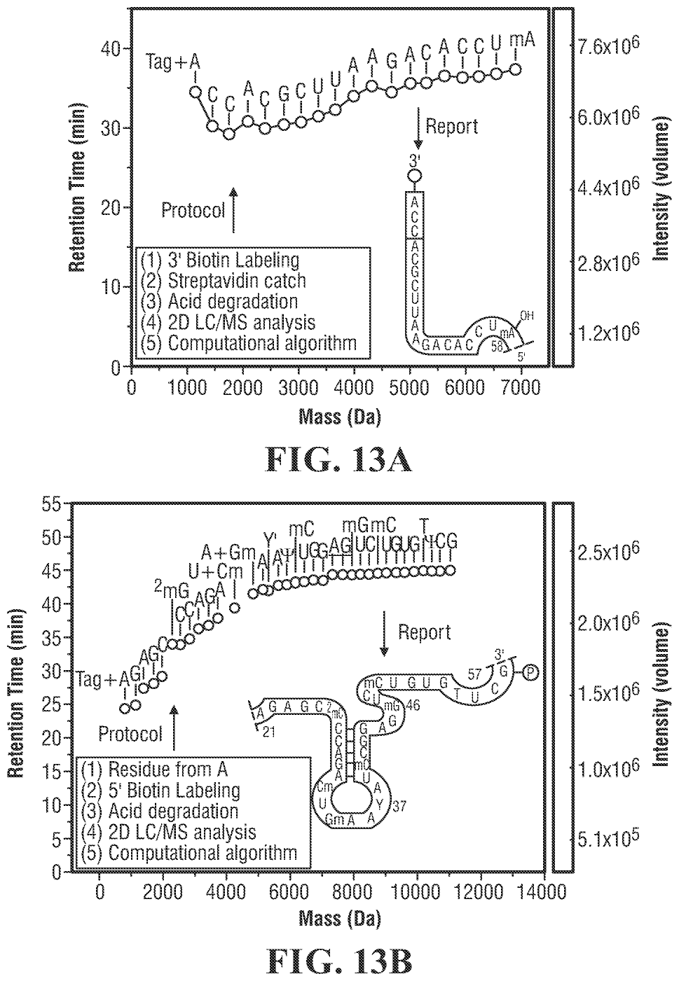

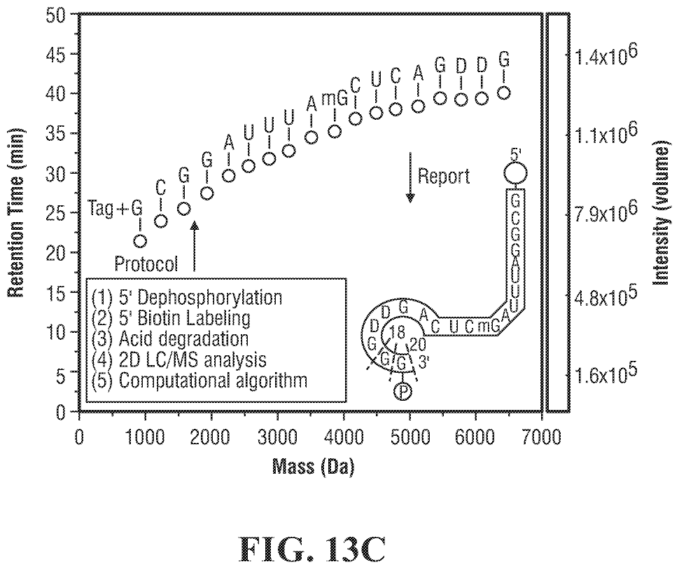

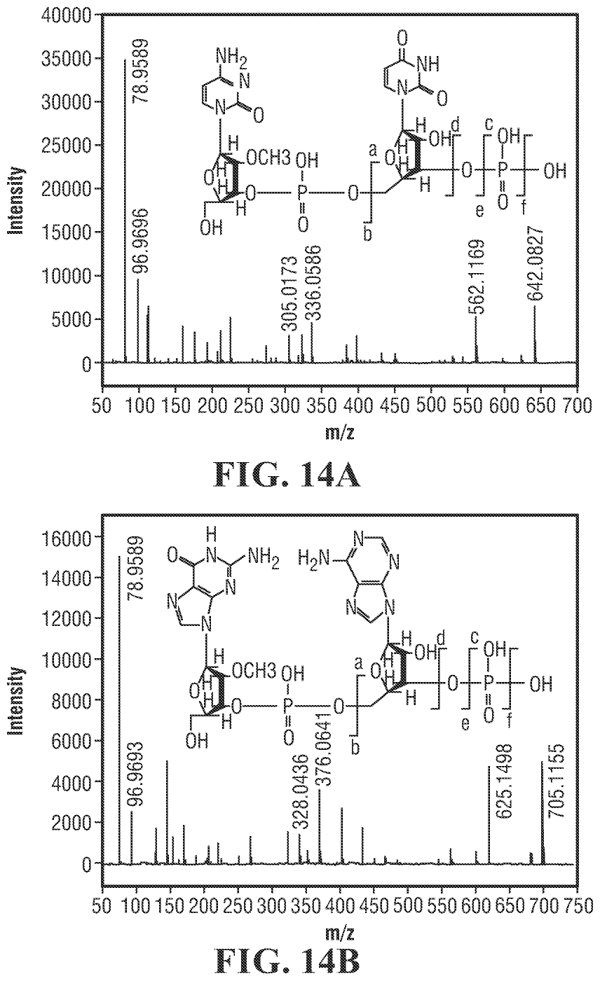

Partial RNase T1 digestion and 3′-biotinylation tRNA (generation of FIG. 8B and FIG. 13A: Approximately 4 μg (150 pmol) of tRNA (phenylalanine specific from brewer's yeast) was digested by 1 μL of 1 U/μL RNase T1 in 1×RNA structure buffer at room temperature for 65 hrs. To maintain the best enzymatic efficacy for T1 digestion, five parallel reactions in total were performed in a small reaction volume (10 μL) separately. The digestion products were purified by Oligo Clean & Concentrator (Zymo Research, Irvine, Calif., USA). The partial digestion was monitored by LC-MS and about 40% tRNA was digested into three segments, which were named segments I, II, and III; the remaining fraction consisted of incompletely digested tRNA fragments and full length tRNA that was not digested at all. After purification by Oligo Clean & Concentrator, the 3′-end of the purified partially digested tRNA was labeled by biotin using a previously published method.¹ After 3′-biotin-labeling and column purification, streptavidin-coupled beads' catch and release was done to harvest the 3′-biotinylated RNase T1 partially digested tRNA, which contains 3′-biotin-labeled segment III and 3′-biotin-labeled full length tRNA as well as a part of unlabeled segments I and II. The reasons for the presence of unlabeled segments I and II are due to 1) the incomplete cut caused the co-existence of segments II and III and 2) the stem-loop intramolecular base pairing between positions 1-7 and 66-72 caused the co-existence of segments I and III. This sample was sequenced by a previously published method after acid degradation followed by LC-MS analysis.¹ Please note that not only can one read out the sequence of segment III (FIG. 13), one can also read out all sequences of segments I, II, and III by the anchor-based algorithm using their specific anchors (FIG. 8B).

In order to confirm the sequences a read out from the above-described sample was done, the residue from streptavidin-coupled beads' catch and release, which contains segment I, segment II, and undigested unlabeled tRNA, was saved for further labeling of segments I and II in the following steps.

Labeling segment II (Generation of FIG. 13): The residue after streptavidin-coupled beads' catch and release from the previous step was concentrated, desalted by oligo concentrator, and used for the 5′-OH biotin-labeling of segment II. 5′-end-labeling was performed in two steps as previously reported.¹ A biotin streptavidin capture method was used to purify the 5′-OH biotin labeled segment II. The residue, which contains segment I and undigested total tRNA, was saved for further labeling of segment I in the next step. Labeled segment II was acid degraded, followed by LC-MS sequencing.¹ The sequence of segment II was read out by the anchor-based algorithm using the biotin anchor (FIG. 13B).

Labeling segment I (Generation of FIG. 13C): The residue of purification products from the previous step was further processed for 5′-dephosphorylation and 5′-OH biotin labeling of segment I. This step can also be accomplished with full-length intact tRNA. 5′-dephosphorylation is needed to generate a 5′-OH before labeling the 5′-end of segment I or full-length intact tRNA. Then, the same procedure was employed to label 5′-OH with the biotin of segment I and full-length intact tRNA. Labeled segment I was acid degraded, followed by LC-MS sequencing.¹ The sequence of segment I was read out by the anchor-based algorithm using the biotin anchor (FIG. 13C). The protocol of 5′-dephosphorylation is as follows: 2 μL of alkaline phosphatase (20 U/μL) was added to the above described tRNA sample containing segment I. The reaction was incubated at 50° C. for 60 min followed by purification by Oligo Clean & Concentrator.

Chemistry for differentiating pseudouridine (ψ) from uridine. The experiments to convert ψ into CMC-ψ adducts were performed using a modified protocol according to reported methods. (Zhang et a/, (2019) Nucleic Acids Res 47, e125; Bakin, A., and Ofengand, J. (1993) Biochemistry 32, 9754-9762), 10 μg (400 pmol) of tRNA after RNase T1 partial digestion was denatured in 5 mM EDTA at 80° C. for 2 min and then placed on ice. The sample was then treated with 0.17 M CMC in 50 mM bicine, pH 8.3, 4 mM EDTA, and 7 M urea at 37° C. for 17 hrs in a total reaction volume of 90 μL. The reaction was stopped by addition of 60 μL of a solution of 1.5 M sodium acetate (NaOAc) and 0.5 mM EDTA, pH 5.6 NaOAc buffer. After purification using Oligo Clean & Concentrator, 60 μL of Na₂CO₃ buffer (0.1 M, pH 10.4) was added to the solution, the solution was brought to a reaction volume of 120 μL by addition of nuclease-free, deionized water, and the sample was then incubated at 55° C. for 2 hrs. The reaction was stopped with 60 μL of NaOAc buffer (1.5 M, pH 5.5) and purified by Oligo Clean & Concentrator for LC-MS analysis.

Chemistry for aniline-induced cleavage at m⁷G (7-methylguanosine). tRNA was treated with borohydride (NaBH₄) and aniline sequentially to generate a site-specific cleavage right after m⁷G, according to reported experimental potocols (Wintermeyer, W., and Zachau, H. G. (1970) Febs Letters 11, 160-164; Marchand, V., Ayadi, L., Ernst, F. G. M., Herder, J., Bourguignon-Igel, V., Galvanin, A., Kotter, A., Helm, M., Lafontaine, D. L. J., and Motorin, Y. (2018), Angew Chem Int Edit 57, 16785-16790). 10 μg (400 pmol) of tRNA was preincubated for 15 min at 37° C. in the following buffer with a total reaction volume of 20 μL: 0.2 M Tris-HCl buffer, pH 7.5, 0.01 M MgCl₂, and 0.2 M KCl. The cooled solution was added to a freshly prepared ice-cold solution of 20 μL NaBH₄ in the same buffer to give final concentrations of 60 μM tRNA and 0.5 M NaBH₄. The reduction was performed at 0° C. in an ice bath under subdued light. The reaction was terminated by pipetting aliquots of the reaction mixture into 4 μL of 6 N acetic acid, followed by subsequent purification by Oligo Clean & Concentrator. Then, the resulting tRNA product was dissolved in 200 μL aniline/acetate solution (aniline/acetic acid/water=1:3:7), and incubated for 10 min at 60° C. 200 μL of 0.3 M sodium acetate, pH 5.5, was then added to the sample, followed by purification by Oligo Clean & Concentrator for LC-MS analysis.

Reverse transcription single base extension (rtSBE). Demethylation: The demethylation reaction was carried out at 37° C. in 50 mM Na-HEPES buffer (pH 8.0) containing 2.5 μg (100 pmol) of tRNA, 4 μg ALKBH3, a 1-methyladenosine (m¹A) demethylase of tRNA (2 μg/μL), 150 μM ammonium iron (II) sulfate (Fe(NH₄)₂(SO₄)₂), 1 mM α-ketoglutarate, 2 mM sodium ascorbate, and 1 mM TCEP (tris(2-carboxyethyl)phosphine) with a total reaction volume of 20 μL for 1 hr. Oligo Clean & Concentrator was applied to remove salts and excessive reactants. A control experiment was performed in the absence of ALKBH3 in order to rule out the possibility of cleavage of the tRNA template induced by hydroxyl radicals, which might be generated under Fenton-like reaction conditions (sodium ascorbate and Fe²⁺) (Ingle, S., Azad, R. N., Jain, S. S., and Tullius, T. D. (2014) Nucleic Acids Res 42, 12758-12767; Costa, M., and Monachello, D. (2014) Methods Mol Biol 1086, 119-142).

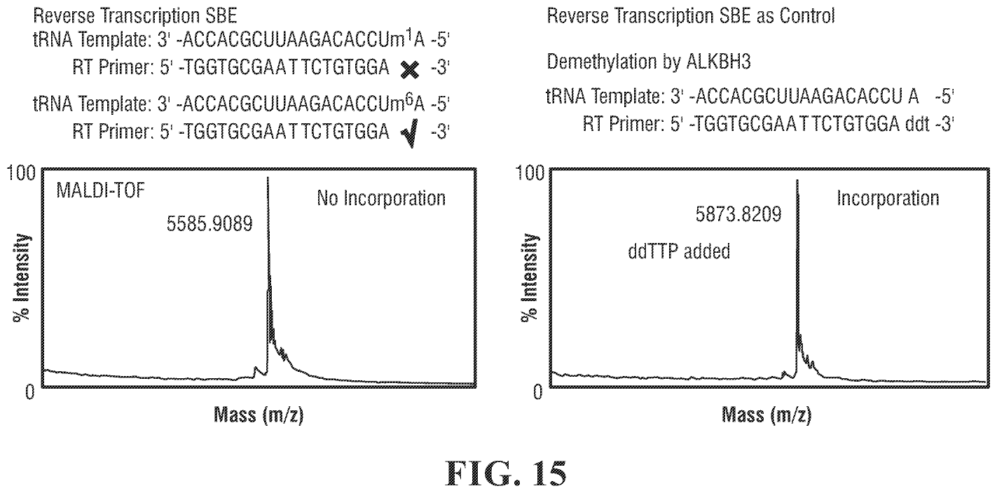

rtSBE: A reverse transcriptase primer (5′-TGGTGCGAATTCTGTGGA-3′ (SEQ ID NO: 7) was designed; the 3′-primer end is adjacent to the m¹A position) using tRNA as a template for m¹A identification, and demethylated tRNA as the control template (FIG. 15). The rtSBE reaction was performed in a 30 μL reaction volume containing 1× SuperScript™ IV RT reaction buffer, 0.625 μg (25 pmol) of tRNA template, 50 pmol primer, 2.5 nmol ddNTPs, 5 mM DTT (dithiothreitol), 2 U RNase inhibitor, and 10 U SuperScript IV reverse transcriptase at 65° C. for 5 min, followed by incubation on ice for 1 min. Then, the full reverse transcription reaction was carried out in a thermal cycler (25 cycles of 45° C. for 30 sec and 55° C. for 1 min). Finally, the reaction was inactivated by incubation at 80° C. for 10 min, followed by application of Oligo Clean & Concentrator to remove all salts and proteins. The rtSBE products were measured on a Voyager DE matrix-assisted laser desorption/ionization (MALDI)-TOF mass spectrometer (Applied Biosystems, Foster City, USA).

LC-MS analysis. LC-MS instrument: a 6550 Q-TOF mass spectrometer coupled to a 1290 Infinity LC system equipped with a MicroAS autosampler and SurveyorMS Pump Plus HPLC (high performance liquid chromatography) system (Agilent Technologies, Santa Clara, Calif., USA) (Hunter College Mass Spectrometry, NY, USA). The LC column is a 50 mm×2.1 mm C18 column with a particle size of 1.7 μm. General LC-MS conditions for analyzing tRNA sequencing ladders were the same as previously reported (Zhang et al., S. (2019) Nucleic Acids Res 47, e125), except that the gradient used was 2-20% buffer B for 60 min, followed by a 2 min 90% buffer B wash step. General MS conditions for the methylated dimers were the same as previously reported except the following: targeted MS/MS was used and the mass range for MS1 was 350-3200 to/z, while the mass range for MS2 was 50-750 m/z. For the CmU dimer (C+U+2′-O-methyl; The 2′-O-methyl renders the phosphodiester bond between C and U nonhydrolyzable), the targeted precursor was 642.0837 m/z (t_{R}=2.95 min). For the GmA dirtier (G+A+2′-O-methyl), the target precursor was 705.1164 m/z (t_{R}=3.50 min and 4.08 min), collision energy (CE) 20. LC conditions: gradient of 2-20% MeOH for 60 min (buffer A: 200 mM hexafluoroisopropanol (HEW), 1.25 mM triethanolamine (TEA) in water). General MS conditions for analyzing single nucleosides or nucleotides were the same as previously reported (Zhang, et al., (2019) Nucleic Acids Res 47, e12) except that a m/z range of 100-2000 was used. LC conditions: 0% buffer B for 5 min, 0-50% buffer B for 30 min, 200 μL/min flow; buffer A: water, 0,1% formic acid and buffer B: acetonitrile (ACN), 0.1% FA; column: Waters Acquity UPLC 2.1×100 (Waters, Milford, Mass., USA). The sample data was processed using the MassHunter Acquisition software (Agilent Technologies, Santa Clara, USA) with the previously described methods. The Molecular Feature Extraction (MFE) workflow in MassHunter Qualitative Analysis (Agilent Technologies, USA) was used to extract relevant spectral and chromatographic information from the LC-MS experiments as described previously (Zhang et al. (2019) Nucleic Acids Res 47, e125).

Anchor-based algorithm with the global hierarchical ranking strategy. The anchor-based sequencing algorithm was developed and used to process the above-mentioned MFE data. To produce RNA sequence reads from the MFE data, the algorithm typically has to go through four essential steps: data pre-processing, base-calling, draft sequence generation, and final sequence identification. In the data pre-processing step, the original MFE dataset was subset by refining the range for both t_{R} and mass value data. By this means, the algorithm focuses on reading out sequence(s) from a specific “zone” at each time, which corresponds to either a labeled or an unlabeled subset of LC-MS data. After subsetting the dataset, the algorithm performs base-calling. The theoretical mass, calculated from the chemical formula, of all known ribonucleotides, including those with modifications to the base, is stored as a list of M_{BASE}. In the first iteration, the algorithm finds the mass corresponding to the molecular tag (anchor), e.g., the 3′-biotin tag in the labeled subset of the MFE data, and sets M_{experimental_i} equal to this mass. The algorithm tests each M_{BASE} from the list by adding it to M_{experimental_i} and generating a theoretical sum mass M_{theoretical_j}. The algorithm searches through the MFE dataset for a mass value that matches with M_{theoretical_j}. If there exists a matching mass value M_{experimental_j}, a tuple (M_{experimental_i}, BASE, M_{experimental_j}) is stored in the result set V. Since the algorithm tests all M_{BASE} in the list and looks for all possible matches, multiple tuples with same M_{experimental_i} but a different BASE identity and M_{experimental_j} may be found and then stored in set V. When the algorithm decides if there is a match, it takes into consideration that the experimental/observed mass in the WIFE data may slightly deviate from the theoretical mass for an identical ribonucleotide unit. A calculated parameter PPM (parts per million) was implemented that allows M be matched M_{experimental_j} to with M_{theoreiical_j} within a customizable range (typically <10 PPM).

The algorithm performs base-calling for all data points in the dataset until all possible tuples are found and stored in set V. Note that each tuple in set V represents an individual base-calling possibility. After base-calling, the algorithm builds trajectories linking tuples in set V to generate draft sequence reads of the RNA.

The fourth and final step of the anchor-based algorithm is the final sequence identification. Because the outputs from LC-MS contain a large number of data points (>500), the algorithm may generate a large quantity of draft sequence reads. To effectively filter out undesired draft reads and to select the desired ones, the global hierarchical ranking strategy was developed. In this strategy, each draft read is ranked hierarchically according to the following criteria: (1) read length (the number of nucleobases in a draft read), (2) average volume, (3) average quality score (QS), and (4) average PPM. Average volume is calculated by summing the volume associated with each data point in a draft read and dividing the sum by read length. Average QS is calculated by dividing the sum of QS by read length. Average PPM is the sum of all PPM values associated with data points contained in a draft read divided by read length. In the end, the draft read with longest read length, highest average volume, highest average QS, and lowest average PPM wins over all other draft reads in the global hierarchical ranking procedure and is identified as the final sequence for the targeted RNA fragment.

Related MFE data and the anchor-based algorithm (including both the web-based sequencing application and the source code) are available upon request and were uploaded to a separate server at Github (https://github.com/rnamodifications/seqapp). All figures and data presented are representative data of multiple experimental trials (n≥3).

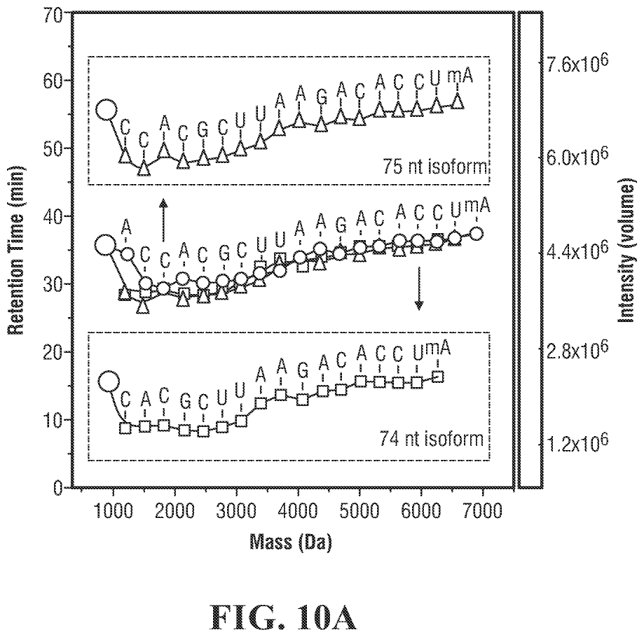

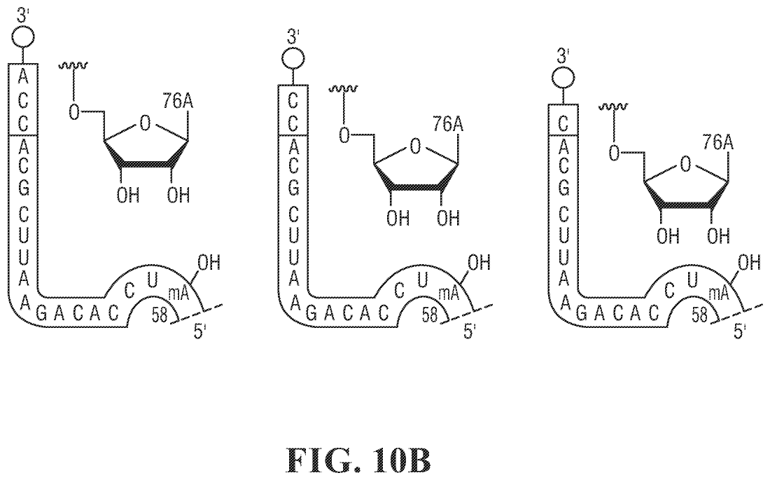

Detection and sequencing of three CCA truncated isoforms. When analyzing the biotinylated 3′-segment of the tRNA (58m¹A-76A), it was found that there is more than one ladder that has the biotin tag as shown in FIG. 10A, indicating that this segment contains more than one sequence. Isoforms of segment III were searched for in the dataset as an additional step to the global hierarchical ranking algorithm. The final output (Tables S1-S3) of the original algorithm is one of the three isoforms and is aligned with all draft reads by a Smith-Waterman alignment 8 to acquire their alignment score. Draft reads with an alignment score above 94.44% are considered candidates of isoforms, and the candidates are ranked by average volume. Six candidates were acquired with a threshold of 94.44%. Because the only variation between the isoforms is that they have different tail lengths and sequences of C, CC, or CCA respectively, the tails of the six candidates were trimmed and a second round of Smith-Waterman alignment was executed. After trimming, draft reads of isoforms had a 100% alignment score with each other, and thus were filtered out from the six candidates.

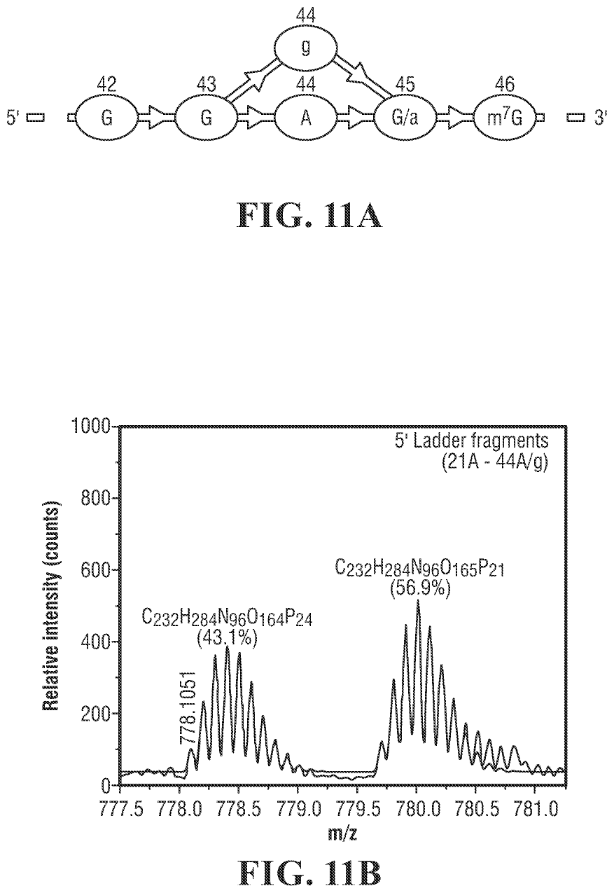

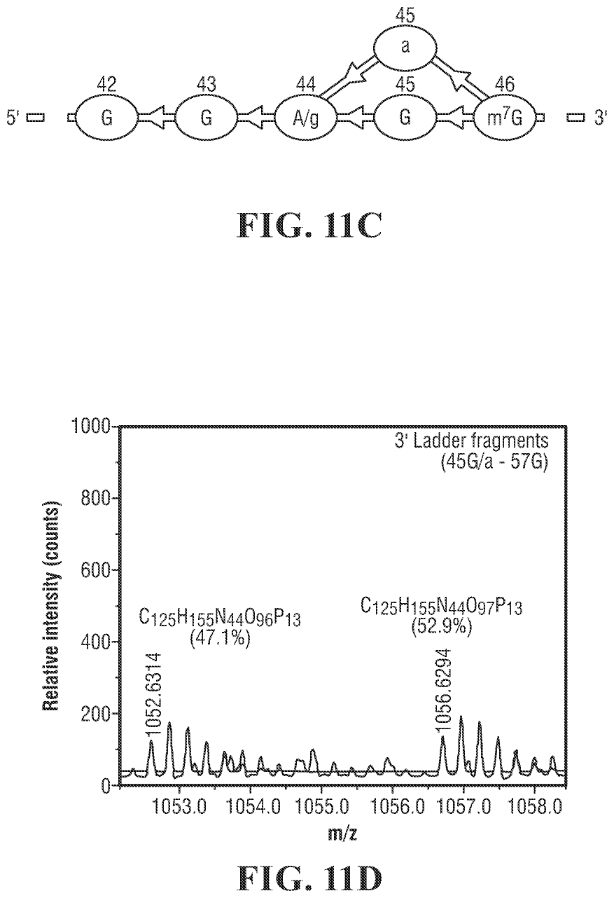

Full-spectral analysis for a new 44g45a isoform. To verify the co-existence of the two mass fragments (44A45G and 44g45a), full-spectral analysis provided by the commercial MassWorks software (version 5.0) (Cerno Bioscience, Las Vegas, USA) was employed to examine the corresponding ions of these two fragments simultaneously and see if they co-exist in one spectrum. MassWorks was used to process the original Agilent LC-MS data files, which was then calibrated for spectral accuracy before further analysis. When reading from the 5′-direction (FIG. 11A-B), two ions (m/z 778.1051 and 779.7068, both with 10 charge states) were found for 14 nt fragments (21A-44A/g) in the t_{R} window (t_{R}=31.9-32.9 min) corresponding to 44A and 44g. Also, two ions (m/z 1052.6314 and 1056.6294, both with 7 charge states) were found during full-spectral analysis for 13 nt fragments (45G/a-57G) (t_{R}=16.5-18.6 min) when reading from the 3′-direction (FIG. 11C-D), confirming that 45G and 45a co-exist.

Stoichiometric quantification of all 11 RNA modifications. The relative percentages of 11 modified nucleotides vs. their corresponding canonical nucleotides at each position were quantified by integrating extracted-ion current (EIC) peaks of their corresponding ladder fragments from tRNA according to the previously reported methods (Zhang et al. (2019) Nucleic Acids Res 47, e125; Zhang et al. (2013) Proc Natl Acad Sci USA 110, 17732-17737). The results in detail in Table S3-19.

### Results

Development of an anchor-based algorithm for 2D-HELS-AA MS Seq. To extend the application of the 2D-HELS MS Seq approach from short synthetic RNAs (Zhang et al. (2019), Nucleic Acids Research 47, e125) to allow sequencing of a tRNA, a computational anchor-based algorithm was developed to automate MS sequencing of RNAs. Due to the complexity of MS data derived from the tRNA, it is very challenging to process all data in a single LC-MS run simultaneously. Instead, data pre-processing was used to select a particular subset of the input dataset for the algorithm to focus on initially. This is feasible because a hydrophobic tag was added to the terminus of each RNA to be sequenced, where it remained even after acid degradation. Additionally, the trends of t_{R} and mass of the tag-containing ladder fragments are known from previous studies (Bjorkbom et al. (2015) Journal of the American Chemical Society 137, 14430-14438; Zhang et al. (2019), Nucleic Acids Research 47, e125). In the 2D mass-t_{R} plot of output LC-MS datasets, data points corresponding to tag-labeled RNA fragments are shifted spatially to a zone with larger t_{R}s than those of their unlabeled counterparts, due to the tag's hydrophobicity. Therefore, the algorithm can “zoom in” on one group, either labeled or unlabeled, in its specific zone of the 2D-plot, to read out the sequence of the selected group first. As such, the algorithm is referred to as “anchor-based”, since it specifies the starting data point corresponding to the terminal tag, which latches down the data points corresponding to the specific ladder fragments that one aims to read out from the whole dataset. The anchor-based algorithm significantly simplified the complicated MS data from the tRNA sample because it only read out the sequence for ladder fragments that had a hydrophobic tag or a specified tag with a known mass, and selectively filtered all non-tag/anchor related data points out of the complicated MS data derived from the tRNA sample.

2D-HELS-AA MS Seq of yeast tRNA. As it was only possible to read segments of up to 35 nt long with a 40K mass resolution LC-MS (Zhang et. al. (2019), Nucleic Acids Research 47, e125) a partial RNase T1 digestion step was incorporated to sequence a tRNA that was commercially available, resulting in a reduction of the 76 nt tRNA to segments of sequenceable sizes for 2D-HELS-AA MS Seq. Subsequently, the entire tRNA was directly sequenced with single-base resolution in one single LC-MS run (FIG. 8). To further verify the complete tRNA sequence obtained from the single run above, the three segments partially digested from the tRNA by RNase T1 were labeled and separated them one by one for 2D-HELS-AA MS Seq in three separate LC-MS runs (FIG. 13A-C). To obtain overlapping segment sequences for assembling the complete tRNA sequence, MS data of the tRNA generated without RNase T1 digestion is included, i.e., 31 nt of the tRNA read from 5′ end using a phosphate (PO₄⁻) as the 5′ anchor, and 32 nt of the tRNA read from its 3′ end using a CCA tag as the 3′ anchor, respectively (FIG. 8C). Taking all draft reads output by the anchor-based algorithm together (See, Table S3-1 through S3-11), a full length tRNA sequence was assembled which was a 100% match to the tRNA^{Phe} reference sequence with more than 2× coverage (FIG. 8C).

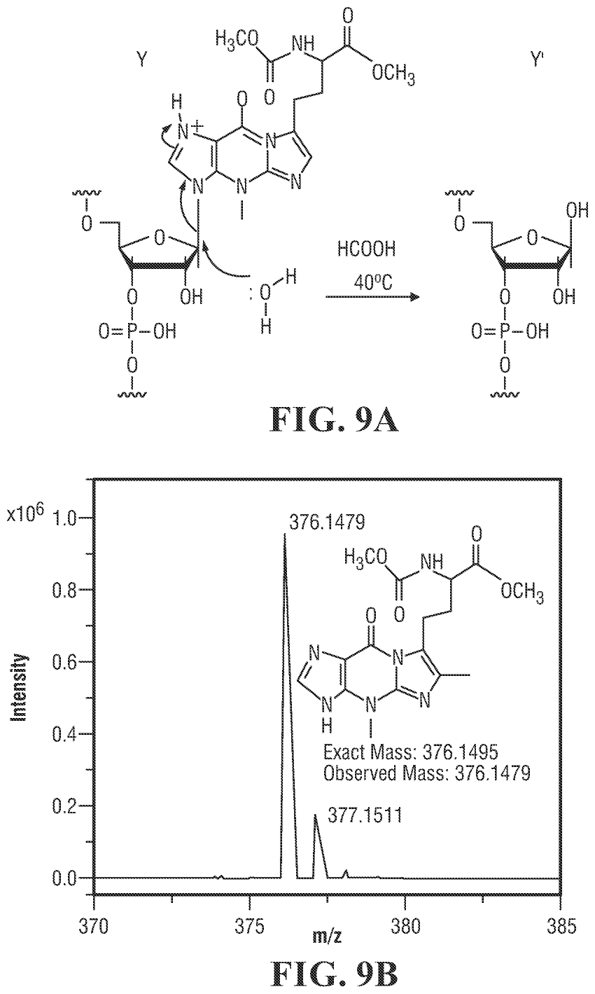

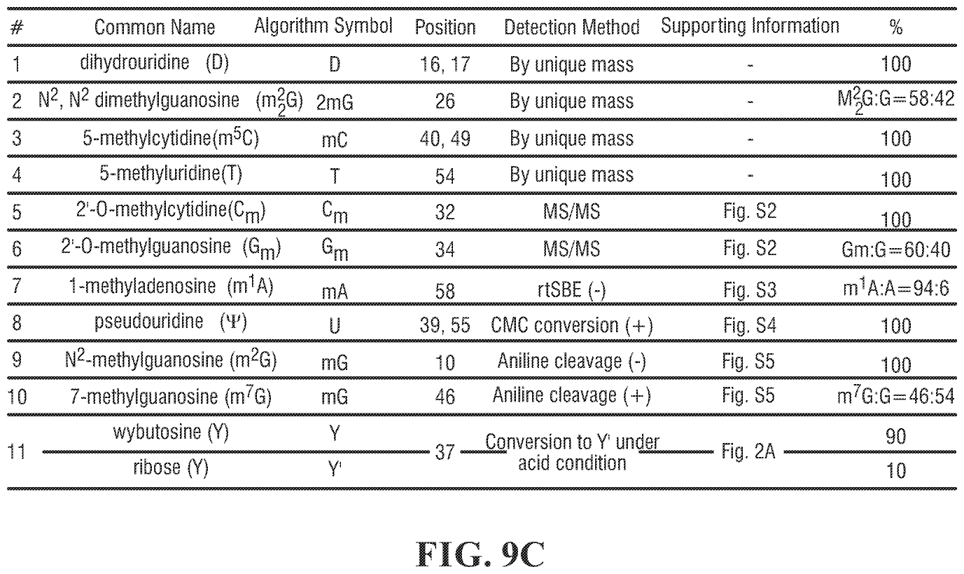

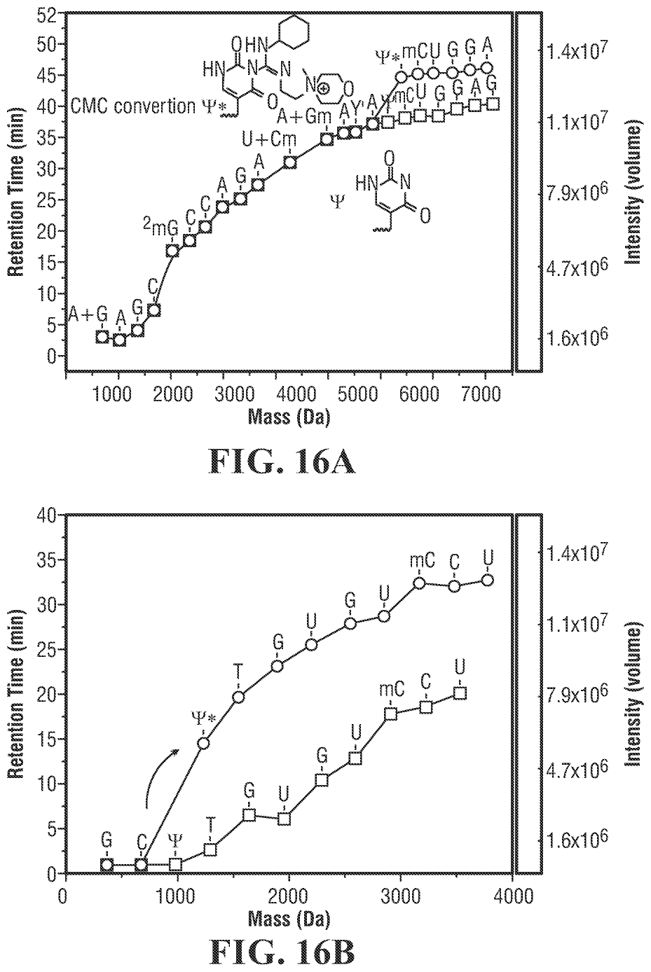

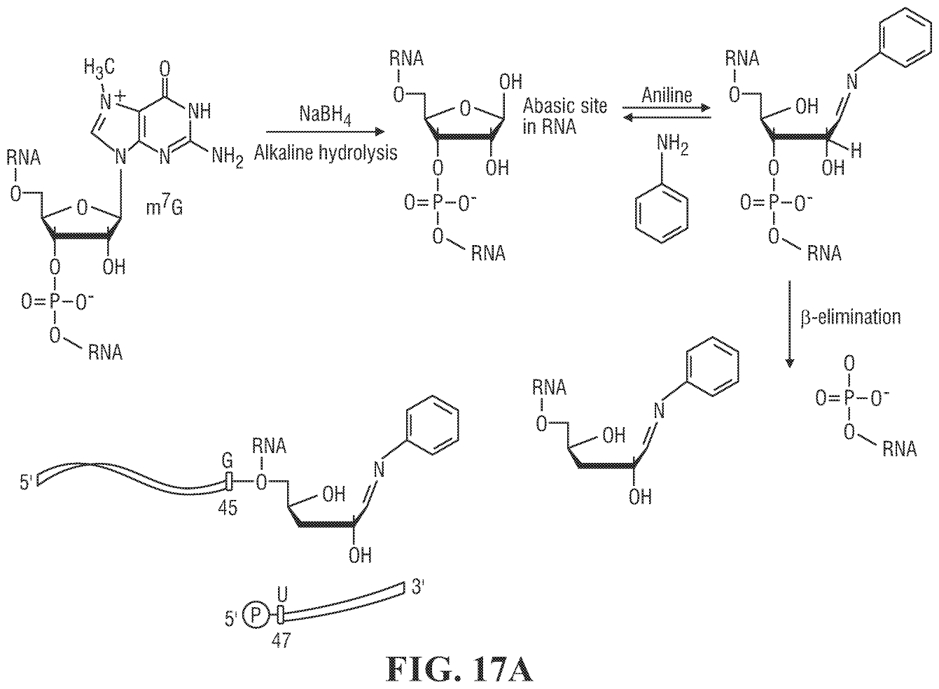

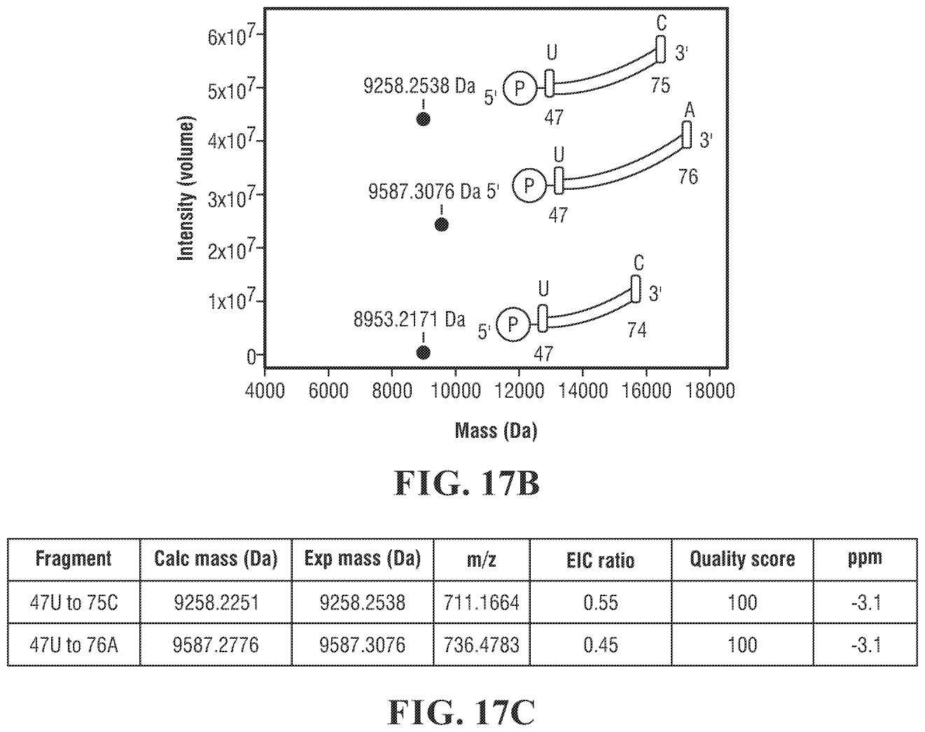

Sequencing of all 11 RNA modifications. During sequencing of the tRNA, successful identification and location of all 11 RNA modifications within the tRNA was achieved (FIG. 9). Four of these modifications could be directly read out by their unique masses: dihydrouridine (D) at positions 16 and 17, N²,N²-dimethylguanosine (m²₂G) at position 26, 5-methylcytidine (m⁵C) at position 40, and 5-methyluridine (T) at position 54. Methylation on the 2′ OH of C (Cm) at position 32 and G (Gm) at position 34 renders the adjacent 3′-5′ phosphodiester linkage non-hydrolyzable, creating a mass gap in both the 5′ and the 3′ mass ladder families larger than 1 nt (Bjorkbom et al., W. (2015), Journal of the American Chemical Society 137, 14430-14438) (FIG. 1B). This gap can be filled in by collision induced dissociation (CID) MS, which determines which of the two unhydrolyzable nucleotides is methylated (Bjorkbom et al., (2015), Journal of the American Chemical Society 137, 14430-14438) (FIG. 9C and FIG. 14). However, other RNA modifications such as pseudouridine (ψ) and U, N²-methylguanosine (m²G) and 7-methylguanosine (m⁷G), and 1-methyladenosine (m¹A) and N⁶-methyladenosine (m⁶A) share identical masses, and LC-MS alone cannot distinguish them. Additional enzymatic/chemical reactions were required to identify them at their particular sites and differentiate them from their corresponding isomers with an identical mass, as shown in the FIG. 9C. To differentiate m¹A at position 58 from its isomeric m⁶A (Chen et al. (2019) Nucleic Acids Res 47, 2533-2545), a reverse transcription/single base extension experiment (rtSBE) was designed, which indicates that m⁶A, but not m¹A, is able to form base-pairing interactions, thus causing a pause during reverse transcription at any m¹A²². The rtSBE results proved that the nucleotide at position 58 is m¹A and not m⁶A (FIG. 15). The demethylation experiment which employed ALKBH3, a m¹A and m³C demethylase of Trna (Chen, Z., Qi, M., Shen, B., Luo, G., Wu, Y., Li, J., Lu, Z., Zheng, Z., Dai, Q., and Wang, H. (2019) Nucleic Acids Res 47, 2533-2545), to convert m¹A to A in tRNA^{Phe} followed by incorporation of ddT based on a positive MALDI result further confirmed that the nucleotide at position 58 is m¹A. In the absence of ALKBH3, the ddT incorporation was not observed. To differentiate ψ from U, the RNA was treated with N-cyclohexyl-N′-(2-morpholinoethyl)-carbodiimide metho-p-toluenesulfonate (CMC) to convert ψ to its CMC adduct (Bakin, A., and Ofengand, J. (1993) Biochemistry 32, 9754-9762), which has a different mass than U/v. The CMC-converted ψ (depicted as ψ*) results in a shift in both t_{R} and mass, allowing facile identification and location of ψ at positions 39 and 55 due to a single drastic shift in the mass-t_{R} ladder at these sites (Zhang et al., (2019), Nucleic Acids Research 47, e125) (FIG. 16) and Tables S3-12 through Table S3-17). To differentiate m⁷G at position 46 from its isomeric m²G at position 10, the tRNA was treated with borohydride (NaBH₄) and aniline sequentially to generate a site-specific cleavage right after m⁷G^{24, 25}. The observed three major mass fragments after the cleavage measured by LC-MS were all a result of cleavage at m⁷G, but in three isoforms with 3′ tails of C, CC, or CCA, respectively (FIG. 17), indicating that there is only one m⁷G in tRNA. A mass fragment induced by a cleavage at m²G at position 10 from either the 5′ end or the 3′ end was not observed. However, the mass fragment from the 5′ end to m⁷G at position 46 after the cleavage was not observed (45 nt long), probably due to mass resolution limitation (Zhang et al. (2019), Nucleic Acids Research 47, e125). Otherwise, no other mass fragments were observed. The unique masses of the cleaved 5′ segments were used to differentiate m7G at position 46 from m²G at position 10, which cannot be cleaved at the same reaction conditions.

The primary task for sequencing is to determine the precise order of the four nucleotides. The method thus extends this capacity to include nucleotide modifications beyond the four canonical nucleotides, based on the unique mass of each RNA modification, and this approach was used to expand beyond synthetic RNA samples examined previously, to directly sequence biological samples for the first time. Only in the case where modifications have isomers with identical masses but different chemical structures, would one require a further RNA modification characterization method to differentiate these isomers following the 2D-HELS-AA MS Seq approach. However, the advantage of the method is that one already knows the mass of the particular nucleotide modification and its location/order without any prior sequence knowledge. This is very different than other RNA characterizing methods that can identify RNA modifications, but must still rely on addition-al established sequencing methods for sequence/location in-formation (Chi, K. R. (2017) Nature 542, 503-506; Sakurai, M., and Suzuki, T. (2011), Methods Mol Biol 718, 89-99; Dominissini, D., Moshitch-Moshkovitz, S., Schwartz, S., Salmon-Divon, M., Ungar, L., Osenberg, S., Cesarkas, K., Jacob-Hirsch, J., Amariglio, N., Kupiec, M., Sorek, R., and Rechavi, G. (2012) Nature 485, 201-206; Meyer, K. D., Saletore, Y., Zumbo, P., Elemento, O., Mason, C. E., and Jaffrey, S. R. (2012) Cell 149, 1635-1646).

Stoichiometric quantification of all 11 RNA modifications. Relative stoichiometries/percentages of modified RNA vs non-modified counterpart RNA can be quantified in partially modified synthetic RNA samples by the technique (Zhang et al. (2019), Nucleic Acids Research 47, e125), and thus stoichiometries/relative percentages of all 11 RNA modifications were quantified at each position of the tRNA (Table S3-19), five of which were not 100% modified (FIG. 9C). The data suggest that there is an abundance of post-transcriptional regulation that can occur in the tRNA at these different positions. For example, the wobble Gm at position 34 was partially modified (60% Gm vs. 40% G), which has important regulatory implications since the lack of Gm could affect binding or stalling in the ribosome (Vendeix, F. A et al. (2008) Biochemistry 47, 6117-6129). 2′-O-methylation is essential for accurate and efficient protein synthesis, and a decreased level of 2′-O-methylation level could lead to an increase in translational infidelity (Erales, J. et al. (2017) Proceedings of the National Academy of Sciences 114, 12934-12939; McCown et al. (2020) Naturally occurring modified ribonucleosides, Wiley Interdisciplinary Reviews: RNA, e1595).

The method revealed unexpected nucleotides in tRNA. Position 26 in tRNA^{Phe} is thought to be m²₂G³²⁻³⁴, however, clear evidence was found that G co-exists at this position, but there is no evidence for any monomethyled G (mG) co-existing at this position. The stoichiometries were quantified by integrating extracted-ion current (EIC) peaks of their corresponding ladder fragments (Zhang et al. (2019), Nucleic Acids Research 47, e125; Wang, X., and He, C. (2014) Mol Cell 56, 5-12) which revealed that m²₂G and G were present at 58% and 42%, respectively (FIG. 9C). Also, both m⁷G at position 46 (46% m⁷G vs. 54% G) in the variable loop and m¹A at position 58 (94% m¹A vs. 6% A) in the TψC loop were partially modified (FIG. 9C), suggesting that the methylation process is highly regulated (Wang, X., and He, C. (2014), Mol Cell 56, 5-12). This is the first time the stoichiometry, identity, and location of these different RNA modifications were all directly measured together in a single study, something no currently available sequencing technologies are capable of, thus providing unique insights that call for further functional studies of these dynamic RNA modifications (Meyer, K. D., and Jaffrey, S. R. (2014) Nat Rev Mol Cell Biol 15, 313-326.)

Identification and quantification of a dynamic change from Y to its depurinated Y′ form. Upon analysis of the sequencing results, the wybutosine (Y) at position 37 was converted to its depurinated product Y′ (ribose form) under acidic degradation conditions (FIG. 9) (RajBhandary, U. L., Faulkner, R. D., and Stuart, A. (1968) Studies on polynucleotides. LXXIX. Yeast phenylalanine transfer ribonucleic acid: products obtained by degradation with pancreatic ribonuclease, J Biol Chem 243, 575-583; Ladner, J. E., and Schweizer, M. P. (1974), Nucleic Acids Res 1, 183-192). Without acid degradation, only 10% of the tRNA contained the depurinated Y′ form at this position, while 90% contained the standard Y form of the base (Table S3-18). However, no Y form was observed in any ladder fragments containing this position after acid degradation, and all of the Y bases were converted to Y′ due to depurination in the acidic conditions (FIG. 9A). As another piece of evidence of the depurination, a mass of 376.1178 Da, corresponding to a cleaved Y nucleobase, was found in the crude products after acid degradation and subsequent MS analysis (FIG. 9B), suggesting that Y′ was originally carried by the tRNA. The fact that the method can identify the dynamic change of Y to Y′ and quantify the relative Y/Y′ ratio could be useful for potential diagnostic assays, as such changes in the Y′/Y ratio could be used as a potential biomarker, e.g., in certain nervous system diseases (Fang, B., Wang, D., Huang, M., Yu, G., and Li, H. (2010)). Hypothesis on the relationship between the change in intracellular pH and incidence of sporadic Alzheimer's disease or vascular dementia, Int J Neurosci 120, 591-595, where the common characteristics are decreased pH at both the tissue and cellular levels. Based on the same principle, the method could potentially probe dynamic changes of other base modifications, acid-labile or not, and quantify variations in their ratios in particular cells or tissues subjected to different biological processes or disease conditions.

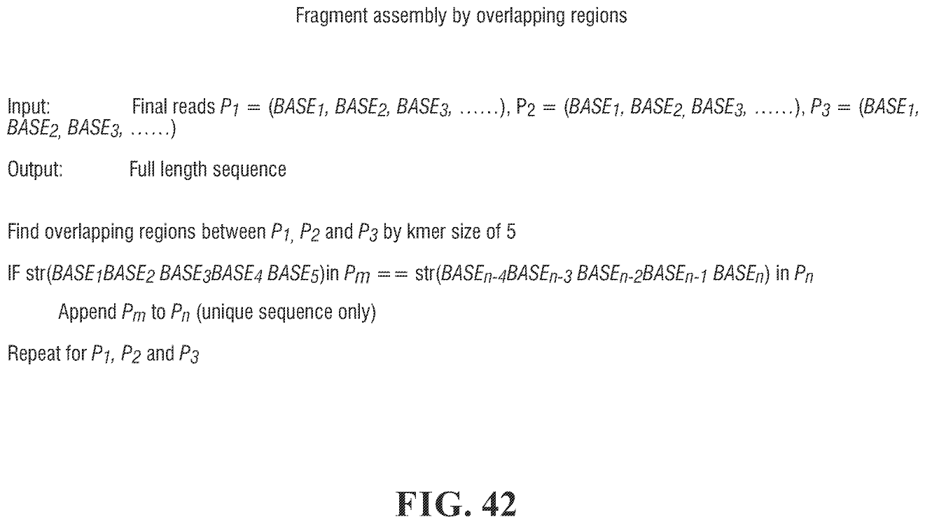

Identification and quantification of two other truncation isoforms (74 nt and 75 nt) at the 3′ end. Unlike its nominal identity according to the supplier, upon sequencing, the commercially-prepared tRNA^{Phe} (phenylalanine specific from brewer's yeast) sample was revealed to be heterogeneous. When analyzing biotinylated 3′ segment of the tRNA (58 m¹A-76A), it was found there is more than one ladder that has the biotin tag as shown in FIG. 10A, indicating that this segment contains more than one sequence. Besides the 76 nt tRNA with a complete post-transcriptionally modified CCA tail, two other incomplete isoforms of the tRNA that are missing an A and a CA at the 3′-CCA tail, respectively, were further identified in a 3′ segment of the tRNA (58m¹A-76A) (FIG. 10) using the anchor algorithm and a revised Smith-Waterman alignment similarity algorithm (See FIG. 42). Surprisingly, the most abundant component was not the nominal 76 nt tRNA^{Phe}, which comprised only 17% of the sample as calculated by integration of the corresponding EIC (Table S3-24). Rather, the 75 nt tRNA^{Phe} with a missing A at the 3′ end was the major component of the sample, at 80%, while the 74 nt tRNA^{Phe} with a missing CA at the 3′ end was a minor component at 3%. The two tail-truncation isoforms cannot be degraded products of longer tRNAs like the 76 nt tRNA^{Phe}, otherwise, they would not contain the free 3′-OH required for the 2D HELS chemistry (Zhang et al., (2019), Nucleic Acids Research 47, e125). The data indicates that 2D-HELS MS Seq is not only able to sequence modified RNA, but it can also identify tail-truncation isoforms that were primarily only studied by polyacrylamide gel electrophoresis methods previously (Merryman, C. et al., (2002) Chem Biol 9, 741-746). As stress-induced tRNA fragmentation has been implicated in cancers and other diseases (Thompson, D. M., and Parker, R. (2009) Stressing Out over tRNA Cleavage, Cell 138, 215-219), further studies into the relationship between the relative abundances of tRNA tail-truncation isoforms and various diseases will assist in understanding the potential role of such isoforms in disease-related biological processes and subsequent treatments (Hou, Y. M. (2010) IUBMB Life 62, 251-260).

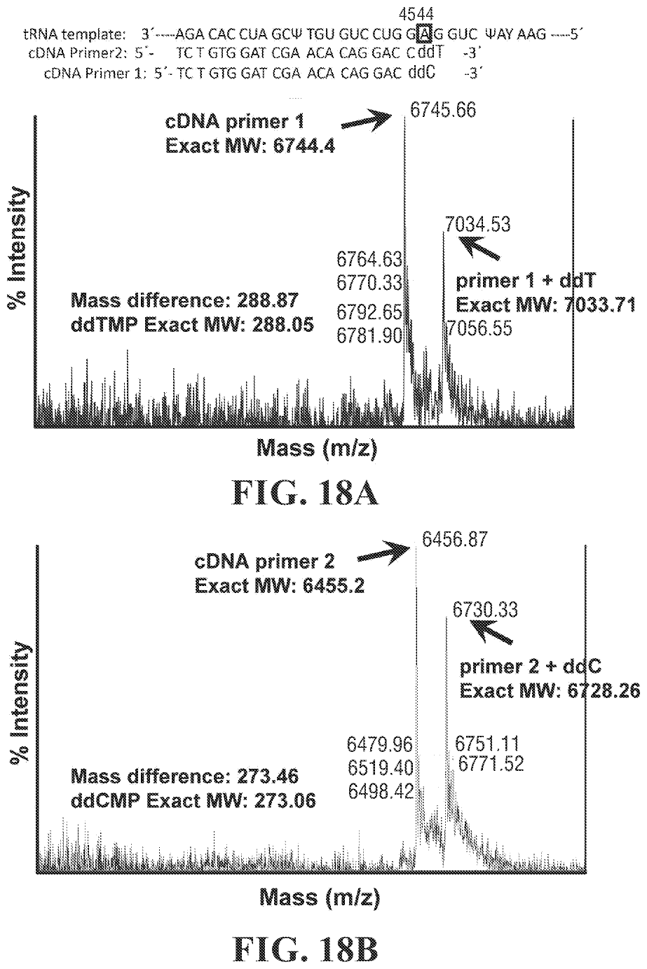

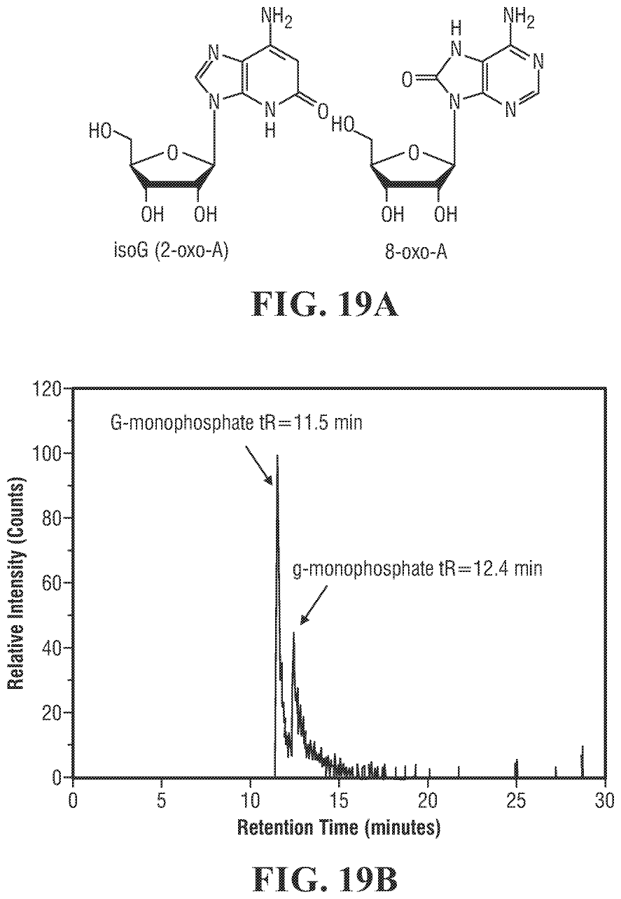

Discovering a new 44g45a isoform at the tRNA's variable loop. A new isoform with an A to G transition at position 44 and a G to A transition at position 45 was also observed, i.e., a 44A45G (wild type, reported previously) (Alzner-DeWeerd, B. et al., (1980) Nucleic Acids Res 8, 1023-1032). to 44g45a transition. Please note that the lower-case letters “g” and “a” in the isoform “44g45a” are used to represent the isomeric nucleotide that shares an identical mass with the canonical nucleotides G and A, respectively, but their exact structures remain to be confirmed. These two reads were revealed first by the anchor-based algorithm, and further verified manually in the original MFE files (FIG. 11, Tables S3-4, Table S3-5, Table S3-8, Table S3-9, and Table S3-19 through Table S3-22). Two distinct mass ladder fragments at position 44 were identified when reading from the 5′ direction, apparently corresponding to sequences containing both 44A and 44g being simultaneously present. However, these two mass ladder fragments merged into one mass ladder fragment at position 45. Such an effect could only occur if two co-existing sequences contained a 45G or a 45a, respectively, thus confirming the coexistence of two co-existing isoforms (FIG. 11A-B). This is consistent with the sequencing results when reading from the opposite direction when performed bi-directional sequencing (Bjorkbom, A., et al., (2015) Journal of the American Chemical Society 137, 14430-14438) (FIG. 11C-D). These two isoforms were observed in all reads which covered positions 44 and 45, and their relative percentages were consistent (˜50% for wild-type, quantified by EIC) (Table S3-25). To further verify the co-existence of the two mass fragments, a full-spectral analysis provided by the commercial MassWorks software (Cerno Bioscience, Las Vegas, USA) to examine the corresponding ions of these two fragments simultaneously in one spectrum. When reading from the 5′direction, two ions (m/z 778.1051 and 779.7068, both with 10 charge states) were found, corresponding to 44A and 44g. Full-spectral analysis also confirmed that 45G and 45a co-exist when reading from the 3′direction (FIG. 11D). Furthermore, the ratios of 44A/44g as compared to 45G/45a quantified by the full-spectral analysis (Wang, Y., and Gu, M. (2010), Anal Chem 82, 7055-7062) are consistent (FIG. 11), indicating that the sequenced 44g and the 45a are indeed from the same RNA strand, while the 44A and 45G are also both from the same RNA strand. All these MS results support the existence of a new isoform, with the sequence 44g45a, co-existing with the wild-type RNA that contains the 44A45G sequence, and that these two isoforms occur at similar levels. To further confirm the co-existence of these two isoforms, a rtSBE was performed on the tRNA^{Phe} sample. For example, if tRNA^{Phe} has an A/g single-nucleotide polymorphism (SNP) at position 44, then the rtSBE assay would be able to incorporate both ddT and ddC, since the two isoforms exist at similar levels. However, the results showed that only ddT could be incorporated at position 44 (FIG. 18A) and only ddC could be incorporated at position 45 (FIG. 18B), indicating that the wildtype 44A45G was the only isoform present. The rtSBE results suggested that RNA reverse transcriptase could not recognize these edited bases well. It is also possible that the mass differences observed in the above A-G transitions at positions 44 and 45 may be caused by oxidation and reduction, e.g., oxidation of A to isoG and/or 8-oxoA at position 44 (FIG. 19A), which both have a mass identical to G and would still allow canonical T incorporation. Complete acid digestion of the tRNA into single nucleotides followed by LC-MS analysis supports this, as two different t_{R}s in the EIC profile of the G monophosphate were found (FIG. 19B), suggesting a co-existing nucleotide of the same mass as G, but a different structure. A similar mechanism could explain the putative G to A transition/editing at position 45.

The 2D-HELS-AA MS Seq expands RNA sequencing capacity beyond the four canonical ribonucleotides, and is able to determine the precise order of both canonical and nucleotide modifications including potentially any modification that an LC-MS instrument can detect. Unlike other successful sequencing technologies, the presently disclosed methods rely on mass differences of two adjacent ladder fragments to report identities of both canonical nucleotides and chemical modifications. Mass is an intrinsic nucleotide property that can be used to identity both known and unknown RNA modifications. This is very different than the use of proxies such as fluorescence or electronic signatures to report the identity of the four canonical nucleotides, which has limited capacity in discovering new and unknown base modifications. It is worth emphasizing that the method is a sequencing method, which includes both identification and location information of each nucleotide, canonical or not. This is very different than other RNA identification/characterization methods, which can only indicate the identity of RNA modifications but must rely on complementary established sequencing methods for sequence/location information. The primary purpose of the currently disclosed methods is to expand the sequencing capacity of this approach beyond the synthetic RNAs reported on previously (Zhang et al., (2019) Nucleic Acids Research 47, e125), to achieve direct and de novo sequencing of biological RNA molecules like tRNA^{Phe}. Further characterization of RNA modifications was only needed when there were isomeric modifications that could not be differentiated by mass alone. The presently disclosed methods are not intended to replace standard structural verification methods such as NMR, X-ray crystallography, and other chemical and enzymatic approaches that are specific to individual nucleotide modifications, which are designed to assess the chemical structure of such base modifications. Rather, these reliable methods are important to further confirm the exact chemical structures of nucleotide modifications that have been revealed initially by their unique masses, such as isomeric base modifications.

Chemically, all RNAs consist of phosphodiester bonds that can be cleaved to generate mass ladders for the 2D-HELS-AA MS Seq. In this seminal study, the focus was to demonstrate that the approach is not limited to short synthetic RNAs (<35 nt) as described previously (Zhang, et al., (2019), Nucleic Acids Research 47, e125); but can indeed be used to sequence real biological samples such as tRNAs. However, in practice, the types of RNA that can be sequenced by this method is not only determined by the acid degradation chemistry for mass ladder generation, but as well the capacity of LC-MS instrument to detect these mass ladders. The upper limit of RNA size that will give adequate resolution is LC-MS instrument-dependent, and the lower limit of RNA sample loading amount is also instrument-sensitive. Both limits remain to be determined and will affect the utility of the approach. However, the aim is to develop a general method that every user can tailor to their own instruments. Clearly, higher end LC-MS instruments provide higher mass resolutions (likely leading to higher read length) and/or higher sensitivity (likely leading to lower sample requirement). Once the method is fully developed, it will not be necessary for every end user to have a top-of-the-line instrument, since almost certainly companies offering the service will emerge, similar to many current vendors that provide NGS services. Nonetheless, the results of the 2D-HELS-AA MS Seq revealed new isoforms, RNA base modifications and editing, as well as their stoichiometries in the tRNA that can't be determined by cDNA-based methods (FIG. 24), opening new opportunities in the field of epitranscriptomics.

### Example 4

### Materials and Methods

Acid hydrolysis degradation of tRNA. Formic acid was applied to degrade tRNA samples, including tRNA-Phe sample (Sigma) and cellular tRNA-Glu sample (see Section of tRNA-Glu sample preparation), for producing mass ladders, according to reported experimental protocols (Yoluc, Y. et al. Crit Rev Biochem Mol Biol 56, 178-204, doi:10.1080/10409238.2021.1887807 (2021); Thomas, B. & Akoulitchev, A. V. Mass spectrometry of RNA. Trends in biochemical sciences 31, 173-181 (2006); Carell, T. et al. Structure and function of noncanonical nucleobases. Angew Chem Int Ed Engl 51, 7110-7131, doi:10.1002/anie.201201193 (2012); Wein, S. et al. Nat Commun 11, 926, doi:10.1038/s41467-020-14665-7 (2020)). In brief, each RNA sample solution was divided into three equal aliquots for formic acid degradation using 50% (v/v) formic acid at 40° C., with one reaction running for 2 min, one for 5 min and one for 15 min. The reaction mixture was immediately frozen on dry ice followed by lyophilization to dryness, which was typically completed within 30 min. The dried samples were combined and suspended in 20 μL nuclease-free, deionized water for LC-MS measurement.

Liquid chromatography-mass spectrometry (LC-MS) analysis. The acid-hydrolyzed tRNA samples were separated and analyzed on a Orbitrap Exploris 240 mass spectrometer coupled to a reversed-phase ion-pair liquid chromatography (ThermoFisher Scientific, USA) using 200 mM HFIP and 10 mM DIPEA as eluent A, and methanol, 7.5 mM HFIP, and 3.75 mM DIPEA as eluent B. A gradient of 2% to 38% B in 15 minutes was used to elute RNA samples across a 2.1×50 mm DNAPac reversed-phase column. The flow rate was 0.4 mL/min, and all separates were performed with the column temperature maintained at 40° C. Injection volumes were 5-25 μL, and sample amounts were 20-200 pmol of tRNA. tRNAs were analyzed in a negative ion full MS mode from 410 m/z to 3200 m/z with a scan rate of 2 spectrum/s at 120 k resolution. The sample data was processed using the Thermo BioPharma Finder 4.0 (ThermoFisher Scientific, USA), and a workflow of compound detection with deconvolution algorithm was used to extract relevant spectral and chromatographic information from the LC-MS experiments as described previously (Yoluc, Y. et al. Crit Rev Biochem Mol Biol 56, 178-204, doi:10.1080/10409238.2021.1887807 (2021); Thomas, B. & Akoulitchev, A. V. Mass spectrometry of RNA. Trends in biochemical sciences 31, 173-181 (2006); Carell, T. et al. Structure and function of noncanonical nucleobases. Angew Chem Int Ed Engl 51, 7110-7131, doi:10.1002/anie.201201193 (2012); Wein, S. et al. Nat Commun 11, 926, doi:10.1038/s41467-020-14665-7 (2020)).

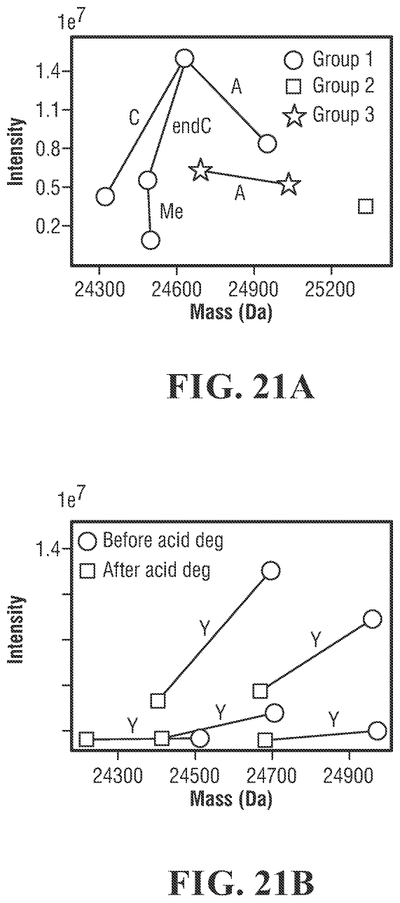

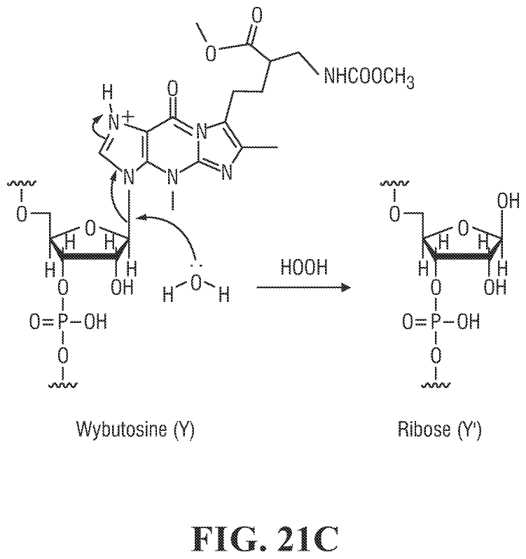

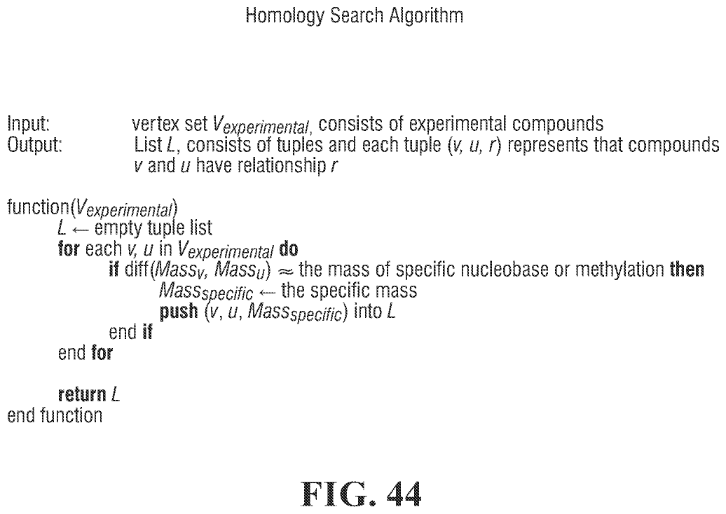

Homology search. Candidate compounds were chosen based on their monoisotopic masses around the ˜24 k Da area from both before and after acid degradation dataset, and then be analyzed using a computational tool implemented in Python (FIG. 44) that divides those compounds into various groups with each group representing one specific RNA species and its related isoforms (FIG. 21A). The tool iterates over each compound in the datasets output from each LC-MS run and exams its correlation with neighbor compounds. Compound pairs with mass differences match to specific nucleotides or modifications, such as A(329.0525 Da), C(305.0413 Da) and Methylation(14.0157 Da) get filtered out as a match, if the monoisotopic mass difference between observed value and theoretical value is within 10 ppm of for the specific known nucleotide or modification in the RNA modification database¹. Because very often, tRNAs are end with CCA at 3′ end, compounds with monoisotopic mass differences match/fit with intact mass difference 329.0525 Da would be considered as related isoforms, corresponding like to one a CCA-tailed and another CC-tailed and thus be placed into the same specific tRNA group. Similarly, compounds with monoisotopic mass differences match/fit intact mass difference 305.0413 Da would be treated as related isoforms, corresponding to CC-tailed tRNA and C-tailed tRNA and thus also be placed into the same specific tRNA group. Partial methylated/modified intact tRNA species with monoisotopic mass differences of 14.0157 Da (or other specific mass value corresponding to a nucleotide modification) would be treated as related isoforms and placed into a group for further sequencing together (FIG. 21A).

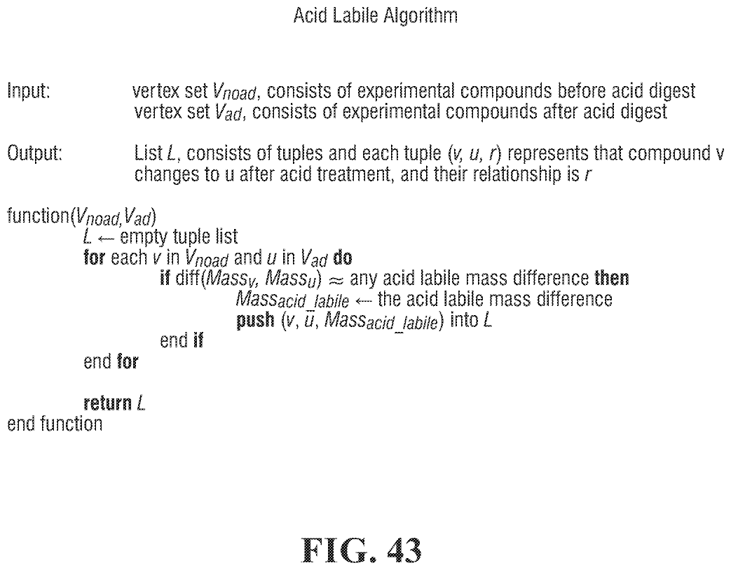

Identify acid-labile nucleotides. Acid-labile nucleotides are identified using another computational tool implemented in Python (FIG. 43). The tool analyzes the connections between the compounds before acid degradation and the ones after acid degradation. For each compound pair, one is before acid degradation and the other is after acid degradation, if the monoisotopic mass difference can match a mass difference calculated from the possible structural change to a specific nucleotide modification during acid hydrolysis or match the mass difference sum of a subset of different acid-labile nucleotide modifications, the compound pair would be selected and further considered that they may contain acid-labile nucleotide modifications (FIG. 21B).

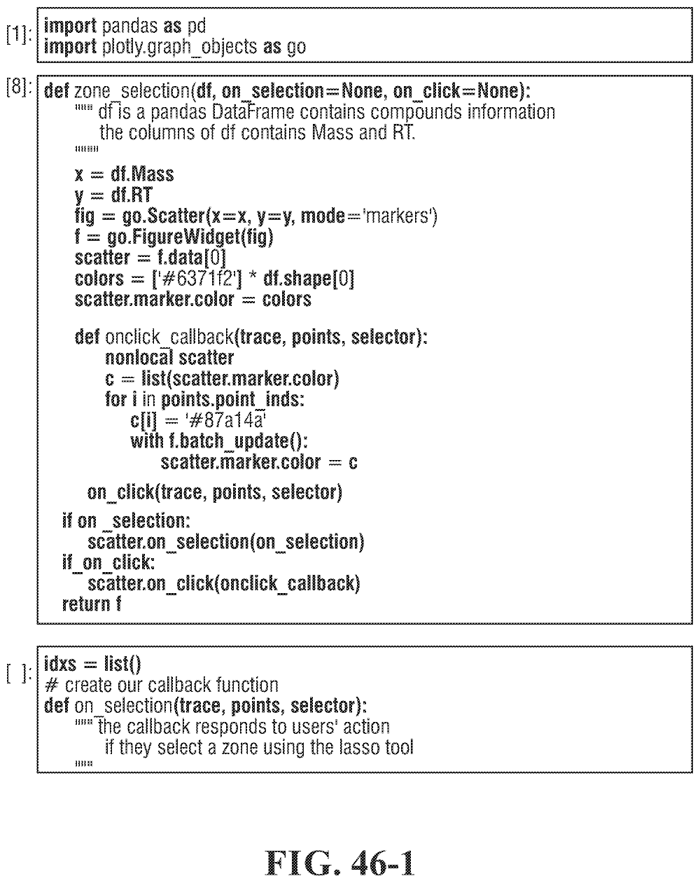

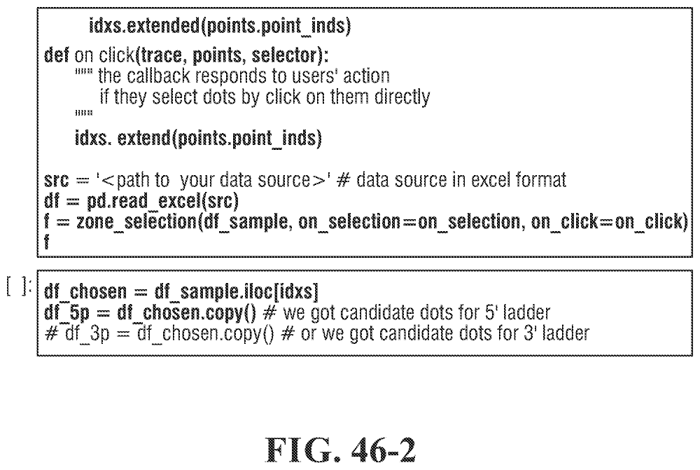

5′- and 3′-Ladder separation. tRNAs and their acid-hydrolyzed ladder fragments in datasets output from each LC-MS run are divided into two portions, one with all 5′-ladder fragments and the other with all 3′-ladder fragments. Because every tRNA 5′ ladder fragments carry with a PO₄H₂ both at the end (5′ and 3′ end), they have relative bigger t_{R} than their counterparts 3′ fragments with the same lengths after LC separation, having an up-shift in the 2D mass-t_{R} plot. As such, most 5′ ladder fragments are located above their 3′ counterparts that have the same length in the 2D mass-t_{R} graph, forming a collective curve toward the upper right corner. Due to large amount of RNA/fragment compounds, the dividing line between two subsets of 5′- and 3′-ladder fragments is not visionally decisive in the 2D plot. Thus, a computational tool (FIG. 46) was developed to separate the 5′ and 3′ fragments. In aspects, the computational tool may be run in a Jupyter notebook environment. The source code may use third-party libraries such as Plotly and/or Pandas. All the compounds in each LC-MS data pool are roughly into two subgroup areas by circling compounds in the top collective curve of the 2D mass-t_{R} plot and marking the compounds as 5′-ladder fragment compounds, while the compounds in the bottom one as 3′-ladder fragment compounds. The purpose of selecting the top area is to include as many 5′ fragment compounds as possible while as few 3′ fragments as possible. Accordingly, the purpose of the second one is to include as many 3′ fragment compounds as possible while as few 5′ fragments as possible. Overlap between two selected ladder subgroups is inevitable, due to limited t_{R} differences between these two subgroups. The aim in the manual selection step is not to separate the 5′ and 3′ fragments with a high precision, but rather use them to be served as two input ladder fragments for another algorithm to output 5′ and 3′ ladder fragments separately for each tRNA isoform/species.

MassSum data separation. MassSum is an algorithm developed based upon the acid degradation principle presented in FIG. 22. Taking advantage of the fact that each fragmented pair from two ladder groups (5′ and 3′ groups) sums up to a constant mass value that is unique to each specific tRNA isoform/specifies, the algorithm can isolate ladder compounds corresponding to a specific tRNA isoform. MassSum simplifies the dataset by grouping mass ladder components into subsets for each tRNA form/species based on its unique intact mass. Since the well-controlled acid degradation reaction cleaves RNA oligonucleotides at one specific site of the phosphodiester bond, on average, one cut per RNA, the masses of two RNA fragments (Mass_{3′ portion} and Mass_{5′ portion}) from the same strand add up to a constant value (Massₛᵤₘ).

Mass_{3′portion}+Mass_{5′portion}=Mass_{intact}+Mass_{H}_{₂}_{O}=Massₛᵤₘ (1)

Taking the advantage of this relation between the 3′ portion and 5′ portion (Equation 1), the algorithm chooses two random compounds from the acid-degraded LC-MS dataset and adds their mass values together, one pair at a time. If the sum of the selected two compounds equals a specific Massₛᵤₘ, these two compounds will be set into the pools accordingly. The process repeats until all compound pairs have been inspected. In the end, MassSum will cluster the dataset into several groups with Mass., each group is a subset that contains 3′ and 5′ ladders of one RNA sequence. MassSum pseudocode can be found in the supplementary information.

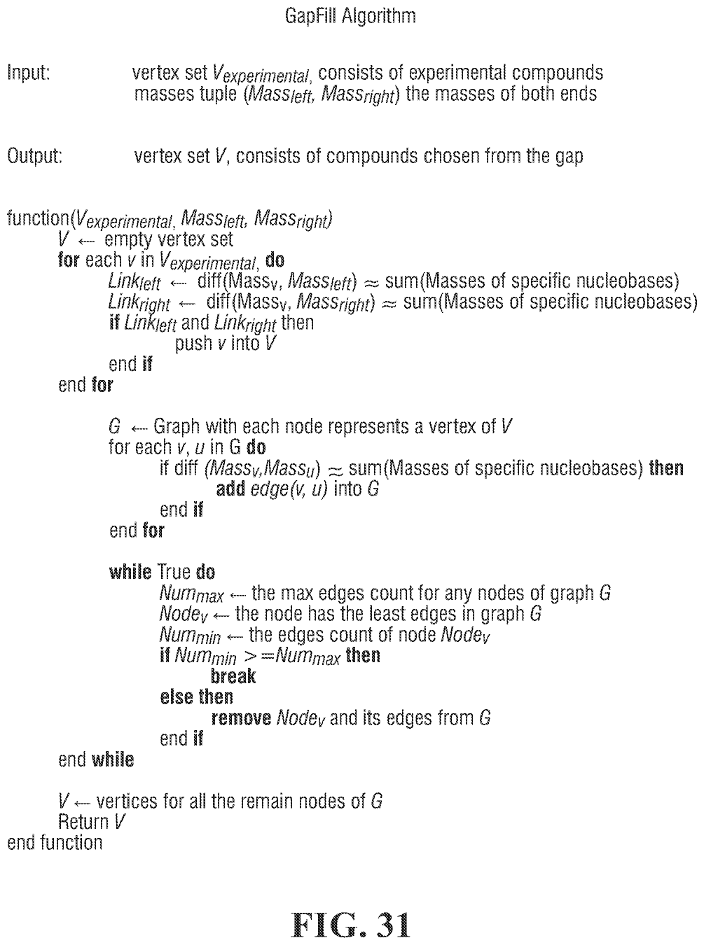

Gap Filling. GapFill is another algorithm developed as a complementary of MassSum (FIG. 31). From the above, it is known that MassSum handles compounds in pair, if one compound was missing from the pair, MassSum will ignore this compound as well. GapFill was designed for this case and can save those compounds have counterparts missing in either 3′- or 5′-ladder (but not both). Suppose Mass_{5′i} and Mass_{5′j} are two non-adjacent compounds from the 5′ ladder, the area between these two ending compounds is defined as a gap. Among the gap there exists many compounds in degraded LC-MS dataset but not one got selected out after MassSum data separation. GapFill iterates over each potential compound in the gap in the original LC-MS dataset before MassSum, exams the mass differences of this compound and the two ending compounds with Mass_{5′i} and Mass_{5′j}. If the mass difference equal to the sum of one or more nucleobase/modifications in the RNA modification database¹, it is defined as a connection. If the compound in the gap has connections with both ending ones, this compound would be kept into a candidate pool in the process later for sequencing. After iteration, GapFill calculates connections of the compounds pairwise in the candidate pool and assigns weights to them based on the frequency of each connection. The compounds that contain the highest weights would be the ones chosen to fill in the gap. (see, Table S4-1 through Table S4-3)

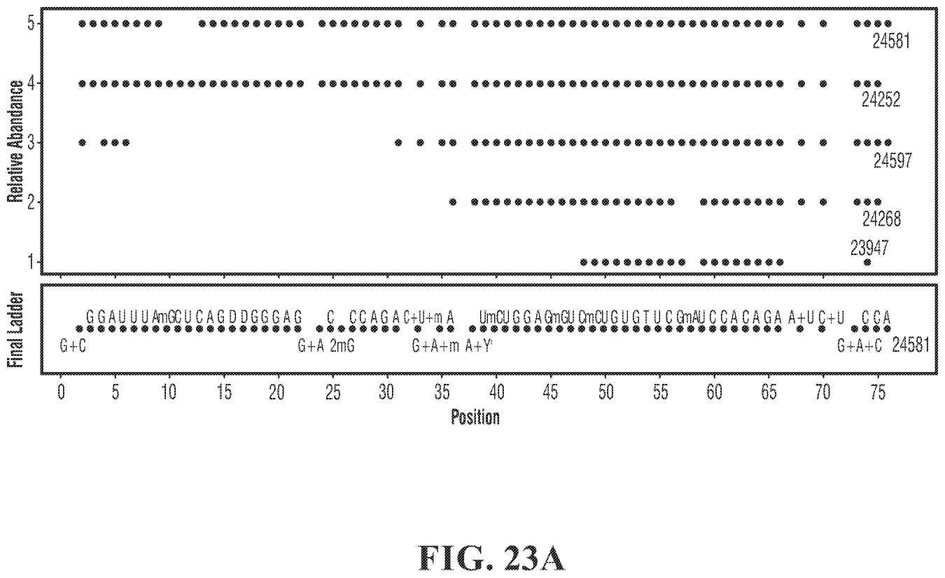

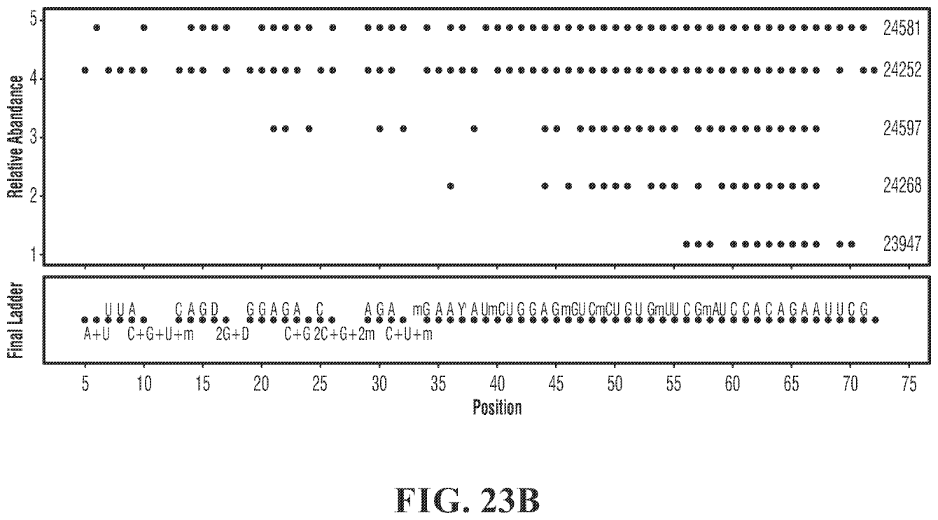

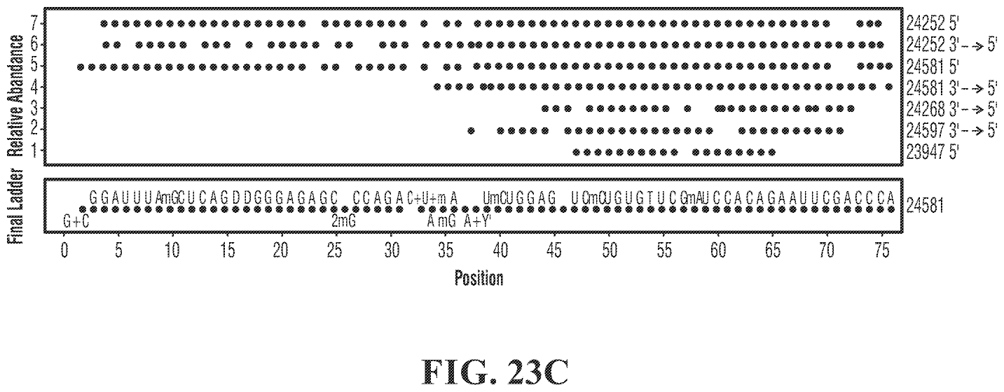

Generation of RNA sequences containing canonical and modified nucleotides and Ladder complementary. After MassSum and GapFilling, each tRNA isoform has its own 5′- and 3′-ladders separately (not combined). Each ladder (5′- or 3′-) consists of a ladder sequence, and one can read out if these ladders are perfect without missing any ladder fragment corresponding to the first to the last nucleotide in the RNA. Otherwise, if not, one can complement ladders from other related isoforms in order to get a more complete ladder needed for sequencing. A computational tool was implemented to align these ladders based on the position from the 5′→3′ direction, as long as the position has a mass/base from any ladder, this base will be called and put into the complementary result (FIG. 45). First, ladder complementarity is done separately on 5′ and 3′ ladders, resulting in one final 5′ ladder and one final 3′ ladder separately (FIG. 23A-B). Besides 5′ and 3′ isoform ladders ladder complementing inside the 5′ or 3′ ladders (without crossing between 5′ and 3′ ladders), one may also computationally convert the 3′ ladder into its 5′ ladder based on the MassSum of each RNA isoform, and complementing converted 5′ ladder with original 5′ ladder of each RNA isoform for a perfect or better ladder needed for MS-based sequencing of RNA (FIG. 23C). Alternatively, the two 5′ and 3′ ladders can be read out separately and their overlapping sequence can be used to re-affirm each other, producing the final sequence ladder.

tRNA-Glu sample preparation. Total RNA from cells with or without RSV infection was extracted using Trizol and followed by pull-down using Biotin-GluCTC probe and streptavidin-beads at 4° C. overnight. After DNase treatment, pull-downed RNA was extracted using Trizol and followed by acid hydrolysis degradation and lyophilization.

NGS sequencing of tRNA-Glu sample. The above-prepared tRNA-Glu sample were delivered to Eureka Genomics (Houston, Tex.) for small RNAs isolation, directional adaptor ligation, cDNA library construction, and sequencing using a Genome Analyzer IIx (Illumina, San Diego, Calif.). About 485 Mb of sequence data with a total of 32,332,590 sequence reads was generated for mock- and RSV-infected samples, using 36 b single-end sequencing reads.

MS sequencing of tRNA-Glu sample. After homology search on tRNA-Glu dataset, it was noticed that most of the tRNA-Glu isoforms are related to each other, and they have either a methylation difference or a 1 Dalton mass shift. After MassSum and GapFill on the degraded dataset, one can de novo read out a couple of sequence segment (see FIG. 24C), e.g., 8U to 24A, and 36C to 44C. With the de novo sequencing information, BLAST NGS sequences dataset was done, and a few matched NGS sequences were found. The one with highest intensity was selected first to calculate theoretical masses for each acid-hydrolyzed ladder fragment in silica. Different mass shifts were applied, based on the patterns of mass differences between observed ladder fragments and NGS-based calculated mass fragments, directly onto the NGS-based sequence ladder fragments and filter out the observed compounds from degraded dataset. As a result, the entire tRNA-Glu can be sequenced with the different modifications from those observed compounds, which contains some novel information that was not previously reported for the tRNA-Glu (see FIG. 24F).

A549/RSV Infected A549 Cell Line tRNA Extract Using Probe

Cell Preparation and Total RNA Extraction. Seed A549 cells were placed into T-150 flasks to be 90% confluent in the next day. After 20-24 h, infect cells with RSV at an MOI of 1 for RSV samples or just change the media for Mock samples (no infection). Then the cells were collected and rinsed with cold 1X phosphate buffered saline (PBS). Trizol reagent was used to extract total RNA. Chloroform (0.2 mL per 1 mL Trizol reagent) was added to the cells and mixed completely. At 4C, the mixture was centrifuged at 12,000×g for 15 min. The upper aqueous phase was then transferred into a new tube and added 0.5 mL 2-propanol, mixed gently and incubated for 10 min at room temperature. Centrifuge at <7500×g was performed on the mixture for 5 min. The supernatant was discarded, and the pellet was washed with 1 mL of 75% EtOH. Centrifuge was performed again at <7500×g for 5 min at 4 C. The supernatant was discarded and the pellet was dried in air for 5-10 min. The pellet was then dissolved in DEPC water. The concentration of extracted total RNA was extracted, 1/10^{th} was saved as an input. (Usually, you can get 1 mg of total RNA from three T-150 flasks. All samples were kept at −80 C.

Hybridization in the Presence of Btn-GluCTC probe. 7504, total RNA (1 mg) in DEPC water was mixed with 250 μL Btn-GluCTC probe (104, of 100 μM stock) in 20×SSC buffer. After 5 μL RNase inhibitor was added, the mixture was incubated and heated for 15 min at 65C and then slowly cooled down in room temperature for 3 h to and complete the hybridization. Another 5 μL RNase inhibitor was added 1h after the mixture was transferred to room temperature.

Precipitation of the Hybrids. Streptavidin-beads (Thermo Scientific, Cat No. 20349) was washed with 5×SSC buffer twice, and 100 μL of them were added to the above mixture of total RNA and Btn-GluCTC probe in 1 mL of 5×SSC buffer. Gentle rotation was applied while the mixture was incubated overnight at 4C. Pellets beads were then collected by centrifuging at 500×g for 1 min at 4C and the supernatant was removed and stored separately at −80 C (just in case). Under gentle rotation, the beads were washed with 1 mL 1×SSC buffer for 5 min at 4 C. The pellets were then submitted to centrifuge 500×g for 1 min at 4 C and the supernatant was discarded. The beads were then washed with 1 ml of 0.1×SSC buffer for 5 min at 4 C using gentle rotation centrifuged. The last wash and centrifuge were repeated twice.

DNase I Treatment, Precipitation and Purification of RNA Extract. DNase I was used to digest DNA probe completely. 200 μL DNase I reaction mixture (NEB, Cat No. M303S) to the beads, and the mixture was incubated at 37 C for 10 min.

| Components | DNase I reaction mixture | |
|---|---|---|
| DNase I Reaction Buffer (10×) | 20 ul | (1×) |
| DNase I (RNase-free) | 10 ul | (20 units) |
| RNase inhibitor | 2 ul | (400 U/ml) |

| | |
|---|---|
| DEPC Water | To 200 ul |

The mixture was subjected to centrifuge at 500×g for 1 min at 4 C, the supernatant was transferred to another tube, to which 0.75 mL of Trizol LS reagents were added. The RNA targeted RNAs were precipitated using the following procedure. 0.2 mL Chloroform was added to the liquid mixture and mixed completely. Centrifuge was performed at 12,000×g for 15 min at 4 C. Then the upper aqueous solution was transferred to a new tube, to which 0.5 mL 2-propanol was added, mixed gently and incubated for 10 min at room temperature to precipitate RNAs out. The mixture was submitted to centrifuge at 12,000×g for 10 min at 4 C. The supernatant was removed carefully, and the pellet was added with 1 mL 75% EtOH. In this step, 1 ul (5 ug) of Linear acrylamide solution (Fisher Scientific, Cat No. NC1781917) was added to visualize the RNA pellet. Centrifuge was performed again at <7500×g for 5 min at 4 C. The supernatant was discarded and the pellet was collected and dried in the air for 5-10 min. The extracted RNA pellet was dissolved in DEPC water and purified using Oligo Clean & Concentrator Kit (Zymo, Cat No. D4060) according to the instruction.

LC-MS analysis. Samples were separated and analyzed on an HPLC coupled to an ThermoFisher Exploris 240 Mass Spectrometer. The dried samples were re-suspended in 100 μL of LCMS grade H2O/1% MeOH, 100 μM EDTA to bring the final concentration to 20 pmol/μL. The HPLC separations were performed on HPLC with (A) as 200 mM HFIP and 10 mM DIPEA aqueous solution (B) as 7.5 mM HFIP and 3.75 mM DIPEA methanol solution across a 2.1×50 mm DNAPac column with a particle size of 4 μm. For acid-degraded yeast tRNA-Phe, mobile phase B was ramped from 20% to 38% in 15 mins. The flow rate was 0.4 mL/min and all the separations were performed with the column temperature maintained at 40° C. Injection volumes were 5-25 μL and sample amounts were 20-200 pmol of tRNA. tRNAs were analyzed in a negative ion mode from 410m/z to 3200 m/z with a scan rate of 2 spectrum/s at 120 k resolution. The data was processed using the Thermo BioPharma Finder 4.0 (ThermoFisher Scientific, USA), and a workflow of compound detection with deconvolution algorithm was used to extract relevant spectral and chromatographic information from the LC-MS experiments.

### Results

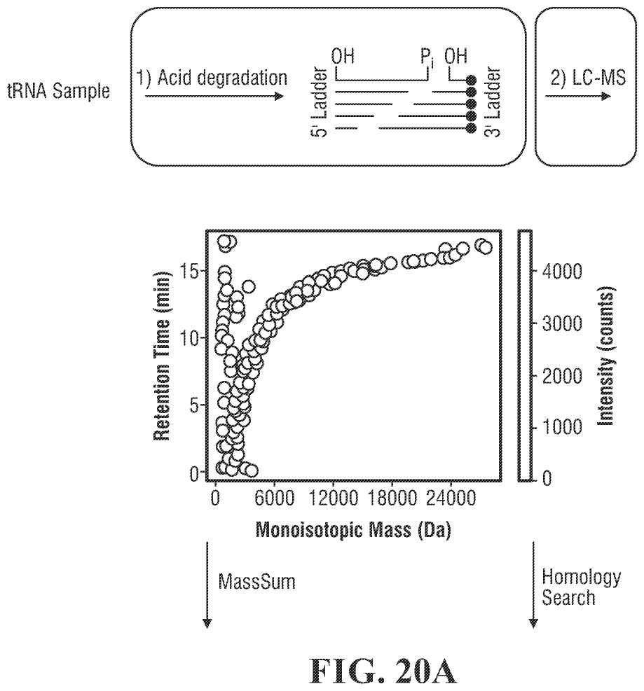

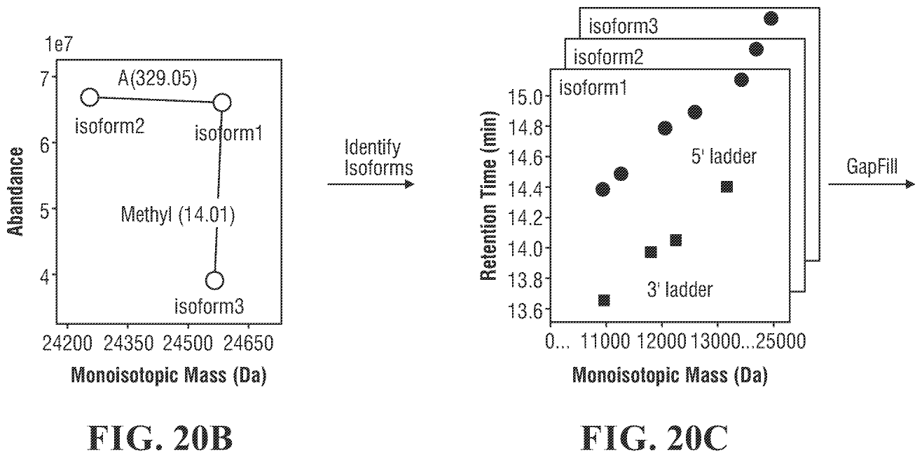

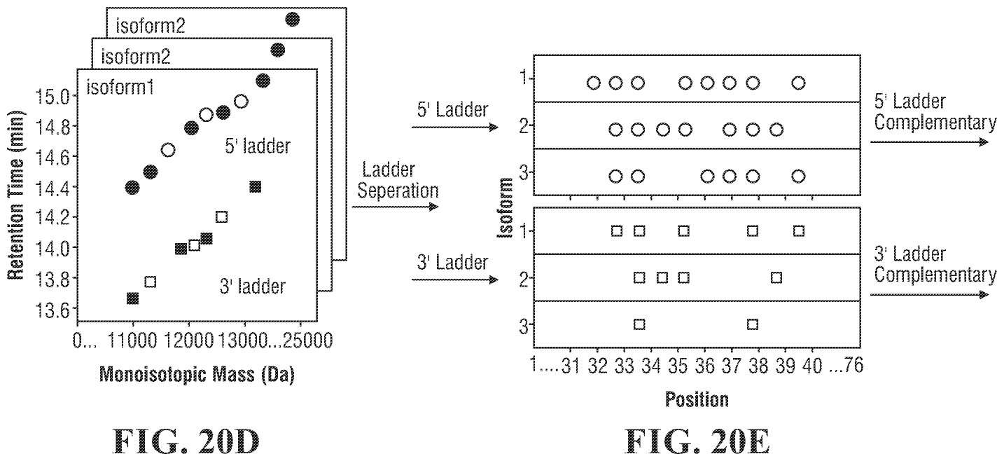

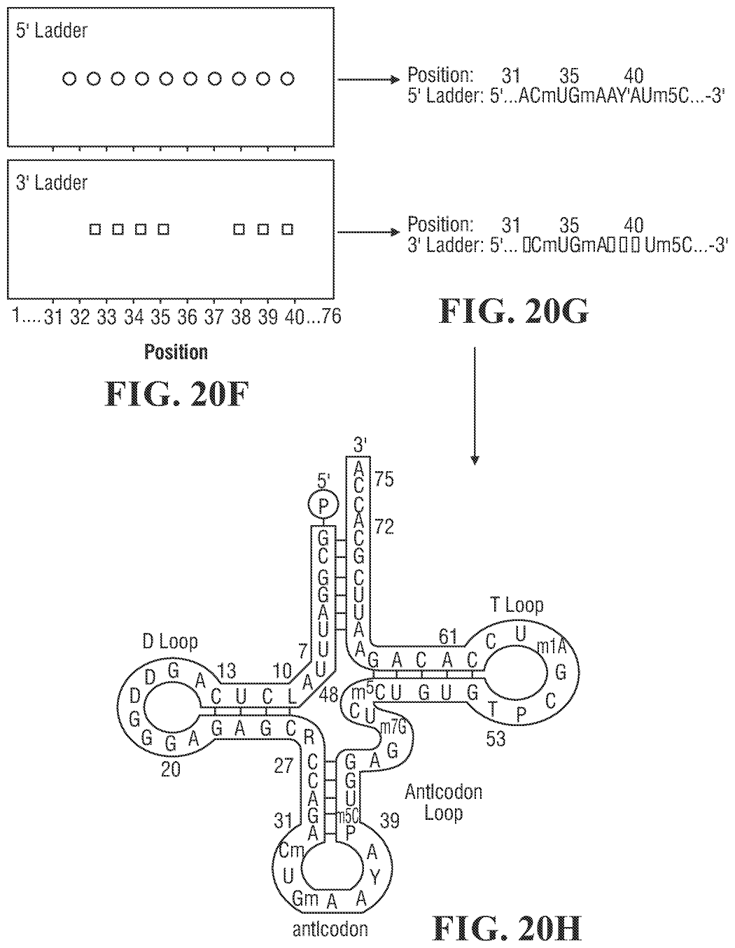

Workflow of de novo sequencing of tRNA isoform mixtures. In order to de novo MS sequence of tRNA isoform mixtures, systematic efforts have been made to overcome the current physical limits, especially in sample preparation, read length, and throughput. As shown in FIG. 20, workflow of the method is easy-operated, and includes three major steps only: 1) acid hydrolysis of tRNA samples (single-stranded or mixed) in well-controlled conditions to general ladder fragments (Zhang, N. et al. Nucleic Acids Res 47, (2019); Zhang, N. et al. ACS Chem Biol 15, 1464-1472 (2020); Bjorkbom, A. et al. Journal of the American Chemical Society 137, 14430-14438 (2015); Zhang et al., J. Vis. Exp. e61281 (2020)), 2) LC-MS detection of the resultant acid-degraded tRNA samples, containing tRNAs (intact or degraded) and all their acid-hydrolyzed fragments, and 3) data processing and generation of sequences made of both canonic and modified nucleotides (if they exist). The last step is apparently the most challenging and requires a complete set of step-wise innovative computational methods/tools, including algorithms mainly for homology search, identifying acid-labile nucleotide, mass-sum-based data separation, gap-filling, ladder separation, ladder complementing, and sequence generation, as described below.

Once output LC-MS data into a 2D mass-retention time (t_{R}) plot, a homology search of intact tRNAs in the mass range of >˜24 k Dalton (or ˜75 nt; on average ˜318 Dalton/nt) is started using an in-house developed algorithm (FIG. 44) to first identify related tRNA isoforms that may share the same ancestry precursor tRNA, but are deferent, e.g., in posttranscriptional modification profiles. Mass differences between two intact tRNA isoforms are calculated and match with the known mass of each nucleotide or nucleotide modification in the database (Bjorkbom, A. et al. Journal of the American Chemical Society 137, 14430-14438, doi:10.1021/jacs.5b09438 (2015)). For example, known mass difference between these intact tRNAs such as 14.0157 Da and 329.0525 Da (with PPM difference <10 ppm) (Brenton, A. G. & Godfrey, A. R. J Am Soc Mass Spectrom 21, 1821-1835, doi:10.1016/j.jasms.2010.06.006 (2010)). can be assigned to a methylation (Me/—CH₂—) and a nucleotide A, respectively. Therefore, these intact tRNAs are assigned to the same tRNA group and considered as specific tRNA isoforms to be further sequenced together.

To read/sequence tRNA isoforms from complex mixtures, a new algorithm was develped, named as MassSum (FIG. 30), based on the fact that the mass sum of any set of paired fragments generated during acid-mediated degradation of RNA by cleavage of one phosphodiester bond is constant (equivalent to the mass of each undegraded RNA plus the mass of a water molecule) (see FIG. 20 and FIG. 22A) (Bjorkbom, A. et al. Journal of the American Chemical Society 137, 14430-14438, doi:10.1021/jacs.5b09438 (2015)). Using this constant and each tRNA isoform's unique mass, one can computationally isolate MS data of all ladder fragments derived/degraded from the same tRNA isoform sequence in both the 5′- and 3′-ladders out of the complex MS data of mixed samples with multiple distinct RNA strands. After MassSum separation and subsequently filling ladder fragments missing in either one of two ladders (5′- or 3′-ladder) with a GapFilling algorithm, one can further computationally separate 5′- and 3′-ladders from each other based up the sigmoidal curve that each ladder (5′- or 3′-) has in the 2D mass-t_{R} plot (Bjorkbom, A. et al Journal of the American Chemical Society 137, 14430-14438, doi:10.1021/jacs.5b09438 (2015)). Once the data separation is accomplished, one can then use the anchor-based algorithm (Zhang, N. et al. ACS Chem Biol 15, 1464-1472, doi:10.1021/acschembio.0c00119 (2020)). to automate sequence generation separately for each tRNA isoform in the mixture. In case it has a perfect ladder (5′- or 3′-), each tRNA isoform can be sequenced twice via bi-directional sequencing (reading 5′- and 3′-ladders), which has been used previously to paired-end read terminal nucleotides (Bjorkbom, A. et al. Journal of the American Chemical Society 137, 14430-14438, doi:10.1021/jacs.5b09438 (2015)), to enhance sequencing accuracy (Zhang, N. et al. Nucleic Acids Res 47 (2019), and to double the read length (Zhang, N. et al. ACS Chem Biol 15, 1464-1472, (2020).

However, very often a perfect ladder for any tRNA isoform after acid degradation does not exit, e.g., due to its sample scarcity and/or low stoichiometry of posttranscriptional modifications, and there are ladder fragments missing. Traditionally this ladder if faulted to some degree was considered as a lethal damage for its MS-based sequencing. Here one is able to fix the ladder damage and thus resume the sequencing by combining the ladder fragments from other isoforms of the same tRNA group cataloged in the above-mentioned homology search. Since each ladder fragment carries position information itself (˜318 Da/nt), after reconciling the mass difference between different isoforms, a ladder fragments missed in one tRNA isoform may get complemented by a counterpart fragment from another tRNA isoform, leading to the completion of a perfect ladder needed for MS sequencing. For example, the 5′-ladder fragment missing at position 34 of Isoform #1 can get fixed site-specifically by the counterpart ladder fragment from Isoform #2, while the ladder fragment missing at position 40 of Isoform #2 can get fixed by the counterpart ladder fragments from both Isoforms #1 and #3 (FIG. 23). As such, a perfect 5′-ladder that does not miss any ladder fragment, can be formed for sequencing of the tRNA group, including all the isoforms #1-3. Dependent on the sample quality and quantity, there are cases where ladder fragments are still missing in the 5′-ladder even if ladder complementing from all other isoforms, 3′-ladder can also be used to fix the missing fragments site-specifically for sequence completion of the tRNA, or fix the missing piece of sequence after reading out sequences from both ladders (5′- and 3′-).

For each tRNA, ladder complementing between different isoforms can be performed inside either 5′-ladder or 3′-ladder; ladders can also get complemented to some extend by crossing between 5′-ladder and 3′-ladder where ladder fragments are responsible to the overlapping sequence of each tRNA isoform. The order of these two types of ladder complementing can be alternate. In some cases, it may not need to have both types of ladder complementing when ladders are in good quality. However, both will become necessary when ladders are in poor quality, like due to sample scarcity or low stoichiometry of RNA modifications. For a very minor tRNA species (with relative abundance <1%), one may not able to achieve completion of a perfect ladder for its sequencing, even with all the above-mentioned ladder complementing measures. However, one is still able to gather all ladder fragments that can be detected by the LC-MS and use them to de novo assemble/produce the tRNA sequence (including modifications) in part, which can be also useful to blast out the entire tRNA sequence, e.g., either from NGS sequencing results performed in parallel or from reported tRNA sequences in literature/databases (FIG. 22E-G). With this way, not only RNA sample in good quality and in high abundance, but also RNA in poor quality and in low abundance can be sequenced simultaneously. Successful implement of the workflow will make it feasible for MS to sequence complex cellular RNAs, and thus pave a way toward de novo MS sequencing of biological RNA in large scale.

Increasing method's read length from ˜35 nt to ˜76 nt per LC-MS run, allowing direct sequencing of any tRNA specifies without T1 digestion/fragmentation. As a way to push the threshold of the method's sequencing read length, the LC-MS instrument with a mass resolution power of 120 k was chosen to analyze the tRNA samples in the manuscript. Previously with a 40K mass resolution LC-MS, it was only possible to read segments of up to ˜35 nt long, and thus a partial RNase T1 digestion step was required in the sample preparation to reduce the tRNA to segments of sequenceable sizes (Zhang, N. et al. ACS Chem Biol 15, 1464-1472, (2020)). When sequencing a 76 nt tRNA-Phe, instead of the entire tRNA, only its segments digested partially by T1 were sequenced. As such, one more extra step would be required to assemble the full-length tRNA-Phe sequence based on overlapping sequence reads from different LC-MS runs. An important improvement for the method would be to increase the read length, allowing the entire tRNA sequence directly without requiring T1 digestion into smaller fragments.

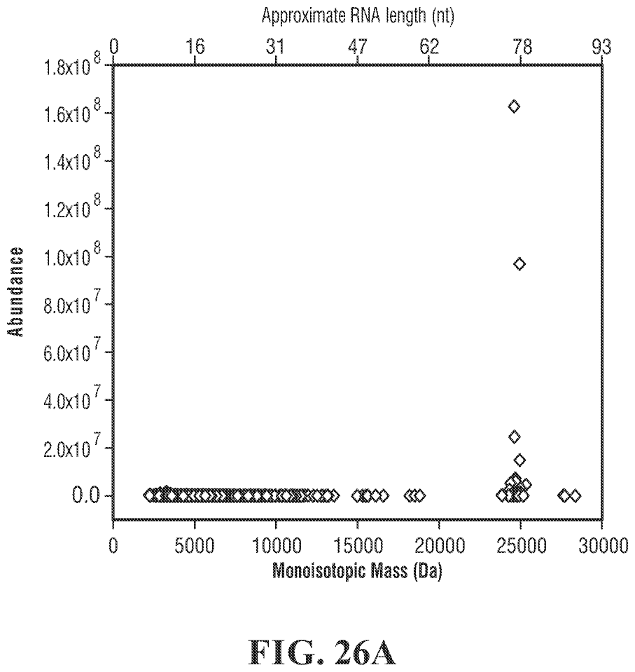

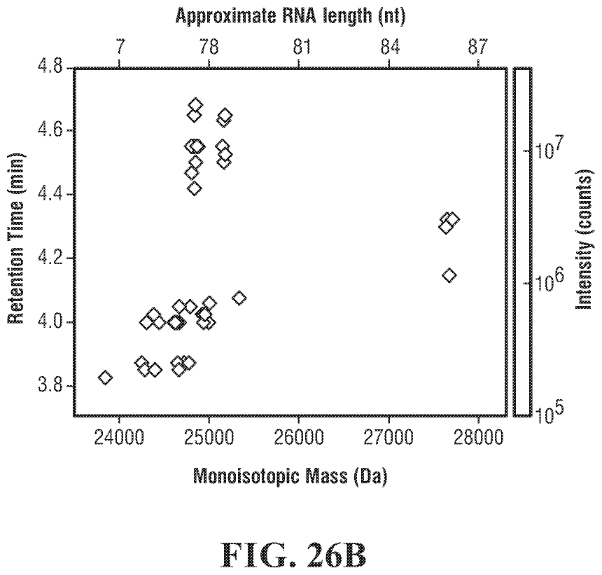

The results demonstrate that one is now able to achieve this milestone mainly by using a state-of-the-art LC/MS Orbitrap with 120K resolution (Thermo Fisher Scientific), which can correctly determine RNAs up to 76 nt (with a mass of −25K Dalton) and maybe longer (to be determined). As shown in the 2D mass-t_{R} plot (FIG. 20A and FIG. 26), the LC-MS can accurately determine monoisotopic masses for intact tRNA species (in the mass range >24K Dalton) and their acid-hydrolyzed ladder fragments (ranging from ˜300 to ˜24 k Dalton), making it possible to read the sequence of the entire tRNA directly. Indeed, after data processing and separation via a mass sum strategy (FIG. 22), the entire 76 nt tRNA-Phe, including all 11 modifications, can be directly sequenced by the anchor-based algorithm without T1 pre-fragmentation.

Although the full potential of the method's read length remains to be explored, the improvement significantly simplifies the sample preparation and makes it much easier for LC-MS to sequence various specific tRNAs, including their different nucleotide modifications, directly in one study. Being able to detect the intact masses of tRNA species makes it possible to find/identify related tRNA isoforms in an RNA sample via homology search, eventually making it possible to utilize ladder fragments between each individual tRNA isoform in a complementary manner toward completion of a perfect ladder for MS sequencing.

Homology search before acid degradation for identifying the related tRNA isoforms. After transcription, tRNAs are processed by multiple post-transcriptional regulatory mechanisms including base editing/modifications and the addition of 3′ terminal bases²¹. For some modifications, every tRNA transcript copy will be modified at a certain position (i.e., 100% stoichiometry), in other cases, the nucleotide modification stoichiometries may be variable²², may be regulated, and may have therefore confer different properties onto the tRNA depending on the modification status (Lyons, S. M., Fay, M. M. & Ivanov, P. FEBS Lett 592, 2828-2844, doi:10.1002/1873-3468.13205 (2018)). Thus, tRNAs can exist as distinct isoforms as a result of different chemical modifications. The CCA trinucleotide is synthesized and maintained by stepwise nucleotide addition to a post-transcribed tRNA by the ubiquitous CCA-adding enzyme without the need for a template (Hou, Y. M. IUBMB Life 62, 251-260, doi:10.1002/iub.301 (2010)), resulting in mature and active tRNA with a CCA-attached tail on the 3′ end. Relative isoform distributions and base modification profiles in tRNA may differ depending on the tissue type, existence of a disease state, or even the age of the tissue due to variations in protein synthesis rate. The percentage of mature tRNA among its precursor isoforms was suggested to be related to the subsequent metabolic rate of protein synthesis, and has implications in many diseases such as obesity, diabetes, and cancers (Mahlab, S., Tuller, T. & Linial, M. RNA 18, 640-652, doi:10.1261/rna.030775.111 (2012); Borek, E. et al. Cancer Res 37, 3362-3366 (1977)).

Homology search are performed between tRNA isoforms that may share the same ancestry precursor tRNA, but are deferent in modification profiles and 3′ end truncations (full-length CCA-tail mature RNA vs. the truncated isoforms). In the mass range of >24K Dalton in the 2D mass-t_{R} plot, an algorithm was developed (FIG. 44) to examine the monoisotopic mass of each intact tRNAs measured on the latest Obitrap LC-MS in order to group each specific tRNA species together with its isoform caused by partial RNA modification or 3′ end truncations. Cataloging of each group is based on the mass differences between any two intact tRNA species/isoforms. If their mass difference matches with a known mass difference for a nucleotide or a modification in the RNA modification database⁸, these two intact tRNAs are assigned to the same tRNA group and considered as potential tRNA isoforms to be further sequenced together. Taking tRNA-Phe (Sigma) measured before acid degradation as an example (FIG. 21A and FIG. 26), intact tRNA isoforms with a monoisotopic mass of 24939.55, 24610.49, 24305.40, 24385.35, and 24399.39 were assigned to the same group (#1), because their mass differences with each other, 329.0525 Da, 305.04 Da, and 14.0157 Da, and (with PPM difference <10 ppm), can be assigned to a nucleotide A, a nucleotide C, a nucleoside C (without a phosphate), and a methylation (Me/—CH₂—) respectively, indicating that they may be three 3′-CCA-tail-truncated tRNA isoforms (each ended with a C, a CC, and a CCA at 3′-end) together with one degraded isoform and its partially methylated isoform. Similarly, intact tRNA isoforms with a monoisotopic mass of 24626.46 and 24955.52 were cataloged into the same group (#3) because their mass difference 320.05 can be assigned to a nucleotide A. The Intact tRNA with a mass of 25334.63 stands alone a group and cannot be related to other tRNA isoforms. A complete list of all monoisotopic masses of intact tRNA species in the tRNA-Phe sample (Sigma) can be found in Table S4-1.

It should be pointed out that the homology search is a non-target pre-selection to group possible tRNA isoforms together for sequencing. However, only one monoisotopic mass difference of intact masses has been used to identify the tRNA isoforms differed by RNA editing/modifications and/or 3′-CCA truncations. Thus, there may be errors when grouping a tRNA isoform that does not belong to this group or the opposite, missing a tRNA isoform when cataloging a group. These errors can be fixed later when sequencing each group of tRNA isoforms, and sequencing results can further verify the inter-connection between isoforms.

The four intact tRNA isoforms in group #1 were further MS sequenced. The three intact tRNA isoforms in group #1 with monoisotopic masses of 24939.55, 24610.49, 24305.40 are indeed the related, and they are 76 nt mature 3′-CCA-tailed tRNA-Phe and its two 3′-truncated isoforms, 75 nt CC-tailed tRNA-Phe and 74 nt C-tailed tRNA-Phe, respectively. The two other isoforms in group #1 with monoisotopic masses of 24385.35 and 24399.39 are also related. The isoform with a monoisotopic mass of 24385.35 Dalton is 75-nt CC-tailed tRNA-Phe but partially degraded and lost a nucleotide C, thus becoming a 74 nt isoform. Unlike the previous three isoforms that have 3′ hydroxyl, this degraded 74 nt isoform has a new monophosphate in the 3′ end with a 80 Dalton mass increase when comparing to that of 74 nt C-tailed tRNA-Phe. The isoform with a monoisotopic mass of 24399.39 Dalton is a methylated isoform of the degraded 74-nt CC-tailed tRNA-Phe. Identification of all related isoforms in the homology search, including methylated and 3′-CCA-tail-truncated, serve as a solid foundation for mass complementary laddering sequencing.

Stoichiometric quantification of the related tRNA isoforms identified in homology search. One can quantify the relative percentage/stoichiometry of these isoforms using their relative abundances together with their extracted ion current (EIC) (Zhang, N. et al. A general LC-MS-based RNA sequencing method for direct analysis of multiple-base modifications in RNA mixtures. Nucleic Acids Res 47, e125, doi:10.1093/nar/gkz731 (2019); Zhang, N. et al. ACS Chem Biol 15, 1464-1472 (2020); Zhang, et al., P Natl Acad Sci USA 110, 17732-17737, (2013)). The most abundance two monoisotopic masses in FIG. 21A are 24610.491 Dalton and 24939.549 Dalton, corresponding to 75 nt and 76 nt tRNA-Phe, respectively. The stoichiometry of the three isoforms can be quantified to be 37:62:1 for 76 nt: 75 nt: 74 nt isoforms, respectively (See, Table S4-3). The tail-truncation 75 nt-CC-ended and 74 nt-C-ended isoforms were not degraded from the complete 76 nt-CCA-tailed form because 1) the sample was directly from the vendor and did not go through acid degradation, and 2) degradation products would have a phosphate at 3′ end, while three 3′-CCA truncated isoforms contain the free 3′-OH. Similarly, stoichiometry can be interpolated for the pair of isoforms: 75 nt CC-tailed tRNA-Phe and its partial methylated isoform (56:44).

Identify each tRNA containing acid-labile nucleotide modifications by comparing the mass changes of the intact tRNA before and after acid degradation. Acid degradation has been used to generate an MS ladders, which is easy to operate and is well-controlled. However, one major concern is the effect of acid hydrolysis used in sample preparation, on structures of nucleotide modification (Yoluc, Y. et al. Crit Rev Biochem Mol Biol 56, 178-204, (2021)). It has been reported that the modified nucleoside N6-threonylcarbamoyladenosine (t6A) is actually present in vivo as the cyclic form (ct6A) and that sample preparation could lead to hydrolysis and ring opening prior to mass spectrometry detection (Matuszewski, M. et al. Nucleic Acids Res 45, 2137-2149(2017)). This concern can be addressed by comparing the mass changes of the intact tRNA before and after acid degradation. If there are acid-labile RNA modifications that are sensitive to the acid treatment, one can piece them together with MS information before and after acid treatment (Zhang, N. et al. ACS Chem Biol 15, 1464-1472, (2020)). This, in turn, can help to identify which tRNA contains acid-labile nucleotide modifications and where they are in the tRNA molecule, and to find the ladder fragments with a mass change caused by acid degradation/hydrolysis for sequencing of the tRNA.

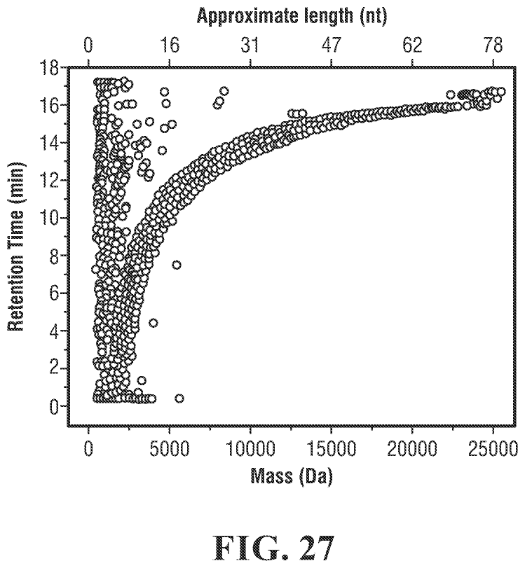

After acid treatment of the tRNA-Phe sample, the first and second abundant masses (24610.491 Da and 24939.549 Da) disappeared completely and two new masses (24252.311 Dalton and 24581.381 Dalton) show up, each producing a difference of 358.168 Dalton, respectively, when comparing to first and second abundant masses before acid degradation (FIG. 21B and FIG. 27). This specific mass difference matches the unique change caused by the conversion of wybutosine (Y) to its depurinated ribose form (Y′) in the acidic conditions (FIG. 21C). Therefore, the acid-labile Y was further confirmed to be in position 37 when sequencing the tRNA and its isoforms. In fact, the monoisotopic masses of all five tRNA-Phe related isoforms identified in the homology are found to decrease 358.168 Dalton (FIG. 21B), corresponding to the conversion of Y to Y′ caused by acid hydrolysis. As such, the depurinated intact mass of these five isoforms, i.e., 24252.311, 24581.381, 24597.35, 24268.30, and 24027.24 Dalton, were used as intact masses in the MassSum algorithm for the searches of mass pairs.

If intact mass did not change after acid degradation, use this intact mass for mass sum. If intact mass did change after acid degradation, identify the acid-labile nucleotides by matching their observed mass differences with theoretical mass differences caused by acid-mediated structural changes of the nucleotide (See, Table S4-2).

Increasing method's throughput via MassSum-based computational data separation, making it possible to directly sequence as many as tRNA species, completely or in part, that LC-MS permits in a single run. In order to utilize ladder fragments from each individual tRNA isoform in a complementary manner for completion of a perfect ladder needed for MS sequencing, each isoform and its ladder fragments in the complex MS data of mixed samples with multiple distinct RNA strands/sequences must be identified. Ideally, all the ladder fragments in either 5′- or 3′-ladder individually can be identified and get separated out collectively as a 5′- and a 3′-ladder for each isoform from the complex MS data. For this purpose, a new algorithm was developed, named as MassSum (FIG. 30), based on the fact that the mass sum of any set of paired fragments generated during acid-mediated degradation of RNA by cleavage of one phosphodiester bond is constant (equivalent to the mass of each undegraded RNA plus the mass of a water molecule) (Bjorkbom, A. et al. Journal of the American Chemical Society 137, 14430-14438, (2015)). Taking a 9 nt RNA strand as an example to illustrate the idea (see FIG. 22A-22B), the two ladder fragments are generated as a result of an acid-mediated cleavage of the phosphodiester bond between 1^{st} nucleotide and 2^{nd} nucleotide of the 9 nt RNA strand. One of them carries the original 5′-end of the RNA strand and has a newly-formed ribonucleotide 3′(2)-monophosphate at its 3′-end (denoting as F1). The other one carries the original 3′-end of the RNA strand and has a newly-formed hydroxyl at its 5′-end (denoting as T8). In the well-controlled acid hydrolysis conditions, the phosphodiester bond cleavage is random but once per RNA strand on average (Bjorkbom, A. et al. Journal of the American Chemical Society 137, 14430-14438, (2015)). As it moves along the RNA strand to cut each of the phosphodiester bond, each cleavage will generate a pair of fragments, such as F2 and T7, F3 and T6, and so on. The mass sum of any one-cut fragment pair, e.g., mass sum of F2 and T7 equal to the mass sum of F1 and T8, is constant and equals to the mass of 9 nt RNA plus the mass of a water molecule. Since the mass sum is unique to each RNA sequence/strand, and it can be used to computationally separate all paired fragments of the RNA sequence/strand out of complex MS datasets.

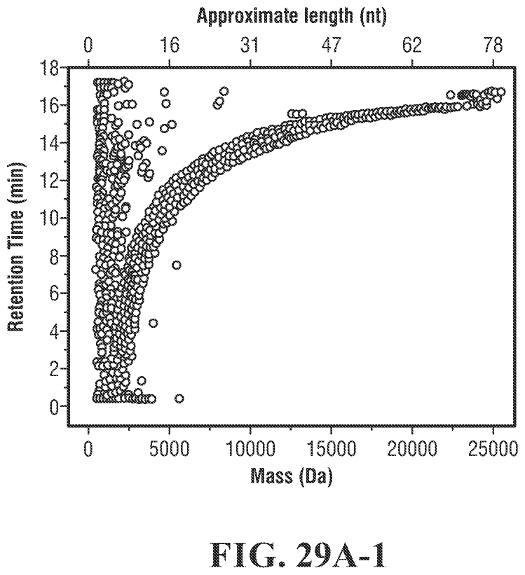

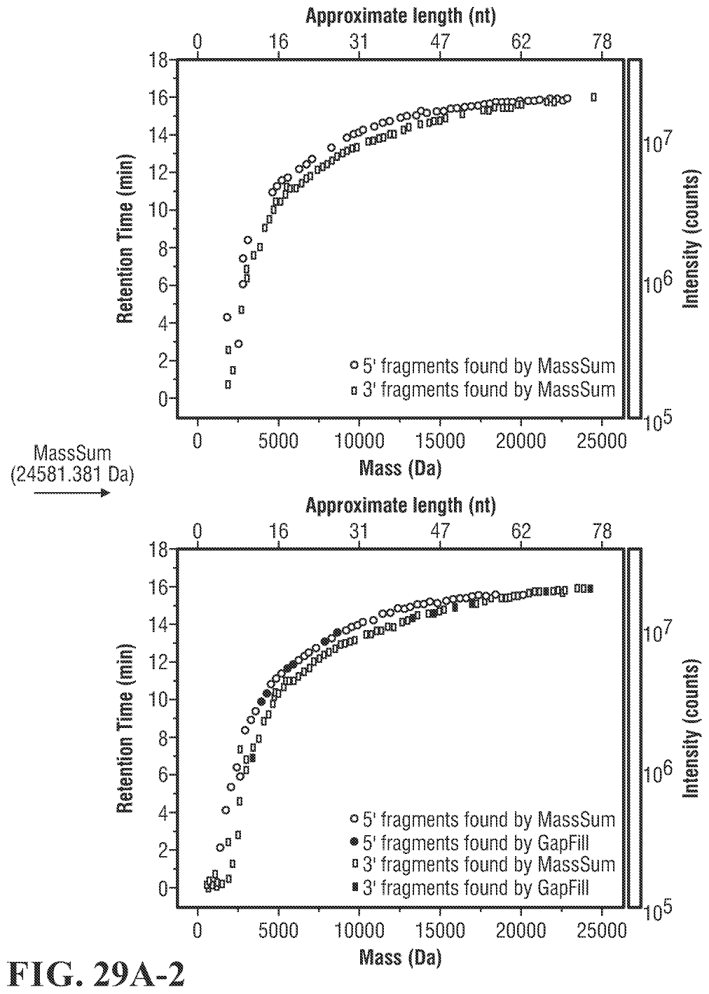

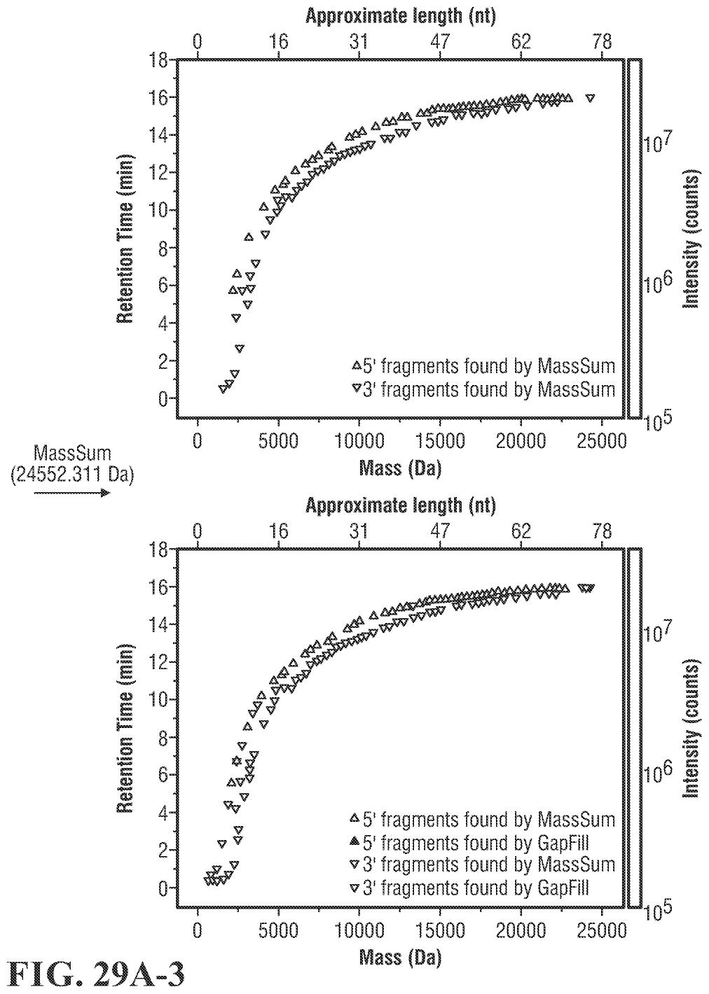

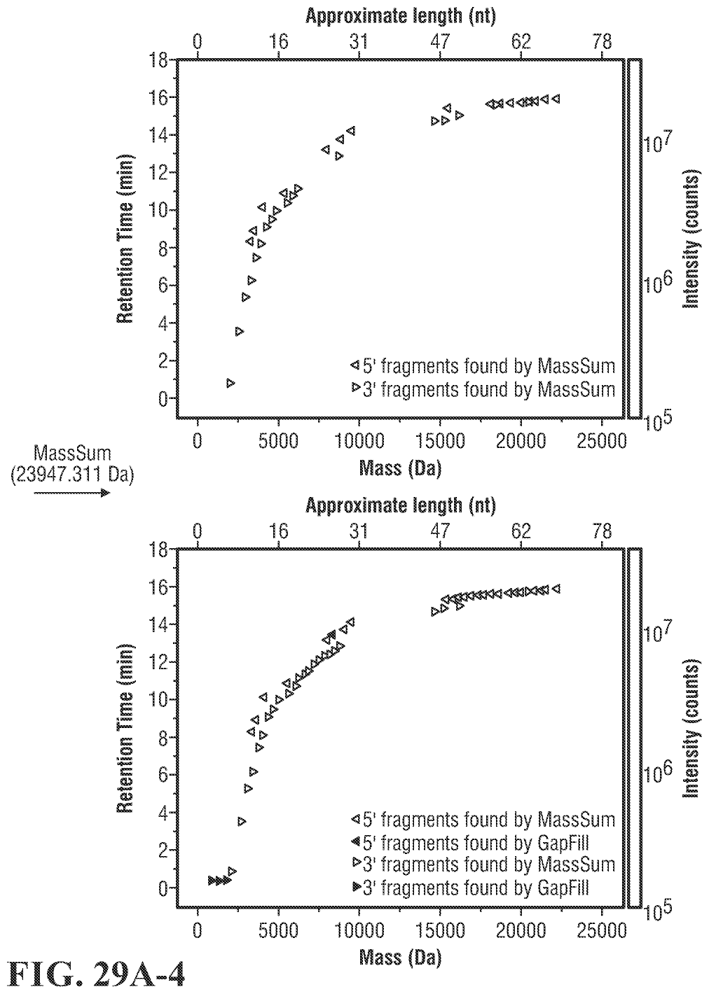

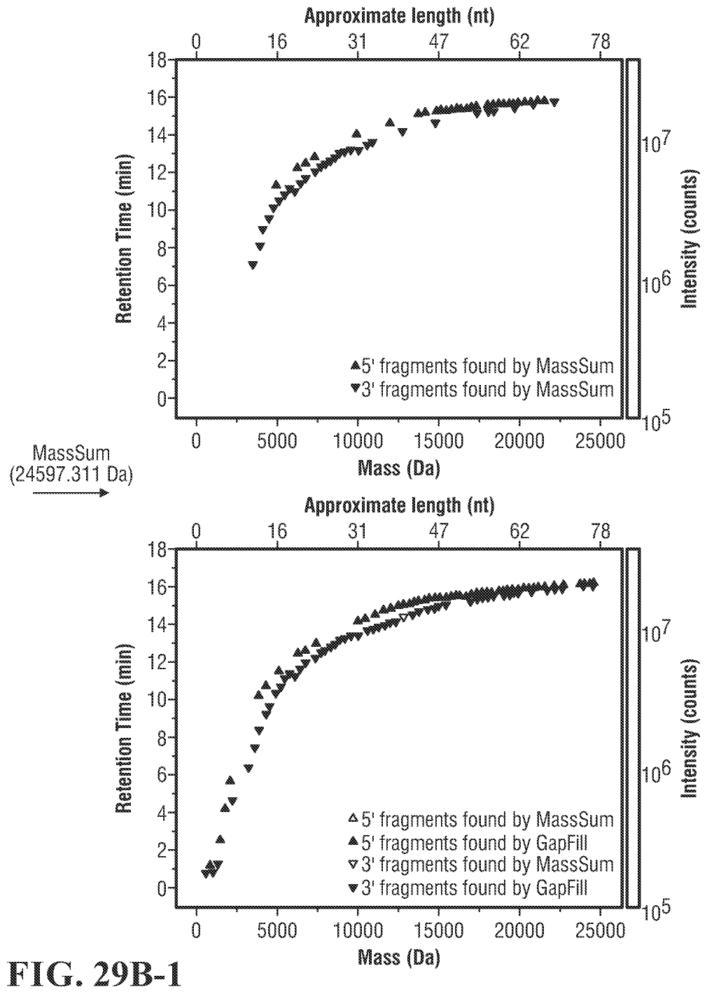

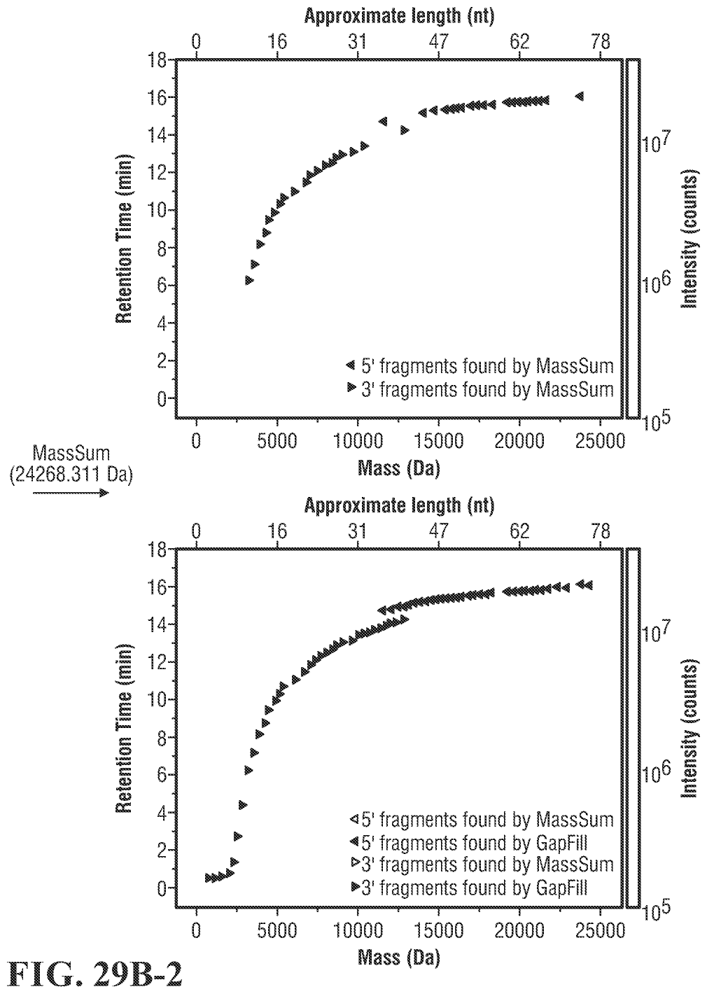

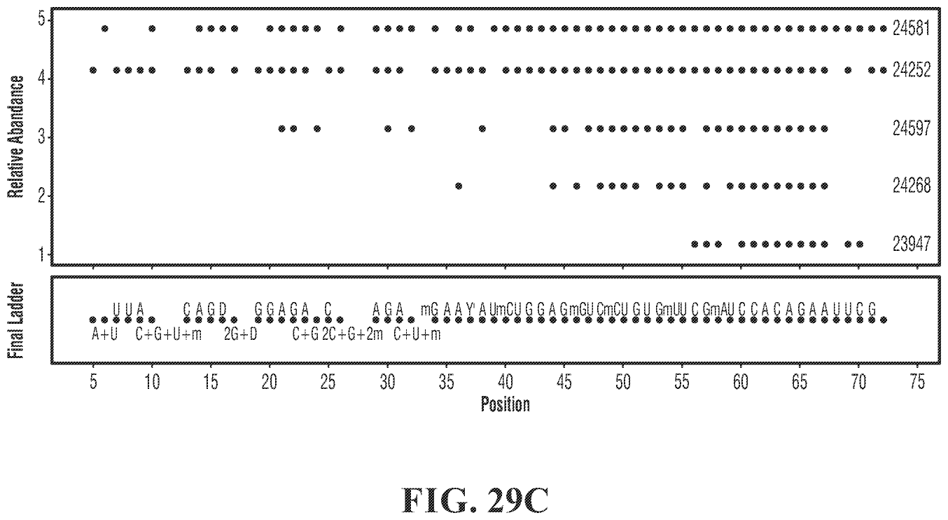

Similarly, using the mass sum constant unique to each tRNA isoform, one can computationally isolate MS data of all ladder fragments derived/degraded from the same tRNA isoform sequence in both the 5′- and 3′-ladders out of the complex MS data of mixed samples with multiple distinct RNA strands (FIG. 22D and FIG. 29). However, in case that one ladder fragment is missing, e.g., in the 5′-ladder, the correspond single-cut ladder fragment, even if it exists in the 3′-ladder, will not call out by the MassSum algorithm. In order to pull out all the ladder fragments out of the complex MS data, a GapFill algorithm (FIG. 31) was designed to rescue these ladder fragments missing by MassSum separation. This is possible because an algorithm can be developed to examine each original mass datapoint before MassSum separation to find fragment compounds that can fix/bridge the gap. After the gap filling, the mass differences between the two adjacent ladder components must satisfy the requirement for base-calling a nucleotide or a modification, otherwise, the mass of the bridge compound cannot fit the gap and the ladder fragments remain lacking at this position. In some cases, more than one bridge compound can fit into one position in the gap, the one that fit better into 2D mass-t_{R} sigmoidal curve over the other ones will be chosen (Bjorkbom, A. et al. Journal of the American Chemical Society 137, 14430-14438, (2015)). This ambiguity can also get addressed later in the step for ladder complementing. The same position in the other tRNA isoform ladders (either 5′- or 3′-ladder) will be examined to ensure the one supported more to get selected (See, Table S4-1 through Table S4-3).

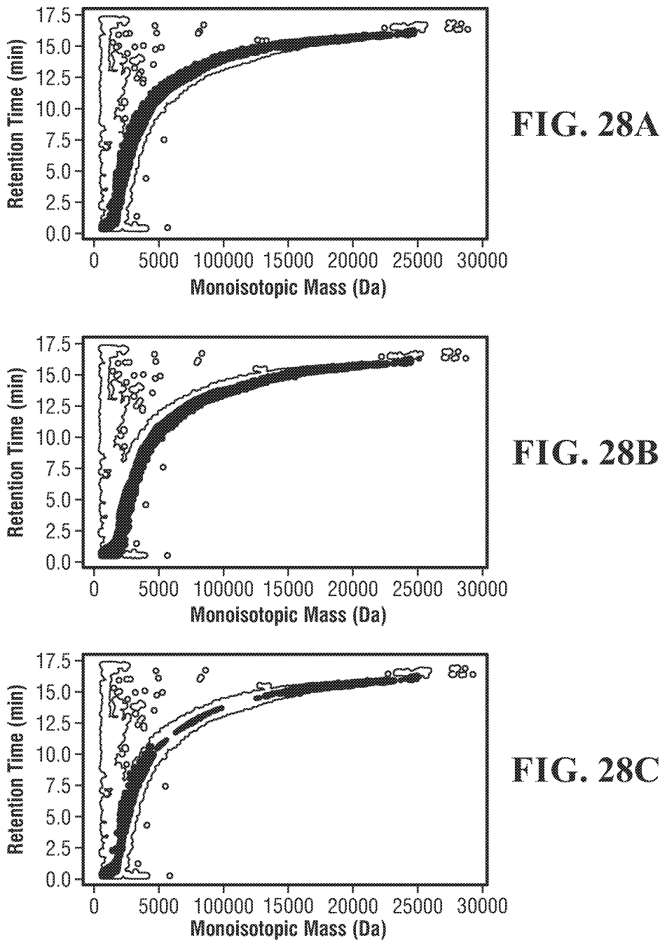

With the MassSum-base data separation strategy, even for the minor tRNA species in the complex RNA samples, no matter they stand alone or have other isoforms, their ladder fragments in 5′- and 3′-ladders become identifiable via their unique individual intact masses, and can also get computationally separated out. tRNA-Phe (2^{nd} isoform) is very minor species in the tRNA-Phe sample (Sigma) and has <1% abundance comparing to the 75 nt tRNA-Phe isoform (FIG. 22C-D). All its ladder fragments in the mixed MS dataset have been identified and isolated out. Apparently, there are many ladder fragments missing in both 5′- and 3′-ladders. However, with these limited ladder fragments fixed by subsequent GapFill, one is still be able to de novo read out the tRNA-Phe (2^{nd} isoform) sequence in part. A base call for a nucleotide or modification can be made when there are two ladder fragments adjacent to each other, and their mass difference match well with the one in the databases (Zhang, N. et al. Nucleic Acids Res 47, e125, (2019); Bjorkbom, A. et al. Journal of the American Chemical Society 137, 14430-14438, (2015)). For example, a short sequence UCCACAGAGUUCG (SEQ ID NO: 8) can be read out. As each ladder fragment also carries position information (˜318/nt), one can locate their positions ranged from position 59-71 in the tRNA-Phe (2^{nd} isoform). Putting together scattered sequences in different locations, they form a unique pattern, and can be used to blast out the entire tRNA-Phe (2^{nd} isoform) sequence, e.g., from reported tRNA sequences in literature/databases, which can be used, in turn, to find more ladder fragments for its consequence verification and modification analysis. Similarly, other minor tRNA species (with <1% abundance comparing to the 75 nt tRNA-Phe isoform) in the sample have been sequenced in part or identified (see FIG. 28).

The full potential of the MassSum strategy remains to be explored. It pushes the limit of the method's throughput to the physical limit an LC-MS instrument imposed on RNA samples, allowing sequencing of unlimited RNA sequences/strands in complicated RNA samples as long as the MS instrument can detect the RNA along with their ladder fragments. In addition, this mass sum strategy can be used for computational data separation of any RNA's MS data from a complex dataset of a mixed sample. Therefore, with further development, the computational data separation strategy could reduce or obviate the need for physical purification or enrichment of specific tRNAs, allowing MS sequencing of any RNA species in a mixture directly, even low abundance RNA species and/or RNAs with low-stoichiometric modifications, as long as there are sufficient amounts of ladder fragments for LC/MS instrument detection. This also pave the way toward MS sequencing of complex mixtures of biological RNA in large scale when using the state-of-the-art LC-MS instruments currently available.

Computational separation of 3′- and 5′-ladders of each tRNA species/isoform. Complementing ladder fragments from each individual tRNA isoform to completion of a perfect ladder for MS sequencing entails another step, separation of 3′- and 5′-ladders of each tRNA isoform. Separation of these two ladders can be achieved further in a computation way after they were collectively isolated from the complex MS data by MassSum. Each 5′-ladder fragment has a two terminal monophosphates with one from the original 5′-end of the tRNA species and the other being a newly-formed ribonucleotide 3′(2)-monophosphate at its 3′-end. As such, the 5′-ladder is the top one and the 3′-ladder is the bottom one of the two sigmoidal curves adjacent to each other in the 2D mass-t_{R} plot (See FIG. 22B). That is because each 5′-ladder fragment has a relatively bigger t_{R} when comparing to the one with the same length in 3′-ladder. The t_{R} differences can be used to further computationally separate these two ladders, breaking two adjacent sigmoidal curves into two isolated curves, one for 3′-ladder and the other for 5′-ladder (FIG. 20E and FIG. 28).

It works the same when alternating the order of MassSum and ladder separation. the complex MS dataset of mixed samples with multiple distinct RNA strands/sequences can be computationally divided into two subsets based on the t_{R} differences with the top one subset for 5′-ladders and the bottom one for 3′-ladders (FIG. 20E and FIG. 28). After the ladder separation, these two subsets are used as inputs for the MassSum algorithm to output 5′-ladder and 3′-ladder separately for each tRNA isoforms/species. The missing the ladder fragments can be fixed by the GapFill algorithm.

Computational separation of 3′- and 5′-ladders of each tRNA species/isoform provides an alternative to identify ladders in mixed RNA samples even without HELS (Zhang, N. et al. Nucleic Acids Res 47, (2019); Zhang, N. et al. ACS Chem Biol 15, 1464-1472, (2020)), and help to simplify RNA sample preparation, enhance sample efficiency significantly, to increase throughput substantially to the physical limit that an LC-MS instrument is imposed on RNA samples.

Completion of a faulted mass ladder by complementing the missing ladders from other isoforms identified in homology search. Having two separated 5′- and 3′-ladders of each tRNA isoform, ladder complementing can be implemented inside 5′- or 3′-ladder without crossing one ladder to the other to contribute toward the completion of a perfect ladder without missing any ladder fragments (FIG. 20F). Each of the tRNA-Phe isoform's ladder are layed out, e.g., 5′-ladder in FIG. 23, on top to each other vertically; the 5′-ladder of each isoform is arranged horizontally according to the position of each ladder fragment corresponding to, ranging from position 1 to 76 nt for tRNA-Phe (˜318 Dalton/nt) (FIG. 23). For example, the 5′-ladder fragment missing at positions 11 and 12 of 76 nt tRNA-Phe isoform (with a monoisotopic mass of 24581.3692 after acid degradation) can get fixed site-specifically by the counterpart ladder fragment from another tRNA-Phe isoform (75 nt with a monoisotopc mass of 24252.3167 after acid degradation). Both these two isoforms have ladder fragments complementary to ladders for other tRNA-Phe isoforms. As such, a perfect 5′-ladder that does not miss any ladder fragment, can be formed for sequencing of the tRNA group, including all the four tRNA-Phe isoforms (FIG. 23C).

Dependent on the sample quality and quantity, there are cases where ladder fragments are still missing in the 5′-ladder even if ladder complementing from all other isoforms, 3′-ladder can also be used to fix the missing fragments site-specifically for sequence completion of the tRNA, or fix the missing piece of sequence after reading out sequences from both ladders (5′- and 3′-) (FIG. 23B). In some cases, it was observed that more than one ladder fragments can fit into one position when complementing ladders from different isoforms, one may look into the same position in the other tRNA isoform ladders (either 5′- or 3′-ladder) to ensure the one with higher confidence (the one supported more by other isoform' ladders) to get selected (see methods and SI). This strategy works well when selecting one of two possible ladder fragments (corresponding to either U or C) fit into the gap in positions 70 and 72 after MassSum separation of MS data of 75 nt CC-tailed tRNA-Phe. The ladder fragment corresponding to U was selected for position 70 and the ladder fragments corresponding to C was selected for position 71 as this sequence match well with other ladders (see FIG. 29). This ambiguity can also get addressed later when using anchor-based sequencing algorithm to read out the final sequence based on a global hierarchical ranking strategy which is tailored to report only top-ranked sequences (Zhang, N. et al. ACS Chem Biol 15, 1464-1472, (2020)).

Complementing ladders between tRNA isoforms can help major isoforms with relative high abundance get more complete ladder and enable minor isoforms with relative low abundance to be sequenced despite of their low abundance.

Sequencing of minor tRNA-Glu isoforms/species (<1% relative abundance) in complex RNA mixture samples prepared from A549 cells (with or without RSV infection). tRNA-derived small RNAs (tsRNAs) is a recently discovered family of small non-coding RNAs (sncRNAs) that has emerged as important players in several other diseases such as neurodevelopmental disorders, metabolic disorders, and infectious diseases (Olvedy, M. et al. Oncotarget, (2016); Liu, S. et al. Sci. Rep 8, 16838, (2018); Wang, Q. et al. Mol. Ther 21, 368-379, (2013); Zhou, J. et al. J. Gen. Virol 98, 1600-1610, (2017); Selitsky, S. R. et al. Sci. Rep 5, 7675, (2015); Ruggero, K. et al. J. Virol 88, 3612-3622; Thompson, D. M., Lu, C., Green, P. J. & Parker, R. RNA 14, 2095-2103 (2008); Chen, Q. et al. Science 351, 397-400, (2016)). They are the most significantly affected sncRNAs in RSV infection (Wang, Q. et al. Mol. Ther 21, 368-379, (2013)). During RSV infection, the most aberrant tRFs are generated from a specific subset of tRNAs cleaved mainly by a specific ribonuclease, angiogenin (ANG). Emerging evidence has identified a variety of RNA modifications in tRFs (Zhang et al., Trends Mol. Med 22, 1025-1034, (2016)). The tRF nt modifications are essential for their function, and are associated with transgenerational epigenetic inheritance, and with diabetes (Chen, Q. et al. Science 351, 397-400, (2016); Yan, M. et al. Anal Chem 85, 12173-12181 (2013)). However, However, data obtained from deep sequencing can provide sequences primarily only, and they did not include RNA modification information. The MS sequencing technique was used to sequence and explore nucleotide modification changes within these tRF-5/tRNAs related to the RSV infection.

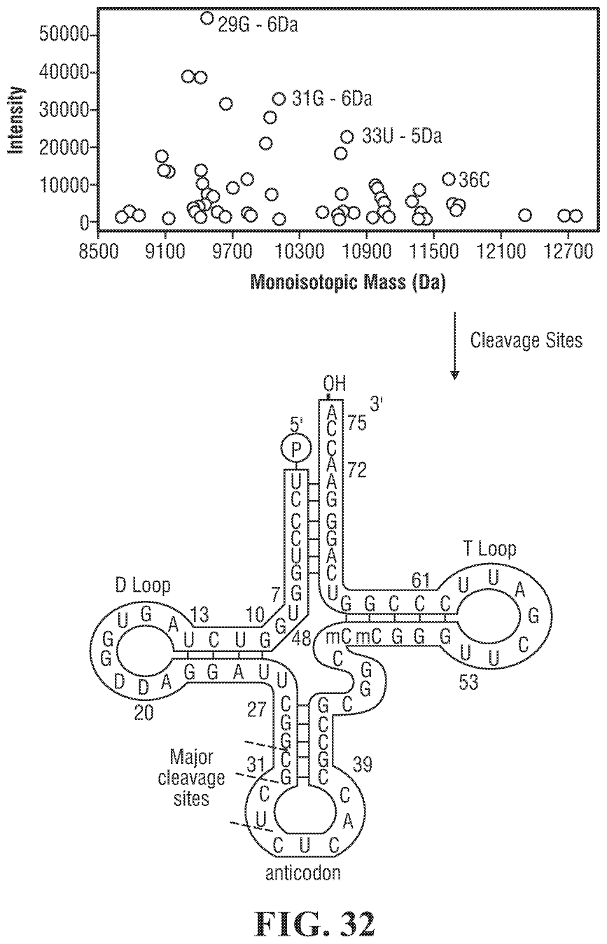

Despite efforts to isolate tRNA-GluCTC by using a probe, the tRNA-Glu-CTC samples purified from the RSV/mock-infected cells were heterogeneous based on the quantitative differences in the mass profiles of the two samples. The infected sample contained less abundant full length tRNA molecules in the mass region (≥21000 Da) and more in the cleavage region mass region (5000-12000 Da) comparing to the uninfected sample (FIG. 32), indicating that during RSV infection, some mature tRNA molecules were cleaved. However, the overall relative abundance of the mature tRNAs (21000+Da) were very low in these samples. Further increase in abundance/amount of the target tRNA-Glu-CTC and its relevant tRFs in the RNA samples would help to improve MS and sequencing results.

Despite of relative low abundance, the tRNA-Glu and its related isoforms were sequenced by MS to identify and locate their different nucleotide modifications (FIG. 24). 2 continuous sequence segments were de novo read out in one of tRNA-Glu isoform (with a monoisotopic mass of 24189.250), corresponding to U7-A24 and C36-C41. With the sequence and location information as input, NGS data performed in parallel were used to blast out one tRNA with a complete 75 nt sequence form massive NGS sequencing results (>10 million reads) (FIG. 24D). This tRNA sequence contains primary sequences without RNA modification information, which can be used to in silico generate a theoretical exact mass for each acid-hydrolyzed ladder fragment corresponding to the 1^{st} to the last nucleotide in the tRNA. These in silico masses were compared to the observed monoisotoptic masses at each position, and any mass shift would indicate a modification. The identify of the nucleotide modification can be extrapolated by the shifted mass difference. As such, one is still able to identify and locate each nucleotide modification in the very minor tRNA species in the complicated cellular sample (FIG. 24F).

The MS sequencing technique was used to sequence and explore nucleotide modification changes within these tRF-5/tRNAs related to the RSV infection. The tRF [5′tRNA-Glu-CTC half molecule (9464.1880 Da)] was found only in the RSV infected sample. This 29 nt long 5′tRNA-Glu-CTC half can only be produced from the mature tRNA since it has a 5′phosphate group and a 3′cyclic phosphate group. The 29 nt 5tRNA-Glu-CTC half molecule may contain the same modifications as the mature tRNA-Glu-CTC. (5′p-UCCCUGGUGm²GUCψAGUGGDψAGGAUUCGG-2′3′ p (SEQ ID NO: 9)). The relative abundance of the 29 nt tRNA half was 0.01 vs. 0.36 in mature tRNA Glu-CTC. The above information is the first detailed description of the 5tRNA-Glu-CTC half. It is expected that this new information will provide further insight to understand the biological functions of the mature tRNA (e.g., stability) and the resulting cleavage product.

Two more interesting findings were obtained. First, a group of masses over 8000 Da were observed, especially in the infected sample (FIG. 32). These mass data did not correlate with any of the fragments from standard tRNA-Glu-CTC sequencing. Second, a group of large oligonucleotides in the full length tRNA mass region with a relative abundance ≥0.36 were observed. They contain substantial methyl group differences compared to the mature tRNA-Glu-CTC. This may reflect the normal methylation dynamics within tRNA. More importantly, the co-existence of a tRNA-Glu-CTC containing an additional methyl group for both samples was discovered that have not been reported previously. It was noticed that this methylated tRNA-Glu-CTC has higher relative abundance than the original tRNA-Glu-CTC in the RSV infected sample while the opposite relative abundance result was observed for the uninfected sample. It was suspected that RSV infection might lead to higher ANG activities in cells and ANG then hydrolyzed cellular tRNA to modulate production of tRFs/activity of methylating enzymes, during the production of mature tRNA. Furthermore, the manual search results and computational search results in acid degraded RSV infected and uninfected samples further confirmed the existence of this extra methylated tRNA since some addition methylated mass ladders were found. It was predicted this methylation occurs within the 5′ stem of the tRNA. However, the exact location of this methylation could not be located, in part because acid degraded fragments below 10 nt were difficult to identify in all the acid-degraded RNA samples. This is maybe due to either limited RNA sample amount or the LC-MS setting that is not favorable to the fragments with masses less than 3200 Dalton.

tRNA is a type of RNA family that current NGS-based methods cannot sequence effectively, due to complication from its rich modification and related isoforms. The method will provide an effective and efficient way to directly sequence tRNA including its different isoforms without the needed to separate each isoform, which is almost impossible due to sequence/structure similarity. The adversity of data complex of mixture of RNA isoforms is reversed into an advantage for MS-based sequencing. Homology search is used to identify and connect different isoforms together and thus are able to complement each isoform ladder for the ladder completion of the same specific tRNA species. Mass sum strategy can computationally isolate each tRNA isoform, even tRNA isoforms with very low relative abundance (<1%), from the RNA mixture, and pushes the limit of the method's throughput to the physical limit an LC-MS instrument is imposed on RNA samples, allowing sequencing of unlimited RNA sequences/strands in complicated RNA samples as long as the MS instrument can detect the RNA along with their ladder fragments.

Being able to handle RNA sample complexity like from different tRNA isoforms and to MS sequence RNA with even faulted mass ladder would greatly expand the method's application, allowing more broader samples that cannot generate perfect ladder, likely due to sample scarcity/low amount/low stoichiometry, to be sequenced for RNA modification studies. This paves a way for de novo MS sequencing of complex biological in a large scale via automation.

Since MS-based sequencing techniques rely on a unique mass value for identifying and locating each nucleotide, in the case where modifications have isomers with identical masses but different chemical structures such as pseudouridine (ψ) from its identical uridine (U) and different methylations, an extra step will be required to differentiate these isomeric nucleotide modifications following the MS sequencing approach as described previously (Zhang, N. et al. ACS Chem Biol 15, 1464-1472, (2020)).

The full potential of the method's sequencing read length and throughput remains to be explored, and it seems instrument dependent, i.e. mass spectrometers with higher resolving powers and better sensitivity may lead to increased read length and throughput, and lower sample requirements. With more advanced LC-MS instruments, one can expect that the read length can be increase more than >˜76 nt per run, allowing direct sequencing other RNA longer than tRNAs beyond tRNA and tRFs presented in the manuscript.

Many efforts have been made to improving MS/MS or MS′, e.g., for analysis of small metabolites and peptide/proteins. If similar efforts could be made to improve primary MS/monoisotopic mass measurement, one may have much better instrumentation and data processing software needed for nucleic acid/RNA sequencing using the method described in the manuscript. The throughput of MS-based sequencing may not be comparable to NGS, which can read >2 billion of DNA/RNA at the same time, but it may read >100 RNA strands/sequences simultaneously with optimized sequencing workflow and improved MS instruments. This throughput can then be comparable to capillary Sanger Sequencing.

Together with improved read length and automation capacity of LC-MS, one may be able to read >4 million base per day on an optimized LC-MS instrument, which would allow many applications in sequencing of a variety of RNA samples, and have at least a comparable impact similar to that of Sanger Sequencing on the community and society. This method will provide a general/sequencing tool for studying RNA modification, which is urgently needed, more than ever especially considering that >40 unidentified nucleotide modifications discovered in SARS-CoV-2 RNA (Kim, D. et al. Cell 181, 914-921 (2020)). Such a method will also be instructive for studying SARS-CoV-2 RNA and other RNAs and to unravel epitranscriptomic roles in COVID and other diseases.

### Example 5

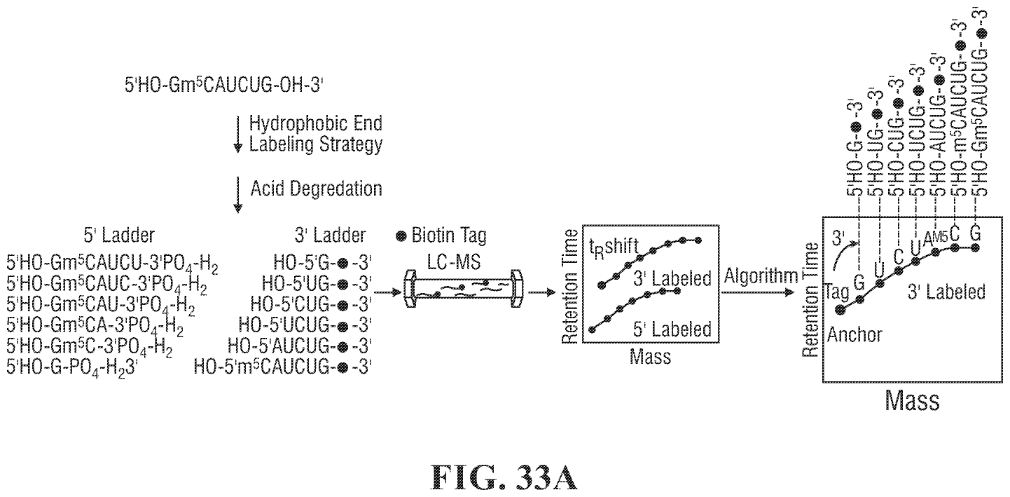

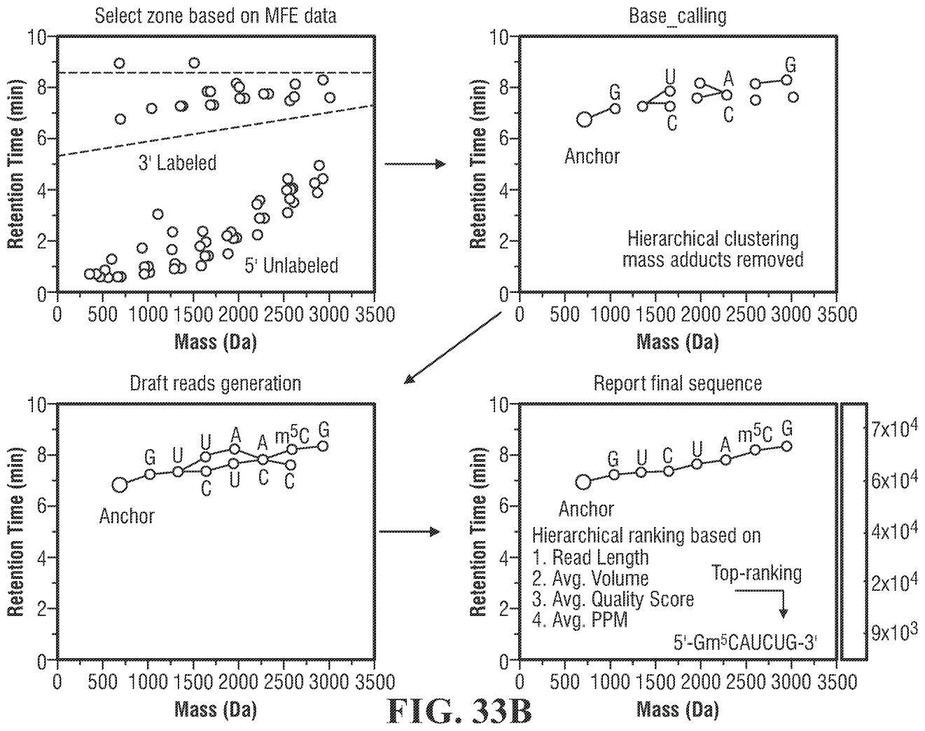

To simplify the data analysis and to be paired with the 2-D HELS, two computational anchor algorithms were developed which innovatively accomplish automated sequencing of RNAs. The signature t_{R}-mass value of the hydrophobic tag specifies the exact starting data point, the anchor, for the algorithm to accurately determine data points corresponding to the desired ladder fragments, significantly simplifying data reduction and enhancing the accuracy of sequence generation. The idea of using an anchor to identify sequence ladder start-points can be generalized and extended to any known chemical moiety beyond hydrophobic tags, e.g., PO₄⁻ at the beginning of the tRNA or any nucleotide with a known mass and can program its mass as a tag mass and use the anchor algorithms for sequencing, addressing the issue of MS data complication and making 2-D HELS MS Seq more robust and accurate (FIG. 33).

As it was possible to read segments of up to 35 nt long with a 40K mass resolution LC-MS (N. Zhang et al., Nucleic Acids Research (2019)), a RNase T1 partial digest step to the tRNA^{Phe} sequencing strategy was incorporated in order to reduce the 76 nt tRNA down to a sequenceable size. Subsequently, it was possible directly sequenced the entire tRNA with single-base resolution in one single LC-MS run (FIG. 8). To further verify the complete tRNA sequence obtained from the single run above, the three segments cut by RNase T1 were labeled and separated them one by one for 2D-HELS-AA MS Seq in three separate LC-MS runs (FIG. 13). In order to obtain overlapping segment sequences for assembling the complete tRNA, 2D-HELS-AA MS Seq data of the tRNA previously generated without RNase T1 digestion was also included (FIG. 8). Taking all draft reads output by the anchor-based algorithm including all the modifications together (Table S5-1 through S5-11), a full length tRNA sequence was assembled and the final sequence was 100% match to the reference sequence tRNA^{Phe} with more than 2× coverage (FIG. 8). Not only was the complete tRNA sequenced, but it was possible to successfully identified and located all 11 RNA modifications within the tRNA (FIG. 9). Among these 11 detected modifications, there are four modifications that can be directly read out by their unique masses, including dihydrouridine (D) at positions 16 and 17, N²,N²-dimethylguanosine (m²₂G) at position 26, 5-methylcytidine (m⁵C) at position 40, and 5-methyluridine (T) at position 54. Methylation on the 2′-OH of the tRNA renders the adjacent 3′-5′-phosphodiester linkage non-hydrolyzable, creating a mass gap in both the 5′- and the 3′-mass ladder families larger than 1 nt (12) (FIG. 8A). This gap can be filled by collision induced dissociation (CID) MS, which determines which one is methylated between two unhydrolyzable nucleotides (A. Bjorkbom et al., J Am Chem Soc 137, 14430-14438 (2015)) (FIG. 14). However, other RNA modifications such as pseudouridine (ψ) and U, N²-methylguanosine (m²G) and 7-methylguanosine (m⁷G), and 1-methyladenosine (m¹A) and N⁶-methyladenosine (m⁶A) share an identical mass, and a mass alone cannot distinguish them. Further enzymatic/chemical reactions were used to differentiate m¹A and m⁶A (14) (by rtSBE, FIG. 15), U and ψ (13) (by converting ψ into CMC-ψ and t_{R}-mass shifts, FIG. 16 and Table S5-12 through S5-17), and m²G and m⁷G (15) (by NaBH₄ reduction and aniline cleavage, FIG. 17).

Upon analysis of the sequence results, three findings relevant to tRNA^{Phe} structure and biochemistry were encountered. First, it was noticed that Y at position 37 was converted to its depurinated product Y′ (ribose) under acid degradation conditions (FIG. 9) (U. L. RajBhandary, R. D. Faulkner, A. Stuart, Studies on polynucleotides. LXXIX. J Biol Chem 243, 575-583 (1968); J. E. Ladner, M. P. Schweizer, Nucleic Acids Res 1, 183-192 (1974)). Initially without acid degradation, only 10% of the tRNA contained the depurinated Y′ form at this position, while the majority (90%) had the regular Y form of the base (Table S5-18). However, no Y form was observed in any of ladder fragments that cover this position after acid degradation, and all of the Y bases were converted to Y′ due to the depurination in the acidic conditions. A mechanism of the acid-assisted depurination was proposed in (FIG. 9). As another piece of evidence of the depurination, a mass of 376.1178 Da, corresponding to a cleaved Y nucleobase, was found in the crude products after acid degradation (FIG. 9), suggesting that Y was originally carried by the tRNA, but was converted to Y′ in the acidic conditions that were used to generate the mass ladders for sequencing. The fact that the method can identify the dynamic change of Y to Y′ and quantify the relative Y/Y′ ratio can be very useful. As certain cancer cells have an acidic pH (R. A. Gatenby, E. T. Gawlinski, A. F. Gmitro, B. Kaylor, R. J. Gillies, Cancer Res 66, 5216-5223 (2006)), where very likely the acid-mediated conversion of Y to Y′ will occur (R. Thiebe, H. G. Zachau, Eur J Biochem 5, 546-555 (1968)), ratio changes of the Y′/Y in the certain cells can be used as a potential biomarker for diagnosis of these cancers. Similarly, it is expected in the same principle that with a proper sample preparation, the method can probe dynamic changes of other base modifications, acid-labile or not, and quantify their ratio changes in different biological processes.

Second, unlike its commercial nominal identity, the commercially-prepared tRNA^{Phe} sample was revealed to be heterogeneous. Beside the 76 nt tRNA with a post-transcriptionally modified CCA tail, two other isoforms of the tRNA that miss an A and an CA at the 3′-CCA tail, respectively (FIG. 8 and FIG. 10), were identified when segment III (58m¹A-76A) was sequenced using the anchor algorithms together with a revised Smith-Waterman alignment algorithm that determines similar regions between two strings of nucleic acid sequences. It was reported that the most abundant component was not the nominal identity of the tRNA from the supplier, 76 nt tRNA^{Phe} (T. Y. Huang, J. Liu, S. A. McLuckey, J Am Soc Mass Spectrom 21, 890-898 (2010)). Not only did the MS results confirm the previous results, but the method also can precisely identify all these three isoforms and quantify their percentages (17%, 80%, and 3% for the 76 nt, 75 nt, and 74 nt RNA, respectively) by integrating their corresponding EIC (Table S5-19). The two tail-truncation isoforms cannot be degraded products of longer tRNAs like the 76nt tRNA^{Phe}, otherwise, they would not have a free 3′-OH required for the 2D HELS chemistry. The data indicates that 2-D HELS MS Seq is a method not only good for sequencing of modified RNA, but it also is reliable for identification and discovery of tail-truncation isoforms that were primarily studied by PAGE gel method (C. Merryman, E. Weinstein, S. F. Wnuk, D. P. Bartel, Chem Biol 9, 741-746 (2002)). The ability to simultaneously identity, locate, and quantify the relative abundances of tRNA tail-truncation isoforms will assist in investigating their role in biological processes related to human disease (Y. M. Hou, IUBMB Life 62, 251-260 (2010)). As stress-induced tRNA truncation has been implicated to cancers and other diseases (D. M. Thompson, R. Parker, Cell 138, 215-219 (2009)) further investigation the CCA tail-truncation isoforms in tRNAs will lead to new ways to treat these diseases.

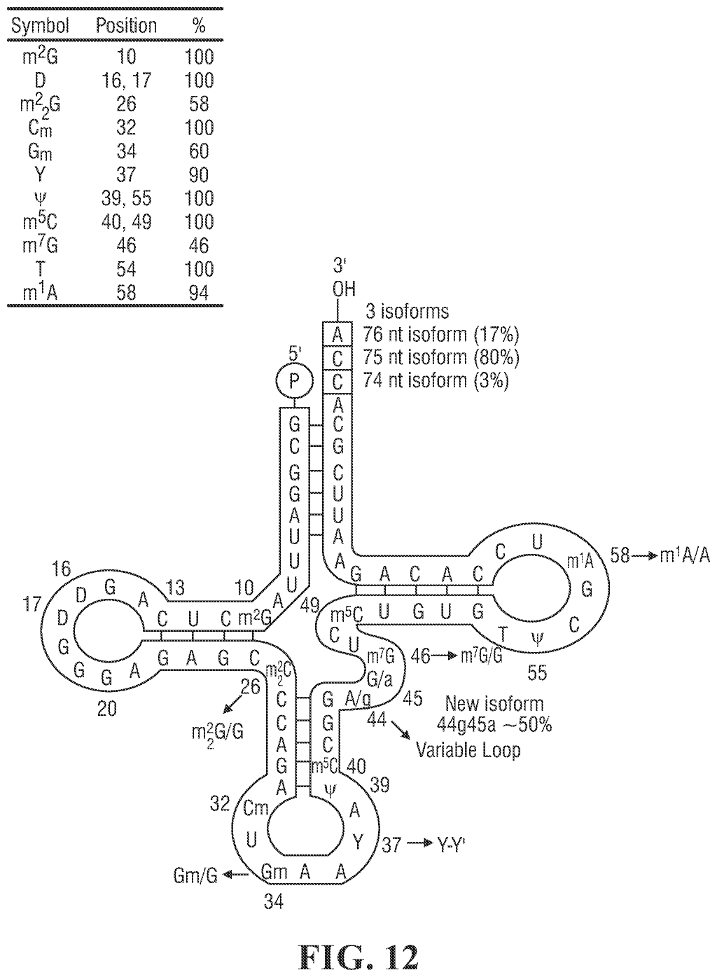

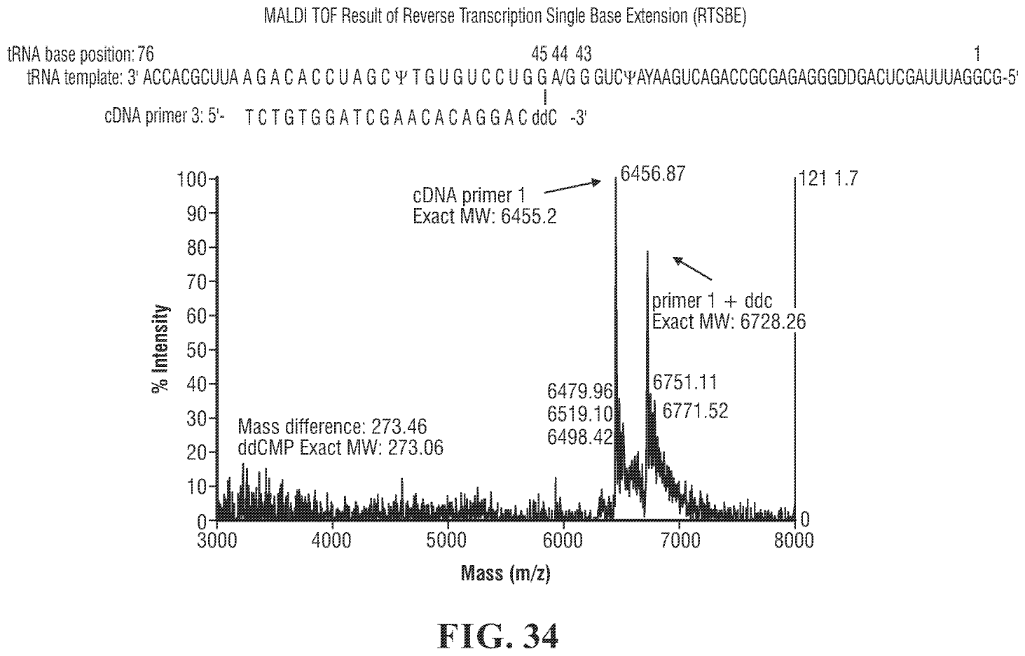

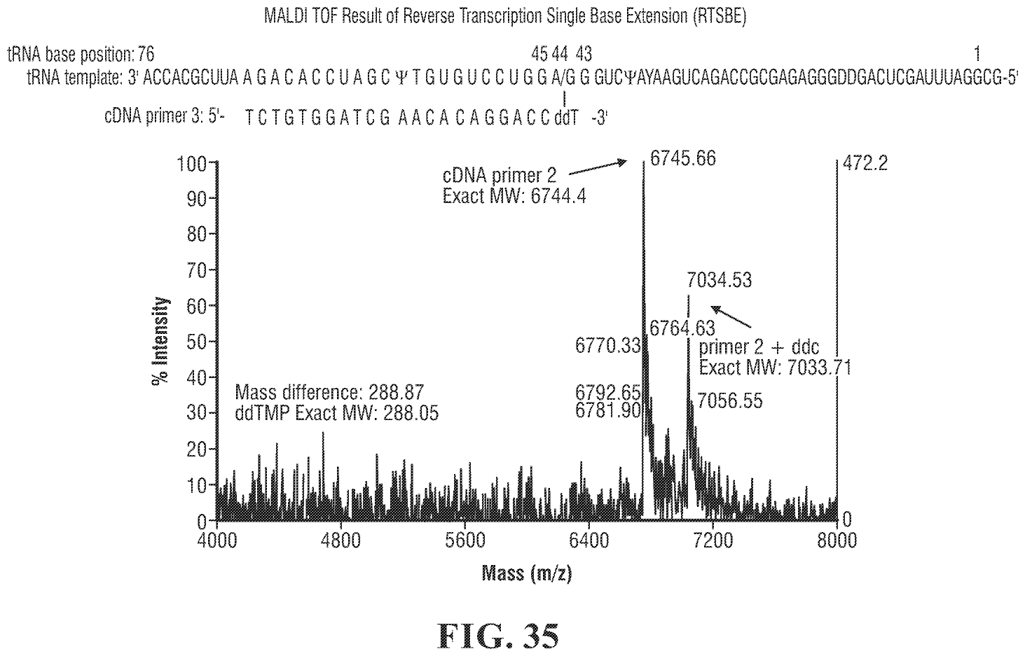

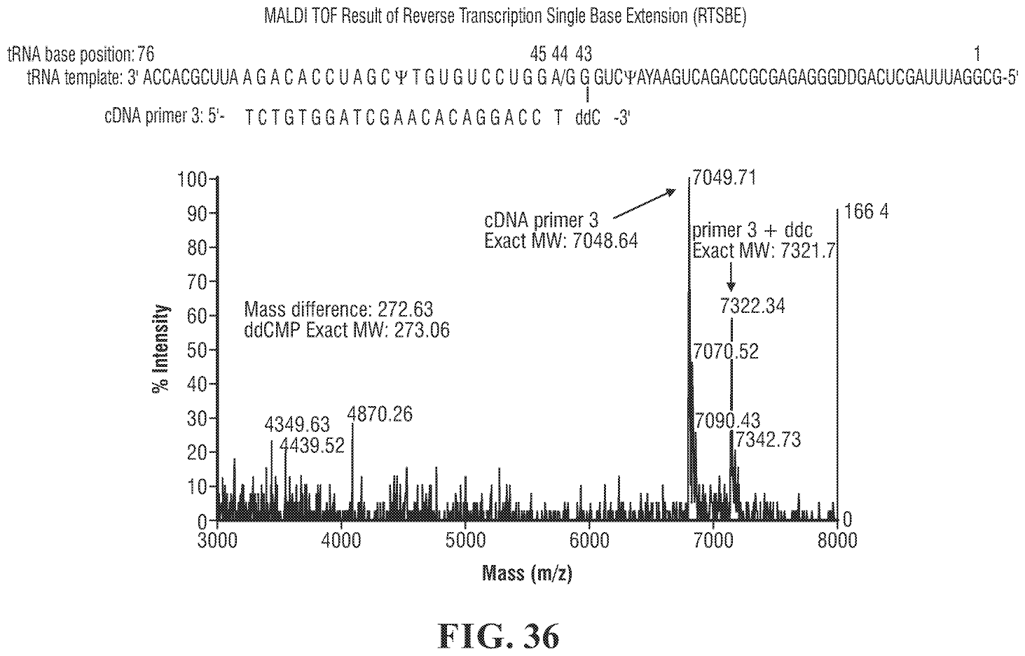

Thirdly, two isoforms with an A to g transition mutation at position 44 and a G to a transition mutation at position 45 were observed, i.e., 44A45G (wild type) (B. Alzner-DeWeerd, L. I. Hecker, W. E. Barnett, U. L. RajBhandary, Nucleic Acids Res 8, 1023-1032 (1980)) and 44g45a (mutated; lower cases g and a used here to differentiate them from non-mutated regular G and A). The two draft reads were reported out first by the algorithm and later verified manually in the original MFE files (FIG. 11, Table S5-4, Table S5-5, Table S5-8, Table S5-9, and Table S5-20 through Table S5-23). Two mass ladder fragments were found at position 44 when reading from 5′ direction, corresponding to 44A and 44g, but the two merged into one mass ladder fragment only at position 45, corresponding to 45G and 45a (FIG. 11). This is also consistent with the sequencing results when reading from the opposite direction as one can perform bi-directional sequencing (A. Bjorkbom et al., J Am Chem Soc 137, 14430-14438 (2015)). Two mass ladder fragments at position 45 were found when reading from 3′ direction, corresponding to 45G and 45a. Similarly, these two merged into one mass ladder fragment only at position 44, corresponding to 44A and 44g (FIG. 11). Two isoforms were observed in all the reads which covered positions 44 and 45, and their ratios keep consistent at an equivalent level (quantified by EIC) (Table S5-24). To further verify the co-existence of the two mass fragments reading from two opposite directions, full-spectral analysis provided by commercial MassWorks (Cerno Bioscience, USA) was employed to examine the ions of these two fragments simultaneously in one spectrum. When reading from 5′ direction, two ions (m/z 778.1051 and 779.7068, 10^{th} charge state) were found, corresponding to 44A and 44g with a good mass accuracy (Y. Wang, M. Gu, Anal Chem 82, 7055-7062 (2010)). Similarly, full-spectral analysis also confirmed that 45G and 45a co-exist when reading from 3′ direction. Furthermore, the percentage ratios of 44A/44g and 45a/45G quantified by full-spectral analysis are consistent, indicating that they are from the same RNAs but reading from two opposite directions (5′ and 3). All these MS results support the finding that there is another isoform with 44g45a co-exist with wild-typed 44A45G, and the newly discovered mutated isoform is at an equivalent level to the wild-type. However, when a rtSBE experiment was performed to confirm this co-existing isoform using 2 primers, adjacent to position 44, and 45, the rtSBE results only supported the wild type form of tRNA^{Phe} (FIG. 34-35), not the mutated isoform. The SBE method was widely used for identifying DNA single nucleotide polymorphism (SNP). For example, if tRNA^{Phe} has A/g SNP at position 44, the rtSBE results could incorporate ddT and ddC since two isoforms have similar ratio. However, the results only showing ddT incorporated supported wild isoform A at position 44 (FIG. 35), so did rtSBE results at 45 position (FIG. 34). This indicates the reverse transcriptase (RT) couldn't well recognize the transition mutated forms of g and a, and the A-g/G-a transitions of tRNA^{Phe} may not occur in genome level because tRNA editing events mostly occurred internally in a tRNA molecule (J. M. Gott, B. H. Somerlot, M. W. Gray, RNA 16, 482-488 (2010)). So far, no study has been reported about the A-g/G-a transitions in tRNAs, and the mechanisms behind dinucleotide transition mutations remain to be explored. It was believed that the transition mutations at variable region may change the tRNA^{Phe} variable loop into a more stable stem (FIG. 12).

### Example 6

### Materials and Methods

Reagent and chemicals: All chemicals were purchased from commercial sources and used without further purification. tRNA (phenylalanine specific from brewer's yeast), RNaseT1, ATPγS and T4 polynucleotide kinase (3′-phosphatase free) were obtained from Sigma-Aldrich (St. Louis, Mo., USA), Formic acid (98-100%) was purchased from Merck KGaA (Darmstadt, Germany). Polynucleotide kinase (3′-phosphatase free) and SuperScript IV reverse transcriptase were purchased from Thermo Fisher Scientific (Waltham, Mass., USA). Adenosine-5′-5′-diphosphate-{5′-(cytidine-2′-O-methyl-3′-phosphate-TEG}-biotin and A(5)pp(5′)Cp-TEG-biotin-3′ synthesized by ChemGenes (Wilmington, Mass., USA). T4 DNA ligase was purchased from New England Biolabs (Ipswich, Mass., USA). Biotin maleimide was purchased from Vector Laboratories (Burlingame, Calif., USA). All other chemicals, including those needed for conversion of pseudouridine such as CMC (N-cyclohexyl-N′-(2-morpholinoethyl)-carbodiimide metho-p-toluenesulfonate), bicine, urea, EDTA, and Na₂CO₃ buffer, were obtained from Sigma-Aldrich unless otherwise stated.

### General Workflow

The general workflow is as follows unless indicated otherwise (N. Zhang et al., Nucleic Acids Research, 1-14 (2019)). tRNA was denatured at 80° C. for 2 min and then placed on ice for 1 min. (A. Bakin, J. Ofengand, Biochemistry 32, 9754-9762 (1993)). RNase T1 partial digestion was performed to fragment tRNA if needed (A. Bjorkbom et al., J Am Chem Soc 137, 14430-14438 (2015)). Biotin tag was chemically labeled on the 3′- or 5′-end of tRNA before or after RNase T1 digestion (T. H. Cormen et al. Introduction to Algorithms. MIT Press and McGraw-Hill, Second Edition, 540-549 (2001)). Biotin streptavidin capture/release and purification (T. F. Smith, M. S. Waterman, J Mol Biol 147, 195-197 (1981)). Acid degradation: labeled or unlabeled tRNA was degraded into a series of short, well-defined fragments (sequence ladder), ideally by random, sequence context-independent and single-cut cleavage of phosphodiester through a 2′-OH-assisted acidic hydrolysis mechanism (Y. Motorin et al., Methods Enzymol 425, 21-53 (2007)). The degradation fragments were then subjected to LC-MS analysis and the deconvoluted masses and retention times (t_{R}) were analyzed to identify each ladder fragment (Y. Motorin, et al., Methods Enzymol 425, 21-53 (2007)). Computation anchor algorithms were applied to automate the data processing and sequence generation process (S. Zhang et al. Proc Natl Acad Sci USA 110, 17732-17737 (2013)). Specific chemistries for identification and differentiation of isomeric modifications if needed.

### RNase T1 Digestion

Approximately 10 μg of tRNA was digested by 1 μL of 1000 U/μL of RNase T1 in 50 mM Tris-HCl (pH 7.5) containing 2 mM EDTA at room temperature for overnight. The digestion was stopped and purified by Oligo Clean & Concentrator (Zymo Research, Irvine, Calif., USA). Three major segments generated from digestion were detected by LC-MS.

Dephosphorylation of 5′ End of tRNA

10 μg of tRNA was digested by 1000 U of RNase T1 followed by purification by Oligo Clean & Concentrator. 20 μL of alkaline phosphatase (20 U/μL, Sigma-Aldrich) was added to the above described tRNA samples and incubated at 50° C. for 60 min followed by purification by Oligo Clean & Concentrator.

### 5′ and 3′-Ends Biotin Labeling and Biotin Streptavidin Capture/Release

5′ and 3′-ends biotin labeling as well as biotin streptavidin capture/release were performed by previously established methods (N. Zhang et al., Nucleic Acids Research, 1-14 (2019)).

Chemistry for Differentiating Pseudouridine (ψ) from Uridine

The experiments to convert ψ into CMC-ψ adducts were performed using a modified protocol according to a reported method (A. Bakin, J. Ofengand, Biochemistry 32, 9754-9762 (1993)). tRNA was denatured in 5 mM EDTA at 80° C. for 2 min and then placed on ice. tRNA (1 nmol) was treated with 0.17 M CMC in 50 mM Bicine (pH 8.3), 4 mM EDTA and 7 M urea at 37° C. for 20 min in a total reaction volume of 90 μL. The reaction was stopped with buffer A (60 μL of 1.5 M sodium acetate and 0.5 mM EDTA, pH 5.6). After purified by Oligo Clean & Concentrator, the resultant product was subsequently treated with 0.05 M Na₂CO₃ buffer (pH 10.4) at 37° C. for 17 h. The reaction was stopped with buffer A, and the crude product was purified by Oligo Clean & Concentrator to remove all the salts

### Chemistry for Aniline Cleavage at m⁷G

tRNA^{Phe} (1.6 nmol) was preincubated for 15 min at 37° C. in buffer (Tris-HCl buffer, pH 7.5, 0.01 M MgCl₂, 0.2 M KCl). The cooled solution was added to a freshly prepared ice-cold solution of NaBH₄ in the same buffer to give final concentrations of 60 μM tRNA and 0.5 M NaBH₄. The reduction was performed at 0° C. under subdued light. The reaction was terminated by pipetting aliquots of the reaction mixture into one tenth volume 6 N acetic acid and subsequent purification by Oligo Clean & Concentrator. Then, the tRNA pellet was dissolved in 200 μL×5 tubes aniline/acetate solution (aniline/acetic acid/water=1:3:7) and incubated for 10 min at 60° C. 10 volumes of 0.3 M sodium acetate, pH 5.5, were added and subsequently the sample was purified by Oligo Clean & Concentrator.

Reverse Transcription Single Base Extension (rtSBE)

Demethylation: ALKBH3 (2 μg/μL) was purchased from Active Motif (CA, USA). The reaction was carried out at 37° C. in 50 mM HEPES buffer (pH 8.0) containing 100 pmol tRNA^{phe}, 4 μg ALKBH3, 150 μM Fe(NH₄)₂(SO₄)₂, 1 mM α-ketoglutarate, 2 mM sodium ascorbate, and 1 mM TCEP for 1 h. Oligo Clean & Concentrator was applied to remove salts and excessive reactants.

rtSBE: A reverse primer 3′primer adjacent to m¹A position 5′-TGGTGCGAATTCTGTGGA-3′ (SEQ ID NO: 7) was designed, using tRNAphe as a template for m¹A detection, and de-methylated tRNA^{phe} as control template. The rtSBE reaction was conducted using SuperScript IV reverse transcriptase in 1×SSIV buffer 30 μl reaction volume contains 25 pmol template, 50 pmol primer, 2.5 nmol ddNTP, 100 mM DTT, 40 U RNase inhibitor, and 200 U SuperScript IV reverse transcriptase at 65° C. for 5 min, and then incubated on ice for 1 min. Then reverse transcription reaction was carried out for 25 cycles at 45° C. for 30 sec and 55° C. for 1 min. Lastly, the reaction was inactivated by incubating at 80° C. for 10 min followed by using Oligo Clean & Concentrator to remove all salts and proteins. The rtSBE products were checked by MALDI-TOF.

### LC-MS Analysis

General LC-MS conditions for analyzing tRNA sequencing ladders were the same as previously reported (N. Zhang et al., Nucleic Acids Research, 1-14 (2019)). except 2-20% buffer B in 60 min followed by a 2 min 90% buffer B wash step.

General MS conditions for the methylated dimers were the same as previously reported (A. Bjorkbom et al., J Am Chem Soc 137, 144:30-14438 (2015)). except the following: targeted ms/ms was used; the mass range for ms1 350-3200 m/z; the mass range for ms2 50-750. For dimer CₘU, the targeted precursor was 642.0837 (t_{R}=2.95 min); For dimer GₘA, the target precursor was 705.1164 (t_{R}=3.5 min and 4.08 min), CE=20. LC conditions: 2-20% MeOH in 60 min (buffer A: 200 mM 1,1,1,3,3,3-hexafluoro-2-propanol, 1.25 mM triethylamine in water).

General MS conditions for analyzing of single nucleosides or nucleotides if needed were the same as previously reported (N. Zhang, et al., Nucleic Acids Research, 1-14 (2019)) except m/z range 100-2000. LC conditions: 0% B for 5 min, 0-50% B for 30 min, 200 μL/min flow; buffer A: water, 0.1% formic acid (FA) and B: acetonitrile (ACM, 0.1% FA, column: Waters Acquity UPLC 2.1×100,

### Computation and Data Analysis

The sample data were acquired using the MassHunter Acquisition software (Agilent Technologies, USA). To extract relevant spectral and chromatographic information from the LC-MS experiments, the Molecular Feature Extraction (MFE) workflow in MassHunter Qualitative Analysis (Agilent Technologies, USA) was used. This proprietary molecular feature extractor algorithm performs untargeted feature finding in the mass and retention time dimensions. In principal, any software capable of compound identification could be used. The MFE settings were optimized to extract as many identified compounds as possible but with a reasonable quality score. The MFE settings applied were as follows: “centroid data format, small molecules (chromatographic), peak with height ≥100, up to a maximum of 1000, quality score ≥30”. However, data reduction was performed to simplify algorithm sequencing if needed. For instance, the numbers of input compounds used for algorithm analysis were generally an order-of-magnitude higher than the number of ladder fragments needed for generating complete sequences, unless indicated otherwise; these input compounds are sorted out of all MFE extracted compounds typically with higher volumes and/or better quality scores.

The formula used to calculate the PPM in the manuscript:

ppm=10⁻⁶×Mass_{theoretical}−Mass_{observed}/Mass_{theoretical}

### Global Hierarchical Ranking and Local Best Algorithm

Data pre-processing is a required step in order for the algorithm to focus on a particular subset of the input dataset at a time. There are two reasons to subset the dataset before parsing into the algorithm. First is to eliminate noise from the dataset. Second is because, experimentally, the RNA material to be sequenced requires fragmentation and labeling with molecular tags. The RNA sample loaded into LC-MS is a mixture of different fragments with some molecular tags. Because of the biochemical properties of the RNA fragments and the tags, in the output dataset from LC-MS, data points corresponding to different RNA fragments are distributed in different groups with distinctive statistics between those groups. The algorithm “zooms in” on one group to read out the sequence of one fragment at a time. Subsetting of the dataset is implemented by refining the RT and mass value of the input dataset in windows, and specifying the starting data point of each fragment. This is feasible because the molecular tag is added to the terminus of each fragment, and the RT and mass feature of the tag is known. Therefore, the algorithm is called “anchor-based”, since specifying the starting data point corresponding to the molecular tag latches down the data points corresponding to the specific fragment that one aims to read out from the whole dataset.

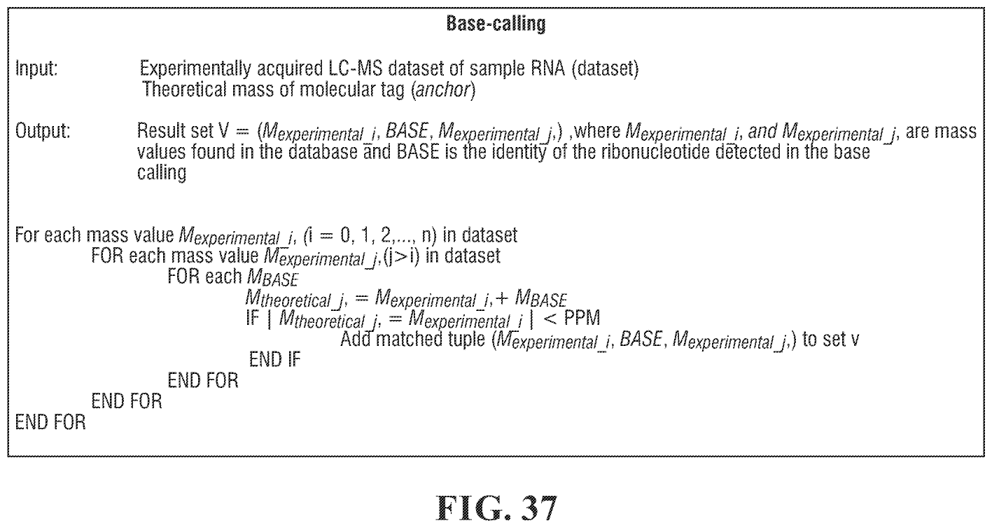

After subsetting the dataset, the algorithm performs base calling (FIG. 37). The theoretical mass, calculated from chemical formula, of all known ribonucleotides including those with modifications to the base is stored as a list of M_{BASE}. In the first iteration, the algorithm finds the mass corresponding to the molecular tag (anchor) and sets M_{experimental_i} equal to this mass. The algorithm tests each M_{BASE} from the list by adding it to M_{experimental_i} and generating a theoretical sum mass M_{theoretical_j}. The algorithm searches through the dataset for a mass value that matches with M_{theoretical_j}. If there exists a matching mass value M_{experimental_j}, a tuple (M_{experimental_i}, BASE, M_{experimental_j}) is stored in the result set V. Since the algorithm tests all M_{BASE} in the list and looks for all possible matches, multiple tuples with same M_{experimental_i} but different BASE identity and M_{experimental_j} are stored in set V. When the algorithm decides if there is a match, it takes into consideration the experimental error that the experimental mass may slightly deviate from the theoretical mass for a same ribonucleotide. A calculated parameter PPM that allows M_{experimental_j} to be matched with M_{theoretical_j} within a customizable range was implemented.

The algorithm performs base calling for all data points until all possible tuples are stored in set V. Note that each tuple in set V represents an individual base-calling possibility.

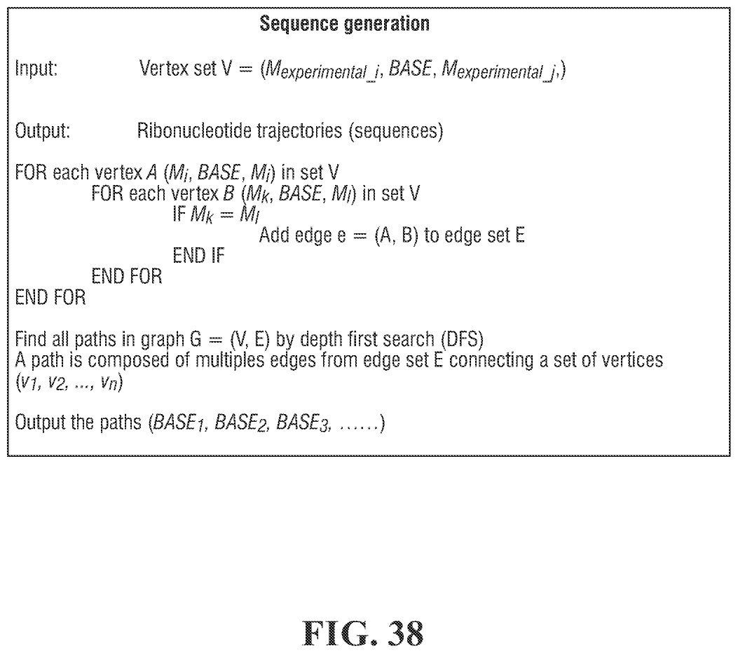

After base calling, the algorithm builds trajectories linking tuples in set V to generate sequences of the RNA fragment (FIG. 38). Taken tuples from set V as vertices, the algorithm finds and stores all edges by examining pairs of tuples such that for a given pair of tuples (Mᵢ, BASE, Mⱼ) and (Mₖ, BASE, Mₗ), Mₖ=Mⱼ. The algorithm generates a graph G=(V, E) while finding the edges. When graph G is completed, the algorithm finds all paths in graph G by depth first search (DFS) (4). All paths are stored as sets of vertices. Since the vertices contained in the path are tuples (M_{experimental_i}, BASE, M_{experimental_j}), BASE can be outputted as a sequence of ribonucleotides.

Because the outputs from LC-MS contains a huge number of data points, graph G contains the same number of vertices and also huge number of edges, resulting in tremendous number of total paths, each representing a draft read. To effectively filter the draft reads, two draft read selection strategies have been developed, namely the global hierarchical ranking strategy and the local best score strategy. Nonetheless, both strategies use same parameters acquired from the LC-MS dataset to score the draft reads such as volume and quality score (QS).

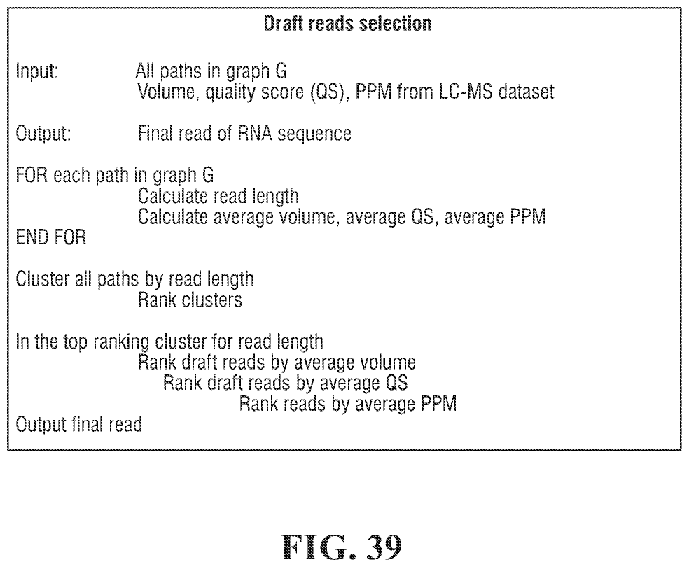

In the global hierarchical ranking strategy (FIG. 39), the draft reads are scored after the sequence generation step with the following criteria: read length, average volume, average QS, and average PPM. Read length is the number of BASE in a draft read. Average volume is calculated by summing the volume associated with each data point in a draft read and diving the sum by read length. Average QS is calculated by dividing the sum of QS by read length for each draft read. Average PPM is the sum of all PPM values associated with data points contained in a draft read divided by read length. The first step of the global hierarchical ranking strategy groups all draft reads into clusters based on their read length, and each cluster is assigned a ranking score for read length. The cluster receiving the highest ranking contains draft reads of the top read length, and the algorithm focuses on this cluster in the following steps. Within this cluster, the draft reads are assigned secondary ranking scores based on average volume values, with drafts reads of higher average volumes receiving higher rankings. In case where more than one draft read have a same read length and average volume value and thus receive a same ranking, the algorithm uses average QS value to re-rank these draft reads, with higher average QS values resulting in higher ranks. If there are still multiple draft reads receiving the same rank, the algorithm uses average PPM value to re-rank these draft reads again, but higher ranks are assigned to draft reads with lower average PPM values since PPM reflects the experimental error associated with each data point from LC-MS. In the end, the draft read with longest read length, highest average volume, highest average QS and lowest average PPM beats all other draft reads in the hierarchical ranking procedure and will be outputted as the final read for the targeted RNA fragment.

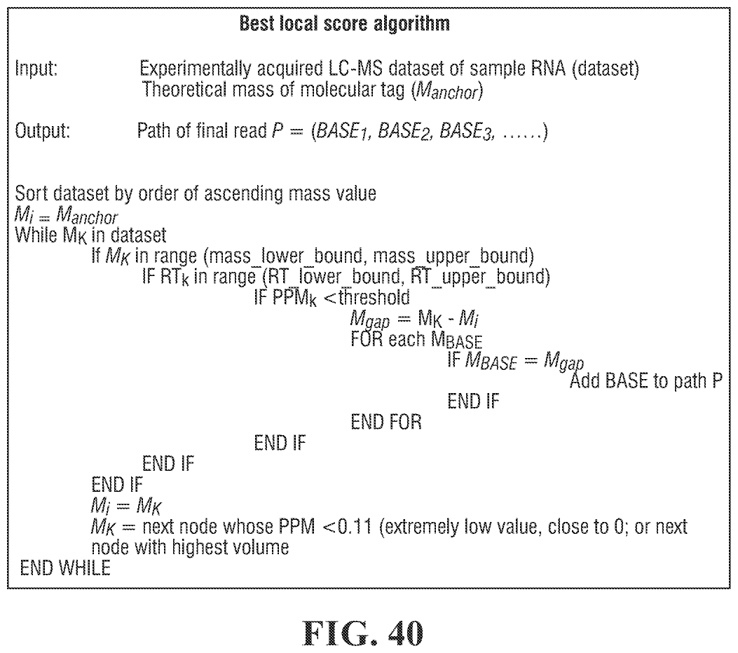

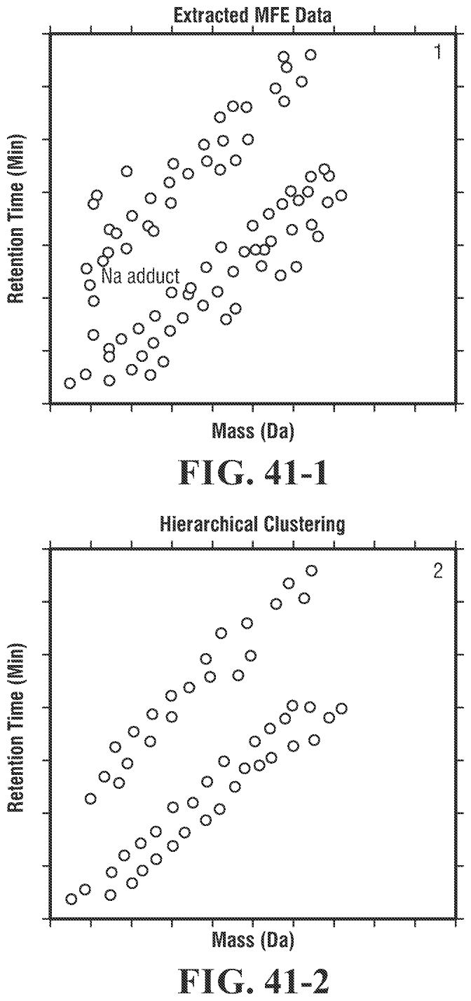

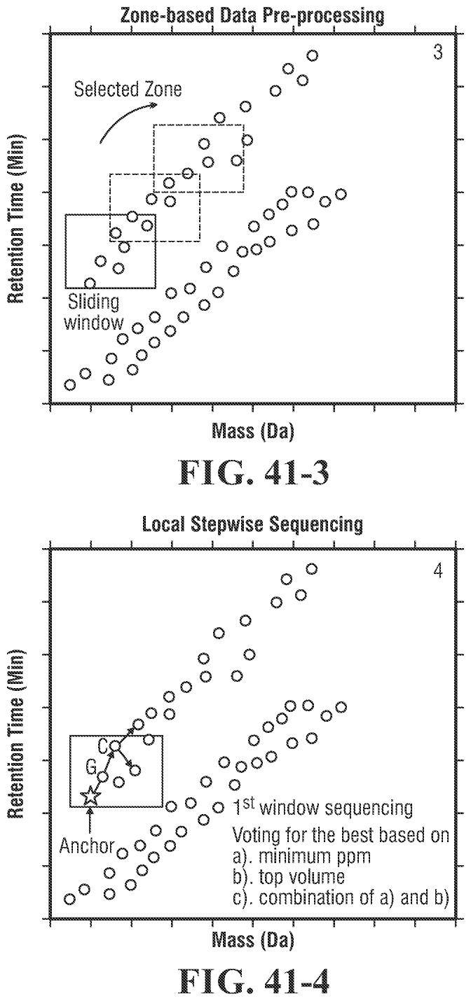

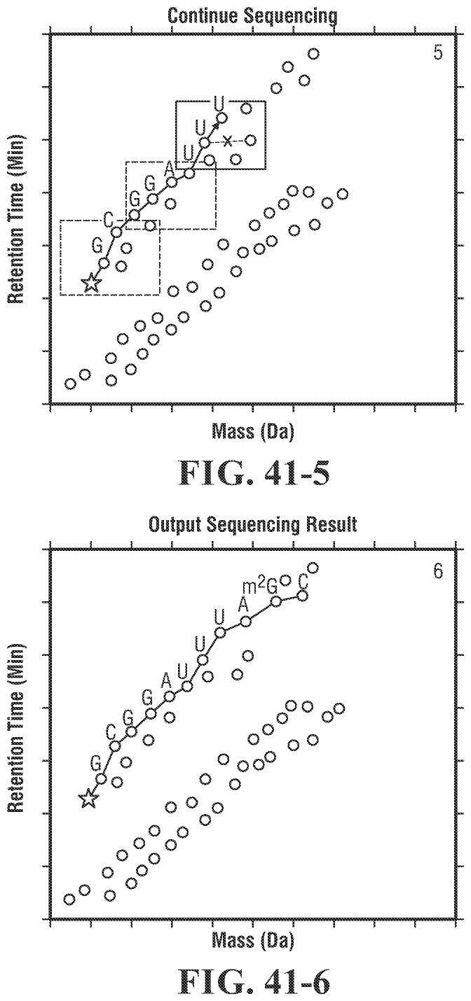

Alternatively, the local best score strategy differs from the previous strategy from the step of base calling (FIG. 40 and FIG. 41). The algorithm of local best score strategy applies the anchor-based method to focus on a specific subset of LC-MS dataset presorted by ascending mass order. It pins down the starting ribonucleotide by user defined anchor mass and locates data points from the entire fragment by the anchor. Focusing on these data points, the algorithm now performs base calling and simultaneously evaluates each data point. All data points in the desired zone are now considered as nodes, and the algorithm completes a single path as the final read based on the evaluation of each node. For a current node, its mass difference from the previously node (initialized as the anchor) is compared to the list of all known ribonucleotide masses for a match of identity. The match is only accepted if the PPM value of this node is below a certain threshold. In the test data with tRNA samples, this threshold was specified as 10, but it should always be customized to the actual LC-MS dataset. After accepting or rejecting the match (or mismatch otherwise), the algorithm stores the identity of the matched ribonucleotide, and moves on to the next node. There are always several possible next nodes based on their RT. The node with the highest volume will be chosen, with the exception that if a node has outstandingly small PPM value (close to 0) then this node will be chosen over other nodes with higher volumes. The algorithm now searches for a match of identity of the chosen node, evaluates the match, and store the ribonucleotide identity. This process is repeated until the full sequence in the desired data zone is read out.

### CCA Truncated Isoforms Detection

Searches for isoforms of Segment III as an additional step to the global hierarchical ranking algorithm were done. The final output (Table S5-1 through Table S5-3) of the original algorithm is one of the three isoforms and is aligned with all draft reads by Smith-Waterman alignment (T. F. Smith, M. S. Waterman, J Mol Biol 147, 195-197 (1981)) to acquire their alignment score. Draft reads with alignment score above 94.44% are considered candidates of isoforms, and the candidates are ranked by average volume. Six candidates were acquired with a cut off at 94.44%. Because the variation between the isoforms is only that they have different tails of C, CC or CCA respectively, the tails of the six candidates were trimmed and a second round of Smith-Waterman alignment was executed. After trimming, draft reads of isoforms had 100% alignment score with each other, and thus were filtered out from the six candidates.

All the final output data referenced by this paper were listed in (Table S5-1 through Table S5-11 and Table S5-13 through Table S5-17). The output data also can be presented by 2D figures (FIG. 13).

### Tables

TABLE S1-1
LC-MS analysis of 3′-biotin-labeled RNA #1 after streptavidin-aided bead
separation followed by subsequent chemical degradation(3′-labeled ladder components of
RNA #1, referring to the top curve in FIG. 1C).

| | | | | Extracted data file after LC/MS analysis | | | | |
|---|---|---|---|---|---|---|---|---|
| Theoretical | | | | | | | Quality | Error |
| Fragments | Theoretical mass | Base mass | Base | MFE mass | t_{R} | Volume | Score | ppm |
| 19 | 6781.0733 | 305.0413 | C | 6781.0413 | 9.752 | 16819442 | 100 | 4.72 |
| 18 | 6476.0320 | 345.0474 | G | 6475.9924 | 9.717 | 247965 | 84 | 6.11 |
| 17 | 6130.9846 | 305.0413 | C | 6130.9398 | 9.662 | 178841 | 80 | 7.31 |
| 16 | 5825.9433 | 329.0525 | A | 5825.9037 | 9.782 | 510096 | 80 | 6.80 |
| 15 | 5496.8908 | 306.0253 | U | 5496.8566 | 9.383 | 262486 | 99 | 6.22 |
| 14 | 5190.8655 | 305.0413 | C | 5190.8364 | 9.241 | 349988 | 100 | 5.61 |
| 13 | 4885.8242 | 306.0253 | U | 4885.7908 | 9.135 | 356118 | 100 | 6.84 |
| 12 | 4579.7989 | 345.0475 | G | 4579.7738 | 9.109 | 386687 | 100 | 5.48 |
| 11 | 4234.7514 | 329.0525 | A | 4234.7271 | 9.145 | 305380 | 100 | 5.74 |
| 10 | 3905.6989 | 305.0413 | C | 3905.6749 | 8.575 | 145505 | 96 | 6.14 |
| 9 | 3600.6576 | 306.0253 | U | 3600.6373 | 8.420 | 195308 | 100 | 5.64 |
| 8 | 3294.6323 | 345.0474 | G | 3294.6165 | 8.370 | 125991 | 100 | 4.80 |
| 7 | 2949.5849 | 329.0525 | A | 2949.5716 | 8.339 | 106993 | 100 | 4.51 |
| 6 | 2620.5324 | 305.0413 | C | 2620.5193 | 7.492 | 90629 | 100 | 5.00 |
| 5 | 2315.4911 | 305.0413 | C | 2315.4814 | 7.299 | 163692 | 100 | 4.19 |
| 4 | 2010.4498 | 329.0525 | A | 2010.4388 | 7.625 | 279963 | 100 | 5.47 |
| 3 | 1681.3973 | 329.0525 | A | 1681.3891 | 7.354 | 183827 | 100 | 4.88 |
| 2 | 1352.3448 | 329.0526 | A | 1352.3378 | 7.303 | 135065 | 100 | 5.18 |
| 1 | 1023.2922 | 29.0525 | A | 1023.2859 | 7.219 | 106700 | 100 | 6.16 |

Output sequence: CGCAUCUGACUGACCAAAA (SEQ ID NO: 10)

TABLE S1-2
LC-MS analysis of 3′-biotin-labeled RNA #1 after streptavidin-aided bead separation followed by subsequent
chemical degradation (5′-unlabeled ladder components of RNA #1, referring to the bottom curve in FIG. 1C).

| Theoretical | | | | Extracted data file after LC/MS analysis | | | | |
|---|---|---|---|---|---|---|---|---|
| | Theoretical | Base | | MFE | | | Quality | Error |
| Fragments | mass | mass | Base | mass | t_{R} | Volume | Score | ppm |
| 19 | 6024.8778 | 249.0862 | A | 6024.8483 | 7.664 | 14325731 | 100 | 4.90 |
| 18 | 5775.7916 | 329.0525 | A | 5775.7522 | 7.701 | 457844 | 87 | 6.82 |
| 17 | 5446.7391 | 329.0525 | A | 5446.6965 | 7.411 | 417145 | 100 | 7.82 |
| 16 | 5117.6866 | 329.0525 | A | 5117.6572 | 7.105 | 490290 | 100 | 5.74 |
| 15 | 4788.6341 | 305.0413 | C | 4788.6060 | 6.685 | 728135 | 100 | 5.87 |
| 14 | 4483.5928 | 305.0413 | C | 4483.5657 | 6.428 | 481770 | 100 | 6.04 |
| 13 | 4178.5515 | 329.0525 | A | 4178.5286 | 6.183 | 297514 | 100 | 5.48 |
| 12 | 3849.4990 | 345.0475 | G | 3849.4787 | 5.653 | 518403 | 100 | 5.27 |
| 11 | 3504.4515 | 306.0253 | U | 3504.4331 | 5.238 | 614494 | 100 | 5.25 |
| 10 | 3198.4262 | 305.0413 | C | 3198.4106 | 4.785 | 524613 | 99 | 4.88 |
| 9 | 2893.3849 | 329.0525 | A | 2893.3714 | 4.341 | 373933 | 100 | 4.67 |
| 8 | 2564.3324 | 345.0474 | G | 2564.3219 | 3.458 | 509219 | 100 | 4.09 |
| 7 | 2219.2850 | 306.0253 | U | 2219.2752 | 2.840 | 579139 | 100 | 4.42 |
| 6 | 1913.2597 | 305.0413 | C | 1913.2521 | 2.081 | 466058 | 100 | 3.97 |
| 5 | 1608.2184 | 306.0253 | U | 1608.2123 | 1.375 | 372038 | 80 | 3.79 |
| 4 | 1302.1931 | 329.0525 | A | 1302.1878 | 0.925 | 240613 | 100 | 4.07 |
| 3 | 973.1406 | 305.0413 | C | 973.1367 | 0.765 | 208989 | 100 | 4.01 |
| 2 | 668.0993 | 345.0474 | G | 668.0955 | 0.652 | 26061 | 100 | 5.69 |
| 1 | 323.0519 | 305.0413 | C | NA* | NA | NA | NA | NA |

*NA: Not Analyzed.
The 350 Da threshold was set to minimize background ions from the elution buffers. Thus, the masses which are smaller than 350 Da were not detected.

TABLE S1-3
LC-MS analysis of 5′-biotin-labeled RNA #1 (5′-labeled ladder
components of RNA #1, referring to the bottom ladder curve in
black in FIG. 1D).

Extracted data file

| | |
|---|---|
| Theoretical | after LC/MS analysis |

| | Theoretical | Base | | MFE | | | Quality | Error |
|---|---|---|---|---|---|---|---|---|
| Fragments | mass | mass | Base | mass | t_{R} | Volume | Score | ppm |
| 19 | 6600.0415 | 249.0862 | A | 6600.0153 | 10.113 | 1468018 | 100 | 3.97 |
| 18 | 6350.9553 | 329.0525 | A | 6350.9006 | 10.094 | 139388 | 80 | 8.61 |
| 17 | 6021.9028 | 329.0525 | A | 6021.8665 | 9.957 | 152155 | 80 | 6.03 |
| 16 | 5692.8503 | 329.0525 | A | 5692.8225 | 9.806 | 122377 | 84 | 4.88 |
| 15 | 5363.7978 | 305.0413 | C | 5363.7567 | 9.594 | 255396 | 100 | 7.66 |
| 14 | 5058.7565 | 305.0413 | C | 5058.7320 | 9.508 | 169499 | 80 | 4.84 |
| 13 | 4753.7152 | 329.0525 | A | 4753.694 | 9.4494 | 121869 | 96 | 4.38 |
| 12 | 4424.6627 | 345.0475 | G | 4424.638 | 9.2049 | 222046 | 100 | 5.38 |
| 11 | 4079.6152 | 306.0253 | U | 4079.5920 | 9.067 | 296271 | 100 | 6.13 |
| 10 | 3773.5899 | 305.0413 | C | 3773.5679 | 8.937 | 249085 | 100 | 5.83 |
| 9 | 3468.5486 | 329.0525 | A | 3468.5308 | 8.838 | 185624 | 100 | 5.13 |
| 8 | 3139.4961 | 345.0474 | G | 3139.4834 | 8.507 | 319911 | 100 | 4.05 |
| 7 | 2794.4487 | 306.0253 | U | 2794.4360 | 8.288 | 380189 | 100 | 4.54 |
| 6 | 2488.4234 | 305.0413 | C | 2488.4134 | 8.073 | 317954 | 100 | 4.02 |
| 5 | 2183.3821 | 306.0253 | U | 2183.3725 | 7.863 | 305479 | 100 | 4.40 |
| 4 | 1877.3568 | 329.0525 | A | 1877.3489 | 7.642 | 222446 | 100 | 4.21 |
| 3 | 1548.3043 | 305.0413 | C | 1548.2982 | 7.088 | 361254 | 100 | 3.94 |
| 2 | 1243.2630 | 345.0474 | G | 1243.2575 | 6.798 | 162972 | 100 | 4.42 |
| 1 | 898.2156 | 305.0413 | C | 898.2105 | 6.880 | 88421 | 100 | 5.68 |

Output sequence: CGCAUCUGACUGACCAAAA (SEQ ID NO: 10)

TABLE S1-4
LC-MS analysis of 5′-biotin-labeled RNA #2 (5′-labeled ladder
components of RNA #2, referring to the top ladder curve in
red in FIG. 1D).

| | |
|---|---|
| | Extracted data file |
| Theoretical | after LC/MS analysis |

| | Theoretical | Base | | MFE | | | Quality | Error |
|---|---|---|---|---|---|---|---|---|
| Fragments | mass | mass | Base | mass | t_{R} | Volume | Score | ppm |
| 20 | 6898.0505 | 225.0750 | C | 6898.0210 | 10.014 | 3995416 | 100 | 4.28 |
| 19 | 6672.9755 | 345.0474 | G | 6673.4755 | 10.115 | 92706 | 80 | −74.9 |
| 18 | 6327.9281 | 305.0413 | C | 6327.8894 | 10.117 | 108088 | 80 | 6.12 |
| 17 | 6022.8868 | 329.0525 | A | 6022.8313 | 10.104 | 133027 | 100 | 9.21 |
| 16 | 5693.8343 | 306.0253 | U | 5693.7870 | 9.920 | 68281 | 80 | 8.31 |
| 15 | 5387.8090 | 305.0413 | C | 5387.7785 | 9.850 | 167081 | 80 | 5.66 |
| 14 | 5082.7677 | 306.0253 | U | 5082.7314 | 9.784 | 170198 | 100 | 7.14 |
| 13 | 4776.7424 | 345.0474 | G | 4776.7210 | 9.695 | 114657 | 99 | 4.48 |
| 12 | 4431.6950 | 329.0526 | A | 4431.6685 | 9.629 | 143358 | 92 | 5.98 |
| 11 | 4102.6424 | 305.0412 | C | 4102.6199 | 9.367 | 245033 | 100 | 5.48 |
| 10 | 3797.6012 | 306.0253 | U | 3797.5819 | 9.264 | 184127 | 100 | 5.08 |
| 9 | 3491.5759 | 345.0475 | G | 3491.5567 | 9.131 | 91691 | 100 | 5.50 |
| 8 | 3146.5284 | 329.0525 | A | 3146.5054 | 9.028 | 187937 | 100 | 7.31 |
| 7 | 2817.4759 | 305.0413 | C | 2817.4633 | 8.675 | 288050 | 100 | 4.47 |
| 6 | 2512.4346 | 305.0413 | C | 2512.4233 | 8.509 | 138698 | 100 | 4.50 |
| 5 | 2207.3933 | 305.0413 | C | 2207.3835 | 8.335 | 192998 | 100 | 4.44 |
| 4 | 1902.3520 | 345.0474 | G | 1902.3433 | 8.161 | 149466 | 100 | 4.57 |
| 3 | 1557.3046 | 329.0525 | A | 1557.2976 | 8.042 | 133349 | 100 | 4.49 |
| 2 | 1228.2521 | 306.0253 | U | 1228.2455 | 7.618 | 188828 | 100 | 5.37 |
| 1 | 922.2268 | 329.0525 | A | 922.2213 | 7.434 | 86674 | 100 | 5.96 |

Output sequence: AUAGCCCAGUCAGUCUACGC (SEQ ID NO: 11)

TABLE S1-5
LC-MS analysis of a 1 ψ-containing RNA #6 (ψ unconverted ladder components
in the 5′ ladder of RNA #6, referring to the bottom ladder curve in black in FIG. 2B).

| Theoretical | | | | Extracted data file after LC/MS analysis | | | | |
|---|---|---|---|---|---|---|---|---|
| | Theoretical | Base | | MFE | | | Quality | Error |
| Fragments | mass | mass | Base | mass | t_{R} | Volume | Score | ppm |
| 20 | 6345.9028 | 265.0811 | G | 6345.9217 | 11.736 | 41088112 | 100 | −2.98 |
| 19 | 6080.8217 | 329.0525 | A | 6080.8255 | 11.769 | 2582596 | 100 | −0.62 |
| 18 | 5751.7692 | 345.0474 | G | 5751.7749 | 11.496 | 2169051 | 100 | −0.99 |
| 17 | 5406.7218 | 306.0253 | U | 5406.7209 | 11.315 | 2126771 | 100 | 0.17 |
| 16 | 5100.6965 | 319.057 | m⁵C | 5100.6941 | 11.167 | 1149416 | 100 | 0.47 |
| 15 | 4781.6395 | 329.0525 | A | 4781.6402 | 10.970 | 2692877 | 100 | −0.15 |
| 14 | 4452.5870 | 306.0253 | U | 4452.5866 | 10.566 | 5448251 | 100 | 0.09 |
| 13 | 4146.5617 | 306.0253 | U | 4146.5603 | 10.343 | 4115258 | 100 | 0.34 |
| 12 | 3840.5364 | 329.0526 | A | 3840.5352 | 10.141 | 2038738 | 100 | 0.31 |
| 11 | 3511.4838 | 305.0413 | C | 3511.4836 | 9.610 | 1167942 | 100 | 0.06 |
| 10 | 3206.4425 | 305.0412 | C | 3206.4401 | 9.331 | 3422282 | 100 | 0.75 |
| 9 | 2901.4013 | 329.0526 | A | 2901.3988 | 9.067 | 2391922 | 100 | 0.86 |
| 8 | 2572.3487 | 306.0253 | Unconverted | 2572.3468 | 8.328 | 4952174 | 100 | 0.74 |
| | | | ψ | | | | | |
| 7 | 2266.3234 | 306.0253 | U | 2266.3215 | 7.944 | 4534905 | 100 | 0.84 |
| 6 | 1960.2981 | 345.0474 | G | 1960.2956 | 7.360 | 3437270 | 100 | 1.28 |
| 5 | 1615.2507 | 305.0413 | C | 1615.2481 | 6.693 | 4151449 | 100 | 1.61 |
| 4 | 1310.2094 | 305.0413 | C | 1310.2062 | 5.915 | 1289241 | 87 | 2.44 |
| 3 | 1005.1681 | 329.0525 | A | 1005.1655 | 4.416 | 913589 | 100 | 2.59 |
| 2 | 676.1156 | 329.0525 | A | 676.1140 | 3.321 | 748977 | 100 | 2.37 |
| 1 | 347.0631 | 329.0525 | A | NA* | NA | NA | NA | NA |

*NA: Not Analyzed. The 350 Da threshold was set to minimize background ions from the elution buffers. Thus, the masses which are smaller than 350 Da were not detected

TABLE S6
LC-MS analysis of a 1 ψ-containing RNA #6 (ladder components with CMC-
converted ψ in the 5′ ladder of RNA #6, referring to the top ladder
curve in red in FIG. 2B)

| | | | | Extracted data file | | | | |
|---|---|---|---|---|---|---|---|---|
| Theoretical | | | | after LC/MS analysis | | | | |
| | Theoretical | Base | | MFE | | | Quality | Error |
| Fragments | mass | mass | Base | mass | t_{R} | Volume | Score | ppm |
| 20 | 6597.1025 | 265.0811 | G | 6597.1125 | 13.985 | 60627484 | 100 | −1.52 |
| 19 | 6332.0214 | 329.0525 | A | 6332.0201 | 13.979 | 1541470 | 100 | 0.21 |
| 18 | 6002.9689 | 345.0474 | G | 6002.9756 | 13.816 | 2147847 | 89 | −1.12 |
| 17 | 5657.9215 | 306.0253 | U | 5657.9243 | 13.742 | 2608610 | 100 | −0.49 |
| 16 | 5351.8962 | 319.057 | m⁵C | 5351.8960 | 13.695 | 2110248 | 100 | 0.04 |
| 15 | 5032.8392 | 329.0525 | A | 5032.8400 | 13.633 | 1907945 | 100 | −0.16 |
| 14 | 4703.7867 | 306.0253 | U | 4703.7861 | 13.394 | 4110706 | 88 | 0.13 |
| 13 | 4397.7614 | 306.0253 | U | 4397.7599 | 13.320 | 2867370 | 100 | 0.34 |
| 12 | 4091.7361 | 329.0526 | A | 4091.7361 | 13.283 | 1855682 | 100 | 0.00 |
| 11 | 3762.6835 | 305.0413 | C | 3762.6830 | 12.962 | 2817838 | 100 | 0.13 |
| 10 | 3457.6422 | 305.0412 | C | 3457.6396 | 12.878 | 1149319 | 100 | 0.75 |
| 9 | 3152.6010 | 329.0526 | A | 3152.5974 | 12.934 | 746862 | 100 | 1.14 |
| 8 | 2823.5485 | 557.2251 | Converted | 2823.5455 | 12.380 | 2149383 | 100 | 1.06 |
| | | | ψ | | | | | |
| 7 | 2266.3234 | 306.0253 | U | 2266.3213 | 7.944 | 4767282 | 100 | 0.93 |
| 6 | 1960.2981 | 345.0474 | G | 1960.2956 | 7.360 | 3433416 | 100 | 1.28 |
| 5 | 1615.2507 | 305.0413 | C | 1615.2481 | 6.694 | 4174772 | 100 | 1.61 |
| 4 | 1310.2094 | 305.0413 | C | 1310.2071 | 5.917 | 806139 | 87 | 1.76 |
| 3 | 1005.1681 | 329.0525 | A | 1005.1655 | 4.416 | 913589 | 100 | 2.59 |
| 2 | 676.1156 | 329.0525 | A | 676.1140 | 3.321 | 743305 | 100 | 2.37 |
| 1 | 347.0631 | 329.0525 | A | NA* | NA | NA | NA | NA |

*NA: Not Analyzed. The 350 Da threshold was set to minimize background ions from the elution buffers. Thus, the masses which are smaller than 350 Da were not detected.
Output sequence: AAACCGUψACCAUUAm⁵CUGAG (SEQ ID NO: 12)

TABLE S1-7
LC-MS analysis of 3′-biotin-labeled RNA #1, showing its ladder
components (referring to the ladder curve in black in FIG. 3).

Extracted data file after

| | |
|---|---|
| Theoretical | LC/MS analysis |

| | Theoretical | Base | | MFE | | | Quality | Error |
|---|---|---|---|---|---|---|---|---|
| Fragments | mass | mass | Base | mass | t_{R} | Volume | Score | ppm |
| 19 | 6781.0733 | 305.0413 | C | 6781.0426 | 9.576 | 35286012 | 100 | 4.53 |
| 18 | 6476.0320 | 345.0474 | G | 6475.9985 | 9.535 | 23351 | 60 | 5.17 |
| 17 | 6130.9846 | 305.0413 | C | 6130.9933 | 9.473 | 50125 | 90 | −1.42 |
| 16 | 5825.9433 | 329.0525 | A | 5825.9244 | 9.634 | 55880 | 80 | 3.24 |
| 15 | 5496.8908 | 306.0253 | U | 5496.8590 | 9.218 | 633795 | 80 | 5.79 |
| 14 | 5190.8655 | 305.0413 | C | 5190.8470 | 9.078 | 849742 | 100 | 3.56 |
| 13 | 4885.8242 | 306.0253 | U | 4885.7976 | 8.976 | 1193120 | 100 | 5.44 |
| 12 | 4579.7989 | 345.0475 | G | 4579.7742 | 8.951 | 1191558 | 100 | 5.39 |
| 11 | 4234.7514 | 329.0525 | A | 4234.7340 | 8.989 | 1196633 | 100 | 4.11 |
| 10 | 3905.6989 | 305.0413 | C | 3905.6808 | 8.420 | 729180 | 100 | 4.63 |
| 9 | 3600.6576 | 306.0253 | U | 3600.6382 | 8.275 | 605689 | 100 | 5.39 |
| 8 | 3294.6323 | 345.0474 | G | 3294.6179 | 8.229 | 935654 | 100 | 4.37 |
| 7 | 2949.5849 | 329.0525 | A | 2949.5713 | 8.210 | 903559 | 100 | 4.61 |
| 6 | 2620.5324 | 305.0413 | C | 2620.5217 | 7.376 | 587699 | 100 | 4.08 |
| 5 | 2315.4911 | 305.0413 | C | 2315.4825 | 7.191 | 700118 | 100 | 3.71 |
| 4 | 2010.4498 | 329.0525 | A | 2010.4378 | 7.527 | 1052796 | 100 | 5.97 |
| 3 | 1681.3973 | 329.0525 | A | 1681.3901 | 7.273 | 714971 | 100 | 4.28 |
| 2 | 1352.3448 | 329.0526 | A | 1352.3387 | 7.230 | 447072 | 100 | 4.51 |
| 1 | 1023.2922 | 329.0525 | A | 1023.2881 | 7.148 | 736463 | 100 | 4.01 |

Output sequence: CGCAUCUGACUGACCAAAA (SEQ ID NO: 10)

TABLE S1-8
LC-MS analysis of 3′-biotin-labeled RNA #2, showing its ladder
components (referring to the ladder curve in red in FIG 3).

| | | | | Extracted data file | | | | |
|---|---|---|---|---|---|---|---|---|
| Theoretical | | | | after LC/MS analysis | | | | |
| | Theoretical | Base | | | | | Quality | Error |
| Fragments | mass | mass | Base | MFE mass | t_{R} | Volume | Score | ppm |
| 20 | 7079.0823 | 329.2088 | A | 7079.0513 | 9.529 | 34343980 | 100 | 4.38 |
| 19 | 6750.0298 | 306.1667 | U | 6749.9875 | 9.259 | 170073 | 78 | 6.27 |
| 18 | 6444.0045 | 329.2088 | A | 6443.9653 | 9.344 | 934361 | 97 | 6.08 |
| 17 | 6114.9519 | 345.2077 | G | 6114.9082 | 9.000 | 176482 | 94 | 7.15 |
| 16 | 5769.9045 | 305.1828 | C | 5769.8590 | 8.867 | 537259 | 80 | 7.89 |
| 15 | 5464.8632 | 305.1828 | C | 5464.8338 | 8.733 | 381043 | 100 | 5.38 |
| 14 | 5159.8219 | 305.1827 | C | 5159.7998 | 8.619 | 939572 | 99 | 4.28 |
| 13 | 4854.7806 | 329.2088 | A | 4854.7556 | 8.734 | 1104050 | 100 | 5.15 |
| 12 | 4525.7281 | 345.2078 | G | 4525.7027 | 8.273 | 799528 | 100 | 5.61 |
| 11 | 4180.6807 | 306.1667 | U | 4180.6575 | 8.047 | 727253 | 100 | 5.55 |
| 10 | 3874.6554 | 305.1828 | C | 3874.6361 | 7.836 | 1007297 | 100 | 4.98 |
| 9 | 3569.6141 | 329.2087 | A | 3569.5985 | 7.960 | 1323892 | 100 | 4.37 |
| 8 | 3240.5616 | 345.2078 | G | 3240.5458 | 7.328 | 854305 | 100 | 4.88 |
| 7 | 2895.5141 | 306.1668 | U | 2895.5009 | 6.991 | 838944 | 100 | 4.56 |
| 6 | 2589.4888 | 305.1827 | C | 2589.4785 | 6.639 | 1076014 | 100 | 3.98 |
| 5 | 2284.4476 | 306.1668 | U | 2284.4388 | 6.433 | 1085561 | 100 | 3.85 |
| 4 | 1978.4223 | 329.2088 | A | 1978.4152 | 6.298 | 1224106 | 100 | 3.59 |
| 3 | 1649.3697 | 305.1827 | C | 1649.3632 | 5.150 | 443067 | 100 | 3.94 |
| 2 | 1344.3284 | 345.2078 | G | 1344.3229 | 5.115 | 530069 | 100 | 4.09 |
| 1 | 999.2810 | 305.1827 | C | 999.2764 | 5.258 | 300175 | 100 | 4.60 |

Output sequence: AUAGCCCAGUCAGUCUACGC (SEQ ID NO: 11)

TABLE S1-9
LC-MS analysis of 3′-biotin-labeled RNA #3, showing its ladder
components (referring to the ladder curve in green in FIG. 3).

| | | | | Extracted data file after | | | | |
|---|---|---|---|---|---|---|---|---|
| Theoretical | | | | LC/MS analysis | | | | |
| | Theoretical | Base | | | | | Quality | Error |
| Fragments | mass | mass | Base | MFE mass | t_{R} | Volume | Score | ppm |
| 20 | 7088.0826 | 329.0525 | A | 7088.0479 | 9.902 | 18422776 | 100 | 4.90 |
| 19 | 6759.0301 | 329.0525 | A | 6758.9878 | 9.816 | 342458 | 82 | 6.26 |
| 18 | 6429.9776 | 329.0525 | A | 6429.9401 | 9.553 | 297978 | 100 | 5.83 |
| 17 | 6100.9251 | 305.0413 | C | 6100.8860 | 9.162 | 176200 | 80 | 6.41 |
| 16 | 5795.8838 | 305.0413 | C | 5795.8502 | 9.059 | 325811 | 100 | 5.80 |
| 15 | 5490.8425 | 345.0475 | G | 5490.8084 | 9.029 | 561379 | 99 | 6.21 |
| 14 | 5145.7950 | 306.0253 | U | 5145.7640 | 8.927 | 543764 | 100 | 6.02 |
| 13 | 4839.7697 | 306.0253 | U | 4839.7382 | 8.852 | 751511 | 100 | 6.51 |
| 12 | 4533.7444 | 329.0525 | A | 4533.7170 | 8.857 | 916467 | 100 | 6.04 |
| 11 | 4204.6919 | 305.0413 | C | 4204.6726 | 8.273 | 363029 | 100 | 4.59 |
| 10 | 3899.6506 | 305.0413 | C | 3899.6323 | 8.164 | 664338 | 100 | 4.69 |
| 9 | 3594.6093 | 329.0525 | A | 3594.5912 | 8.300 | 1247513 | 100 | 5.04 |
| 8 | 3265.5568 | 306.0253 | U | 3265.5400 | 7.653 | 597972 | 100 | 5.14 |
| 7 | 2959.5315 | 306.0253 | U | 2959.5186 | 7.464 | 985122 | 100 | 4.36 |
| 6 | 2653.5062 | 329.0525 | A | 2653.4963 | 7.431 | 1500526 | 100 | 3.73 |
| 5 | 2324.4537 | 305.0413 | C | 2324.4444 | 6.486 | 663475 | 100 | 4.00 |
| 4 | 2019.4124 | 306.0253 | U | 2019.4039 | 6.101 | 752760 | 100 | 4.21 |
| 3 | 1713.3871 | 345.0474 | G | 1713.3811 | 5.973 | 1299628 | 100 | 3.50 |
| 2 | 1368.3397 | 329.0525 | A | 1368.3335 | 6.144 | 379728 | 100 | 4.53 |
| 1 | 1039.2872 | 345.0474 | G | 1039.2820 | 5.644 | 273139 | 100 | 5.00 |

Output sequence: AAACCGUUACCAUUACUGAG (SEQ ID NO: 13)

TABLE S1-10
LC-MS analysis of 3′-biotin-labeled RNA #4, showing its ladder
components (referring to the ladder curve in pink in FIG. 3).

| | | | | Extracted data file after | | | | |
|---|---|---|---|---|---|---|---|---|
| Theoretical | | | | LC/MS analysis | | | | |
| | Theoretical | Base | | MFE | | | Quality | Error |
| Fragments | mass | mass | Base | mass | t_{R} | Volume | Score | ppm |
| 20 | 6954.9836 | 345.0475 | G | 6954.9478 | 9.243 | 16978916 | 100 | 5.15 |
| 19 | 6609.9361 | 305.0412 | C | 6609.8899 | 9.131 | 184784 | 80 | 6.99 |
| 18 | 6304.8949 | 345.0475 | G | 6304.8568 | 9.109 | 510790 | 80 | 6.04 |
| 17 | 5959.8474 | 306.0253 | U | 5959.7956 | 9.056 | 393186 | 90 | 8.69 |
| 16 | 5653.8221 | 329.0525 | A | 5653.7838 | 9.059 | 830821 | 100 | 6.77 |
| 15 | 5324.7696 | 305.0413 | C | 5324.7319 | 8.701 | 496925 | 98 | 7.08 |
| 14 | 5019.7283 | 329.0525 | A | 5019.6982 | 8.848 | 1059427 | 100 | 6.00 |
| 13 | 4690.6758 | 306.0253 | U | 4690.6470 | 8.345 | 581020 | 82 | 6.14 |
| 12 | 4384.6505 | 305.0413 | C | 4384.6245 | 8.185 | 852527 | 100 | 5.93 |
| 11 | 4079.6092 | 306.0253 | U | 4079.5872 | 8.071 | 872930 | 100 | 5.39 |
| 10 | 3773.5839 | 306.0253 | U | 3773.5632 | 7.884 | 880358 | 100 | 5.49 |
| 9 | 3467.5586 | 305.0413 | C | 3467.5339 | 7.639 | 168485 | 97 | 7.12 |
| 8 | 3162.5173 | 305.0413 | C | 3162.4881 | 7.411 | 503294 | 100 | 9.23 |
| 7 | 2857.4760 | 305.0413 | C | 2857.4625 | 7.156 | 851140 | 100 | 4.72 |
| 6 | 2552.4347 | 305.0412 | C | 2552.4231 | 6.920 | 1065610 | 100 | 4.54 |
| 5 | 2247.3935 | 306.0253 | U | 2247.3838 | 6.690 | 1189236 | 100 | 4.32 |
| 4 | 1941.3682 | 306.0253 | U | 1941.3605 | 6.350 | 1445336 | 100 | 3.97 |
| 3 | 1635.3429 | 306.0254 | U | 1635.3384 | 6.009 | 22256 | 85 | 2.75 |
| 2 | 1329.3175 | 329.0525 | A | 1329.3120 | 6.598 | 1296266 | 100 | 4.14 |
| 1 | 1000.2650 | 306.0253 | U | 1000.2606 | 5.604 | 422194 | 100 | 4.40 |

Output sequence: GCGUACAUCUUCCCCUUUAU (SEQ ID NO: 14)

TABLE S1-11
LC-MS analysis of 3′-biotin-labeled RNA #5, showing its ladder components
(referring to the ladder curve in light blue in FIG. 3).

| | | | | Extracted data file after | | | | |
|---|---|---|---|---|---|---|---|---|
| Theoretical | | | | LC/MS analysis | | | | |
| | Theoretical | | | | | | Quality | Error |
| Fragments | mass | Base mass | Base | MFE mass | t_{R} | Volume | Score | ppm |
| 21 | 7522.1050 | 345.0475 | G | 7522.0681 | 9.519 | 21361914 | 100 | 4.91 |
| 20 | 7177.0575 | 305.0413 | C | 7176.9933 | 9.405 | 68800 | 60 | 8.95 |
| 19 | 6872.0162 | 345.0474 | G | 6871.9775 | 9.363 | 252280 | 88 | 5.63 |
| 18 | 6526.9688 | 345.0474 | G | 6526.9161 | 9.345 | 403291 | 100 | 8.07 |
| 17 | 6181.9214 | 329.0526 | A | 6181.8847 | 9.425 | 1246921 | 100 | 5.94 |
| 16 | 5852.8688 | 306.0253 | U | 5852.8226 | 9.054 | 263228 | 92 | 7.89 |
| 15 | 5546.8435 | 306.0253 | U | 5546.8116 | 8.935 | 1204009 | 100 | 5.75 |
| 14 | 5240.8182 | 306.0253 | U | 5240.7914 | 8.839 | 944494 | 100 | 5.11 |
| 13 | 4934.7929 | 329.0525 | A | 4934.7693 | 8.917 | 796848 | 100 | 4.78 |
| 12 | 4605.7404 | 345.0474 | G | 4605.7119 | 8.465 | 673185 | 100 | 6.19 |
| 11 | 4260.6930 | 305.0413 | C | 4260.6681 | 8.290 | 729523 | 100 | 5.84 |
| 10 | 3955.6517 | 306.0253 | U | 3955.6308 | 8.107 | 803678 | 100 | 5.28 |
| 9 | 3649.6264 | 305.0413 | C | 3649.6084 | 7.894 | 1056834 | 100 | 4.93 |
| 8 | 3344.5851 | 329.0525 | A | 3344.5687 | 7.990 | 1336987 | 100 | 4.90 |
| 7 | 3015.5326 | 345.0474 | G | 3015.5131 | 7.343 | 882742 | 100 | 6.47 |
| 6 | 2670.4852 | 306.0253 | U | 2670.4731 | 6.959 | 659989 | 100 | 4.53 |
| 5 | 2364.4599 | 306.0253 | U | 2364.4502 | 6.560 | 845446 | 100 | 4.10 |
| 4 | 2058.4346 | 345.0475 | G | 2058.4278 | 6.256 | 752026 | 100 | 3.30 |
| 3 | 1713.3871 | 345.0474 | G | 1713.3811 | 5.973 | 1299628 | 100 | 3.50 |
| 2 | 1368.3397 | 345.0475 | G | 1368.3335 | 6.144 | 379728 | 100 | 4.53 |
| 1 | 1023.2922 | 329.0525 | A | 1023.2881 | 7.148 | 736463 | 100 | 4.01 |

Output sequence: GCGGAUUUAGCUCAGUUGGGA (SEQ ID NO: 15)

TABLE S2-1
3′_biotin_RNA#_1_052118s04. Sequencing of
3′-biotin-labeled RNA #1 by an anchor-based
algorithm. The output sequence is indicated
below.

| Fragment | Mass | RT | Base | Volume | PPM |
|---|---|---|---|---|---|
| 1 | 694.2354 | 6.810 | 3′Tag | 55672 | 6.19 |
| 2 | 1023.2859 | 7.219 | A | 106700 | 6.16 |
| 3 | 1352.3378 | 7.303 | A | 135065 | 5.10 |
| 4 | 1681.3891 | 7.354 | A | 183827 | 4.82 |
| 5 | 2010.4388 | 7.625 | A | 279963 | 5.42 |
| 6 | 2315.4814 | 7.299 | C | 163692 | 4.15 |
| 7 | 2620.5193 | 7.492 | C | 90629 | 4.96 |
| 8 | 2949.5716 | 8.339 | A | 106993 | 4.48 |
| 9 | 3294.6165 | 8.370 | G | 125991 | 4.77 |
| 10 | 3600.6373 | 8.420 | U | 195308 | 5.61 |
| 11 | 3905.6749 | 8.575 | C | 145505 | 6.12 |
| 12 | 4234.7271 | 9.145 | A | 305380 | 5.71 |
| 13 | 4579.7738 | 9.109 | G | 386687 | 5.44 |
| 14 | 4885.7908 | 9.135 | U | 356118 | 6.80 |
| 15 | 5190.8364 | 9.241 | C | 349988 | 5.57 |
| 16 | 5496.8566 | 9.383 | U | 262486 | 6.19 |
| 17 | 5825.9037 | 9.782 | A | 510096 | 6.76 |
| 18 | 6130.9398 | 9.662 | C | 178841 | 7.27 |
| 19 | 6475.9924 | 9.717 | G | 247965 | 6.08 |
| 20 | 6781.0413 | 9.752 | C | 16819442 | 4.69 |

Output sequence: 5′-CGCAUCUGACUGACCAAAA-3′ (SEQ ID NO: 10)

TABLE S2-2
5′_OH_RNA#1_052118s04. Sequencing of the
5′-unlabeled mass ladders in 3′-biotin-labeled
RNA #1 by an anchor-based algorithm. The
output sequence is indicated below.

| Fragment | Mass | RT | Base | Volume | PPM |
|---|---|---|---|---|---|
| 1 | 668.0955 | 0.734 | G + C | 32710 | 5.69 |
| 2 | 973.1367 | 0.846 | C | 224370 | 4.01 |
| 3 | 1302.1878 | 1.006 | A | 261489 | 4.07 |
| 4 | 1608.2123 | 1.453 | U | 380380 | 3.79 |
| 5 | 1913.2522 | 2,161 | C | 498149 | 3.92 |
| 6 | 2219.2752 | 2.920 | U | 619956 | 4.42 |
| 7 | 2564.3220 | 3.538 | G | 557419 | 4.06 |
| 8 | 2893.3714 | 4.421 | A | 447008 | 4.67 |
| 9 | 3198.4107 | 4.866 | C | 629698 | 4.85 |
| 10 | 3504.4332 | 5.319 | U | 693526 | 5.22 |
| 11 | 3849.4786 | 5.733 | G | 601890 | 5.27 |
| 12 | 4178.5284 | 6.264 | A | 387527 | 5.50 |
| 13 | 4483.5665 | 6.509 | C | 602277 | 5.84 |
| 14 | 4788.6073 | 6.766 | C | 861658 | 5.58 |
| 15 | 5117.6579 | 7.186 | A | 642289 | 5.59 |
| 16 | 5446.6979 | 7,492 | A | 535900 | 7.55 |
| 17 | 5775.7534 | 7.781 | A | 591675 | 6.60 |
| 18 | 6024.8481 | 7.743 | A-end | 13740135 | 4.91 |

Output sequence: 5′-CGCAUCUGACUGACCAAAA-3′ (SEQ ID NO: 10)

TABLE S2-3
3′_OH_RNA#6_122718s07. Sequencing of non-
converted ψ mass ladders in CMC-converted
RNA #6 by an anchor-based algorithm. The
output sequence is indicated below.

| Fragment | Mass | RT | Base | Volume | PPM |
|---|---|---|---|---|---|
| 1 | 612.1432 | 1.334 | G + A | 609338 | 1.63 |
| 2 | 957.1909 | 1.354 | G | 1030368 | 0.73 |
| 3 | 1263.2160 | 1.390 | U | 992187 | 0.71 |
| 4 | 1582.2710 | 4.694 | mC | 2365111 | 1.77 |
| 5 | 1911.3250 | 6.426 | A | 6820867 | 0.68 |
| 6 | 2217.3496 | 6.547 | U | 5142524 | 0.90 |
| 7 | 2523.3752 | 7.060 | U | 3639095 | 0.67 |
| 8 | 2852.4279 | 8.384 | A | 6732016 | 0.53 |
| 9 | 3157.4687 | 8.247 | C | 4281684 | 0.63 |
| 10 | 3462.5110 | 8.533 | C | 2959433 | 0.29 |
| 11 | 3791.5638 | 9.613 | A | 6450776 | 0.18 |
| 12 | 4097.5897 | 9.281 | U | 2438044 | 0.02 |
| 13 | 4403.6162 | 9.655 | U | 1017645 | 0.25 |
| 14 | 4748.6638 | 10.082 | G | 2832083 | 0.27 |
| 15 | 5053.7053 | 10.247 | C | 1906586 | 0.30 |
| 16 | 5358.7538 | 10.493 | C | 1095672 | 1.62 |
| 17 | 5687.8032 | 11.149 | A | 1349414 | 0.98 |
| 18 | 6016.8560 | 11.603 | A | 2102227 | 0.98 |
| 19 | 6345.9139 | 11.737 | A | 90102376 | 1.78 |

Output sequence: 5′-AAACCGUψACCAUUAm5CUGAG-3′ (SEQ ID NO: 12)

TABLE S2-4
3′_OH_RNA#6_122718s07. Sequencing of Ψ-converted
mass ladders in CMC-converted RNA #6 by an anchor-
based algorithm. The output sequence is indicated
below.

| Fragment | Mass | RT | Base | Volume | PPM |
|---|---|---|---|---|---|
| 1 | 4348.7878 | 12.747 | Mod-Psi | 1061149 | 0.44 |
| 2 | 4654.8165 | 12.976 | U | 1028627 | 0.32 |
| 3 | 4999.8628 | 13.090 | G | 1603456 | 0.08 |
| 4 | 5304.9018 | 12.950 | C | 1145236 | 0.36 |
| 5 | 5609.9509 | 13.027 | C | 550752 | 1.05 |
| 6 | 5939.0021 | 13.618 | A | 919334 | 0.77 |
| 7 | 6268.0571 | 13.936 | A | 2514888 | 1.13 |
| 8 | 6597.1125 | 13.985 | A | 60627484 | 1.52 |

Output sequence: 5′-AAACCGUψ-3′ (Mod-Psi was designated for Ψ when output from the algorithm-processed sequences)

TABLE S2-5
LC-MS analysis of 3′-biotin-labeled RNA #1, showing its mass ladder components
(refers to the dataset for FIG. 7B). The output sequence is indicated below.

| | | | | Extracted data file after LC-MS | | | | |
|---|---|---|---|---|---|---|---|---|
| | Theoretical | | | analysis | | | | |
| | Theoretical | Base | | | | | Quality | Error |
| Fragments | mass | mass | Base | MFE mass | t_{R} | Volume | Score | ppm |
| 19 | 6781.0733 | 305.0413 | C | 6781.0426 | 9.576 | 35286012 | 100 | 4.53 |
| 18 | 6476.0320 | 345.0474 | G | 6475.9985 | 9.535 | 23351 | 60 | 5.17 |
| 17 | 6130.9846 | 305.0413 | C | 6130.9933 | 9.473 | 50125 | 90 | −1.42 |
| 16 | 5825.9433 | 329.0525 | A | 5825.9244 | 9.634 | 55880 | 80 | 3.24 |
| 15 | 5496.8908 | 306.0253 | U | 5496.8590 | 9.218 | 633795 | 80 | 5.79 |
| 14 | 5190.8655 | 305.0413 | C | 5190.8470 | 9.078 | 849742 | 100 | 3.56 |
| 13 | 4885.8242 | 306.0253 | U | 4885.7976 | 8.976 | 1193120 | 100 | 5.44 |
| 12 | 4579.7989 | 345.0475 | G | 4579.7742 | 8.951 | 1191558 | 100 | 5.39 |
| 11 | 4234.7514 | 329.0525 | A | 4234.7340 | 8.989 | 1196633 | 100 | 4.11 |
| 10 | 3905.6989 | 305.0413 | C | 3905.6808 | 8.420 | 729180 | 100 | 4.63 |
| 9 | 3600.6576 | 306.0253 | U | 3600.6382 | 8.275 | 605689 | 100 | 5.39 |
| 8 | 3294.6323 | 345.0474 | G | 3294.6179 | 8.229 | 935654 | 100 | 4.37 |
| 7 | 2949.5849 | 329.0525 | A | 2949.5713 | 8.210 | 903559 | 100 | 4.61 |
| 6 | 2620.5324 | 305.0413 | C | 2620.5217 | 7.376 | 587699 | 100 | 4.08 |
| 5 | 2315.4911 | 305.0413 | C | 2315.4825 | 7.191 | 700118 | 100 | 3.71 |
| 4 | 2010.4498 | 329.0525 | A | 2010.4378 | 7.527 | 1052796 | 100 | 5.97 |
| 3 | 1681.3973 | 329.0525 | A | 1681.3901 | 7.273 | 714971 | 100 | 4.28 |
| 2 | 1352.3448 | 329.0526 | A | 1352.3387 | 7.230 | 447072 | 100 | 4.51 |
| 1 | 1023.2922 | 329.0525 | A | 1023.2881 | 7.148 | 736463 | 100 | 4.01 |

Output sequence: 5′-CGCAUCUGACUGACCAAAA-3′ (SEQ ID NO: 10)

TABLE S2-6
LC-MS analysis of 3′-biotin-labeled RNA #2, showing its mass ladder components
(refers to the dataset for FIG.7B). The output sequence is indicated below.

| | | | | Extracted data file after LC-MS | | | | |
|---|---|---|---|---|---|---|---|---|
| | Theoretical | | | analysis | | | | |
| | Theoretical | | | | | | Quality | Error |
| Fragments | mass | Base mass | Base | MFE mass | t_{R} | Volume | Score | ppm |
| 20 | 7079.0823 | 329.2088 | A | 7079.0513 | 9.529 | 34343980 | 100 | 4.38 |
| 19 | 6750.0298 | 306.1667 | U | 6749.9875 | 9.259 | 170073 | 78 | 6.27 |
| 18 | 6444.0045 | 329.2088 | A | 6443.9653 | 9.344 | 934361 | 97 | 6.08 |
| 17 | 6114.9519 | 345.2077 | G | 6114.9082 | 9.000 | 176482 | 94 | 7.15 |
| 16 | 5769.9045 | 305.1828 | C | 5769.8590 | 8.867 | 537259 | 80 | 7.89 |
| 15 | 5464.8632 | 305.1828 | C | 5464.8338 | 8.733 | 381043 | 100 | 5.38 |
| 14 | 5159.8219 | 305.1827 | C | 5159.7998 | 8.619 | 939572 | 99 | 4.28 |
| 13 | 4854.7806 | 329.2088 | A | 4854.7556 | 8.734 | 1104050 | 100 | 5.15 |
| 12 | 4525.7281 | 345.2078 | G | 4525.7027 | 8.273 | 799528 | 100 | 5.61 |
| 11 | 4180.6807 | 306.1667 | U | 4180.6575 | 8.047 | 727253 | 100 | 5.55 |
| 10 | 3874.6554 | 305.1828 | C | 3874.6361 | 7.836 | 1007297 | 100 | 4.98 |
| 9 | 3569.6141 | 329.2087 | A | 3569.5985 | 7.960 | 1323892 | 100 | 4.37 |
| 8 | 3240.5616 | 345.2078 | G | 3240.5458 | 7.328 | 854305 | 100 | 4.88 |
| 7 | 2895.5141 | 306.1668 | U | 2895.5009 | 6.991 | 838944 | 100 | 4.56 |
| 6 | 2589.4888 | 305.1827 | C | 2589.4785 | 6.639 | 1076014 | 100 | 3.98 |
| 5 | 2284.4476 | 306.1668 | U | 2284.4388 | 6.433 | 1085561 | 100 | 3.85 |
| 4 | 1978.4223 | 329.2088 | A | 1978.4152 | 6.298 | 1224106 | 100 | 3.59 |
| 3 | 1649.3697 | 305.1827 | C | 1649.3632 | 5.150 | 443067 | 100 | 3.94 |
| 2 | 1344.3284 | 345.2078 | G | 1344.3229 | 5.115 | 530069 | 100 | 4.09 |
| 1 | 999.2810 | 305.1827 | C | 999.2764 | 5.258 | 300175 | 100 | 4.60 |

Output sequence: 5′ -AUAGCCCAGUCAGUCUACGC-3′ (SEQ′ ID NO: 11)

TABLE S2-7
LC-MS analysis of 3′-biotin-labeled RNA #3, showing its mass ladder components
(refers to the dataset for FIG.7B). The output sequence is indicated below.

| | | | | Extracted data file after LC-MS | | | | |
|---|---|---|---|---|---|---|---|---|
| | Theoretical | | | analysis | | | | |
| | Theoretical | Base | | MFE | | | Quality | Error |
| Fragments | mass | mass | Base | mass | t_{R} | Volume | Score | ppm |
| 20 | 7088.0826 | 329.0525 | A | 7088.0479 | 9.902 | 18422776 | 100 | 4.90 |
| 19 | 6759.0301 | 329.0525 | A | 6758.9878 | 9.816 | 342458 | 82 | 6.26 |
| 18 | 6429.9776 | 329.0525 | A | 6429.9401 | 9.553 | 297978 | 100 | 5.83 |
| 17 | 6100.9251 | 305.0413 | C | 6100.8860 | 9.162 | 176200 | 80 | 6.41 |
| 16 | 5795.8838 | 305.0413 | C | 5795.8502 | 9.059 | 325811 | 100 | 5.80 |
| 15 | 5490.8425 | 345.0475 | G | 5490.8084 | 9.029 | 561379 | 99 | 6.21 |
| 14 | 5145.7950 | 306.0253 | U | 5145.7640 | 8.927 | 543764 | 100 | 6.02 |
| 13 | 4839.7697 | 306.0253 | U | 4839.7382 | 8.852 | 751511 | 100 | 6.51 |
| 12 | 4533.7444 | 329.0525 | A | 4533.7170 | 8.857 | 916467 | 100 | 6.04 |
| 11 | 4204.6919 | 305.0413 | C | 4204.6726 | 8.273 | 363029 | 100 | 4.59 |
| 10 | 3899.6506 | 305.0413 | C | 3899.6323 | 8.164 | 664338 | 100 | 4.69 |
| 9 | 3594.6093 | 329.0525 | A | 3594.5912 | 8.300 | 1247513 | 100 | 5.04 |
| 8 | 3265.5568 | 306.0253 | U | 3265.5400 | 7.653 | 597972 | 100 | 5.14 |
| 7 | 2959.5315 | 306.0253 | U | 2959.5186 | 7.464 | 985122 | 100 | 4.36 |
| 6 | 2653.5062 | 329.0525 | A | 2653.4963 | 7.431 | 1500526 | 100 | 3.73 |
| 5 | 2324.4537 | 305.0413 | C | 2324.4444 | 6.486 | 663475 | 100 | 4.00 |
| 4 | 2019.4124 | 306.0253 | U | 2019.4039 | 6.101 | 752760 | 100 | 4.21 |
| 3 | 1713.3871 | 345.0474 | G | 1713.3811 | 5.973 | 1299628 | 100 | 3.50 |
| 2 | 1368.3397 | 329.0525 | A | 1368.3335 | 6.144 | 379728 | 100 | 4.53 |
| 1 | 1039.2872 | 345.0474 | G | 1039.2820 | 5.644 | 273139 | 100 | 5.00 |

Output sequence: 5′-AAACCGUUACCAUUACUGAG-3′ (SEQ ID NO: 13)

TABLE S2-8
LC-MS analysis of 3′-biotin-labeled RNA #4, showing its mass ladder components
(refers to the dataset for FIG.7B). The output sequence is indicated below.

| | | | | Extracted data file after LC-MS | | | | |
|---|---|---|---|---|---|---|---|---|
| | Theoretical | | | analysis | | | | |
| | Theoretical | Base | | MFE | | | Quality | Error |
| Fragments | mass | mass | Base | mass | t_{R} | Volume | Score | ppm |
| 20 | 6954.9836 | 345.0475 | G | 6954.9478 | 9.243 | 16978916 | 100 | 5.15 |
| 19 | 6609.9361 | 305.0412 | C | 6609.8899 | 9.131 | 184784 | 80 | 6.99 |
| 18 | 6304.8949 | 345.0475 | G | 6304.8568 | 9.109 | 510790 | 80 | 6.04 |
| 17 | 5959.8474 | 306.0253 | U | 5959.7956 | 9.056 | 393186 | 90 | 8.69 |
| 16 | 5653.8221 | 329.0525 | A | 5653.7838 | 9.059 | 830821 | 100 | 6.77 |
| 15 | 5324.7696 | 305.0413 | C | 5324.7319 | 8.701 | 496925 | 98 | 7.08 |
| 14 | 5019.7283 | 329.0525 | A | 5019.6982 | 8.848 | 1059427 | 100 | 6.00 |
| 13 | 4690.6758 | 306.0253 | U | 4690.6470 | 8.345 | 581020 | 82 | 6.14 |
| 12 | 4384.6505 | 305.0413 | C | 4384.6245 | 8.185 | 852527 | 100 | 5.93 |
| 11 | 4079.6092 | 306.0253 | U | 4079.5872 | 8.071 | 872930 | 100 | 5.39 |
| 10 | 3773.5839 | 306.0253 | U | 3773.5632 | 7.884 | 880358 | 100 | 5.49 |
| 9 | 3467.5586 | 305.0413 | C | 3467.5339 | 7.639 | 168485 | 97 | 7.12 |
| 8 | 3162.5173 | 305.0413 | C | 3162.4881 | 7.411 | 503294 | 100 | 9.23 |
| 7 | 2857.4760 | 305.0413 | C | 2857.4625 | 7.156 | 851140 | 100 | 4.72 |
| 6 | 2552.4347 | 305.0412 | C | 2552.4231 | 6.920 | 1065610 | 100 | 4.54 |
| 5 | 2247.3935 | 306.0253 | U | 2247.3838 | 6.690 | 1189236 | 100 | 4.32 |
| 4 | 1941.3682 | 306.0253 | U | 1941.3605 | 6.350 | 1445336 | 100 | 3.97 |
| 3 | 1635.3429 | 306.0254 | U | 1635.3384 | 6.009 | 22256 | 85 | 2.75 |
| 2 | 1329.3175 | 329.0525 | A | 1329.3120 | 6.598 | 1296266 | 100 | 4.14 |
| 1 | 1000.2650 | 306.0253 | U | 1000.2606 | 5.604 | 422194 | 100 | 4.40 |

Output sequence: 5′ -GCGUACAUCUUCCCCUUUAU-3′ (SEQ ID NO: 14)

TABLE S2-9
LC-MS analysis of 3′-biotin-labeled RNA #5, showing its mass ladder components
(refers to the dataset for FIG.7B). The output sequence is indicated below.

| | | | | Extracted data file after LC-MS | | | | |
|---|---|---|---|---|---|---|---|---|
| | Theoretical | | | analysis | | | | |
| | Theoretical | Base | | MFE | | | Quality | Error |
| Fragments | mass | mass | Base | mass | t_{R} | Volume | Score | ppm |
| 21 | 7522.1050 | 345.0475 | G | 7522.0681 | 9.519 | 21361914 | 100 | 4.91 |
| 20 | 7177.0575 | 305.0413 | C | 7176.9933 | 9.405 | 68800 | 60 | 8.95 |
| 19 | 6872.0162 | 345.0474 | G | 6871.9775 | 9.363 | 252280 | 88 | 5.63 |
| 18 | 6526.9688 | 345.0474 | G | 6526.9161 | 9.345 | 403291 | 100 | 8.07 |
| 17 | 6181.9214 | 329.0526 | A | 6181.8847 | 9.425 | 1246921 | 100 | 5.94 |
| 16 | 5852.8688 | 306.0253 | U | 5852.8226 | 9.054 | 263228 | 92 | 7.89 |
| 15 | 5546.8435 | 306.0253 | U | 5546.8116 | 8.935 | 1204009 | 100 | 5.75 |
| 14 | 5240.8182 | 306.0253 | U | 5240.7914 | 8.839 | 944494 | 100 | 5.11 |
| 13 | 4934.7929 | 329.0525 | A | 4934.7693 | 8.917 | 796848 | 100 | 4.78 |
| 12 | 4605.7404 | 345.0474 | G | 4605.7119 | 8.465 | 673185 | 100 | 6.19 |
| 11 | 4260.6930 | 305.0413 | C | 4260.6681 | 8.290 | 729523 | 100 | 5.84 |
| 10 | 3955.6517 | 306.0253 | U | 3955.6308 | 8.107 | 803678 | 100 | 5.28 |
| 9 | 3649.6264 | 305.0413 | C | 3649.6084 | 7.894 | 1056834 | 100 | 4.93 |
| 8 | 3344.5851 | 329.0525 | A | 3344.5687 | 7.990 | 1336987 | 100 | 4.90 |
| 7 | 3015.5326 | 345.0474 | G | 3015.5131 | 7.343 | 882742 | 100 | 6.47 |
| 6 | 2670.4852 | 306.0253 | U | 2670.4731 | 6.959 | 659989 | 100 | 4.53 |
| 5 | 2364.4599 | 306.0253 | U | 2364.4502 | 6.560 | 845446 | 100 | 4.10 |
| 4 | 2058.4346 | 345.0475 | G | 2058.4278 | 6.256 | 752026 | 100 | 3.30 |
| 3 | 1713.3871 | 345.0474 | G | 1713.3811 | 5.973 | 1299628 | 100 | 3.50 |
| 2 | 1368.3397 | 345.0475 | G | 1368.3335 | 6.144 | 379728 | 100 | 4.53 |
| 1 | 1023.2922 | 329.0525 | A | 1023.2881 | 7.148 | 736463 | 100 | 4.01 |

Output sequence: 5′-GCGGAUUUAGCUCAGUUGGGA-3′ (SEQ ID NO: 15)

TABLE S2-10
LC-MS analysis of 3′-biotin-labeled RNA #1 after isolation by streptavidin
beads followed by subsequent chemical degradation (3′-labeled mass ladder components of
RNA #1, which refers to the dataset for FIG.7B). The output sequence is indicated below.

| | | | | Extracted data file after LC-MS | | | | |
|---|---|---|---|---|---|---|---|---|
| | Theoretical | | | analysis | | | | |
| | Theoretical | | | | | | Quality | Error |
| Fragments | mass | Base mass | Base | MFE mass | t_{R} | Volume | Score | ppm |
| 19 | 6781.0733 | 305.0413 | C | 6781.0413 | 9.752 | 16819442 | 100 | 4.72 |
| 18 | 6476.0320 | 345.0474 | G | 6475.9924 | 9.717 | 247965 | 84 | 6.11 |
| 17 | 6130.9846 | 305.0413 | C | 6130.9398 | 9.662 | 178841 | 80 | 7.31 |
| 16 | 5825.9433 | 329.0525 | A | 5825.9037 | 9.782 | 510096 | 80 | 6.80 |
| 15 | 5496.8908 | 306.0253 | U | 5496.8566 | 9.383 | 262486 | 99 | 6.22 |
| 14 | 5190.8655 | 305.0413 | C | 5190.8364 | 9.241 | 349988 | 100 | 5.61 |
| 13 | 4885.8242 | 306.0253 | U | 4885.7908 | 9.135 | 356118 | 100 | 6.84 |
| 12 | 4579.7989 | 345.0475 | G | 4579.7738 | 9.109 | 386687 | 100 | 5.48 |
| 11 | 4234.7514 | 329.0525 | A | 4234.7271 | 9.145 | 305380 | 100 | 5.74 |
| 10 | 3905.6989 | 305.0413 | C | 3905.6749 | 8.575 | 145505 | 96 | 6.14 |
| 9 | 3600.6576 | 306.0253 | U | 3600.6373 | 8.420 | 195308 | 100 | 5.64 |
| 8 | 3294.6323 | 345.0474 | G | 3294.6165 | 8.370 | 125991 | 100 | 4.80 |
| 7 | 2949.5849 | 329.0525 | A | 2949.5716 | 8.339 | 106993 | 100 | 4.51 |
| 6 | 2620.5324 | 305.0413 | C | 2620.5193 | 7.492 | 90629 | 100 | 5.00 |
| 5 | 2315.4911 | 305.0413 | C | 2315.4814 | 7.299 | 163692 | 100 | 4.19 |
| 4 | 2010.4498 | 329.0525 | A | 2010.4388 | 7.625 | 279963 | 100 | 5.47 |
| 3 | 1681.3973 | 329.0525 | A | 1681.3891 | 7.354 | 183827 | 100 | 4.88 |
| 2 | 1352.3448 | 329.0526 | A | 1352.3378 | 7.303 | 135065 | 100 | 5.18 |
| 1 | 1023.2922 | 329.0525 | A | 1023.2859 | 7.219 | 106700 | 100 | 6.16 |

Output sequence: 5′-CGCAUCUGACUGACCAAAA-3′ (SEQ ID NO: 10)

TABLE S2-11
LC-MS analysis of 3′-biotin-labeled RNA #1 after isolation by streptavidin beads
followed by subsequent chemical degradation (5′-unlabeled mass ladder components of RNA
#1, which refers to the dataset for FIG.7A). The output sequence is indicated below.

| | | | | Extracted data file after LC-MS | | | | |
|---|---|---|---|---|---|---|---|---|
| | Theoretical | | | analysis | | | | |
| | Theoretical | Base | | | | | Quality | Error |
| Fragments | mass | mass | Base | MFE mass | t_{R} | Volume | Score | ppm |
| 19 | 6024.8778 | 249.0862 | A | 6024.8483 | 7.664 | 14325731 | 100 | 4.90 |
| 18 | 5775.7916 | 329.0525 | A | 5775.7522 | 7.701 | 457844 | 87 | 6.82 |
| 17 | 5446.7391 | 329.0525 | A | 5446.6965 | 7.411 | 417145 | 100 | 7.82 |
| 16 | 5117.6866 | 329.0525 | A | 5117.6572 | 7.105 | 490290 | 100 | 5.74 |
| 15 | 4788.6341 | 305.0413 | C | 4788.6060 | 6.685 | 728135 | 100 | 5.87 |
| 14 | 4483.5928 | 305.0413 | C | 4483.5657 | 6.428 | 481770 | 100 | 6.04 |
| 13 | 4178.5515 | 329.0525 | A | 4178.5286 | 6.183 | 297514 | 100 | 5.48 |
| 12 | 3849.4990 | 345.0475 | G | 3849.4787 | 5.653 | 518403 | 100 | 5.27 |
| 11 | 3504.4515 | 306.0253 | U | 3504.4331 | 5.238 | 614494 | 100 | 5.25 |
| 10 | 3198.4262 | 305.0413 | C | 3198.4106 | 4.785 | 524613 | 99 | 4.88 |
| 9 | 2893.3849 | 329.0525 | A | 2893.3714 | 4.341 | 373933 | 100 | 4.67 |
| 8 | 2564.3324 | 345.0474 | G | 2564.3219 | 3.458 | 509219 | 100 | 4.09 |
| 7 | 2219.2850 | 306.0253 | U | 2219.2752 | 2.840 | 579139 | 100 | 4.42 |
| 6 | 1913.2597 | 305.0413 | C | 1913.2521 | 2.081 | 466058 | 100 | 3.97 |
| 5 | 1608.2184 | 306.0253 | U | 1608.2123 | 1.375 | 372038 | 80 | 3.79 |
| 4 | 1302.1931 | 329.0525 | A | 1302.1878 | 0.925 | 240613 | 100 | 4.07 |
| 3 | 973.1406 | 305.0413 | C | 973.1367 | 0.765 | 208989 | 100 | 4.01 |
| 2 | 668.0993 | 345.0474 | G | 668.0955 | 0.652 | 26061 | 100 | 5.69 |
| 1 | 323.0519 | 305.0413 | C | NA* | NA | NA | NA | NA |

*NA: Not Analyzed. The 350 Da threshold was set to minimize background ions from the elution buffers. Otherwise, we would predominantly detect only HFIP and DPA ions. Thus, masses smaller than 350 Da were not detected. The output sequence is indicated below. Output sequence: 5′-CGCAUCUGACUGACCAAAA-3′ (SEQ ID NO: 10)

TABLE S2-12
LC-MS analysis of a single ψ-containing RNA #6 (ψ unconverted mass ladder
components from 3′ to 5′ of RNA #6, which refers to the dataset for FIG.7C). The output
sequence is indicated below.

| | | | | Extracted data file after LC-MS | | | | |
|---|---|---|---|---|---|---|---|---|
| | Theoretical | | | analysis | | | | |
| | Theoretical | Base | | MFE | | | Quality | Error |
| Fragments | mass | mass | Base | mass | t_{R} | Volume | Score | ppm |
| 20 | 6345.9028 | 329.0525 | A | 6345.9217 | 11.736 | 41088112 | 61.1 | −2.98 |
| 19 | 6016.8503 | 329.0525 | A | 6016.8560 | 11.603 | 2102227 | 96 | −0.95 |
| 18 | 5687.7978 | 329.0525 | A | 5687.8032 | 11.149 | 1349414 | 100 | −0.95 |
| 17 | 5358.7453 | 305.0413 | C | 5358.7538 | 10.493 | 1095672 | 100 | −1.59 |
| 16 | 5053.7040 | 305.0413 | C | 5053.7053 | 10.247 | 1906586 | 100 | −0.26 |
| 15 | 4748.6627 | 345.0475 | G | 4748.6638 | 10.082 | 2832083 | 100 | −0.23 |
| 14 | 4403.6152 | 306.0253 | U | 4403.6162 | 9.655 | 1017645 | 100 | −0.23 |
| 13 | 4097.5899 | 306.0253 | ψ | 4097.5897 | 9.281 | 2438044 | 100 | 0.05 |
| 12 | 3791.5646 | 329.0525 | A | 3791.5638 | 9.613 | 6450776 | 100 | 0.21 |
| 11 | 3462.5121 | 305.0413 | C | 3462.5110 | 8.533 | 2959433 | 100 | 0.32 |
| 10 | 3157.4708 | 305.0413 | C | 3157.4687 | 8.247 | 4281684 | 100 | 0.67 |
| 9 | 2852.4295 | 329.0525 | A | 2852.4279 | 8.384 | 6732016 | 100 | 0.56 |
| 8 | 2523.3770 | 306.0253 | U | 2523.3752 | 7.060 | 3639095 | 100 | 0.71 |
| 7 | 2217.3517 | 306.0253 | U | 2217.3496 | 6.547 | 5142524 | 100 | 0.95 |
| 6 | 1911.3264 | 329.0525 | A | 1911.3234 | 5.628 | 148978 | 100 | 1.57 |
| 5 | 1582.2739 | 319.0570 | m⁵C | 1582.2710 | 4.694 | 2365111 | 100 | 1.83 |
| 4 | 1263.2169 | 306.0253 | U | 1263.2160 | 1.392 | 1025750 | 100 | 0.71 |
| 3 | 957.1916 | 345.0474 | G | 957.1909 | 1.354 | 1030368 | 100 | 0.73 |
| 2 | 612.1442 | 329.0525 | A | 612.1432 | 1.334 | 609338 | 100 | 1.63 |
| 1 | 283.0917 | 345.0475 | G | NA* | NA | NA | NA | NA |

*NA: Not Analyzed. The 350 Da threshold was set to minimize background ions from the elution buffers. Otherwise, we would predominantly detect only HFIP and DPA ions. Thus, masses smaller than 350 Da were not detected. The output sequence is indicated below. Output sequence: 5′-AAACCGUψACCAUUAm5CUGAG3′ (SEQ ID NO: 12)

TABLE S2-13
LC-MS analysis of a 1 ψ-containing RNA #6 (mass ladder components with
CMC-converted ψ from 3′ to 5′, RNA #6, refers to the dataset for FIG.7C). The output
sequence is indicated at the bottom.

| | | | | Extracted data file after LC-MS | | | | |
|---|---|---|---|---|---|---|---|---|
| | Theoretical | | | analysis | | | | |
| | Theoretical | Base | | MFE | | | Quality | Error |
| Fragments | mass | mass | Base | mass | t_{R} | Volume | Score | ppm |
| 20 | 6597.1025 | 329.0525 | A | 6597.1125 | 13.985 | 60627484 | 100 | −1.52 |
| 19 | 6268.0500 | 329.0525 | A | 6268.0571 | 13.936 | 2514888 | 95.7 | −1.13 |
| 18 | 5938.9975 | 329.0525 | A | 5939.0021 | 13.618 | 919334 | 80 | −0.77 |
| 17 | 5609.9450 | 305.0413 | C | 5609.9509 | 13.027 | 550752 | 100 | −1.05 |
| 16 | 5304.9037 | 305.0413 | C | 5304.9018 | 12.95 | 1145236 | 100 | 0.36 |
| 15 | 4999.8624 | 345.0475 | G | 4999.8628 | 13.09 | 1603456 | 100 | −0.08 |
| 14 | 4654.8150 | 306.0253 | U | 4654.8165 | 12.976 | 1028627 | 100 | −0.32 |
| 13 | 4348.7897 | 557.2251 | Converted | 4348.7878 | 12.747 | 1061149 | 100 | 0.44 |
| | | | ψ | | | | | |
| 12 | 3791.5646 | 329.0525 | A | 3791.5638 | 9.613 | 6450776 | 100 | 0.21 |
| 11 | 3462.5121 | 305.0413 | C | 3462.511 | 8.533 | 2959433 | 100 | 0.32 |
| 10 | 3157.4708 | 305.0413 | C | 3157.4687 | 8.247 | 4281684 | 100 | 0.67 |
| 9 | 2852.4295 | 329.0525 | A | 2852.4279 | 8.384 | 6732016 | 100 | 0.56 |
| 8 | 2523.3770 | 306.0253 | U | 2523.3752 | 7.06 | 3639095 | 100 | 0.71 |
| 7 | 2217.3517 | 306.0253 | U | 2217.3496 | 6.547 | 5142524 | 100 | 0.95 |
| 6 | 1911.3264 | 329.0525 | A | 1911.3234 | 5.628 | 148978 | 100 | 1.57 |
| 5 | 1582.2739 | 319.0570 | m⁵C | 1582.271 | 4.694 | 2365111 | 100 | 1.83 |
| 4 | 1263.2169 | 306.0253 | U | 1263.216 | 1.392 | 1025750 | 100 | 0.71 |
| 3 | 957.1916 | 345.0474 | G | 957.1909 | 1.355 | 1052036 | 100 | 0.73 |
| 2 | 612.1442 | 329.0525 | A | 612.1432 | 1.334 | 609338 | 100 | 1.63 |
| 1 | 283.0917 | 345.0475 | G | NA* | NA | NA | NA | NA |

*NA: Not Analyzed. The 350 Da threshold was set to minimize background ions from the elution buffers. Otherwise, we would predominantly detect HFIP and DPA ions. Thus, the masses which are smaller than 350 Da were not detected. Output sequence: 5′AAACCGUψACCAUUAm⁵CUGAG3′ (SEQ ID NO: 12)

TABLE S3-1
3′_biotin_tRNA_T1_SIII_111418s05_76A.
Sequencing of 3′-biotin-labeled tRNA segment III
from 58m¹A to 76A using the global hierarchical
ranking algorithm and a revised Smith-Waterman
alignment similarity algorithm (alignment score:
95.0%). The output sequence is indicated at the
bottom.

| Fragment | Mass | RT | Base | Volume | PPM |
|---|---|---|---|---|---|
| 1 | 826.3164 | 35.809 | Tag | 2645323 | 2.42 |
| 2 | 1155.3679 | 34.555 | A | 580850 | 2.60 |
| 3 | 1460.4116 | 30.202 | C | 259583 | 0.41 |
| 4 | 1765.4505 | 29.311 | C | 4875476 | 1.70 |
| 5 | 2094.5027 | 30.921 | A | 560348 | 1.58 |
| 6 | 2399.5455 | 30.024 | C | 241970 | 0.75 |
| 7 | 2744.5948 | 30.494 | G | 365785 | 0.04 |
| 8 | 3049.6138 | 30.755 | C | 245795 | 7.28 |
| 9 | 3355.6561 | 31.57 | U | 377273 | 1.55 |
| 10 | 3661.6854 | 32.93 | U | 4226311 | 0.33 |
| 11 | 3990.7364 | 34.122 | A | 4968527 | 0.68 |
| 12 | 4319.7918 | 35.332 | A | 245329 | 0.05 |
| 13 | 4664.8388 | 34.606 | G | 4756748 | 0.04 |
| 14 | 4993.8992 | 35.504 | A | 307359 | 1.54 |
| 15 | 5298.9333 | 35.691 | C | 4083332 | 0.09 |
| 16 | 5627.9522 | 35.501 | A | 160811 | 5.88 |
| 17 | 5933.0022 | 35.649 | C | 157328 | 4.11 |
| 18 | 6238.0838 | 36.541 | C | 89737 | 2.55 |
| 19 | 6544.1101 | 36.202 | U | 672814 | 2.58 |
| 20 | 6887.1727 | 37.539 | mA | 1193510 | 1.66 |

Ts 1 Output Sequence: 5′-mAUCCACAGAAUUCGCACCA-3′ (SEQ ID NO: 16) mA is a symbol used in the global hierarchical ranking algorithm to designate a nucleobase modification that has the same mass value as a methylated A.

TABLE S3-2
3′_biotin_tRNA_T1_SIII_111418s05_75C.
Sequencing of 3′-biotin-labeled tRNA segment III
from 58m¹A to 75C using the global hierarchical
ranking algorithm and a revised Smith-Waterman
alignment similarity algorithm (alignment score:
100%). The output sequence is indicated at the
bottom.

| Fragment | Mass | RT | Base | Volume | PPM |
|---|---|---|---|---|---|
| 1 | 826.3164 | 35.809 | Tag | 2645323 | 2.42 |
| 2 | 1131.3573 | 28.724 | C | 2536602 | 2.12 |
| 3 | 1436.3979 | 26.748 | C | 1504369 | 2.16 |
| 4 | 1765.4505 | 29.311 | A | 4875476 | 1.70 |
| 5 | 2070.4898 | 27.904 | C | 1807879 | 2.41 |
| 6 | 2415.5392 | 28.436 | G | 4919858 | 1.24 |
| 7 | 2720.5806 | 28.781 | C | 4403013 | 1.07 |
| 8 | 3026.6061 | 29.745 | U | 5263366 | 0.89 |
| 9 | 3332.6311 | 30.654 | U | 3654432 | 0.90 |
| 10 | 3661.6854 | 32.930 | A | 4226311 | 0.33 |
| 11 | 3990.7364 | 34.122 | A | 4968527 | 0.68 |
| 12 | 4335.7879 | 33.348 | G | 2855812 | 0.32 |
| 13 | 4664.8388 | 34.606 | A | 4756748 | 0.04 |
| 14 | 4969.8783 | 34.250 | C | 2303352 | 0.40 |
| 15 | 5298.9333 | 35.691 | A | 4083332 | 0.09 |
| 16 | 5603.9769 | 35.502 | C | 2292626 | 0.50 |
| 17 | 5909.0178 | 35.637 | C | 2429322 | 0.41 |
| 18 | 6215.0412 | 36.088 | U | 860704 | 0.08 |
| 19 | 6558.1157 | 36.751 | mA | 16787962 | 1.05 |

Ts 2 Output Sequence: 5′-mAUCCACAGAAUUCGCACC-3′ (SEQ ID NO: 17)

TABLE S3-3
3′_biotin_tRNA_T1_SIII_111418s05_74C.
Sequencing of 3′-biotin-labeled tRNA segment III
from 58m¹A to 74C using the global hierarchical
ranking algorithm and a revised Smith-Waterman
alignment similarity algorithm (alignment score:
94.7%). The output sequence is indicated at the
bottom.

| Fragment | Mass | RT | Base | Volume | PPM |
|---|---|---|---|---|---|
| 1 | 826.3164 | 35.809 | Tag | 2645323 | 2.42 |
| 2 | 1131.3573 | 28.724 | C | 2536602 | 2.12 |
| 3 | 1460.4116 | 30.202 | A | 259583 | 0.41 |
| 4 | 1765.4505 | 29.311 | C | 4875476 | 1.70 |
| 5 | 2110.4918 | 27.882 | G | 356221 | 4.31 |
| 6 | 2415.5392 | 28.436 | C | 4919858 | 1.24 |
| 7 | 2721.5695 | 29.145 | U | 239635 | 0.73 |
| 8 | 3027.5972 | 30.047 | U | 68400 | 1.45 |
| 9 | 3356.6432 | 32.543 | A | 189932 | 0.63 |
| 10 | 3685.6934 | 33.833 | A | 159564 | 1.19 |
| 11 | 4030.7417 | 33.004 | G | 82558 | 0.87 |
| 12 | 4359.8007 | 34.352 | A | 289735 | 0.69 |
| 13 | 4664.8388 | 34.606 | C | 4756748 | 0.04 |
| 14 | 4993.8992 | 35.504 | A | 307359 | 1.54 |
| 15 | 5298.9333 | 35.691 | C | 4083332 | 0.09 |
| 16 | 5603.9769 | 35.502 | C | 2292626 | 0.50 |
| 17 | 5910.0206 | 35.639 | U | 98526 | 3.59 |
| 18 | 6253.0697 | 36.605 | mA | 181155 | 0.35 |

Ts 3 Output Sequence: 5′-mAUCCACAGAAUUCGC-3′ (SEQ ID NO: 18)

TABLE S3-4
5′_OH_tRNA_T1_SII_111418s05_44A45G.
Sequencing of 5′-OH tRNA segment II from 21A to
57G by the global hierarchical ranking algorithm.
The output sequence is indicated at the bottom.

| Frag- | | | | | |
|---|---|---|---|---|---|
| ment | Mass | RI | Base | Volume | PPM |
| 1 | 692.1081 | 0.945 | A + G | 448392 | 3.47 |
| 2 | 1021.1592 | 0.996 | A | 612623 | 3.72 |
| 3 | 1366.2059 | 1.023 | G | 1163701 | 3.29 |
| 4 | 1671.2489 | 1.112 | C | 1917190 | 1.68 |
| 5 | 2044.3269 | 8.858 | 2mG | 2025885 | 1.71 |
| 6 | 2349.3682 | 10.309 | C | 3120462 | 1.49 |
| 7 | 2654.4101 | 12.749 | C | 6309574 | 1.09 |
| 8 | 2983.4617 | 16.073 | A | 5462129 | 1.27 |
| 9 | 3328.5102 | 17.647 | G | 6892234 | 0.81 |
| 10 | 3657.5632 | 19.875 | A | 4203490 | 0.60 |
| 11 | 4282.6476 | 23.391 | U + Cm | 11059167 | 0.02 |
| 12 | 4970.7632 | 26.996 | A + Gm | 8957192 | 2.23 |
| 13 | 5299.8175 | 28.115 | A | 9137581 | 2.45 |
| 14 | 5511.8281 | 28.449 | Y′ | 9044373 | 2.70 |
| 15 | 5840.8796 | 29.718 | A | 7213450 | 8.82 |
| 16 | 6146.9082 | 30.061 | U | 12938074 | 8.92 |
| 17 | 6465.9647 | 30.688 | mC | 6445803 | 6.50 |
| 18 | 6771.9918 | 31.161 | U | 6802824 | 0.55 |
| 19 | 7117.0401 | 31.251 | G | 3468612 | 0.39 |
| 20 | 7462.0865 | 32.049 | G | 2834683 | 5.86 |
| 21 | 7791.1394 | 32.735 | A | 2239278 | 0.44 |
| 22 | 8136.1981 | 33.016 | G | 3437631 | 0.97 |
| 23 | 8495.2645 | 33.131 | mG | 2251492 | 6.91 |
| 24 | 8801.2888 | 33.439 | U | 3178250 | 6.56 |
| 25 | 9106.3319 | 33.677 | C | 3146668 | 7.88 |
| 26 | 9425.3892 | 33.961 | mC | 3341188 | 2.50 |
| 27 | 9731.4100 | 34.135 | U | 3700286 | 1.96 |
| 28 | 10076.4607 | 34.378 | G | 2776140 | 2.21 |
| 29 | 10382.4798 | 34.582 | U | 2849708 | 1.56 |
| 30 | 10727.5480 | 34.793 | G | 2740634 | 3.45 |
| 31 | 11047.5761 | 35.136 | T | 781981 | 2.18 |
| 32 | 11353.6241 | 35.183 | U | 4303300 | 4.11 |
| 33 | 11658.6776 | 35.364 | C | 1498752 | 5.05 |
| 34 | 12003.6973 | 35.531 | G | 6123452 | 2.60 |

Ts 4 Output Sequence: 5′-AGAGC2mGCCAGACmUGmAAY′AUmCUGGAGmGUCmCUGUGTUCG-3′ (SEQ ID NO: 19) 2mG, Gm, and mG are symbols used in the global hierarchical ranking algorithm to designate m²₂G (N², N²-dimethylguanosine), 2′-O-methylated G, and a nucleobase modification that has the same mass value as a methylated G (such as m²G and m⁷G), respectively.

TABLE S3-5
5′_OH_tRNA_T1_SII_111418s05_44g45a.
Sequencing of 5′-OH tRNA segment II from 21A to
57G by the global hierarchical ranking algorithm.
The output sequence is indicated at the bottom

| Fragment | Mass | RT | Base | Volume | PPM |
|---|---|---|---|---|---|
| 1 | 692.1081 | 0.945 | A + G | 448392 | 3.47 |
| 2 | 1021.1592 | 0.996 | A | 612623 | 3.72 |
| 3 | 1366.2059 | 1.023 | G | 1163701 | 3.29 |
| 4 | 1671.2489 | 1.112 | C | 1917190 | 1.68 |
| 5 | 2044.3269 | 8.858 | 2mG | 2025885 | 1.71 |
| 6 | 2349.3682 | 10.309 | C | 3120462 | 1.49 |
| 7 | 2654.4121 | 12.749 | C | 6309574 | 1.09 |
| 8 | 2983.4617 | 16.073 | A | 5462129 | 1.27 |
| 9 | 3328.5102 | 17.647 | G | 6892234 | 0.81 |
| 10 | 3657.5632 | 19.875 | A | 4203490 | 0.60 |
| 11 | 4282.6476 | 23.391 | U + Cm | 11059167 | 0.02 |
| 12 | 4970.7632 | 26.996 | A + Gm | 8957192 | 2.23 |
| 13 | 5299.8175 | 28.115 | A | 9137581 | 2.45 |
| 14 | 5511.8281 | 28.449 | Y′ | 9044373 | 2.70 |
| 15 | 5840.8796 | 29.718 | A | 7213450 | 8.82 |
| 16 | 6146.9082 | 30.061 | U | 12938074 | 8.92 |
| 17 | 6465.9647 | 30.688 | mC | 6445803 | 6.50 |
| 18 | 6771.9918 | 31.161 | U | 6802824 | 0.55 |
| 19 | 7117.0401 | 31.251 | G | 3468612 | 0.39 |
| 20 | 7462.0865 | 32.049 | G | 2834683 | 5.86 |
| 21 | 7807.1332 | 32.101 | G | 2248564 | 5.51 |
| 22 | 8136.1981 | 33.016 | A | 3437631 | 6.81 |
| 23 | 8495.2645 | 33.131 | mG | 2251492 | 6.91 |
| 24 | 8801.2888 | 33.439 | U | 3178250 | 6.56 |
| 25 | 9106.3319 | 33.677 | C | 3146668 | 7.88 |
| 26 | 9425.3892 | 33.961 | mC | 3341188 | 2.50 |
| 27 | 9731.4100 | 34.135 | U | 3700286 | 1.96 |
| 28 | 10076.4607 | 34.378 | G | 2776140 | 2.21 |
| 29 | 10382.4798 | 34.582 | U | 2849708 | 1.56 |
| 30 | 10727.5480 | 34.793 | G | 2740634 | 3.45 |
| 31 | 11047.5761 | 35.136 | T | 781981 | 2.18 |
| 32 | 11353.6241 | 35.183 | U | 4303300 | 4.11 |
| 33 | 11658.6776 | 35.364 | C | 1498752 | 5.05 |
| 34 | 12003.6973 | 35.531 | G | 6123452 | 2.60 |

Ts 5 Output Sequence: 5′-AGAGC2mGCCAGACmUGmAAY′AUmCUGGGAmGUCmCUGUGTUCG-3′ (SEQ ID NO 20) mC is a symbol used in the global hierarchical ranking algorithm to designate a nucleobase modification that has the same mass value as a methylated C.

TABLE S3-6
5′_pG_tRNA_T1_SI_111418s05. Sequencing of
5′-pG tRNA segment I from 1G to 20G by the
global hierarchical ranking algorithm.
The output sequence is indicated at the
bottom.

| Fragment | Mass | RT | Base | Volume | PPM |
|---|---|---|---|---|---|
| 1 | 443.0222 | 0.968 | pG | 32204 | 4.74 |
| 2 | 748.0626 | 0.935 | C | 327973 | 4.01 |
| 3 | 1093.1092 | 0.963 | G | 247078 | 3.48 |
| 4 | 1438.1583 | 1.010 | G | 1953624 | 1.46 |
| 5 | 1767.2105 | 2.512 | A | 6646248 | 1.36 |
| 6 | 2073.2377 | 4.800 | U | 11078570 | 0.24 |
| 7 | 2379.2611 | 7.664 | U | 13653044 | 1.01 |
| 8 | 2685.2874 | 9.948 | U | 13651928 | 0.52 |
| 9 | 3014.3399 | 13.244 | A | 8446589 | 0.46 |
| 10 | 3373.3974 | 16.657 | mG | 5400820 | 2.08 |
| 11 | 3678.4462 | 17.883 | C | 6427287 | 0.14 |
| 12 | 3984.4711 | 19.330 | U | 10498687 | 0.03 |
| 13 | 4289.5141 | 20.432 | C | 13067020 | 0.42 |
| 14 | 4618.5661 | 22.240 | A | 9336602 | 0.28 |
| 15 | 4963.6167 | 23.110 | G | 19445698 | 0.91 |
| 16 | 5271.6368 | 23.792 | D | 6241383 | 3.11 |
| 17 | 5579.6992 | 24.454 | 0 | 7740033 | 0.90 |
| 18 | 5924.7535 | 25.268 | G | 104745696 | 2.01 |
| 19 | 6269.8003 | 25.980 | G | 3057757 | 1.80 |
| 20 | 6614.8364 | 26.615 | G | 673220 | 0.00 |

Ts 6 Output Sequence: 5′-GCGGAUUUAmGCUCAGDDGGG-3′ (SEQ ID NO: 21)
D: dihydrouridine

TABLE S3-7
5′_biotin_tRNA_T1_SI_042519s07. Sequencing
of 5′-biotin-labeled tRNA segment I from 1G
to 18G by the global hierarchical ranking
algorithm. The output sequence is indicated
at the bottom.

| Fragment | Mass | RT | Base | Volume | PPM |
|---|---|---|---|---|---|
| 1 | 938.2184 | 21.449 | Tag + G | 403806 | 3.41 |
| 2 | 1243.2600 | 23.971 | C | 277726 | 2.33 |
| 3 | 1588.3060 | 25.493 | G | 238503 | 2.71 |
| 4 | 1933.3518 | 27.433 | G | 44902 | 3.05 |
| 5 | 2262.4042 | 29.682 | A | 35264 | 2.65 |
| 6 | 2568.4387 | 30.807 | U | 64428 | 1.25 |
| 7 | 2874.4631 | 31.835 | U | 219666 | 0.80 |
| 8 | 3180.4871 | 32.783 | U | 173234 | 0.31 |
| 9 | 3509.5467 | 34.465 | A | 67573 | 2.31 |
| 10 | 3868.6148 | 35.174 | mG | 226704 | 3.39 |
| 11 | 4173.6443 | 36.794 | C | 63409 | 0.31 |
| 12 | 4479.6520 | 37.559 | U | 12772 | 3.64 |
| 13 | 4784.7078 | 38.002 | C | 14478 | 0.38 |
| 14 | 5113.7758 | 38.479 | A | 69348 | 2.68 |
| 15 | 5458.8177 | 39.347 | G | 1588901 | 1.50 |
| 16 | 5766.8095 | 39.208 | D | 25595 | 7.11 |
| 17 | 6074.9000 | 39.440 | D | 118414 | 1.40 |
| 18 | 6419.9573 | 40.140 | G | 383672 | 2.87 |

Ts 7 Output Sequence: 5′-GCGGAUUUAmGCUCAGDDG-3′ (SEQ ID NO: 22)

TABLE S3-8
5′_biotin_tRNA_T1_SII_032919s07_44A45G.
Sequencing of 5′-biotin-labeled segment II
from 21A to 57G by the global hierarchical
ranking algorithm. The output sequence is
indicated at the bottom.

| Fragment | Mass | RT | Base | Volume | PPM |
|---|---|---|---|---|---|
| 1 | 922.2241 | 25.229 | Tag + A | 745215 | 3.04 |
| 2 | 1267.2710 | 25.756 | G | 577150 | 2.60 |
| 3 | 1596.3229 | 28.405 | A | 472089 | 2.44 |
| 4 | 1941.3702 | 29.167 | G | 591742 | 2.06 |
| 5 | 2246.4125 | 30.221 | C | 930358 | 1.34 |
| 6 | 2619.4912 | 35.055 | 2mG | 276858 | 1.15 |
| 7 | 2924.5312 | 35.109 | C | 937840 | 1.47 |
| 8 | 3229.5745 | 35.989 | C | 1389357 | 0.71 |
| 9 | 3558.6244 | 37.535 | A | 944505 | 1.38 |
| 10 | 3903.6768 | 38.016 | G | 1334405 | 0.03 |
| 11 | 4232.7261 | 39.120 | A | 899666 | 0.73 |
| 12 | 4857.8097 | 40.778 | U + Cm | 2369525 | 0.37 |
| 13 | 5545.9261 | 42.941 | A + Gm | 1777156 | 0.18 |
| 14 | 5874.9889 | 43.512 | A | 1527490 | 1.58 |
| 15 | 6086.9945 | 43.461 | Y′ | 2278504 | 1.03 |
| 16 | 6416.0477 | 44.268 | A | 1366254 | 1.09 |
| 17 | 6722.0827 | 44.327 | U | 1049995 | 2.48 |
| 18 | 7041.1313 | 44.591 | mC | 1297495 | 1.19 |
| 19 | 7347.1602 | 44.775 | U | 1560416 | 1.63 |
| 20 | 7692.2118 | 45.013 | G | 1319384 | 2.11 |
| 21 | 8037.2549 | 45.410 | G | 1009813 | 1.48 |
| 22 | 8366.3413 | 45.858 | A | 271843 | 5.47 |
| 23 | 8711.3823 | 45.865 | G | 1226283 | 4.52 |
| 24 | 9070.4677 | 45.822 | mG | 520562 | 6.80 |
| 25 | 9376.4389 | 45.871 | U | 416614 | 0.81 |
| 26 | 9681.5649 | 45.921 | C | 587268 | 9.54 |
| 27 | 10000.5521 | 46.069 | mC | 504658 | 2.27 |
| 28 | 10306.6258 | 46.099 | U | 925998 | 6.90 |
| 29 | 10651.5989 | 46.183 | G | 672326 | 0.31 |
| 30 | 10957.6318 | 46.200 | U | 320227 | 0.39 |
| 31 | 11302.6636 | 46.313 | G | 962623 | 1.00 |
| 32 | 11622.6493 | 46.492 | T | 325162 | 8.85 |
| 33 | 11928.6903 | 46.401 | U | 2182861 | 4.27 |
| 34 | 12233.7642 | 46.449 | C | 463444 | 1.50 |
| 35 | 12578.8603 | 46.548 | G | 2766678 | 0.47 |

Ts 8 Output Sequence: 5′-AGAGC2mGCCAGACmUGmAAY′AUmCUGGAGmGUCmCUGUGTUCG-3′ (SEQ ID NO 23)
Y′: a depurination product (ribose form) of the wybutosine (Y) at position 37.

TABLE S3-9
5′_biotin_tRNA_T1_SII_032919s07_44g45a.
Sequencing of 5′-biotin-labeled tRNA segment
II from 21A to 57G by the global hierarchical
ranking algorithm. The output sequence is
indicated at the bottom.

| Fragment | Mass | RT | Base | Volume | PPM |
|---|---|---|---|---|---|
| 1 | 922.2241 | 25.229 | Tag + A | 745215 | 3.04 |
| 2 | 1267.2710 | 25.756 | G | 577150 | 2.60 |
| 3 | 1596.3229 | 28.405 | A | 472089 | 2.44 |
| 4 | 1941.3702 | 29.167 | G | 591742 | 2.06 |
| 5 | 2246.4125 | 30.221 | C | 930358 | 1.34 |
| 6 | 2619.4912 | 35.055 | 2mG | 276858 | 1.15 |
| 7 | 2924.5312 | 35.109 | C | 937840 | 1.47 |
| 8 | 3229.5745 | 35.989 | C | 1389357 | 0.71 |
| 9 | 3558.6244 | 37.535 | A | 944505 | 1.38 |
| 10 | 3903.6768 | 38.016 | G | 1334405 | 0.03 |
| 11 | 4232.7261 | 39.120 | A | 899666 | 0.73 |
| 12 | 4857.8097 | 40.778 | U + Cm | 2369525 | 0.37 |
| 13 | 5545.9261 | 42.941 | A + Gm | 1777156 | 0.18 |
| 14 | 5874.9889 | 43.512 | A | 1527490 | 1.58 |
| 15 | 6086.9945 | 43.461 | Y′ | 2278504 | 1.03 |
| 16 | 6416.0477 | 44.268 | A | 1366254 | 1.09 |
| 17 | 6722.0827 | 44.327 | U | 1049995 | 2.48 |
| 18 | 7041.1313 | 44.591 | mC | 1297495 | 1.19 |
| 19 | 7347.1602 | 44.775 | U | 1560416 | 1.63 |
| 20 | 7692.2118 | 45.013 | G | 1319384 | 2.11 |
| 21 | 8037.2549 | 45.410 | G | 1009813 | 1.48 |
| 22 | 8382.2778 | 45.275 | G | 200964 | 1.49 |
| 23 | 8711.3823 | 45.865 | A | 1226283 | 4.51 |
| 24 | 9070.4677 | 45.822 | mG | 520562 | 6.80 |
| 25 | 9376.4389 | 45.871 | U | 416614 | 0.81 |
| 26 | 9681.5649 | 45.921 | C | 587268 | 9.54 |
| 27 | 10000.5521 | 46.069 | mC | 504658 | 2.27 |
| 28 | 10306.6258 | 46.099 | U | 925998 | 6.90 |
| 29 | 10651.5989 | 46.183 | G | 672326 | 0.31 |
| 30 | 10957.6318 | 46.200 | U | 320227 | 0.39 |
| 31 | 11302.6636 | 46.313 | G | 962623 | 1.00 |
| 32 | 11622.6493 | 46.492 | T | 325162 | 8.85 |
| 33 | 11928.6903 | 46.401 | U | 2182861 | 4.27 |
| 34 | 12233.7642 | 46.449 | C | 463444 | 1.50 |
| 35 | 12578.8603 | 46.548 | G | 2766678 | 0.47 |

Ts 9 Output Sequence: 5′-AGAGC2mGCCAGACmUGmAAY′AUmCUGGGAmGUCmCUGUGTUCG-3′ (SEQ ID NO: 24)

TABLE S3-10
3′_tRNA_100918s06. Sequencing of acid degraded
tRNA from 45G to 76A by the global hierarchical
ranking algorithm. The output sequence is
indicated at the bottom.

| Fragment | Mass | RT | Base | Volume | PPM |
|---|---|---|---|---|---|
| 1 | 877.1786 | 1.270 | A + C + C | 1022495 | 0.91 |
| 2 | 1206.2286 | 2.926 | A | 1172115 | 2.74 |
| 3 | 1511.2689 | 2.572 | C | 819385 | 2.85 |
| 4 | 1856.3153 | 3.218 | G | 1266301 | 2.86 |
| 5 | 2161.3551 | 3.798 | C | 1544446 | 3.15 |
| 6 | 2467.3789 | 4.806 | U | 2083726 | 3.36 |
| 7 | 2773.4042 | 6.085 | U | 3053673 | 2.99 |
| 8 | 3102.4553 | 7.075 | A | 5583907 | 3.13 |
| 9 | 3431.5054 | 7.910 | A | 2247902 | 3.53 |
| 10 | 3776.5516 | 7.745 | G | 5639286 | 3.52 |
| 11 | 4105.6016 | 8.447 | A | 2679354 | 3.85 |
| 12 | 4410.6408 | 8.523 | C | 4702025 | 4.06 |
| 13 | 4739.6917 | 9.123 | A | 2963739 | 4.11 |
| 14 | 5044.7319 | 9.175 | C | 2073512 | 4.08 |
| 15 | 5349.7949 | 9.288 | C | 1906782 | 0.21 |
| 16 | 5655.7967 | 9.545 | U | 914935 | 3.96 |
| 17 | 5998.8627 | 9.818 | mA | 2160204 | 4.08 |
| 18 | 6343.9049 | 9.900 | G | 2309111 | 4.68 |
| 19 | 6648.9464 | 9.893 | C | 3092250 | 4.45 |
| 20 | 6954.9754 | 9.838 | U | 1201050 | 3.72 |
| 21 | 7275.0127 | 10.396 | T | 2267279 | 4.07 |
| 22 | 7620.0765 | 10.498 | G | 1762814 | 1.73 |
| 23 | 7926.1455 | 10.423 | U | 1562423 | 3.85 |
| 24 | 8271.1067 | 10.603 | G | 1920966 | 6.73 |
| 25 | 8577.2011 | 10.660 | U | 1709835 | 1.56 |
| 26 | 8896.1598 | 11.550 | mC | 875226 | 9.53 |
| 27 | 9201.2581 | 11.313 | C | 769527 | 3.02 |
| 28 | 9507.2765 | 11.082 | U | 572956 | 3.65 |
| 29 | 9866.3028 | 11.030 | mG | 412887 | 7.25 |
| 30 | 10211.3522 | 11.073 | G | 709961 | 6.81 |

Ts 10 Output Sequence: 5′-GmGUCmCUGUGTUCGmAUCCACAGAAUUCGCACCA-3′ (SEQ ID NO: 25)

TABLE S3-11
5′_pG_tRNA_100918s06. Sequencing of 5′-pG tRNA
from 1G to 31A by the global hierarchical ranking
algorithm. The output sequence is indicated at
the bottom

| Fragment | Mass | RT | Base | Volume | PPM |
|---|---|---|---|---|---|
| 1 | 443.0274 | 0.931 | pG | 233231 | 7.00 |
| 2 | 748.0684 | 1.039 | C | 883929 | 3.74 |
| 3 | 1093.1105 | 1.800 | G | 2062278 | 2.29 |
| 4 | 1438.1575 | 3.239 | G | 3687690 | 2.02 |
| 5 | 1767.2087 | 4.484 | A | 4522172 | 2.38 |
| 6 | 2073.2354 | 5.369 | U | 8131266 | 1.35 |
| 7 | 2379.2590 | 6.043 | U | 8862830 | 1.89 |
| 8 | 2685.2836 | 6.593 | U | 9612100 | 1.94 |
| 9 | 3014.3343 | 7.355 | A | 6218090 | 2.32 |
| 10 | 3373.3964 | 8.120 | mG | 2974994 | 2.37 |
| 11 | 3678.4380 | 8.403 | C | 3957178 | 2.09 |
| 12 | 3984.4601 | 8.709 | U | 6419872 | 2.74 |
| 13 | 4289.5007 | 8.942 | C | 8348561 | 2.70 |
| 14 | 4618.5517 | 9.346 | A | 3797284 | 2.84 |
| 15 | 4963.6043 | 9.522 | G | 217686 | 1.59 |
| 16 | 5271.6374 | 9.631 | D | 3108073 | 3.00 |
| 17 | 5579.6773 | 9.748 | D | 3781679 | 3.03 |
| 18 | 5924.7327 | 9.944 | G | 689750 | 1.50 |
| 19 | 6269.7714 | 10.091 | G | 2753572 | 2.81 |
| 20 | 6614.8124 | 10.232 | G | 1506355 | 3.63 |
| 21 | 6943.8650 | 10.468 | A | 1708708 | 3.44 |
| 22 | 7288.9012 | 10.601 | G | 779104 | 4.82 |
| 23 | 7617.9417 | 10.826 | A | 852001 | 6.18 |
| 24 | 7963.0075 | 10.910 | G | 2445671 | 3.60 |
| 25 | 8268.0027 | 11.143 | C | 1087860 | 9.05 |
| 26 | 8641.1310 | 11.694 | 2mG | 207499 | 2.92 |
| 27 | 8946.1664 | 11.727 | C | 1364582 | 1.86 |
| 28 | 9251.2074 | 11.743 | C | 1059830 | 1.76 |
| 29 | 9580.2455 | 11.864 | A | 1450228 | 0.20 |
| 30 | 9925.3349 | 11.871 | G | 2494820 | 4.42 |
| 31 | 10254.2927 | 11.993 | A | 155606 | 4.95 |

Ts 11 Output Sequence: 5′-GCGGAUUUAmGCUCAGDDGGGAGAGC2mGCCAGA-3′ (SEQ ID NO: 26)

TABLE S3-12
Yield of CMC conversion occurring at pseudouridine measured by LC-MS.

| | | Calc mass | Exp mass | | EIC | | | |
|---|---|---|---|---|---|---|---|---|
| Conversion state | Fragment | (Da) | (Da) | m/z | ratio | QS | Yield | ppm |
| Non-converted | 21A to 44A | 7791.1320 | 7791.1787 | 778.1111 | 0.21 | 80 | 79% | −5.99 |
| CMC-converted | 21A to 44A | 8042.3318 | 8042.3492 | 803.2263 | 0.79 | 80 | | −2.16 |
| Non-converted | 57G to 47U | 3526.4344 | 3526.4333 | 586.7314 | 0.24 | 100 | 76% | 0.31 |
| CMC-converted | 57G to 47U | 3777.6342 | 3777.6332 | 628.5979 | 0.76 | 100 | | 0.26 |

TABLE S3-13
5′_tRNA_T1_nonCMC_SII_042519s04_44A45G.
Sequencing of 5′-non-CMC-converted tRNA
segment II from 21A to 45G by the global
hierarchical ranking algorithm. The output
sequence is indicated at the bottom.

| Fragment | Mass | RT | Base | Volume | PPM |
|---|---|---|---|---|---|
| 1 | 692.1076 | 1.032 | A + | 121835 | 4.19 |
| | | | G | | |
| 2 | 1021.1576 | 1.264 | A | 548483 | 5.29 |
| 3 | 1366.2072 | 4.020 | G | 2219430 | 2.34 |
| 4 | 1671.2480 | 7.304 | C | 3142702 | 2.21 |
| 5 | 2044.3269 | 16.800 | 2mG | 1700693 | 1.71 |
| 6 | 2349.3689 | 18.430 | C | 2431764 | 1.19 |
| 7 | 2654.4105 | 20.727 | C | 6691067 | 0.94 |
| 8 | 2983.4639 | 23.756 | A | 9276684 | 0.54 |
| 9 | 3328.5120 | 25.192 | G | 10673175 | 0.27 |
| 10 | 3657.5668 | 27.417 | A | 5126136 | 0.38 |
| 11 | 4282.6486 | 30.874 | U + | 15880661 | 0.21 |
| | | | Cm | | |
| 12 | 4970.7665 | 34.609 | A + | 10873309 | 0.64 |
| | | | Gm | | |
| 13 | 5299.8210 | 35.684 | A | 12807606 | 1.02 |
| 14 | 5511.8306 | 35.900 | Y′ | 13088146 | 1.16 |
| 15 | 5840.8850 | 37.167 | A | 3623732 | 3.32 |
| 16 | 6146.9096 | 37.460 | U | 1897334 | 3.04 |
| 17 | 6465.9704 | 38.006 | mC | 2463925 | 1.78 |
| 18 | 6771.9928 | 38.393 | U | 3706693 | 1.26 |
| 19 | 7117.0453 | 38.873 | G | 3506106 | 3.47 |
| 20 | 7462.0964 | 39.527 | G | 2455794 | 3.81 |
| 21 | 7791.1787 | 40.196 | A | 1226259 | 7.47 |
| 22 | 8136.1916 | 40.385 | G | 1925167 | 2.91 |

Ts 13 Output Sequence: 5′-AGAGC2mGCCAGACmUGmAAY′AUmCUGGAG-3′ (SEQ ID NO: 27)

TABLE S3-14
5′_tRNA_T1_nonCMC_SII_042519s04_44g45a. Sequencing
of 5′-non-CMC-converted tRNA segment II from 21A
to 45A by the global hierarchical ranking
algorithm. The output sequence is indicated at
the bottom

| Fragment | Mass | RT | Base | Volume | PPM |
|---|---|---|---|---|---|
| 1 | 692.1076 | 1.032 | A + G | 121835 | 4.19 |
| 2 | 1021.1576 | 1.264 | A | 548483 | 5.29 |
| 3 | 1366.2072 | 4.020 | G | 2219430 | 2.34 |
| 4 | 1671.2480 | 7.304 | C | 3142702 | 2.21 |
| 5 | 2044.3269 | 16.800 | 2mG | 1700693 | 1.71 |
| 6 | 2349.3689 | 18.430 | C | 2431764 | 1.19 |
| 7 | 2654.4105 | 20.727 | C | 6691067 | 0.94 |
| 8 | 2983.4639 | 23.756 | A | 9276684 | 0.54 |
| 9 | 3328.5120 | 25.192 | G | 10673175 | 0.27 |
| 10 | 3657.5668 | 27.417 | A | 5126136 | 0.38 |
| 11 | 4282.6486 | 30.874 | U + Cm | 15880661 | 0.21 |
| 12 | 4970.7665 | 34.609 | A + Gm | 10873309 | 0.64 |
| 13 | 5299.8210 | 35.684 | A | 12807606 | 1.02 |
| 14 | 5511.8306 | 35.900 | Y′ | 13088146 | 1.16 |
| 15 | 5840.8850 | 37.167 | A | 3623732 | 3.32 |
| 16 | 6146.9096 | 37.460 | U | 1897334 | 3.04 |
| 17 | 6465.9704 | 38.006 | mC | 2463925 | 1.78 |
| 18 | 6771.9928 | 38.393 | U | 3706693 | 1.26 |
| 19 | 7117.0453 | 38.873 | G | 3506106 | 3.47 |
| 20 | 7462.0964 | 39.527 | G | 2455794 | 3.81 |
| 21 | 7807.1385 | 39.523 | G | 835117 | 1.52 |
| 22 | 8136.1916 | 40.385 | A | 1925167 | 1.54 |

Ts 14 Output Sequence: 5′-AGAGC2mGCCAGACmUGmAAY′AUmCUGGGA-3′ (SEQ ID NO: 28)

TABLE S3-15
5′_tRNA_T1_CMC_SII_042519s04. Sequencing of
5′-CMC-converted tRNA segment II from 39ψ to
44A by the global hierarchical ranking algorithm.
The output sequence is indicated at the bottom.

| Fragment | Mass | RT | Base | Volume | PPM |
|---|---|---|---|---|---|
| 1 | 6398.1211 | 44.707 | Mod-Psi | 1295323 | 2.97 |
| 2 | 6717.1789 | 45.223 | mC | 2506731 | 2.96 |
| 3 | 7023.1878 | 45.283 | U | 3037253 | 0.50 |
| 4 | 7368.2361 | 45.446 | G | 8115206 | 0.58 |
| 5 | 7713.3006 | 45.574 | G | 4221938 | 2.77 |
| 6 | 8042.3492 | 46.255 | A | 3190026 | 2.18 |

Ts 15 Output Sequence: 5′-ψmCUGGA-3′
Mod-Psi is a symbol used in the global hierarchical ranking algorithm to designate pseudouridine (ψ).

TABLE S3-16
3′_tRNA_T1_nonCMC_SII_042519s04. Sequencing
of 3′-non-CMC-converted tRNA segment II from
57G to 47U by the global hierarchical ranking
algorithm. The output sequence is indicated at
the bottom

| Fragment | Mass | RT | Base | Volume | PPM |
|---|---|---|---|---|---|
| 1 | 668.0943 | 0.968 | G + C | 79549 | 7.33 |
| 2 | 974.1302 | 0.915 | U | 826458 | 5.85 |
| 3 | 1294.1594 | 2.732 | T | 403523 | 4.71 |
| 4 | 1639.2089 | 6.500 | G | 789168 | 2.44 |
| 5 | 1945.2357 | 6.129 | U | 190380 | 1.29 |
| 6 | 2290.2818 | 10.466 | G | 1584520 | 1.66 |
| 7 | 2596.3069 | 12.965 | U | 1100858 | 1.54 |
| 8 | 2915.3646 | 17.907 | mC | 1557574 | 1.10 |
| 9 | 3220.4052 | 18.523 | C | 773618 | 1.21 |
| 10 | 3526.4333 | 20.318 | U | 2252901 | 0.31 |

Ts 16 Output Sequence: 5′-UCmCUGUGTUCG-3′ (SEQ ID NO: 29)

TABLE S3-17
3′_tRNA_T1_CMC_SII_042519s04. Sequencing of
3′-CMC converted tRNA segment II from 57G to
47U by the global hierarchical ranking algorithm.
The output sequence is indicated at the bottom

| Fragment | Mass | RT | Base | Volume | PPM |
|---|---|---|---|---|---|
| 1 | 1225.3215 | 14.484 | Mod-Psi | 882395 | 2.29 |
| 2 | 1545.3611 | 19.764 | T | 78086 | 2.72 |
| 3 | 1890.4097 | 27.200 | G | 1324986 | 1.59 |
| 4 | 2196.4340 | 25.561 | U | 33874 | 1.82 |
| 5 | 2541.4824 | 27.899 | G | 3029272 | 1.18 |
| 6 | 2847.5087 | 28.729 | U | 2275337 | 0.70 |
| 7 | 3166.5661 | 32.358 | mC | 2499558 | 0.47 |
| 8 | 3471.6055 | 32.073 | C | 2485944 | 1.01 |
| 9 | 3777.6332 | 32.777 | U | 4553148 | 0.29 |

Ts 17 Output Sequence: 5′-UCmCUGUGTψ-3′

TABLE S3-18
Detection of Y′ in the presence of tRNA before (in full-
length tRNA) and after (as an isolated base) acid degradation.

| | Calc mass | Exp mass | | EIC | | | |
|---|---|---|---|---|---|---|---|
| In a form of segment II | (Da) | (Da) | m/z | ratio | Percent | QS | ppm |
| Y before acid degradation | 12361.805 | 12361.841 | 823.1141 | 0.90 | 90% | 80 | −2.9 |
| Y′ before acid degradation | 12003.666 | 12003.762 | 922.359 | 0.10 | 10% | 48 | −7.9 |
| Y′ after acid degradation | 376.1495 | 376.1479 | 375.1409 | 1.0 | 100% | 100 | 4.3 |

TABLE S3-19
The relative percentages of 11 modifications at each position were quantified
by integrating the EIC peaks of their corresponding ladder fragments from tRNA.

| Position | Modification | Fragment | Formula | Mass (Da) | m/z | EIC | Percent |
|---|---|---|---|---|---|---|---|
| 10 | m²G | 1 G-10 m²G | C97H122N39O75P11 | 3373.4045 | 673.6734 | 1.0 | 100% |
| | G | 1 G-10 G | C96H120N39O75P11 | 3359.3889 | — | — | — |
| 16 | D | 1 G-16 D | C153H194N59O118P17 | 5271.6533 | 657.9480 | 1.0 | 100% |
| | U | 1 G-16 U | C153H192N59O118P17 | 5269.6376 | — | — | — |
| 17 | D | 1 G-17 D | C162H207N61O126P18 | 5579.6942 | 696.4556 | 1.0 | 100% |
| | U | 1 G-17 U | C162H205N61O126P18 | 5577.6786 | — | — | — |
| 26 | m₂²G | 21 A-26 m₂²G 57 G | C61H78N28O41P6 | 2044.3305 | 680.4350 | 0.58 | 58% |
| | mG | 21 A-26 mG | C60H76N28O41P6 | 2030.3148 | — | — | — |
| | G | 21 A-26 G | C59H74N28O41P6 | 2016.2992 | 503.0664 | 0.42 | 42% |
| 32 | Cm | 21 A-32 Cm+U | C128H163N54O89P13 | 4282.6478 | 610.7992 | 1.0 | 100% |
| | C | 21 A-32 C | C118H150N52O81P12P1 [symbol] | 3962.6068 | — | — | — |
| 34 | Gm | 21 A-34 Gm+A | C149H189N64O102P15 | 4970.7634 | 709.1020 | 0.60 | 60% |
| | G | 21 A-34 G | C138H175N59O96P14 | 4627.6952 | 660.0906 | 0.40 | 40% |
| 37 | Y | 21 A-57 G | C373H469N146O264P37 | 12361.8050 | 823.1141 | 0.90 | 90% |
| | Y′ | 21 A-57 G | C357H451N140O260P37 | 12003.6660 | 922.359 | 0.10 | 10% |
| 39 | CMC-converted | 21 A-44 A | C246H319N99O165P24 | 8042.3318 | 803.2263 | 0.79 | 79% |
| | [symbol] | | | | | | |
| | Non-converted | 21 A-44 A | C232H294N96O164P24 | 7791.1320 | 778.1111 | 0.21 | 21% |
| | [symbol] | | | | | | |
| | Calibrated | 21 A-44 A | C232H294N96O164P24 | 7791.1320 | — | — | 100%* |
| | Ψ | | | | | | |
| 40 | m⁵C | 21 A-40 m⁵C | C193H247N79O136P20 | 6465.9592 | 717.4318 | 1.0 | 100% |
| | C | 21A-40 C | C192H245N79O136P20 | 6451.9436 | — | — | — |
| 46 | m⁷G | 21 A-46 m⁷G | C253H320N106O178P26 | 8495.2425 | 771.2866 | 0.46 | 46% |
| | G | 21 A-46 G | C252H318N106O178P26 | 8481.2268 | 770.0160 | 0.54 | 54% |
| 49 | m⁵C | 21 A-49 m5C | C281H357N114O200P29 | 9425.3660 | 784.5296 | 1.0 | 100% |
| | C | 21 A-49 C | C280H355N114O200P29 | 9411.3503 | — | — | — |
| 54 | T | 21 A-54 T | C329H416N130O238P34 | 11047.5524 | 848.8105 | 1.0 | 100% |
| | U | 21 A-54 U | C328H414N130O238P34 | 11033.5368 | — | — | — |
| 55 | CMC-converted | 47 U-57 G | C118H158N37O84P11 | 3777.6342 | 628.5979 | 0.76 | 76% |
| | [symbol] | | | | | | |
| | Non-converted | 47 U-57 G | C104H133N34O83P11 | 3526.4344 | 586.7314 | 0.24 | 24% |
| | Calibrated | 47 U-57 G | C104H133N34O83P11 | 3526.4344 | — | — | 100%* |
| | Ψ | | | | | | |
| 58 | m¹A | 58 m¹A-75 C | C203H270N73O138P19S | 6558.1089 | 727.6724 | 0.94 | 94% |
| | | | C203H270N73O138P19S | | | | |
| | A | 58 A-75 C | C202H268N73O138P19S | 6544.0933 | 722.7936 | 0.06 | 6% |

*Please note:
Integration of the EIC peak of CMC-Ψ-containing ladder fragment was used for the percentage quantification, but when we factored in the yield of the conversion of the Ψ to the CMC-Ψ (~70%), this position would be ~100% of Ψ. Parts highlighted in pink are related to partially modified nucleotides. [symbol]
indicates data missing or illegible when filed

TABLE S3-20
3′_OH_tRNA_T1_SII_111418s05_44A45G. LC-MS analysis of segment II from
34Gm to 55ψ. Below are all sequence ladder components when reading
from 3′- to 5′-direction. The sequence was manually verified and
is displayed at the bottom.

| | | | | Extracted data file | | | | |
|---|---|---|---|---|---|---|---|---|
| Theoretical | | | | after LC/MS analysis | | | | |
| | Theoretical | Base | | | | | Quality | Error |
| Fragments | mass | mass | Base | MFE mass | t_{R} | Volume | Score | ppm |
| 21 | 7739.0291 | 688.1156 | A + Gm | 7739.0198 | 28.919 | 572629 | 80 | 1.20 |
| 20 | 7050.9135 | 329.0525 | A | 7050.9277 | 26.539 | 413840 | 60 | −2.01 |
| 19 | 6721.8610 | 212.0086 | Y′ | 6721.8635 | 24.741 | 381223 | 72.8 | −0.37 |
| 18 | 6509.8524 | 329.0525 | A | 6509.8604 | 25.336 | 1019699 | 80 | −1.23 |
| 17 | 6180.7999 | 306.0253 | y | 6180.8037 | 23.079 | 707995 | 77.8 | −0.61 |
| 16 | 5874.7746 | 319.0570 | m⁵C | 5874.7783 | 23.641 | 2167527 | 100 | −0.63 |
| 15 | 5555.7176 | 306.0253 | U | 5555.7209 | 21.539 | 1146864 | 98.5 | −0.59 |
| 14 | 5249.6923 | 345.0474 | G | 5249.6958 | 20.605 | 1609784 | 100 | −0.67 |
| 13 | 4904.6449 | 345.0475 | G | 4904.6446 | 19.764 | 1791176 | 100 | 0.06 |
| 12 | 4559.5974 | 329.0525 | A | 4559.5918 | 19.341 | 974223 | 80 | 1.23 |
| 11 | 4230.5449 | 345.0474 | G | 4230.5449 | 16.828 | 1254040 | 99.7 | 0.00 |
| 10 | 3885.4975 | 359.0631 | m⁷G | 3885.4957 | 15.319 | 1940572 | 95.7 | 0.46 |
| 9 | 3526.4344 | 306.0253 | U | 3526.4327 | 13.475 | 1011995 | 100 | 0.48 |
| 8 | 3220.4091 | 305.0413 | C | 3220.4066 | 11.393 | 2082145 | 100 | 0.78 |
| 7 | 2915.3678 | 319.0569 | m⁵C | 2915.3648 | 10.586 | 3108932 | 100 | 1.03 |
| 6 | 2596.3109 | 306.0253 | U | 2596.3066 | 6.488 | 523377 | 42.8 | 1.66 |
| 5 | 2290.2856 | 345.0475 | G | 2290.2828 | 3.961 | 2464626 | 94.7 | 1.22 |
| 4 | 1945.2381 | 306.0253 | U | 1945.2379 | 1.074 | 637786 | 83.4 | 0.10 |
| 3 | 1639.2128 | 345.0474 | G | 1639.2106 | 1.034 | 2301078 | 100 | 1.34 |
| 2 | 1294.1654 | 320.0409 | T | 1294.1737 | 8.127 | 78112 | 67.5 | −6.41 |
| 1 | 974.1245 | 306.0253 | ψ | 974.1240 | 0.936 | 143886 | 79.1 | 0.51 |

Ts 20 Output Sequence: 5′-GmAAY′AψmCUGGAGmGUCmCUGUGTψ-3′ (SEQ ID NO: 30)

TABLE S3-21
3′_OH_tRNA_T1_SII_111418s05_44g45a. LC-MS analysis of segment II from
34Gm to 55ψ. Below are all sequence ladder components when reading from
3′- to 5′-direction. The sequence was manually verified and is
displayed at the bottom.

| | | | | Extracted data file | | | | |
|---|---|---|---|---|---|---|---|---|
| Theoretical | | | | after LC/MS analysis | | | | |
| | Theoretical | Base | | | | | Quality | Error |
| Fragments | mass | mass | Base | MFE mass | t_{R} | Volume | Score | ppm |
| 21 | 7739.0291 | 688.1156 | A + Gm | 7739.0198 | 28.919 | 572629 | 80 | 1.20 |
| 20 | 7050.9135 | 329.0525 | A | 7050.9277 | 26.539 | 413840 | 60 | −2.01 |
| 19 | 6721.8610 | 212.0086 | Y′ | 6721.8635 | 24.741 | 381223 | 72.8 | −0.37 |
| 18 | 6509.8524 | 329.0525 | A | 6509.8604 | 25.336 | 1019699 | 80 | −1.23 |
| 17 | 6180.7999 | 306.0253 | ψ | 6180.8037 | 23.079 | 707995 | 77.8 | −0.61 |
| 16 | 5874.7746 | 319.0570 | m⁵C | 5874.7783 | 23.641 | 2167527 | 100 | −0.63 |
| 15 | 5555.7176 | 306.0253 | U | 5555.7209 | 21.539 | 1146864 | 98.5 | −0.59 |
| 14 | 5249.6923 | 345.0474 | G | 5249.6958 | 20.605 | 1609784 | 100 | −0.67 |
| 13 | 4904.6449 | 345.0475 | G | 4904.6446 | 19.764 | 1791176 | 100 | 0.06 |
| 12 | 4559.5974 | 345.0474 | G | 4559.5918 | 19.341 | 974223 | 80 | −2.94 |
| 11 | 4214.5500 | 329.0525 | A | 4214.5624 | 18.424 | 273170 | 79.6 | 0.46 |
| 10 | 3885.4975 | 359.0631 | m⁷G | 3885.4957 | 15.319 | 1940572 | 95.7 | 0.46 |
| 9 | 3526.4344 | 306.0253 | U | 3526.4327 | 13.475 | 1011995 | 100 | 0.48 |
| 8 | 3220.4091 | 305.0413 | C | 3220.4066 | 11.393 | 2082145 | 100 | 0.78 |
| 7 | 2915.3678 | 319.0569 | m⁵C | 2915.3648 | 10.586 | 3108932 | 100 | 1.03 |
| 6 | 2596.3109 | 306.0253 | U | 2596.3066 | 6.488 | 523377 | 42.8 | 1.66 |
| 5 | 2290.2856 | 345.0475 | G | 2290.2828 | 3.961 | 2464626 | 94.7 | 1.22 |
| 4 | 1945.2381 | 306.0253 | U | 1945.2379 | 1.074 | 637786 | 83.4 | 0.10 |
| 3 | 1639.2128 | 345.0474 | G | 1639.2106 | 1.034 | 2301078 | 100 | 1.34 |
| 2 | 1294.1654 | 320.0409 | T | 1294.1737 | 8.127 | 78112 | 67.5 | −6.41 |
| 1 | 974.1245 | 306.0253 | ψ | 974.1240 | 0.936 | 143886 | 79.1 | 0.51 |

Ts 21 Output Sequence: 5′-GmAAY′AψmCUGGGAmGUCmCUGUGTψ-3′ (SEQ ID NO: 31)

TABLE S3-22
3′_OH_tRNA_T1_SII_032919s07_44A45G. LC-MS analysis of segment II
from 30G to 55ψ. Below are all sequence ladder components when
reading from 3′- to 5′-direction. The sequence was manually
verified and is displayed at the bottom.

| | | | | Extracted data file | | | | |
|---|---|---|---|---|---|---|---|---|
| Theoretical | | | | after LC/MS analysis | | | | |
| | Theoretical | Base | | | | | Quality | Error |
| Fragments | mass | mass | Base | MFE mass | t_{R} | Volume | Score | ppm |
| 24 | 9038.2113 | 345.0474 | G | 9038.133 | 37.926 | 394860 | 60.8 | 8.66 |
| 23 | 8693.1639 | 329.0525 | A | 8693.1871 | 38.113 | 174673 | 41.4 | −2.67 |
| 22 | 8364.1114 | 625.0823 | U + Cm | 8364.1502 | 37.005 | 133633 | 41.9 | −4.64 |
| 21 | 7739.0291 | 688.1156 | A + Gm | 7739.0557 | 35.391 | 650792 | 77.4 | −3.44 |
| 20 | 7050.9135 | 329.0525 | A | 7050.9339 | 32.627 | 590137 | 78.5 | −2.89 |
| 19 | 6721.8610 | 212.0086 | Y' | 6721.8845 | 30.813 | 764391 | 80 | −3.50 |
| 18 | 6509.8524 | 329.0525 | A | 6509.864 | 31.762 | 1166876 | 80 | −1.78 |
| 17 | 6180.7999 | 306.0253 | ψ | 6180.7968 | 29.159 | 148437 | 65.9 | 0.50 |
| 16 | 5874.7746 | 319.0570 | m⁵C | 5874.7784 | 30.31 | 1368105 | 79.9 | −0.65 |
| 15 | 5555.7176 | 306.0253 | U | 5555.7219 | 27.737 | 1148576 | 80 | −0.77 |
| 14 | 5249.6923 | 345.0474 | G | 5249.7098 | 26.957 | 1297236 | 80 | −3.33 |
| 13 | 4904.6449 | 345.0475 | G | 4904.6497 | 26.195 | 1021939 | 90 | −0.98 |
| 12 | 4559.5974 | 329.0525 | A | 4559.5974 | 25.942 | 1209559 | 99 | 0.00 |
| 11 | 4230.5449 | 345.0474 | G | 4230.5461 | 23.338 | 927818 | 92.3 | −0.28 |
| 10 | 3885.4975 | 359.0631 | m⁷G | 3885.4975 | 21.811 | 1357508 | 90.5 | 0.00 |
| 9 | 3526.4344 | 306.0253 | U | 3526.4332 | 20.034 | 1078413 | 98.3 | 0.34 |
| 8 | 3220.4091 | 305.0413 | C | 3220.4063 | 18.209 | 1434999 | 100 | 0.87 |
| 7 | 2915.3678 | 319.0569 | m⁵C | 2915.366 | 17.589 | 2388681 | 100 | 0.62 |
| 6 | 2596.3109 | 306.0253 | U | 2596.308 | 12.655 | 1592241 | 100 | 1.12 |
| 5 | 2290.2856 | 345.0475 | G | 2290.2828 | 10.189 | 2053112 | 100 | 1.22 |
| 4 | 1945.2381 | 306.0253 | U | 1945.2371 | 6.47 | 1359480 | 77.8 | 0.51 |
| 3 | 1639.2128 | 345.0474 | G | 1639.21 | 4.723 | 1598482 | 100 | 1.71 |
| 2 | 1294.1654 | 320.0409 | T | 1294.1615 | 2.282 | 620026 | 100 | 3.01 |
| 1 | 974.1245 | 306.0253 | ψ | 974.1225 | 0.875 | 221837 | 90.6 | 2.05 |

Ts 22 Output Sequence: 5′-GACmUGmAAY′AUmCUGGAGmGUCmCUGUGTU-3′ (SEQ ID NO: 32)

TABLE S3-23
3′_OH_tRNA_T1_SII_032919s07_44g45a. LC-MS analysis of segment II from
30G to 55ψ. Below are all sequence ladder components when reading from
3′- to 5′-direction. The sequence was manually verified and is displayed
at the bottom.

| | | | | Extracted data file | | | | |
|---|---|---|---|---|---|---|---|---|
| Theoretical | | | | after LC/MS analysis | | | | |
| | Theoretical | Base | | | | | Quality | Error |
| Fragments | mass | mass | Base | MFE mass | t_{R} | Volume | Score | ppm |
| 24 | 9038.2113 | 345.0474 | G | 9038.133 | 37.926 | 394860 | 60.8 | 8.66 |
| 23 | 8693.1639 | 329.0525 | A | 8693.1871 | 38.113 | 174673 | 41.4 | −2.67 |
| 22 | 8364.1114 | 625.0823 | U + Cm | 8364.1502 | 37.005 | 133633 | 41.9 | −4.64 |
| 21 | 7739.0291 | 688.1156 | A + Gm | 7739.0557 | 35.391 | 650792 | 77.4 | −3.44 |
| 20 | 7050.9135 | 329.0525 | A | 7050.9339 | 32.627 | 590137 | 78.5 | −2.89 |
| 19 | 6721.8610 | 212.0086 | Y′ | 6721.8845 | 30.813 | 764391 | 80 | −3.50 |
| 18 | 6509.8524 | 329.0525 | A | 6509.864 | 31.762 | 1166876 | 80 | −1.78 |
| 17 | 6180.7999 | 306.0253 | ψ | 6180.7968 | 29.159 | 148437 | 65.9 | 0.50 |
| 16 | 5874.7746 | 319.0570 | m⁵C | 5874.7784 | 30.31 | 1368105 | 79.9 | −0.65 |
| 15 | 5555.7176 | 306.0253 | U | 5555.7219 | 27.737 | 1148576 | 80 | −0.77 |
| 14 | 5249.6923 | 345.0474 | G | 5249.7098 | 26.957 | 1297236 | 80 | −3.33 |
| 13 | 4904.6449 | 345.0475 | G | 4904.6497 | 26.195 | 1021939 | 90 | −0.98 |
| 12 | 4559.5974 | 345.0474 | G | 4559.5974 | 25.942 | 1209559 | 99 | 0.00 |
| 11 | 4214.5500 | 329.0525 | A | 4214.5534 | 24.918 | 299777 | 60 | −0.81 |
| 10 | 3885.4975 | 359.0631 | m⁷G | 3885.4975 | 21.811 | 1357508 | 90.5 | 0.00 |
| 9 | 3526.4344 | 306.0253 | U | 3526.4332 | 20.034 | 1078413 | 98.3 | 0.34 |
| 8 | 3220.4091 | 305.0413 | C | 3220.4063 | 18.209 | 1434999 | 100 | 0.87 |
| 7 | 2915.3678 | 319.0569 | m⁵C | 2915.366 | 17.589 | 2388681 | 100 | 0.62 |
| 6 | 2596.3109 | 306.0253 | U | 2596.308 | 12.655 | 1592241 | 100 | 1.12 |
| 5 | 2290.2856 | 345.0475 | G | 2290.2828 | 10.189 | 2053112 | 100 | 1.22 |
| 4 | 1945.2381 | 306.0253 | U | 1945.2371 | 6.47 | 1359480 | 77.8 | 0.51 |
| 3 | 1639.2128 | 345.0474 | G | 1639.21 | 4.723 | 1598482 | 100 | 1.71 |
| 2 | 1294.1654 | 320.0409 | T | 1294.1615 | 2.282 | 620026 | 100 | 3.01 |
| 1 | 974.1245 | 306.0253 | ψ | 974.1225 | 0.875 | 221837 | 90.6 | 2.05 |

Ts 23 Output Sequence: 5′-GACmUGmAAY′AUmCUGGGAmGUCmCUGUGTU-3′ (SEQ ID NO: 33)

TABLE S3-24
Quantification of the relative population of the three isoforms of tRNA
based on integration of EIC of RNase T1 digested products of tRNA.⁸

| | Calc mass | Exp mass | | EIC | | | |
|---|---|---|---|---|---|---|---|
| Fragment | (Da) | (Da) | m/z | ratio | Percent | QS | ppm |
| 58m¹A to 74C | 5364.7935 | 5364.7939 | 595.0800 | 0.03 | 3% | 98 | −0.1 |
| 58m¹A to 75C | 5669.8348 | 5669.8403 | 628.9753 | 0.80 | 80% | 100 | −1.0 |
| 58m¹A to 76A | 5998.8873 | 5998.8845 | 598.8828 | 0.17 | 17% | 100 | 0.5 |

TABLE S3-25
Detection of wild type (44A45G) and transition/edited form (44g45a) tRNA,
respectively, in three datasets by the global hierarchical ranking algorithm
(refer to output files in Tables S4, S5, S8, S9, S13, and S14).

| | Wild type (I) | | Transition form (II) | | | I | | II |
|---|---|---|---|---|---|---|---|---|
| | | EIC ratio | | EIC ratio | I | Mean ± | II | Mean ± |
| Dataset | m/z | (44A) | m/z | (44g) | % | SEM | % | SEM |
| Labeled | 836.1243 | 0.54 | 837.6269 | 0.46 | 54 | | 46 | |
| segment II | | | | | | | | |
| Unlabeled | 778.4074 | 0.44 | 780.0080 | 0.56 | 44 | 50.4 ± 3.2% | 56 | 49.6 ± 3.2% |
| segment II | | | | | | | | |
| Non-CMC- | 778.4077 | 0.53 | 779.7066 | 0.47 | 53 | | 47 | |
| converted | | | | | | | | |
| segment II | | | | | | | | |

*Form I: 44A45G;
Form II: 44g45a
Form I % = EIC (44A)/[EIC (44A) + EIC (44g)];
Form II % = EIC (44g)/[EIC (44A) + EIC (44g)]

TABLE S4-1
A list of all the masses from the deconvoluted mass spectrum of yeast tRNA-Phe and the homology search result based on the masses:

| Monoisotopic | Average | Sum | Start | Stop | Apex | | | | |
|---|---|---|---|---|---|---|---|---|---|
| Mass | Mass | Intensity | Time (n | Time | RT | | Comments | Comments2 | Possible tRNA |
| 24851.687 | 24863.29 | 1.79E+04 | 4.567 | 4.780 | 4.665 | | unknownG | | |
| 24835.675 | 24847.27 | 1.29E+04 | 4.583 | 4.780 | 4.665 | | unknownG+A-G | | |
| 25180.751 | 25192.51 | 3.13E+03 | 4.583 | 4.731 | 4.665 | | unknownG+A | | |
| 24899.658 | 24911.29 | 4.45E+03 | 4.468 | 4.665 | 4.583 | | unknown F+p-1 | | |
| 24881.655 | 24893.27 | 1.90E+04 | 4.468 | 4.714 | 4.5665 | | unknown F+p-1-18.003 | | |
| 24820.694 | 24832.29 | 3.21E+05 | 4.337 | 4.829 | 4.5337 | | unknown F | | |
| 25149.725 | 25161.47 | 1.63E+05 | 4.337 | 4.780 | 4.5337 | | unknownF+A | | |
| 24836.682 | 24848.28 | 5.54E+04 | 4.403 | 4.665 | 4.5337 | | unknownF+O? | | |
| 25165.737 | 25177.49 | 2.76E+04 | 4.403 | 4.665 | 4.5337 | | unknownF+G | | |
| 24805.639 | 24817.22 | 9.77E+03 | 4.468 | 4.616 | 4.5337 | | unknownF-15 | | |
| 25181.749 | 25193.51 | 1.39E+04 | 4.403 | 4.665 | 4.5173 | | unknownF+G+16.012 | unknownG+A+1H | |
| 24852.700 | 24864.31 | 5.37E+04 | 4.353 | 4.632 | 4.4845 | −329.049 | unknownF+G+16.012-A | | |
| 24806.662 | 24818.25 | 1.44E+04 | 4.353 | 4.534 | 4.4353 | | unknownF-15-1 | | |
| 27710.822 | 27723.77 | 1.67E+04 | 4.211 | 4.403 | 4.3382 | | | | |
| 27650.910 | 27663.83 | 2.34E+05 | 4.178 | 4.435 | 4.3058 | | unknown K | | Leu2-C-p+C5H8+2H? |
| 27635.895 | 27648.81 | 5.02E+04 | 4.211 | 4.403 | 4.3058 | | unknownK-NH | | |
| 28363.921 | 28377.18 | 6.50E+03 | 4.178 | 4.337 | 4.2575 | | unknown K+A+C+p+1 | A to i6A? | Leu2-CCA+C5H8+1? |
| 27672.819 | 27685.75 | 4.77E+04 | 4.088 | 4.227 | 4.1606 | | | | |
| 25334.618 | 25346.46 | 4.59E+06 | 3.961 | 4.211 | 4.0865 | | | | Tyr-CCA |
| 25006.486 | 25018.16 | 1.29E+06 | 3.990 | 4.178 | 4.0598 | | | | Tyr-CC +1 |
| 24792.469 | 24804.05 | 7.96E+05 | 3.931 | 4.135 | 4.0388 | | | | Thr-CCA+2H |
| 24610.491 | 24621.99 | 1.63E+08 | 3.856 | 5.991 | 4.0092 | | 75mer | | Phe-CC |
| 24939.549 | 24951.19 | 9.69E+07 | 3.889 | 5.630 | 4.0092 | | 76mer | | Phe=CCA |
| 24639.459 | 24650.97 | 1.21E+06 | 3.931 | 4.088 | 4.0092 | | 28.98 | | |
| 24461.430 | 24472.86 | 6.51E+05 | 3.931 | 4.135 | 4.0092 | | | | Thr-CC |
| 24995.505 | 25007.18 | 6.50E+05 | 3.889 | 4.163 | 3.9599 | | unknownD+A=1H | −272.20 | |
| 24667.377 | 24678.90 | 9.52E+05 | 3.835 | 3.990 | 3.9393 | | unknownD | 56.047 | lle-C+rA |
| 24723.424 | 24734.97 | 1.79E+05 | 3.758 | 3.961 | 3.8619 | | | | lle-CC |
| 24650.419 | 24661.93 | 1.41E+05 | 3.758 | 3.931 | 3.8619 | | unknownD+A-1H-G | | |
| 24251.368 | 24262.70 | 1.24E+05 | 3.758 | 3.931 | 3.8619 | | | Acid-Degraded | Phe-CC |
| 24581.434 | 24592.92 | 8.78E+04 | 3.789 | 3.931 | 3.8619 | | | Acid Degraded | Phe-CCA |
| 23847.169 | 23858.31 | 3.27E+05 | 3.709 | 3.931 | 3.8391 | | unknown C | | |
| 24459.389 | 24470.82 | 5.22E+04 | 3.758 | 3.911 | 3.8391 | | | | Thr-CC-2H |
| 24287.329 | 24298.68 | 4.80E+04 | 3.742 | 3.911 | 3.8391 | | | | lle-C-Gu+2H |
| 23862.175 | 23873.33 | 3.60E+04 | 3.742 | 3.889 | 3.8072 | | unknown C+15 | | |

TABLE S4-2
Masses that were found potentially related before
and after acid degradation and the acid labile
nucleotides correlated to mass changes.

| | Before acid | After acid | |
|---|---|---|---|
| | degradation | degradation | Acid-labile |
| 0 | 24610.49 | 24252.31 | Y |
| 1 | 24939.55 | 24581.38 | Y |
| 2 | 24626.46 | 24268.3 | Y |
| 3 | 24955.52 | 24597.35 | Y |
| 4 | 24385.35 | 24027.24 | Y |
| 5 | 24955.52 | 24610.42 | Gr(p) |
| 6 | 24385.35 | 24252.31 | cnm5U |
| 7 | 24385.35 | 24267.31 | I |
| 8 | 24305.4 | 24087.24 | g6A |
| 9 | 24670.45 | 24280.31 | o2yW |
| 10 | 24639.46 | 24331.29 | ms2t6A |
| 11 | 24792.46 | 24597.35 | acp3U/cmnm5Um |
| 12 | 24792.46 | 24610.42 | mcmo5U |

TABLE S4-3
The ratio of 74 nt, 75 nt and 76 nt
tRNA-Phe before acid-degradation.

| tRNA | Theoretical | Experimental | | Sum | |
|---|---|---|---|---|---|
| Phe | mass | mass | ppm | Intensity | Percentage |
| 74 nt | 24305.71869 | 24305.410 | 12.7187 | 2.58E+06 | 1.0 |
| 75 nt | 24610.75989 | 24610.491 | 10.9273 | 1.63E+08 | 62.1 |
| 76 nt | 24939.8124 | 24939.549 | 10.5791 | 9.69E+07 | 37.0 |

TABLE S5-1
3′_biotin_tRNA_T1_SIII_111418s05_76A. Sequencing
of 3′ biotin labeled tRNA segment III from 58m¹A
to 76A by global hierarchical ranking algorithm.

| Fragment | Mass | RT | Base | Volume | PPM |
|---|---|---|---|---|---|
| 1 | 826.3164 | 35.809 | Tag | 2645323 | 2.42 |
| 2 | 1155.3679 | 34.555 | A | 580850 | 2.60 |
| 3 | 1460.4116 | 30.202 | C | 259583 | 0.41 |
| 4 | 1765.4505 | 29.311 | C | 4875476 | 1.70 |
| 5 | 2094.5027 | 30.921 | A | 560348 | 1.58 |
| 6 | 2399.5455 | 30.024 | C | 241970 | 0.75 |
| 7 | 2744.5948 | 30.494 | G | 365785 | 0.04 |
| 8 | 3049.6138 | 30.755 | C | 245795 | 7.28 |
| 9 | 3355.6561 | 31.570 | U | 377273 | 1.58 |
| 10 | 3661.6854 | 32.930 | U | 4226311 | 0.38 |
| 11 | 3990.7364 | 34.122 | A | 4968527 | 0.73 |
| 12 | 4319.7918 | 35.332 | A | 245329 | 0.00 |
| 13 | 4664.8388 | 34.606 | G | 4756748 | 0.09 |
| 14 | 4993.8992 | 35.504 | A | 307359 | 1.50 |
| 15 | 5298.9333 | 35.691 | C | 4083332 | 0.06 |
| 16 | 5627.9522 | 35.501 | A | 160811 | 5.92 |
| 17 | 5933.0022 | 35.649 | C | 157328 | 4.15 |
| 18 | 6238.0838 | 36.541 | C | 89737 | 2.52 |
| 19 | 6544.1101 | 36.202 | U | 672814 | 2.54 |
| 20 | 6887.1727 | 37.539 | mA | 1193510 | 1.61 |

TABLE S5-2
3′_biotin_tRNA_T1_SIII_111418s05_75C. Sequencing
of 3′ biotin labeled tRNA segment III from 58m¹A
to 75C by global hierarchical ranking algorithm.

| Fragment | Mass | RT | Base | Volume | PPM |
|---|---|---|---|---|---|
| 1 | 826.3164 | 35.809 | Tag | 2645323 | 2.42 |
| 2 | 1131.3573 | 28.724 | C | 2536602 | 2.12 |
| 3 | 1436.3979 | 26.748 | C | 1504369 | 2.16 |
| 4 | 1765.4505 | 29.311 | A | 4875476 | 1.70 |
| 5 | 2070.4898 | 27.904 | C | 1807879 | 2.41 |
| 6 | 2415.5392 | 28.436 | G | 4919858 | 1.24 |
| 7 | 2720.5806 | 28.781 | C | 4403013 | 1.07 |
| 8 | 3026.6061 | 29.745 | U | 5263366 | 0.93 |
| 9 | 3332.6311 | 30.654 | U | 3654432 | 0.96 |
| 10 | 3661.6854 | 32.930 | A | 4226311 | 0.38 |
| 11 | 3990.7364 | 34.122 | A | 4968527 | 0.73 |
| 12 | 4335.7879 | 33.348 | G | 2855812 | 0.28 |
| 13 | 4664.8388 | 34.606 | A | 4756748 | 0.09 |
| 14 | 4969.8783 | 34.250 | C | 2303352 | 0.44 |
| 15 | 5298.9333 | 35.691 | A | 4083332 | 0.06 |
| 16 | 5603.9769 | 35.502 | C | 2292626 | 0.46 |
| 17 | 5909.0178 | 35.637 | C | 2429322 | 0.37 |
| 18 | 6215.0412 | 36.088 | U | 860704 | 0.03 |
| 19 | 6558.1157 | 36.751 | mA | 16787962 | 1.01 |

TABLE S5-3
3′_biotin_tRNA_T1_SIII_111418s05_74C. Sequencing
of 3′ biotin labeled tRNA segment III from 58m¹A
to 74C by global hierarchical ranking algorithm.

| Fragment | Mass | RT | Base | Volume | PPM |
|---|---|---|---|---|---|
| 1 | 826.3164 | 35.809 | Tag | 2645323 | 2.42 |
| 2 | 1131.3573 | 28.724 | C | 2536602 | 2.12 |
| 3 | 1460.4116 | 30.202 | A | 259583 | 0.41 |
| 4 | 1765.4505 | 29.311 | C | 4875476 | 1.70 |
| 5 | 2110.4918 | 27.882 | G | 356221 | 4.31 |
| 6 | 2415.5392 | 28.436 | C | 4919858 | 1.24 |
| 7 | 2721.5695 | 29.145 | U | 239635 | 0.70 |
| 8 | 3027.5972 | 30.047 | U | 68400 | 1.39 |
| 9 | 3356.6432 | 32.543 | A | 189932 | 0.69 |
| 10 | 3685.6934 | 33.833 | A | 159564 | 1.25 |
| 11 | 4030.7417 | 33.004 | G | 82558 | 0.92 |
| 12 | 4359.8007 | 34.352 | A | 289735 | 0.64 |
| 13 | 4664.8388 | 34.606 | C | 4756748 | 0.09 |
| 14 | 4993.8992 | 35.504 | A | 307359 | 1.50 |
| 15 | 5298.9333 | 35.691 | C | 4083332 | 0.06 |
| 16 | 5603.9769 | 35.502 | C | 2292626 | 0.46 |
| 17 | 5910.0206 | 35.639 | U | 98526 | 3.54 |
| 18 | 6253.0697 | 36.605 | mA | 181155 | 0.30 |

TABLE S5-4
5′_OH_tRNA_T1_SII_111418s05_44A45G.
Sequencing of 5′ OH tRNA segment II from 21A
to 57G by global hierarchical ranking algorithm.

| Fragment | Mass | RT | Base | Volume | PPM |
|---|---|---|---|---|---|
| 1 | 692.1081 | 0.945 | A + G | 448392 | 3.47 |
| 2 | 1021.1592 | 0.996 | A | 612623 | 3.72 |
| 3 | 1366.2059 | 1.023 | G | 1163701 | 3.29 |
| 4 | 1671.2489 | 1.112 | C | 1917190 | 1.68 |
| 5 | 2044.3269 | 8.858 | 2mG | 2025885 | 1.71 |
| 6 | 2349.3682 | 10.309 | C | 3120462 | 1.49 |
| 7 | 2654.4101 | 12.749 | C | 6309574 | 1.09 |
| 8 | 2983.4617 | 16.073 | A | 5462129 | 1.27 |
| 9 | 3328.5102 | 17.647 | G | 6892234 | 0.81 |
| 10 | 3657.5632 | 19.875 | A | 4203490 | 0.60 |
| 11 | 4282.6476 | 23.391 | U + Cm | 11059167 | 0.02 |
| 12 | 4970.7632 | 26.996 | A + Gm | 8957192 | 0.02 |
| 13 | 5299.8175 | 28.115 | A | 9137581 | 0.32 |
| 14 | 5511.8281 | 28.449 | Y′ | 9044373 | 0.67 |
| 15 | 5840.8796 | 29.718 | A | 7213450 | 0.46 |
| 16 | 6146.9082 | 30.061 | U | 12938074 | 0.98 |
| 17 | 6465.9647 | 30.688 | mC | 6445803 | 0.87 |
| 18 | 6771.9918 | 31.161 | U | 6802824 | 1.09 |
| 19 | 7117.0401 | 31.251 | G | 3468612 | 1.17 |
| 20 | 7462.0865 | 32.049 | G | 2834683 | 0.98 |
| 21 | 7791.1394 | 32.735 | A | 2239278 | 1.00 |
| 22 | 8136.1981 | 33.016 | G | 3437631 | 2.35 |
| 23 | 8495.2645 | 33.131 | mG | 2251492 | 2.62 |
| 24 | 8801.2888 | 33.439 | U | 3178250 | 2.42 |
| 25 | 9106.3319 | 33.677 | C | 3146668 | 2.54 |
| 26 | 9425.3892 | 33.961 | mC | 3341188 | 2.49 |
| 27 | 9731.4100 | 34.135 | U | 3700286 | 1.95 |
| 28 | 10076.4607 | 34.378 | G | 2776140 | 2.21 |
| 29 | 10382.4798 | 34.582 | U | 2849708 | 1.55 |
| 30 | 10727.5480 | 34.793 | G | 2740634 | 3.44 |
| 31 | 11047.5761 | 35.136 | T | 781981 | 2.17 |
| 32 | 11353.6241 | 35.183 | U | 4303300 | 4.11 |
| 33 | 11658.6776 | 35.364 | C | 1498752 | 5.05 |
| 34 | 12003.6973 | 35.531 | G | 6123452 | 2.60 |

TABLE S5-5
5′_OH_tRNA_T1_SII_111418s05_44g45a.
Sequencing of 5′ OH tRNA segment II from
21A to 57G by global hierarchical ranking algorithm.

| Fragment | Mass | RT | Base | Volume | PPM |
|---|---|---|---|---|---|
| 1 | 692.1081 | 0.945 | A + G | 448392 | 3.47 |
| 2 | 1021.1592 | 0.996 | A | 612623 | 3.72 |
| 3 | 1366.2059 | 1.023 | G | 1163701 | 3.29 |
| 4 | 1671.2489 | 1.112 | C | 1917190 | 1.68 |
| 5 | 2044.3269 | 8.858 | 2mG | 2025885 | 1.71 |
| 6 | 2349.3682 | 10.309 | C | 3120462 | 1.49 |
| 7 | 2654.4101 | 12.749 | C | 6309574 | 1.09 |
| 8 | 2983.4617 | 16.073 | A | 5462129 | 1.27 |
| 9 | 3328.5102 | 17.647 | G | 6892234 | 2.55 |
| 10 | 3657.5632 | 19.875 | A | 4203490 | 0.60 |
| 11 | 4282.6476 | 23.391 | U + Cm | 11059167 | 0.02 |
| 12 | 4970.7632 | 26.996 | A + Gm | 8957192 | 0.02 |
| 13 | 5299.8175 | 28.115 | A | 9137581 | 0.34 |
| 14 | 5511.8281 | 28.449 | Y′ | 9044373 | 0.69 |
| 15 | 5840.8796 | 29.718 | A | 7213450 | 0.48 |
| 16 | 6146.9082 | 30.061 | U | 12938074 | 0.98 |
| 17 | 6465.9647 | 30.688 | mC | 6445803 | 0.87 |
| 18 | 6771.9918 | 31.161 | U | 6802824 | 1.08 |
| 19 | 7117.0401 | 31.251 | G | 3468612 | 1.15 |
| 20 | 7462.0865 | 32.049 | G | 2834683 | 0.96 |
| 21 | 7807.1332 | 32.101 | G | 2248564 | 0.83 |
| 22 | 8136.1981 | 33.016 | A | 3437631 | 2.32 |
| 23 | 8495.2645 | 33.131 | mG | 2251492 | 2.61 |
| 24 | 8801.2888 | 33.439 | U | 3178250 | 2.40 |
| 25 | 9106.3319 | 33.677 | C | 3146668 | 2.51 |
| 26 | 9425.3892 | 33.961 | mC | 3341188 | 2.47 |
| 27 | 9731.4100 | 34.135 | U | 3700286 | 1.92 |
| 28 | 10076.4607 | 34.378 | G | 2776140 | 2.18 |
| 29 | 10382.4798 | 34.582 | U | 2849708 | 1.51 |
| 30 | 10727.5480 | 34.793 | G | 2740634 | 3.40 |
| 31 | 11047.5761 | 35.136 | T | 781981 | 2.14 |
| 32 | 11353.6241 | 35.183 | U | 4303300 | 4.07 |
| 33 | 11658.6776 | 35.364 | C | 1498752 | 5.01 |
| 34 | 12003.6973 | 35.531 | G | 6123452 | 2.56 |

TABLE S5-6
5′_pG_tRNA_T1_SI_111418s05.
Sequencing of 5′ pG tRNA segment I from 1G
to 20G by global hierarchical ranking algorithm.

| Fragment | Mass | RT | Base | Volume | PPM |
|---|---|---|---|---|---|
| 1 | 443.0222 | 0.968 | pG | 32204 | 4.74 |
| 2 | 748.0626 | 0.935 | C | 327973 | 4.01 |
| 3 | 1093.1092 | 0.963 | G | 247078 | 3.48 |
| 4 | 1438.1583 | 1.010 | G | 1953624 | 1.46 |
| 5 | 1767.2105 | 2.512 | A | 6646248 | 1.36 |
| 6 | 2073.2377 | 4.800 | U | 11078570 | 0.24 |
| 7 | 2379.2611 | 7.664 | U | 13653044 | 1.01 |
| 8 | 2685.2874 | 9.948 | U | 13651928 | 0.52 |
| 9 | 3014.3399 | 13.244 | A | 8446589 | 0.46 |
| 10 | 3373.3974 | 16.657 | mG | 5400820 | 2.08 |
| 11 | 3678.4462 | 17.883 | C | 6427287 | 0.14 |
| 12 | 3984.4711 | 19.330 | U | 10498687 | 0.03 |
| 13 | 4289.5141 | 20.432 | C | 13067020 | 0.42 |
| 14 | 4618.5661 | 22.240 | A | 9336602 | 0.28 |
| 15 | 4963.6167 | 23.110 | G | 19445698 | 0.91 |
| 16 | 5271.6368 | 23.792 | D | 6241383 | 3.11 |
| 17 | 5579.6992 | 24.454 | D | 7740033 | 0.90 |
| 18 | 5924.7535 | 25.268 | G | 104745696 | 2.01 |
| 19 | 6269.8003 | 25.980 | G | 3057757 | 1.80 |
| 20 | 6614.8364 | 26.615 | G | 673220 | 0.00 |

TABLE S5-7
5′_biotin_tRNA_T1_SI_042519s07.
Sequencing of 5′ biotin labeled tRNA segment
I from 1G to 18G by global hierarchical ranking algorithm.

| Fragment | Mass | RT | Base | Volume | PPM |
|---|---|---|---|---|---|
| 1 | 938.2184 | 21.449 | Tag + G | 403806 | 3.41 |
| 2 | 1243.2600 | 23.971 | C | 277726 | 2.33 |
| 3 | 1588.3060 | 25.493 | G | 238503 | 2.71 |
| 4 | 1933.3518 | 27.433 | G | 44902 | 3.05 |
| 5 | 2262.4042 | 29.682 | A | 35264 | 2.65 |
| 6 | 2568.4387 | 30.807 | U | 64428 | 1.21 |
| 7 | 2874.4631 | 31.835 | U | 219666 | 0.73 |
| 8 | 3180.4871 | 32.783 | U | 173234 | 0.22 |
| 9 | 3509.5467 | 34.465 | A | 67573 | 2.22 |
| 10 | 3868.6148 | 35.174 | mG | 226704 | 3.31 |
| 11 | 4173.6443 | 36.794 | C | 63409 | 0.24 |
| 12 | 4479.6520 | 37.559 | U | 12772 | 3.73 |
| 13 | 4784.7078 | 38.002 | C | 14478 | 0.46 |
| 14 | 5113.7758 | 38.479 | A | 69348 | 2.60 |
| 15 | 5458.8177 | 39.347 | G | 1588901 | 1.43 |
| 16 | 5766.8095 | 39.208 | D | 25595 | 7.18 |
| 17 | 6074.9000 | 39.440 | D | 118414 | 1.33 |
| 18 | 6419.9573 | 40.140 | G | 383672 | 2.80 |

TABLE S5-8
5′_biotin_tRNA_T1_SII_032919s07_44A45G.
Sequencing of 5′ biotin labeled segment II from
21A to 57G by global hierarchical ranking algorithm.

| Fragment | Mass | RT | Base | Volume | PPM |
|---|---|---|---|---|---|
| 1 | 922.2241 | 25.229 | Tag + A | 745215 | 3.04 |
| 2 | 1267.2710 | 25.756 | G | 577150 | 2.60 |
| 3 | 1596.3229 | 28.405 | A | 472089 | 2.44 |
| 4 | 1941.3702 | 29.167 | G | 591742 | 2.06 |
| 5 | 2246.4125 | 30.221 | C | 930358 | 1.34 |
| 6 | 2619.4912 | 35.055 | 2mG | 276858 | 1.15 |
| 7 | 2924.5312 | 35.109 | C | 937840 | 1.47 |
| 8 | 3229.5745 | 35.989 | C | 1389357 | 0.71 |
| 9 | 3558.6244 | 37.535 | A | 944505 | 1.38 |
| 10 | 3903.6768 | 38.016 | G | 1334405 | 0.03 |
| 11 | 4232.7261 | 39.120 | A | 899666 | 0.73 |
| 12 | 4857.8097 | 40.778 | U + Cm | 2369525 | 0.37 |
| 13 | 5545.9261 | 42.941 | A + Gm | 1777156 | 0.18 |
| 14 | 5874.9889 | 43.512 | A | 1527490 | 1.60 |
| 15 | 6086.9945 | 43.461 | Y′ | 2278504 | 1.05 |
| 16 | 6416.0477 | 44.268 | A | 1366254 | 1.11 |
| 17 | 6722.0827 | 44.327 | U | 1049995 | 2.48 |
| 18 | 7041.1313 | 44.591 | mC | 1297495 | 1.19 |
| 19 | 7347.1602 | 44.775 | U | 1560416 | 1.62 |
| 20 | 7692.2118 | 45.013 | G | 1319384 | 2.09 |
| 21 | 8037.2549 | 45.410 | G | 1009813 | 1.47 |
| 22 | 8366.3413 | 45.858 | A | 271843 | 5.46 |
| 23 | 8711.3823 | 45.865 | G | 1226283 | 4.51 |
| 24 | 9070.4677 | 45.822 | mG | 520562 | 6.79 |
| 25 | 9376.4389 | 45.871 | U | 416614 | 0.79 |
| 26 | 9681.5649 | 45.921 | C | 587268 | 9.51 |
| 27 | 10000.5521 | 46.069 | mC | 504658 | 2.24 |
| 28 | 10306.6258 | 46.099 | U | 925998 | 6.86 |
| 29 | 10651.5989 | 46.183 | G | 672326 | 0.34 |
| 30 | 10957.6318 | 46.200 | U | 320227 | 0.36 |
| 31 | 11302.6636 | 46.313 | G | 962623 | 1.04 |
| 32 | 11622.6493 | 46.492 | T | 325162 | 5.76 |
| 33 | 11928.6903 | 46.401 | U | 2182861 | 4.31 |
| 34 | 12233.7642 | 46.449 | C | 463444 | 1.54 |
| 35 | 12578.8603 | 46.548 | G | 2766678 | 2.38 |

TABLE S5-9
5′_biotin_tRNA_T1_SII_032919s07_44g45a.
Sequencing of 5′ biotin labeled tRNA segment
II from 21A to 57G by global hierarchical ranking algorithm.

| Fragment | Mass | RT | Base | Volume | PPM |
|---|---|---|---|---|---|
| 1 | 922.2241 | 25.229 | Tag + A | 745215 | 3.04 |
| 2 | 1267.2710 | 25.756 | G | 577150 | 2.60 |
| 3 | 1596.3229 | 28.405 | A | 472089 | 2.44 |
| 4 | 1941.3702 | 29.167 | G | 591742 | 2.06 |
| 5 | 2246.4125 | 30.221 | C | 930358 | 1.34 |
| 6 | 2619.4912 | 35.055 | 2mG | 276858 | 1.15 |
| 7 | 2924.5312 | 35.109 | C | 937840 | 1.47 |
| 8 | 3229.5745 | 35.989 | C | 1389357 | 0.71 |
| 9 | 3558.6244 | 37.535 | A | 944505 | 1.38 |
| 10 | 3903.6768 | 38.016 | G | 1334405 | 0.03 |
| 11 | 4232.7261 | 39.120 | A | 899666 | 0.73 |
| 12 | 4857.8097 | 40.778 | U + Cm | 2369525 | 0.37 |
| 13 | 5545.9261 | 42.941 | A + Gm | 1777156 | 0.18 |
| 14 | 5874.9889 | 43.512 | A | 1527490 | 1.58 |
| 15 | 6086.9945 | 43.461 | Y′ | 2278504 | 1.03 |
| 16 | 6416.0477 | 44.268 | A | 1366254 | 1.09 |
| 17 | 6722.0827 | 44.327 | U | 1049995 | 2.48 |
| 18 | 7041.1313 | 44.591 | mC | 1297495 | 1.19 |
| 19 | 7347.1602 | 44.775 | U | 1560416 | 1.63 |
| 20 | 7692.2118 | 45.013 | G | 1319384 | 2.11 |
| 21 | 8037.2549 | 45.410 | G | 1009813 | 1.48 |
| 22 | 8382.2778 | 45.275 | G | 200964 | 1.50 |
| 23 | 8711.3823 | 45.865 | A | 1226283 | 4.53 |
| 24 | 9070.4677 | 45.822 | mG | 520562 | 6.80 |
| 25 | 9376.4389 | 45.871 | U | 416614 | 0.81 |
| 26 | 9681.5649 | 45.921 | C | 587268 | 9.53 |
| 27 | 10000.5521 | 46.069 | mC | 504658 | 2.26 |
| 28 | 10306.6258 | 46.099 | U | 925998 | 6.89 |
| 29 | 10651.5989 | 46.183 | G | 672326 | 0.31 |
| 30 | 10957.6318 | 46.200 | U | 320227 | 0.39 |
| 31 | 11302.6636 | 46.313 | G | 962623 | 1.00 |
| 32 | 11622.6493 | 46.492 | T | 325162 | 5.73 |
| 33 | 11928.6903 | 46.401 | U | 2182861 | 4.27 |
| 34 | 12233.7642 | 46.449 | C | 463444 | 1.50 |
| 35 | 12578.8603 | 46.548 | G | 2766678 | 2.42 |

TABLE S5-10
3′_tRNA_1009s06. Sequencing of acid degraded tRNA from
45G to 76A by global hierarchical ranking algorithm.

| Fragment | Mass | RT | Base | Volume | PPM |
|---|---|---|---|---|---|
| 1 | 877.1786 | 1.270 | A + C + C | 1022495 | 0.80 |
| 2 | 1206.2286 | 2.926 | A | 1172115 | 2.65 |
| 3 | 1511.2689 | 2.572 | C | 819385 | 2.78 |
| 4 | 1856.3153 | 3.218 | G | 1266301 | 2.80 |
| 5 | 2161.3551 | 3.798 | C | 1544446 | 3.10 |
| 6 | 2467.3789 | 4.806 | U | 2083726 | 3.36 |
| 7 | 2773.4034 | 5.685 | U | 1696734 | 3.32 |
| 8 | 3102.4553 | 7.075 | A | 5583907 | 3.16 |
| 9 | 3431.5054 | 7.910 | A | 2247902 | 3.56 |
| 10 | 3776.5516 | 7.745 | G | 5639286 | 3.55 |
| 11 | 4105.6016 | 8.447 | A | 2679354 | 3.87 |
| 12 | 4410.6408 | 8.523 | C | 4702025 | 4.08 |
| 13 | 4739.6917 | 9.123 | A | 2963739 | 4.14 |
| 14 | 5044.7319 | 9.175 | C | 2073512 | 4.10 |
| 15 | 5349.7949 | 9.288 | C | 1906782 | 0.19 |
| 16 | 5655.7967 | 9.545 | U | 914935 | 4.00 |
| 17 | 5998.8627 | 9.818 | mA | 2160204 | 4.12 |
| 18 | 6343.9049 | 9.900 | G | 2309111 | 4.71 |
| 19 | 6648.9464 | 9.893 | C | 3092250 | 4.47 |
| 20 | 6954.9754 | 9.838 | U | 1201050 | 3.75 |
| 21 | 7275.0127 | 10.396 | T | 2267279 | 4.10 |
| 22 | 7620.0765 | 10.498 | G | 1762814 | 1.76 |
| 23 | 7926.1455 | 10.423 | U | 1562423 | 3.81 |
| 24 | 8271.1067 | 10.603 | G | 1920966 | 6.77 |
| 25 | 8577.2011 | 10.660 | U | 1709835 | 1.52 |
| 26 | 8896.1598 | 11.550 | mC | 875226 | 9.58 |
| 27 | 9201.2581 | 11.313 | C | 769527 | 3.06 |
| 28 | 9507.2765 | 11.082 | U | 572956 | 3.70 |
| 29 | 9866.3028 | 11.030 | mG | 412887 | 7.30 |
| 30 | 10211.3522 | 11.073 | G | 709961 | 6.86 |

TABLE S5-11
5′_pG_tRNA_100918s06. Sequencing of 5′pG tRNA from
1G to 31A by global hierarchical ranking algorithm.

| Fragment | Mass | RT | Base | Volume | PPM |
|---|---|---|---|---|---|
| 1 | 443.0274 | 0.931 | pG | 233231 | 7.00 |
| 2 | 748.0684 | 1.039 | C | 883929 | 3.74 |
| 3 | 1093.1105 | 1.800 | G | 2062278 | 2.29 |
| 4 | 1438.1575 | 3.239 | G | 3687690 | 2.02 |
| 5 | 1767.2087 | 4.484 | A | 4522172 | 2.38 |
| 6 | 2073.2354 | 5.369 | U | 8131266 | 1.35 |
| 7 | 2379.2590 | 6.043 | U | 8862830 | 1.89 |
| 8 | 2685.2836 | 6.593 | U | 9612100 | 1.94 |
| 9 | 3014.3343 | 7.355 | A | 6218090 | 2.32 |
| 10 | 3373.3964 | 8.120 | mG | 2974994 | 2.37 |
| 11 | 3678.4380 | 8.403 | C | 3957178 | 2.09 |
| 12 | 3984.4601 | 8.709 | U | 6419872 | 2.74 |
| 13 | 4289.5007 | 8.942 | C | 8348561 | 2.70 |
| 14 | 4618.5517 | 9.346 | A | 3797284 | 2.84 |
| 15 | 4963.6043 | 9.522 | G | 217686 | 1.59 |
| 16 | 5271.6374 | 9.631 | D | 3108073 | 3.00 |
| 17 | 5579.6773 | 9.748 | D | 3781679 | 3.03 |
| 18 | 5924.7327 | 9.944 | G | 689750 | 1.50 |
| 19 | 6269.7714 | 10.091 | G | 2753572 | 2.81 |
| 20 | 6614.8124 | 10.232 | G | 1506355 | 3.63 |
| 21 | 6943.8650 | 10.468 | A | 1708708 | 3.44 |
| 22 | 7288.9012 | 10.601 | G | 779104 | 4.82 |
| 23 | 7617.9417 | 10.826 | A | 852001 | 6.18 |
| 24 | 7963.0075 | 10.910 | G | 2445671 | 3.60 |
| 25 | 8268.0027 | 11.143 | C | 1087860 | 9.05 |
| 26 | 8641.1310 | 11.694 | 2mG | 207499 | 2.92 |
| 27 | 8946.1664 | 11.727 | C | 1364582 | 3.48 |
| 28 | 9251.2074 | 11.743 | C | 1059830 | 3.39 |
| 29 | 9580.2455 | 11.864 | A | 1450228 | 4.78 |
| 30 | 9925.3349 | 11.871 | G | 2494820 | 0.38 |
| 31 | 10254.2927 | 11.993 | A | 155606 | 9.61 |

TABLE S5-12
Yield of CMC conversion occurring at pseudouridine measured by LC-MS.

| Conversion state | Fragment | Calc mass | Exp mass | m/z | EIC | QS | ppm |
|---|---|---|---|---|---|---|---|
| Non-converted | 21A to 44A | 7791.1320 | 7791.1787 | 778.1111 | 1129053 | 80 | −5.99 |
| CMC-converted | 21A to 44A | 8042.3318 | 8042.3492 | 803.2263 | 4123573 | 80 | −2.16 |
| Non-converted | 57G to 47U | 3526.4344 | 3526.4333 | 586.7314 | 1176461 | 100 | 0.31 |
| CMC-converted | 57G to 47A | 3777.6342 | 3777.6332 | 628.5979 | 3779411 | 100 | 0.26 |

TABLE S5-13
5′_tRNA_T1_nonCMC_SII_042519s04_44A45G.
Sequencing of 5′ non-CMC converted tRNA segment
II from 21A to 45G by global hierarchical ranking algorithm.

| Fragment | Mass | RT | Base | Volume | PPM |
|---|---|---|---|---|---|
| 1 | 692.1076 | 1.032 | A + G | 121835 | 4.19 |
| 2 | 1021.1576 | 1.264 | A | 548483 | 5.29 |
| 3 | 1366.2072 | 4.020 | G | 2219430 | 2.34 |
| 4 | 1671.2480 | 7.304 | C | 3142702 | 2.21 |
| 5 | 2044.3269 | 16.800 | 2mG | 1700693 | 1.71 |
| 6 | 2349.3689 | 18.430 | C | 2431764 | 1.19 |
| 7 | 2654.4105 | 20.727 | C | 6691067 | 0.94 |
| 8 | 2983.4639 | 23.756 | A | 9276684 | 0.54 |
| 9 | 3328.5120 | 25.192 | G | 10673175 | 0.27 |
| 10 | 3657.5668 | 27.417 | A | 5126136 | 0.38 |
| 11 | 4282.6486 | 30.874 | U + Cm | 15880661 | 0.21 |
| 12 | 4970.7665 | 34.609 | A + Gm | 10873309 | 0.64 |
| 13 | 5299.8210 | 35.684 | A | 12807606 | 0.98 |
| 14 | 5511.8306 | 35.900 | Y′ | 13088146 | 1.12 |
| 15 | 5840.8850 | 37.167 | A | 3623732 | 1.39 |
| 16 | 6146.9096 | 37.460 | U | 1897334 | 1.20 |
| 17 | 6465.9704 | 38.006 | mC | 2463925 | 1.75 |
| 18 | 6771.9928 | 38.393 | U | 3706693 | 1.24 |
| 19 | 7117.0453 | 38.873 | G | 3506106 | 1.90 |
| 20 | 7462.0964 | 39.527 | G | 2455794 | 2.30 |
| 21 | 7791.1787 | 40.196 | A | 1226259 | 6.03 |
| 22 | 8136.1916 | 40.385 | G | 1925167 | 1.54 |

TABLE S5-14
5′_tRNA_T1_nonCMC_SII_042519s04_44g45a.
Sequencing of 5′ non-CMC converted tRNA segment
II from 21A to 45A by global hierarchical ranking algorithm.

| Fragment | Mass | RT | Base | Volume | PPM |
|---|---|---|---|---|---|
| 1 | 692.1076 | 1.032 | A + G | 121835 | 4.19 |
| 2 | 1021.1576 | 1.264 | A | 548483 | 5.29 |
| 3 | 1366.2072 | 4.020 | G | 2219430 | 2.34 |
| 4 | 1671.2480 | 7.304 | C | 3142702 | 2.21 |
| 5 | 2044.3269 | 16.800 | 2mG | 1700693 | 1.71 |
| 6 | 2349.3689 | 18.430 | C | 2431764 | 1.19 |
| 7 | 2654.4105 | 20.727 | C | 6691067 | 0.94 |
| 8 | 2983.4639 | 23.756 | A | 9276684 | 0.54 |
| 9 | 3328.5120 | 25.192 | G | 10673175 | 0.27 |
| 10 | 3657.5668 | 27.417 | A | 5126136 | 0.38 |
| 11 | 4282.6486 | 30.874 | U + Cm | 15880661 | 0.21 |
| 12 | 4970.7665 | 34.609 | A + Gm | 10873309 | 0.64 |
| 13 | 5299.8210 | 35.684 | A | 12807606 | 0.98 |
| 14 | 5511.8306 | 35.900 | Y′ | 13088146 | 1.12 |
| 15 | 5840.8850 | 37.167 | A | 3623732 | 1.39 |
| 16 | 6146.9096 | 37.460 | U | 1897334 | 1.20 |
| 17 | 6465.9704 | 38.006 | mC | 2463925 | 1.75 |
| 18 | 6771.9928 | 38.393 | U | 3706693 | 1.24 |
| 19 | 7117.0453 | 38.873 | G | 3506106 | 1.90 |
| 20 | 7462.0964 | 39.527 | G | 2455794 | 2.30 |
| 21 | 7807.1385 | 39.523 | G | 835117 | 1.52 |
| 22 | 8136.1916 | 40.385 | A | 1925167 | 1.54 |

TABLE S5-15
5′_tRNA_T1_CMC_SII_042519s04.
Sequencing of 5′ CMC converted tRNA segment
II from 39ψ to 44A by global hierarchical ranking algorithm.

| Fragment | Mass | RT | Base | Volume | PPM |
|---|---|---|---|---|---|
| 1 | 6398.1211 | 44.707 | Mod-Psi | 1295323 | 2.97 |
| 2 | 6717.1789 | 45.223 | mC | 2506731 | 2.96 |
| 3 | 7023.1878 | 45.283 | U | 3037253 | 0.50 |
| 4 | 7368.2361 | 45.446 | G | 8115206 | 0.60 |
| 5 | 7713.3006 | 45.574 | G | 4221938 | 2.79 |
| 6 | 8042.3492 | 46.255 | A | 3190026 | 2.19 |

TABLE S5-16
3′_tRNA_T1_nonCMC_SII_042519s04.
Sequencing of 3′ non-CMC converted tRNA segment
II from 57G to 47U by global hierarchical ranking algorithm.

| Fragment | Mass | RT | Base | Volume | PPM |
|---|---|---|---|---|---|
| 1 | 668.0943 | 0.968 | G + C | 79549 | 7.33 |
| 2 | 974.1302 | 0.915 | U | 826458 | 5.85 |
| 3 | 1294.1594 | 2.732 | T | 403523 | 4.71 |
| 4 | 1639.2089 | 6.500 | G | 789168 | 2.44 |
| 5 | 1945.2357 | 6.129 | U | 190380 | 1.29 |
| 6 | 2290.2818 | 10.466 | G | 1584520 | 1.66 |
| 7 | 2596.3069 | 12.965 | U | 1100858 | 1.54 |
| 8 | 2915.3646 | 17.907 | mC | 1557574 | 1.10 |
| 9 | 3220.4052 | 18.523 | C | 773618 | 1.21 |
| 10 | 3526.4333 | 20.318 | U | 2252901 | 0.31 |

TABLE S5-17
3′_tRNA_T1_CMC_SII_042519s04.
Sequencing of 3′ CMC converted tRNA segment
II from 57G to 47U by global hierarchical ranking algorithm.

| Fragment | Mass | RT | Base | Volume | PPM |
|---|---|---|---|---|---|
| 1 | 1225.3215 | 14.484 | Mod-Psi | 882395 | 2.29 |
| 2 | 1545.3611 | 19.764 | T | 78086 | 2.72 |
| 3 | 1890.4097 | 27.200 | G | 1324986 | 1.59 |
| 4 | 2196.4340 | 25.561 | U | 33874 | 1.82 |
| 5 | 2541.4824 | 27.899 | G | 3029272 | 1.18 |
| 6 | 2847.5087 | 28.729 | U | 2275337 | 0.70 |
| 7 | 3166.5661 | 32.358 | mC | 2499558 | 0.47 |
| 8 | 3471.6055 | 32.073 | C | 2485944 | 0.98 |
| 9 | 3777.6332 | 32.777 | U | 4553148 | 0.26 |

TABLE S5-18
Detection of Y′ in the presence of tRNA before (in full-
length tRNA) and after (as isolated base) acid degradation.

| In a form of segment II | Calc mass | Exp mass | m/z | EIC | Percent | QS | ppm |
|---|---|---|---|---|---|---|---|
| Y before acid degradation | 12361.805 | 12361.841 | 823.1141 | 2324857 | 90% | 80 | −2.9 |
| Y′ before acid degradation | 12003.666 | 12003.762 | 922.359 | 230727 | 10% | 48 | −7.9 |
| Isolated Y′ after acid degradation | 376.1495 | 376.1479 | 375.1409 | 49059213 | 100% | 100 | 4.3 |

TABLE S5-19
RNase T1 digestion products of tRNA measured by LC-MS. Among them, three major segments
were observed which have the strongest peak volume. The relative quantities of different
product species were quantified by integrating the extracted ion current (EIC) (1, 7).

| Fragment | Calc mass | Exp mass | m/z | EIC (Area) | Percent | Quality score | ppm |
|---|---|---|---|---|---|---|---|
| 58 m¹A to 74C | 5364.7935 | 5364.7939 | 595.0800 | 226450 | 3% | 98 | −0.1 |
| 58 m¹A to 75C | 5669.8348 | 5669.8403 | 628.9753 | 6242830 | 80% | 100 | −1.0 |
| 58 m¹A to 76A | 5998.8873 | 5998.8845 | 598.8828 | 1323018 | 17% | 100 | 0.5 |

TABLE S5-20
5′_OH_tRNA_T1_SII_111418s05_44A45G.
LC-MS analysis of segment II from 34Gm to 55ψ(mass ladder components from 3′ to 5′).

| Theoretical | | | | Extracted data file after LC/MS analysis | | | | |
|---|---|---|---|---|---|---|---|---|
| | Theoretical | Base | | MFE | | | Quality | Error |
| Fragments | mass | mass | Base | mass | t_{R} | Volume | Score | ppm |
| 21 | 7739.0291 | 688.1156 | A + Gm | 7739.0198 | 28.919 | 572629 | 80 | 1.20 |
| 20 | 7050.9135 | 329.0525 | A | 7050.9277 | 26.539 | 413840 | 60 | −2.01 |
| 19 | 6721.8610 | 212.0086 | Y′ | 6721.8635 | 24.741 | 381223 | 72.8 | −0.37 |
| 18 | 6509.8524 | 329.0525 | A | 6509.8604 | 25.336 | 1019699 | 80 | −1.23 |
| 17 | 6180.7999 | 306.0253 | ψ | 6180.8037 | 23.079 | 707995 | 77.8 | −0.61 |
| 16 | 5874.7746 | 319.0570 | m5C | 5874.7783 | 23.641 | 2167527 | 100 | −0.63 |
| 15 | 5555.7176 | 306.0253 | U | 5555.7209 | 21.539 | 1146864 | 98.5 | −0.59 |
| 14 | 5249.6923 | 345.0474 | G | 5249.6958 | 20.605 | 1609784 | 100 | −0.67 |
| 13 | 4904.6449 | 345.0475 | G | 4904.6446 | 19.764 | 1791176 | 100 | 0.06 |
| 12 | 4559.5974 | 329.0525 | A | 4559.5918 | 19.341 | 974223 | 80 | 1.23 |
| 11 | 4230.5449 | 345.0474 | G | 4230.5449 | 16.828 | 1254040 | 99.7 | 0.00 |
| 10 | 3885.4975 | 359.0631 | m7G | 3885.4957 | 15.319 | 1940572 | 95.7 | 0.46 |
| 9 | 3526.4344 | 306.0253 | U | 3526.4327 | 13.475 | 1011995 | 100 | 0.48 |
| 8 | 3220.4091 | 305.0413 | C | 3220.4066 | 11.393 | 2082145 | 100 | 0.78 |
| 7 | 2915.3678 | 319.0569 | m5C | 2915.3648 | 10.586 | 3108932 | 100 | 1.03 |
| 6 | 2596.3109 | 306.0253 | U | 2596.3066 | 6.488 | 523377 | 42.8 | 1.66 |
| 5 | 2290.2856 | 345.0475 | G | 2290.2828 | 3.961 | 2464626 | 94.7 | 1.22 |
| 4 | 1945.2381 | 306.0253 | U | 1945.2379 | 1.074 | 637786 | 83.4 | 0.10 |
| 3 | 1639.2128 | 345.0474 | G | 1639.2106 | 1.034 | 2301078 | 100 | 1.34 |
| 2 | 1294.1654 | 320.0409 | T | 1294.1737 | 8.127 | 78112 | 67.5 | −6.41 |
| 1 | 974.1245 | 306.0253 | ψ | 974.1240 | 0.936 | 143886 | 79.1 | 0.51 |

TABLE S5-21
5′_OH_tRNA_T1_SII_111418s05_44g45a.
LC-MS analysis of segment II from 34Gm to 55ψ(mass ladder components from 3′ to 5′).

| Theoretical | | | | Extracted data file after LC/MS analysis | | | | |
|---|---|---|---|---|---|---|---|---|
| | Theoretical | Base | | MFE | | | Quality | Error |
| Fragments | mass | mass | Base | mass | t_{R} | Volume | Score | ppm |
| 21 | 7739.0291 | 688.1156 | A + Gm | 7739.0198 | 28.919 | 572629 | 80 | 1.20 |
| 20 | 7050.9135 | 329.0525 | A | 7050.9277 | 26.539 | 413840 | 60 | −2.01 |
| 19 | 6721.8610 | 212.0086 | Y′ | 6721.8635 | 24.741 | 381223 | 72.8 | −0.37 |
| 18 | 6509.8524 | 329.0525 | A | 6509.8604 | 25.336 | 1019699 | 80 | −1.23 |
| 17 | 6180.7999 | 306.0253 | ψ | 6180.8037 | 23.079 | 707995 | 77.8 | −0.61 |
| 16 | 5874.7746 | 319.0570 | m5C | 5874.7783 | 23.641 | 2167527 | 100 | −0.63 |
| 15 | 5555.7176 | 306.0253 | U | 5555.7209 | 21.539 | 1146864 | 98.5 | −0.59 |
| 14 | 5249.6923 | 345.0474 | G | 5249.6958 | 20.605 | 1609784 | 100 | −0.67 |
| 13 | 4904.6449 | 345.0475 | G | 4904.6446 | 19.764 | 1791176 | 100 | 0.06 |
| 12 | 4559.5974 | 345.0474 | G | 4559.5918 | 19.341 | 974223 | 80 | 100 |
| 11 | 4214.5500 | 329.0525 | A | 4214.5624 | 18.424 | 273170 | 79.6 | 100 |
| 10 | 3885.4975 | 359.0631 | m7G | 3885.4957 | 15.319 | 1940572 | 95.7 | 0.46 |
| 9 | 3526.4344 | 306.0253 | U | 3526.4327 | 13.475 | 1011995 | 100 | 0.48 |
| 8 | 3220.4091 | 305.0413 | C | 3220.4066 | 11.393 | 2082145 | 100 | 0.78 |
| 7 | 2915.3678 | 319.0569 | m5C | 2915.3648 | 10.586 | 3108932 | 100 | 1.03 |
| 6 | 2596.3109 | 306.0253 | U | 2596.3066 | 6.488 | 523377 | 42.8 | 1.66 |
| 5 | 2290.2856 | 345.0475 | G | 2290.2828 | 3.961 | 2464626 | 94.7 | 1.22 |
| 4 | 1945.2381 | 306.0253 | U | 1945.2379 | 1.074 | 637786 | 83.4 | 0.10 |
| 3 | 1639.2128 | 345.0474 | G | 1639.2106 | 1.034 | 2301078 | 100 | 1.34 |
| 2 | 1294.1654 | 320.0409 | T | 1294.1737 | 8.127 | 78112 | 67.5 | −6.41 |
| 1 | 974.1245 | 306.0253 | ψ | 974.1240 | 0.936 | 143886 | 79.1 | 0.51 |

TABLE S5-22
5′_biotin_tRNA_T1_SII_032919s07_44A45G.
LC-MS analysis of segment II from 30G to 55ψ(mass ladder components from 3′ to 5′).

| Theoretical | | | | Extracted data file after LC/MS analysis | | | | |
|---|---|---|---|---|---|---|---|---|
| | Theoretical | Base | | MFE | | | Quality | Error |
| Fragments | mass | mass | Base | mass | t_{R} | Volume | Score | ppm |
| 24 | 9038.2113 | 345.0474 | G | 9038.133 | 37.926 | 394860 | 60.8 | 8.66 |
| 23 | 8693.1639 | 329.0525 | A | 8693.1871 | 38.113 | 174673 | 41.4 | −2.67 |
| 22 | 8364.1114 | 625.0823 | U + Cm | 8364.1502 | 37.005 | 133633 | 41.9 | −4.64 |
| 21 | 7739.0291 | 688.1156 | A + Gm | 7739.0557 | 35.391 | 650792 | 77.4 | −3.44 |
| 20 | 7050.9135 | 329.0525 | A | 7050.9339 | 32.627 | 590137 | 78.5 | −2.89 |
| 19 | 6721.8610 | 212.0086 | Y′ | 6721.8845 | 30.813 | 764391 | 80 | −3.50 |
| 18 | 6509.8524 | 329.0525 | A | 6509.864 | 31.762 | 1166876 | 80 | −1.78 |
| 17 | 6180.7999 | 306.0253 | ψ | 6180.7968 | 29.159 | 148437 | 65.9 | 0.50 |
| 16 | 5874.7746 | 319.0570 | m5C | 5874.7784 | 30.31 | 1368105 | 79.9 | −0.65 |
| 15 | 5555.7176 | 306.0253 | U | 5555.7219 | 27.737 | 1148576 | 80 | −0.77 |
| 14 | 5249.6923 | 345.0474 | G | 5249.7098 | 26.957 | 1297236 | 80 | −3.33 |
| 13 | 4904.6449 | 345.0475 | G | 4904.6497 | 26.195 | 1021939 | 90 | −0.98 |
| 12 | 4559.5974 | 329.0525 | A | 4559.5974 | 25.942 | 1209559 | 99 | 0.00 |
| 11 | 4230.5449 | 345.0474 | G | 4230.5461 | 23.338 | 927818 | 92.3 | −0.28 |
| 10 | 3885.4975 | 359.0631 | m7G | 3885.4975 | 21.811 | 1357508 | 90.5 | 0.00 |
| 9 | 3526.4344 | 306.0253 | U | 3526.4332 | 20.034 | 1078413 | 98.3 | 0.34 |
| 8 | 3220.4091 | 305.0413 | C | 3220.4063 | 18.209 | 1434999 | 100 | 0.87 |
| 7 | 2915.3678 | 319.0569 | m5C | 2915.366 | 17.589 | 2388681 | 100 | 0.62 |
| 6 | 2596.3109 | 306.0253 | U | 2596.308 | 12.655 | 1592241 | 100 | 1.12 |
| 5 | 2290.2856 | 345.0475 | G | 2290.2828 | 10.189 | 2053112 | 100 | 1.22 |
| 4 | 1945.2381 | 306.0253 | U | 1945.2371 | 6.47 | 1359480 | 77.8 | 0.51 |
| 3 | 1639.2128 | 345.0474 | G | 1639.21 | 4.723 | 1598482 | 100 | 1.71 |
| 2 | 1294.1654 | 320.0409 | T | 1294.1615 | 2.282 | 620026 | 100 | 3.01 |
| 1 | 974.1245 | 306.0253 | ψ | 974.1225 | 0.875 | 221837 | 90.6 | 2.05 |

TABLE S5-23
5′_biotin_tRNA_T1_SII_032919s07_44g45a.
LC-MS analysis of segment II from 30G to 55ψ(mass ladder components from 3′ to 5′).

| Theoretical | | | | Extracted data file after LC/MS analysis | | | | |
|---|---|---|---|---|---|---|---|---|
| | Theoretical | Base | | MFE | | | Quality | Error |
| Fragments | mass | mass | Base | mass | t_{R} | Volume | Score | ppm |
| 24 | 9038.2113 | 345.0474 | G | 9038.133 | 37.926 | 394860 | 60.8 | 8.66 |
| 23 | 8693.1639 | 329.0525 | A | 8693.1871 | 38.113 | 174673 | 41.4 | −2.67 |
| 22 | 8364.1114 | 625.0823 | U + Cm | 8364.1502 | 37.005 | 133633 | 41.9 | −4.64 |
| 21 | 7739.0291 | 688.1156 | A + Gm | 7739.0557 | 35.391 | 650792 | 77.4 | −3.44 |
| 20 | 7050.9135 | 329.0525 | A | 7050.9339 | 32.627 | 590137 | 78.5 | −2.89 |
| 19 | 6721.8610 | 212.0086 | Y′ | 6721.8845 | 30.813 | 764391 | 80 | −3.50 |
| 18 | 6509.8524 | 329.0525 | A | 6509.864 | 31.762 | 1166876 | 80 | −1.78 |
| 17 | 6180.7999 | 306.0253 | ψ | 6180.7968 | 29.159 | 148437 | 65.9 | 0.50 |
| 16 | 5874.7746 | 319.0570 | m5C | 5874.7784 | 30.31 | 1368105 | 79.9 | −0.65 |
| 15 | 5555.7176 | 306.0253 | U | 5555.7219 | 27.737 | 1148576 | 80 | −0.77 |
| 14 | 5249.6923 | 345.0474 | G | 5249.7098 | 26.957 | 1297236 | 80 | −3.33 |
| 13 | 4904.6449 | 345.0475 | G | 4904.6497 | 26.195 | 1021939 | 90 | −0.98 |
| 12 | 4559.5974 | 345.0474 | G | 4559.5974 | 25.942 | 1209559 | 99 | 0.00 |
| 11 | 4214.5500 | 329.0525 | A | 4214.5534 | 24.918 | 299777 | 60 | −0.81 |
| 10 | 3885.4975 | 359.0631 | m7G | 3885.4975 | 21.811 | 1357508 | 90.5 | 0.00 |
| 9 | 3526.4344 | 306.0253 | U | 3526.4332 | 20.034 | 1078413 | 98.3 | 0.34 |
| 8 | 3220.4091 | 305.0413 | C | 3220.4063 | 18.209 | 1434999 | 100 | 0.87 |
| 7 | 2915.3678 | 319.0569 | m5C | 2915.366 | 17.589 | 2388681 | 100 | 0.62 |
| 6 | 2596.3109 | 306.0253 | U | 2596.308 | 12.655 | 1592241 | 100 | 1.12 |
| 5 | 2290.2856 | 345.0475 | G | 2290.2828 | 10.189 | 2053112 | 100 | 1.22 |
| 4 | 1945.2381 | 306.0253 | U | 1945.2371 | 6.47 | 1359480 | 77.8 | 0.51 |
| 3 | 1639.2128 | 345.0474 | G | 1639.21 | 4.723 | 1598482 | 100 | 1.71 |
| 2 | 1294.1654 | 320.0409 | T | 1294.1615 | 2.282 | 620026 | 100 | 3.01 |
| 1 | 974.1245 | 306.0253 | ψ | 974.1225 | 0.875 | 221837 | 90.6 | 2.05 |

TABLE S5-24
Detection of form I (44A45G) and form II (44g45a), respectively, in three datasets by global
hierarchical ranking algorithm (refer to output files Table S12, 13, 14, 15, 18 and 19).

| | Form I | | | | Form II | | | | | | | |
|---|---|---|---|---|---|---|---|---|---|---|---|---|
| | | EIC | | EIC | | EIC | | EIC | I | Mean ± | II | Mean ± |
| Dataset | m/z | (44A) | m/z | (45G) | m/z | (44G) | m/z | (45A) | % | SEM | % | SEM |
| Labeled | 836.1243 | 2308326 | 870.4306 | 1994979 | 837.6269 | 1932380 | 870.4306 | 1994979 | 54 | 50.4 ± 3.2% | 46 | 49.6 ± 3.2% |
| segment II | | | | | | | | | | | | |
| Unlabeled | 778.4074 | 2077840 | 812.9122 | 1608093 | 780.0080 | 2630985 | 812.9122 | 1608093 | 44 | | 56 | |
| segment II | | | | | | | | | | | | |
| Non-CMC- | 778.4077 | 1385023 | 813.0133 | 1770337 | 779.7066 | 1245805 | 813.0133 | 1770337 | 53 | | 47 | |
| converted | | | | | | | | | | | | |
| segment II | | | | | | | | | | | | |

*Form I % = EIC(44A)/EIC(44A) + EIC(44G);
Form II % = EIC(44G)/EIC(44A) + EIC(44G)

What is claimed:

1. A method for generating the sequence of one or more RNA molecules and detecting the presence, identity, location, and quantity of RNA nucleotide modifications on said one or more RNA molecules, said method RNA comprising the steps of (i) controlled fragmentation of the RNA to form sequencable ladder fragments such as 5′ and 3′ MS ladder fragments; (ii) mass measurement of resultant degraded RNA samples containing RNAs and their fragmented fragments; and (iii) data processing, including identification and separation of 3′ and/or 5′ MS ladder fragments thereby generating the sequence of one or more RNA molecules and detecting the presence, identity, location, and quantity of RNA nucleotide modifications.

2. The method of claim 1 wherein the controlled fragmentation of the RNA is achieved by chemical degradation, enzymatic degradation, or physical degradation.

3. The method of claim 1, wherein the mass measurement is achieved by LC-MS, gas chromatography, capillary electrophoresis, ion mobility spectrometry, or other methods coupled with mass spectrometry.

4. The method of claim 1, wherein the data processing includes homology searching before, or after, fragmentation of RNA for identification of related RNA isoforms.

5. The method of claim 1, wherein a MassSum data processing step identifies and isolates the 3′, 5′ ladder fragments as well as other related fragments into subsets for each RNA in a mixed sample.

6. The method of claim 5, further comprising the step of Gap Filling data processing to rescue 3′ and 5′ ladder fragments missed by Mass/Sum separation.

7. The method of claim 1, wherein the data processing includes the step of ladder complementation where the ladder fragments from one or more related RNA isoforms are used to perfect an imperfect ladder.

8. The method of claim 1, wherein the data processing includes the step of identifying acid labile nucleotide modifications by comparing the mass change of intact RNA before and after acid degradation.

9. A method for generating the sequence of one or more RNA molecules and detecting the presence, identity, location, and quantity of RNA nucleotide modifications on said one or more RNA molecules, said method RNA comprising the steps of (i) identifying a specific chemical moiety associated with the RNA or labeling the RNA with a tag thereby imparting an identifiable property on the RNA (ii) controlled fragmentation of the RNA to form 5′ and 3′ MS ladder fragments; (iii) mass measurement of resultant degraded RNA samples containing RNAs and their degraded fragments; and (iv) data processing, including identification of 3′ and/or 5′ MS ladder fragments thereby generating the sequence of one or more RNA molecules and detecting the presence, identity, location, and quantity of RNA nucleotide modifications.

10. The method of claim 9, wherein the specific chemical moiety or the labeling tag has a known mass.

11. The method of claim 10, wherein the chemical moiety is a 5′ phosphate and 3′ CCA of tRNA.

12. The method of claim 10, wherein the identifiable property results in an alteration in mass measurement.

13. The method of claim 9, wherein the chemical moiety results in a change in retention time and/or mass/MS.

14. The method of claim 9, wherein the label is selected from the group consisting of a hydrophobic tag, biotin, a Cy3 tag, a Cy5 tag and a cholesterol.

15. The method of claim 9, wherein the controlled fragmentation of the RNA is achieved by chemical degradation, enzymatic degradation, or physical degradation.

16. The method of claim 9, wherein the mass measurement is achieved by LC-MS, gas chromatography, capillary electrophoresis, ion mobility spectrometry or others coupled with mass spectrometry.

17. The method of claim 9, wherein the data processing step identifies the RNA fragments based on the specific chemical moiety associated with the RNA or the labeled tag thereby imparting an identifiable property on the RNA and/or fragments.

18. The method of claim 9, wherein the data processing step includes implementation of the anchoring-based algorithm to identify the labeled RNA and/or fragments.

19. The method of claim 1, further comprising the implementation of non-MS-based sequencing methods such as next generation sequencing (NGS) methods.

20. A kit for use in generating the sequence of one or more RNA molecules and detecting the presence, identity, location, and quantity of RNA nucleotide modifications on said one or more RNA molecules, said kit comprising one or more components for performance of the method of claim 1.

21. A kit for use in generating the sequence of one or more RNA molecules and detecting the presence, identity, location, and quantity of RNA nucleotide modifications on said one or more RNA molecules, said kit comprising one or more components for performance of the method of claim 9.

22. A MS based sequencing instrument for use in generating the sequence of one or more RNA molecules and detecting the presence, identity, location, and quantity of RNA nucleotide modifications on said one or more RNA molecules, said instrument comprising one or more components for performance of the method of claim 1.

23. A MS based sequencing instrument for use in generating the sequence of one or more RNA molecules and detecting the presence, identity, location, and quantity of RNA nucleotide modifications on said one or more RNA molecules, said instrument comprising one or more components for performance of the method of claim 9.

24. A non-transitory computer-readable medium storing instructions that, when executed by a processor, cause the processor to perform method for generating the sequence of one or more RNA molecules and detecting the presence, identity, location, and quantity of RNA nucleotide modifications on said one or more RNA molecules, said method RNA comprising the steps of (i) controlled fragmentation of the RNA to form 5′ and 3′ MS ladder fragments; (ii) mass measurement of resultant degraded RNA samples containing RNAs and their fragmented fragments; and (iii) data processing, including identification and separation of 3′ and/or 5′ MS ladder fragments thereby generating the sequence of one or more RNA molecules and detecting the presence, identity, location, and quantity of RNA nucleotide modifications.

25. A non-transitory computer-readable medium storing instructions that, when executed by a processor, cause the processor to perform a method for generating the sequence of one or more RNA molecules and detecting the presence, identity, location, and quantity of RNA nucleotide modifications on said one or more RNA molecules, the method comprising the steps of (i) identifying a specific chemical moiety associated with the RNA or labeling the RNA with a tag thereby imparting an identifiable property on the RNA (ii) controlled fragmentation of the RNA to form 5′ and 3′ MS ladder fragments; (iii) mass measurement of resultant degraded RNA samples containing RNAs and their degraded fragments; and (iv) data processing, including identification of 3′ and/or 5′ MS ladder fragments thereby generating the sequence of one or more RNA molecules and detecting the presence, identity, location, and quantity of RNA nucleotide modifications.
